(19) United States
(12) Patent Application Publication
(10) Pub. No.: US 2007/0078135 A1
(43) Pub. Date: Apr. 5, 2007

(54) Substituted heteroaryl CB1 antagonists

(75) Inventors: Jun Yuan, Guilford, CT (US); Qin Guo, Waterford, CT (US); He Zhao, Madison, CT (US); Shaojing Hu, Hamden, CT (US); Darren L. Whitehouse, Westbrook, CT (US); Wallace C. Pringle, Guilford, CT (US); Jianmin Mao, Madison, CT (US); George D. Maynard, Clinton, CT (US); Jack D. Hammer, Hamden, CT (US); David J. Wustrow, Guilford, CT (US); Hongbin Li, Branford, CT (US)

Correspondence Address: EDWARDS & ANGELL, LLP, P.O. BOX 55874, BOSTON, MA, 02205, US

(73) Assignee: Neurogen Corporation, Branford, CT (US)

(21) Appl. No.: 11/406,532

(22) Filed: Apr. 18, 2006

Related U.S. Application Data
(60) Provisional application No. 60/672,452, filed on Apr. 18, 2005.

(51) Int. Cl. A61K 31/53; A61K 31/497; A61K 31/4965; C07D 403/02

(52) U.S. Cl. 514/241; 514/255.05; 544/183; 544/405; 514/217.05

(57) ABSTRACT

Compounds of Formula I are provided. 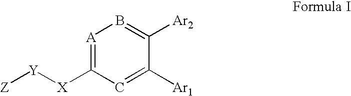 In which the variables are as described herein. Such compounds may be used to modulate CB1 activity in vivo or in vitro, and are particularly useful in the treatment of conditions responsive to CB1 modulation in humans, domesticated companion animals and livestock animals, including appetite disorders, obesity and addictive disorders. Pharmaceutical compositions and methods for using them to treat such disorders are provided, as are methods for using such ligands for receptor localization studies and various in vitro assays.

### FIELD OF THE INVENTION

This invention relates generally to substituted heteroaryl compounds, and to the use of such compounds to treat conditions responsive to cannabinoid receptor-1 (CB1) activation. The invention further relates to the use of such compounds as reagents for the identification of other agents that bind to CB1, and as probes for the detection and localization of CB1.

### BACKGROUND OF THE INVENTION

Obesity is the most common nutritional problem in developed countries. This condition is often both harmful and costly, as it increases the likelihood of developing serious health conditions (such as cardiovascular diseases and diabetes) and complicates numerous chronic conditions such as respiratory diseases, osteoarthritis, osteoporosis, gall bladder disease and dyslipidernias. Fortunately, however, many of the conditions caused or exacerbated by obesity can be resolved or dramatically improved by weight loss.

Once considered merely a behavioral problem (i.e., the result of voluntary hyperphagia), obesity is now recognized as a complex multifactorial disease involving defective regulation of food intake, food-induced energy expenditure and the balance between lipid and lean body anabolism. Both environmental and genetic factors play a role in the development of obesity. As a result, treatment programs that focus entirely on behavior modification have limited efficacy and are associated with recidivism rates exceeding 95%. Pharmacotherapy is now seen as a critical component of weight loss and subsequent weight management.

Currently available prescription drugs for managing obesity generally reduce weight by inducing satiety or decreasing dietary fat absorption. Such drugs, however, often have unacceptable side effects. Several, such as the older weight-loss drugs (e.g., amphetamine, methamphetamine, and phenmetrazine, are no longer recommended because of the risk of their abuse. Fenfluramine and dexfenfluramine, both serotonergic agents used to regulate appetite, are also no longer available for use.

Thus, there exists a need for more effective agents for promoting weight loss and for reducing or preventing weight-gain. In addition, there exists an unmet need for more effective agents for the treatment of alcohol and tobacco dependence. The present invention fulfills this need, and provides further related advantages.

### SUMMARY OF THE INVENTION

The present invention provides substituted heteroaryl CB1 antagonists that satisfy Formula I: 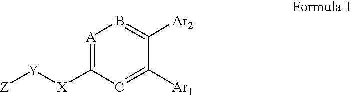
or are a pharmaceutically acceptable salt, solvate or ester of such a compound.
Within Formula I:
A, B and C are independently optionally substituted carbon (e.g., CR₁) or nitrogen, such that at least one of A, B and C is nitrogen;
Ar₁ and Ar₂ are independently chosen from 5- to 10-membered carbocycles and heterocycles, each of which is optionally substituted, and each of which is preferably substituted with from 0 to 6 substituents independently chosen from R_{A}; 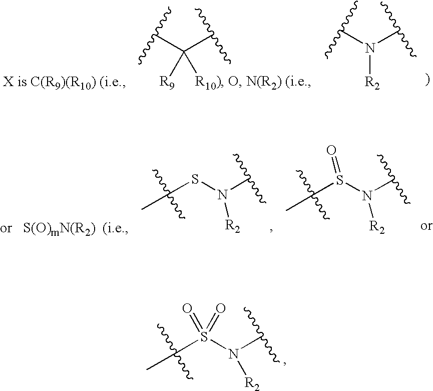
   in which the N is directly bonded to the aromatic ring that comprises A, B and C, and the S is bonded to “Y”);
Y is a single covalent bond or C₁-C₄alkylene that is optionally substituted, and is preferably substituted with from 0 to 2 substituents independently chosen from R_{B};
Z is hydrogen, 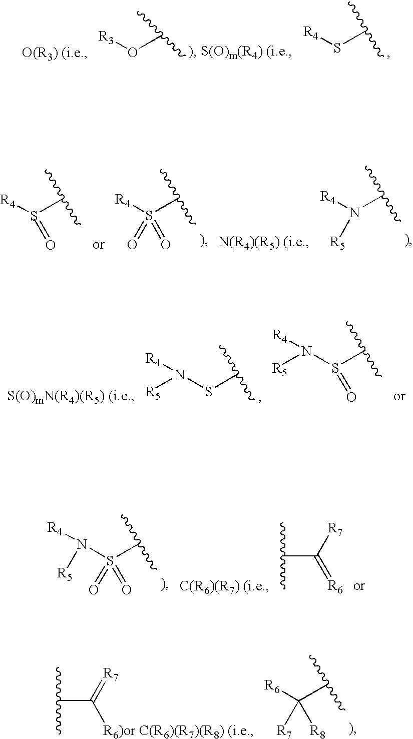
   wherein m is 0, 1 or 2;
Each R_{A} is independently chosen from:
   (i) halogen, hydroxy, cyano, amino, nitro, aminocarbonyl, aminosulfonyl and —COOH; and
   (ii) C₁-C₆alkyl, C₂-C₆alkenyl, C₂-C₆alkynyl, (C₃-C₈cycloalkyl)C₀-C₄alkyl, C₁-C₆alkoxy, C₁-C₆alkylthio, C₁-C₆alkylsulfinyl, C₁-C₆alkoxycarbonyl, C₁-C₆alkylsulfonylC₀-C₄alkyl, mono- or di-(C₁-C₆alkyl)aminoC₀-C₄alkyl, mono- or di-(C₁-C₆alkyl)aminosulfonylC₀-C₄alkyl, mono- or di-(C₁-C₆alkyl)aminocarbonylC₀-C₄alkyl, phenylC₀-C₄alkyl, (4- to 8-membered heterocycle)C₀-C₄alkyl and (4- to 8-membered heterocycle)C₁-C₄alkoxy; each of which is optionally substituted, and each of which is preferably substituted with from 0 to 6 substituents independently chosen from R_{E};
or two R_{A} groups attached to adjacent ring carbon atoms are taken together to form a fused 5- to 7-membered carbocycle or heterocycle that is optionally substituted and is preferably substituted with from 0 to 6 substituents independently chosen from R_{E};
Each R_{B} is:
   (i) halogen, hydroxy, —COOH, aminocarbonyl, oxo, C₁-C₄alkyl, C₁-C₄alkoxy, C₁-C₄haloalkyl, C₁-C₄aminoalkyl, mono- or di-(C₁-C₆alkyl)aminoC₀-C₄alkyl or mono- or di-(C₁-C₆alkyl)aminocarbonyl; or
   (ii) taken together with R₃, R₄ or R₆ to form a 4- to 10-membered carbocycle or heterocycle that is optionally substituted, and is preferably substituted with from 0 to 4 substituents independently chosen from R_{D};
or two R_{B} are taken together to form a C₃-C₈cycloalkyl or a 4- to 8-membered heterocycloalkyl;
Each R_{D} is independently chosen from:
   (i) hydroxy, halogen, cyano, amino, oxo, nitro, —COOH, aminocarbonyl and aminosulfonyl; and
   (ii) C₁-C₆alkyl, C₁-C₆alkenyl, C₁-C₆alkynyl, (C₃-C₈cycloalkyl)C₀-C₄alkyl, C₁-C₆alkoxy, C₁-C₆alkylthio, C₂-C₆alkyl ether, mono- or di-(C₁-C₆alkyl)amino, mono- or di-(C₁-C₆alkyl)aminocarbonyl, mono- or di-(C₁-C₆alkyl)aminosulfonyl, C₁-C₆alkylsulfinyl, C₁-C₆alkylsulfonyl, (4- to 8-membered heterocycle)C₀-C₄alkyl and phenylC₀-C₄alkyl, each of which is optionally substituted, and each of which is preferably substituted with from 0 to 4 substituents independently chosen from R_{E};
Each R_{E} is independently chosen from oxo, halogen, hydroxy, cyano, amino, nitro, aminocarbonyl, aminosulfonyl, —COOH, C₁-C₆alkyl, C₁-C₆haloalkyl, C₁-C₆alkoxy, C₁-C₆haloalkoxy, C₁-C₆alkylthio, C₁-C₆alkoxycarbonyl, C₁-C₆alkanoyloxy, C₃-C₆alkanone, mono- or di-(C₁-C₆alkyl)amino, C₁-C₆alkylsulfonyl, mono- or di-(C₁-C₆alkyl)aminosulfonyl and mono- or di-(C₁-C₆alkyl)aminocarbonyl;
Each R₁ is independently:
   (i) hydrogen, halogen, hydroxy, cyano, amino, nitro, —COOH, aminocarbonyl or aminosulfonyl;
   (ii) C₁-C₆alkyl, C₂-C₆alkenyl, C₂-C₆alkynyl, (C₃-C₈cycloalkyl)C₀-C₄alkyl, C₁-C₆alkoxy, C₁-C₆alkoxycarbonyl, C₁-C₆alkylsulfonyl C₀-C₄alkyl, C₁-C₆alkylaminosulfonylC₀-C₄alkyl, mono- or di-(C₁-C₆alkyl)aminoC₀-C₄alkyl, or mono- or di-(C₁-C₆alkyl)aminocarbonylC₀-C₄alkyl or a (4- to 8-membered heterocycle)C₀-C₄alkyl; each of which is optionally substituted, and each of which is preferably substituted with from 0 to 6 substituents independently chosen from R_{E}; or
   (iii) if located at the A or C position, taken together with R₂ or R₉ to form a fused 5- to 8-membered carbocycle or heterocycle that is optionally substituted, and is preferably substituted with from 0 to 4 substituents independently chosen from R_{E};
R₂ is:
   (i) hydrogen or aminocarbonyl;
   (ii) C₁-C₆alkyl, C₂-C₆alkenyl, C₂-C₆alkynyl, (C₃-C₈cycloalkyl)C₀-C₄alkyl, C₂-C₆alkyl ether, C₁-C₆alkoxycarbonyl, mono- or di-(C₁-C₄alkyl)aminocarbonyl, mono- or di-(C₁-C₄alkyl)aminosulfonyl, mono- or di-(C₁-C₄alkyl)aminoC₀-C₄alkyl, C₁-C₆alkylsulfonyl or (4- to 8-membered heterocycle)C₀-C₄alkyl; each of which is optionally substituted, and each of which is preferably substituted with from 0 to 6 substituents independently chosen from R_{E};
   (iii) taken together with R₃, R₄ or R₆ to form a 4- to 10-membered heterocycle that is optionally substituted, and is preferably substituted with from 0 to 3 substituents independently chosen from R_{D}; or
   (iv) taken together with R₁ at the A or C position to form an optionally substituted, fused, 5- to 8-membered heterocycle;
R₃ is:
   (i) hydrogen;
   (ii) C₁-C₈alkyl, C₂-C₈alkenyl, C₂-C₈alkynyl, C₁-C₈alkanone, C₂-C₈alkyl ether, mono- or di-(C₁-C₆alkyl)aminoC₁-C₄alkyl, (C₃-C₈cycloalkyl)C₀-C₄alkyl, mono- or di-(C₁-C₆alkyl) amino carbonyl, (5- to 7-membered heterocycle)-C(═O)—, phenylC₀-C₄alkyl, or (4- to 8-membered heterocycloalkyl)C₀-C₄alkyl; each of which is optionally substituted, and each of which is preferably substituted with from 0 to 6 substituents independently chosen from R_{E}; or
   (iii) taken together with R₂, R₉ or R_{B} to form a 4- to 10-membered heterocycle that is optionally substituted, and is preferably substituted with from 0 to 4 substituents independently chosen from R_{D};
R₄ is:
   (i) hydrogen or aminocarbonyl;
   (ii) C₁-C₈alkyl, C₂-C₈alkenyl, C₂-C₈alkynyl, (C₃-C₈cycloalkyl)C₀-C₄alkyl, C₁-C₆alkylsulfonyl, (C₃-C₈cycloalkyl)sulfonyl, C₁-C₆alkoxycarbonyl, C₂-C₈alkyl ether, mono- or di-(C₁-C₆alkyl)aminosulfonyl, mono- or di-(C₁-C₆alkyl)aminocarbonyl or (5- or 6-membered heterocycle)C₀-C₄alkyl; each of which is optionally substituted, and each of which is preferably substituted with from 0 to 6 substituents independently chosen from R_{E}; or
   (iii) taken together with one or two of R₂, R₅, R₉ or R_{B} to form a 4- to 10-membered heterocycle that is optionally substituted, and is preferably substituted with from 0 to 4 substituents independently chosen from R_{D};
R₅ is:
   (i) hydrogen, cyano or aminocarbonyl;
   (ii) C₁-C₈alkyl, C₂-C₈alkenyl, C₂-C₈alkynyl, (C₃-C₈cycloalkyl)C₀-C₄alkyl, C₁-C₆alkylsulfonyl, (C₃-C₈cycloalkyl)sulfonyl, C₁-C₆alkoxycarbonyl, C₂-C₈alkyl ether, mono- or di-(C₁-C₆alkyl)aminosulfonyl, mono- or di-(C₁-C₆alkyl)aminocarbonyl, mono- or di-(C₁-C₆alkyl)aminoC₀-C₄alkyl, phenylC₀-C₄alkyl or (5- or 6-membered heterocycle)C₀-C₄alkyl; each of which is optionally substituted, and each of which is preferably substituted with from 0 to 6 substituents independently chosen from phenyl and R_{E}; or
   (iii) taken together with R₄ to form an optionally substituted 4- to 10-membered heterocycloalkyl;
R₆ is:
   (i) hydrogen, hydroxy, halogen, cyano, amino, aminocarbonyl, aminosulfonyl or —COOH;
   (ii) C₁-C₈alkyl, C₂-C₈alkenyl, C₂-C₈alkynyl, (C₃-C₈cycloalkyl)C₀-C₄alkyl, C₁-C₆alkoxy, C₁-C₆alkylthio, C₁-C₆alkylsulfinyl, C₁-C₆alkylsulfonyl, (C₃-C₈cycloalkyl)sulfonyl, C₁-C₆alkoxycarbonyl, mono- or di-(C₁-C₆alkyl)aminosulfonyl, mono- or di-(C₁-C₆alkyl)aminocarbonyl, mono- or di-(C₁-C₆alkyl)aminoC₀-C₄alkyl or (5- or 6-membered heterocycle)C₀-C₄alkyl; each of which is optionally substituted, and each of which is preferably substituted with from 0 to 6 substituents independently chosen from R_{E}; or
   (iii) taken together with one or two of R₂, R₇, R₈, R₉ or R_{B} to form a 4- to 10-membered carbocycle or heterocycle that is optionally substituted, and is preferably substituted with from 0 to 3 substituents independently chosen from R_{D};
R₇ and R₈ are independently:
   (i) hydrogen, hydroxy, halogen, cyano, amino, aminocarbonyl, aminosulfonyl or —COOH;
   (ii) C₁-C₈alkyl, C₂-C₈alkenyl, C₂-C₈alkynyl, (C₃-C₈cycloalkyl)C₀-C₄alkyl, C₁-C₆alkoxy, C₁-C₆alkanoyl, C₁-C₈alkylthio, C₁-C₈alkylsulfinyl, C₁-C₆alkylsulfonyl, (C₃-C₈cycloalkyl)sulfonyl, C₁-C₆alkoxycarbonyl, mono- or di-(C₁-C₆alkyl)amino, mono- or di-(C₁-C₆alkyl)aminosulfonyl, mono- or di-(C₁-C₆alkyl)aminocarbonyl, phenylC₀-C₄alkyl or (5- or 6-membered heterocycle)C₀-C₄alkyl; each of which is optionally substituted, and each of which is preferably substituted with from 0 to 6 substituents independently chosen from phenyl and R_{E}; or
   (iii) taken together with R₆ to form an optionally substituted carbocycle or heterocycle;
or R₇ and R₈ are taken together to form a 4- to 8-membered carbocycle or heterocycle that is optionally substituted, and is preferably substituted with from 0 to 3 substituents independently chosen from R_{D};
R₉ is:
   (i) hydrogen, hydroxy, halogen, cyano or amino;
   (ii) C₁-C₈alkyl, C₂-C₈alkenyl, C₂-C₈alkynyl, C₁-C₈alkoxy, C₁-C₈alkylthio, C₁-C₈alkylsulfinyl, C₁-C₈alkylamino, (C₃-C₈cycloalkyl)C₀-C₄alkyl or C₁-C₆alkylsulfonyl; each of which is optionally substituted, and each of which is preferably substituted with from 0 to 6 substituents independently chosen from R_{E};
   (iii) taken together with R₃, R₄ or R₆ to form an optionally substituted 4- to 10-membered carbocycle or heterocycle; or
   (iv) taken together with R₁ at the A position to form an optionally substituted, fused 5- to 8-membered carbocycle or heterocycle; and
R₁₀ is:
   (i) hydrogen, hydroxy, halogen, cyano or amino; or
   (ii) C₁-C₈alkyl, C₂-C₈alkenyl, C₂-C₈alkynyl, C₁-C₈alkoxy, C₁-C₈alkylthio, C₁-C₈alkylsulfinyl, C₁-C₈alkylamino, (C₃-C₈cycloalkyl)C₀-C₄alkyl or C₁-C₆alkylsulfonyl; each of which is optionally substituted, and each of which is preferably substituted with from 0 to 6 substituents independently chosen from R_{E}.

In certain aspects, substituted heteroaryl CB1 antagonists of Formula I that further satisfy Formula II are provided: 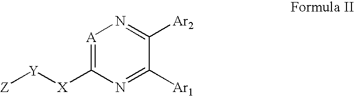
Within Formula II:
A is CR₁ or N;
Ar₁ and Ar₂ are independently chosen from 5- to 10-membered carbocycles and heterocycles, each of which is optionally substituted, and each of which is preferably substituted with from 0 to 6 substituents independently chosen from R_{A};
   such that at least one of Ar₁ and Ar₂ is an optionally substituted 5- or 6-membered heterocycle;
Y is C₁-C₄alkylene that is unsubstituted or substituted with one or two substituents independently chosen from R_{B};
Z is O(R₃), S(O)ₘ(R₄), N(R₄)(R₅), S(O)ₘN(R₄)(R₅), C(R₆)(R₇) or C(R₆)(R₇)(R₈); wherein m is 0, 1 or 2;
Each R_{B} is:
   (i) halogen, hydroxy, —COOH, aminocarbonyl, C₁-C₄alkyl, C₁-C₄alkoxy, C₁-C₄haloalkyl, C₁-C₄aminoalkyl, mono- or di-(C₁-C₆alkyl)aminoC₀-C₄alkyl, or mono- or di-(C₁-C₆alkyl)aminocarbonyl; or
   (ii) taken together with R₃, R₄ or R₆ to form a 4- to 10-membered heterocycloalkyl that is optionally substituted, and is preferably substituted with from 0 to 3 substituents independently chosen from R_{D}; such that if R_{B} forms a heterocycloalkyl with R₃, then the heterocycloalkyl is not substituted with C₁-C₄alkoxycarbonyl;
or two R_{B} are taken together to form a C₃-C₈cycloalkyl or a 4- to 8-membered heterocycloalkyl;
R₁ is:
   (i) hydrogen, halogen, cyano, nitro, —COOH or aminosulfonyl; or
   (ii) C₁-C₆alkyl, C₂-C₆alkenyl, C₂-C₆alkynyl, (C₃-C₈cycloalkyl)C₀-C₄alkyl, C₁-C₆alkoxy, C₁-C₆alkoxycarbonyl, C₁-C₆alkylsulfonylC₀-C₄alkyl, C₁-C₆alkylaminosulfonylC₀-C₄alkyl, mono- or di-(C₁-C₆alkyl)aminoC₀-C₄alkyl, or mono- or di-(C₁-C₆alkyl)aminocarbonylC₀-C₄alkyl or a (4- to 8-membered heterocycle)C₀-C₄alkyl; each of which is optionally substituted, and each of which is preferably substituted with from 0 to 6 substituents independently chosen from R_{E}; or
   (iii) taken together with R₂ or R₉ to form a fused 5- to 8-membered heterocycle that is optionally substituted, and is preferably substituted with from 0 to 4 substituents independently chosen from R_{E};
R₂ is:
   (i) hydrogen or aminocarbonyl;
   (ii) C₁-C₆alkyl, C₂-C₆alkenyl, C₂-C₆alkynyl, (C₃-C₈cycloalkyl)C₀-C₄alkyl, C₂-C₆alkyl ether, C₁-C₆alkoxycarbonyl, mono- or di-(C₁-C₄alkyl)aminocarbonyl, mono- or di-(C₁-C₄alkyl)aminosulfonyl, mono- or di-(C₁-C₄alkyl)aminoC₀-C₄alkyl, C₁-C₆alkylsulfonyl or (4- to 8-membered heterocycle)C₀-C₄alkyl; each of which is optionally substituted, and each of which is preferably substituted with from 0 to 6 substituents independently chosen from R_{E};
   (iii) taken together with R₃, R₄ or R₆ to form a 4- to 10-membered heterocycle that is optionally substituted, and is preferably substituted with from 0 to 3 substituents independently chosen from R_{D}; or
   (iv) taken together with R₁ to form an optionally substituted fused 5- to 8-membered heterocycle;
R₃ is:
   (i) hydrogen;
   (ii) C₁-C₈alkyl, C₂-C₈alkenyl, C₂-C₈alkynyl, C₁-C₆alkanone, C₂-C₈alkyl ether, mono- or di-(C₁-C₆alkyl)aminoC₁-C₄alkyl, (C₃-C₈cycloalkyl)C₀-C₄alkyl, mono- or di-(C₁-C₆alkyl) amino carbonyl, (5- to 7-membered heterocycle)-C(═O)—, phenylC₀-C₄alkyl or (4- to 8-membered heterocycle)C₀-C₄alkyl; each of which is optionally substituted, and each of which is preferably substituted with from 0 to 3 substituents independently chosen from hydroxy, halogen, amino, C₁-C₆alkyl or C₁-C₆alkoxy; or
   (iii) taken together with R₂, R₉ or R_{B} to form a 4- to 10-membered heterocycle that is optionally substituted, and is preferably substituted with from 0 to 3 substituents independently chosen from R_{D};
R₄ is:
   (i) hydrogen;
   (ii) C₁-C₈alkyl, C₂-C₈alkenyl, C₂-C₈alkynyl, (C₃-C₈cycloalkyl)C₀-C₄alkyl, C₁-C₆alkylsulfonyl, (C₃-C₈cycloalkyl)sulfonyl, C₁-C₆alkoxycarbonyl, C₂-C₈alkyl ether, mono- or di-(C₁-C₆alkyl)aminosulfonyl, mono- or di-(C₁-C₆alkyl)aminocarbonyl or (5- or 6-membered heterocycle)C₀-C₄alkyl; each of which is optionally substituted, and each of which is preferably substituted with from 0 to 6 substituents independently chosen from halogen, hydroxy, oxo, cyano, amino, aminosulfonyl, aminocarbonyl, C₁-C₆alkyl, C₁-C₆alkoxy, C₁-C₆alkylsulfonyl, mono- or di-(C₁-C₆alkyl)aminosulfonyl, mono- or di-(C₁-C₆alkyl)amino and mono- or di-(C₁-C₆alkyl)aminocarbonyl; or
   (iii) taken together with R₂, R₅, R₉ or R_{B} to form a 4- to 10-membered heterocycle that is optionally substituted, and is preferably substituted with from 0 to 4 substituents independently chosen from R_{D};
R₅ is:
   (i) hydrogen, cyano or aminocarbonyl;
   (ii) C₁-C₈alkyl, C₂-C₈alkenyl, C₂-C₈alkynyl, (C₃-C₈cycloalkyl)C₀-C₄alkyl, C₁-C₆alkylsulfonyl, (C₃-C₈cycloalkyl)sulfonyl, C₁-C₆alkoxycarbonyl, C₂-C₈alkyl ether, mono- or di-(C₁-C₆alkyl)aminosulfonyl, mono- or di-(C₁-C₆alkyl)aminocarbonyl, mono- or di-(C₁-C₆alkyl)aminoC₀-C₄alkyl, phenylC₀-C₄alkyl or (5- or 6-membered heterocycle)C₀-C₄alkyl; each of which is optionally substituted, and each of which is preferably substituted with from 0 to 6 substituents independently chosen from halogen, hydroxy, oxo, cyano, amino, —COOH, aminosulfonyl, aminocarbonyl, C₁-C₆alkyl, C₁-C₆haloalkyl, C₁-C₆alkoxy, C₁-C₆alkylsulfonyl, mono- or di-(C₁-C₆alkyl)aminosulfonyl, mono- or di-(C₁-C₆alkyl)amino, mono- or di-(C₁-C₆alkyl)aminocarbonyl, 4- to 7-membered heterocycle, and phenyl; or
   (iii) taken together with R₄ to form an optionally substituted 4- to 8-membered heterocycloalkyl;
R₇ is:
   (i) hydrogen, hydroxy, halogen, cyano, amino, aminocarbonyl, aminosulfonyl or —COOH;
   (ii) C₁-C₈alkyl, C₂-C₈alkenyl, C₂-C₈alkynyl, (C₃-C₈cycloalkyl)C₀-C₄alkyl, C₁-C₆alkoxy, C₁-C₈alkylthio, C₁-C₈alkylsulfinyl, C₁-C₆alkylsulfonyl, (C₃-C₈cycloalkyl)sulfonyl, mono- or di-(C₁-C₆alkyl)amino, mono- or di-(C₁-C₆alkyl)aminosulfonyl, or mono- or di-(C₁-C₆alkyl)aminocarbonyl, each of which is substituted optionally substituted, and each of which is preferably with from 0 to 4 substituents independently chosen from R_{E}; such that R₇ is not C₁-C₆hydroxyalkyl; or
   (iii) taken together with R₆ to form an optionally substituted carbocycle or heterocycle;
R₈ is:
   (i) halogen, cyano, amino, aminosulfonyl or —COOH;
   (ii) C₁-C₈alkyl, C₂-C₈alkenyl, C₂-C₈alkynyl, (C₃-C₈cycloalkyl)C₀-C₄alkyl, C₁-C₆alkoxy, C₁-C₆alkanoyl, C₁-C₈alkylthio, C₁-C₈alkylsulfinyl, C₁-C₆alkylsulfonyl, (C₃-C₈cycloalkyl)sulfonyl, mono- or di-(C₁-C₆alkyl)amino, mono- or di-(C₁-C₆alkyl)aminosulfonyl, or mono- or di-(C₁-C₆alkyl)aminocarbonyl, each of which is optionally substituted, and each of which is preferably substituted with from 0 to 4 substituents independently chosen from hydroxy, halogen, cyano, amino, nitro, aminocarbonyl, aminosulfonyl, —COOH, C₁-C₆alkyl, C₁-C₆haloalkyl, C₁-C₆alkoxy, C₁-C₆haloalkoxy, C₁-C₆alkylthio, C₁-C₆alkoxycarbonyl, C₁-C₆alkanoyloxy, C₃-C₆alkanone, mono- or di-(C₁-C₆alkyl)amino, C₁-C₆alkylsulfonyl, mono- or di-(C₁-C₆alkyl)aminosulfonyl, and mono- or di-(C₁-C₆alkyl)aminocarbonyl; or
   (iii) taken together with R₆ to form an optionally substituted carbocycle or heterocycle;
R₁₀ is:
   (i) hydrogen, hydroxy, halogen, cyano or amino; or
   (ii) C₁-C₈alkyl, C₂-C₈alkenyl, C₂-C₈alkynyl, C₁-C₈alkoxy, C₁-C₈alkylthio, C₁-C₈alkylsulfinyl, C₁-C₈alkylamino, (C₃-C₈cycloalkyl)C₀-C₄alkyl or C₁-C₆alkylsulfonyl; each of which is optionally substituted, and each of which is preferably substituted with from 0 to 6 substituents independently chosen from R_{E}; and
X, R₆, R₉, R₁₀ and each R_{A}, R_{D} and R_{E} are as described for Formula I.

In certain aspects, substituted heteroaryl CB1 antagonists of Formula I that further satisfy Formula III are provided: 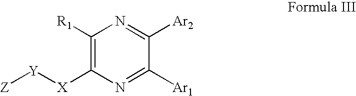
Within Formula III:
Z is O(R₃), S(O)ₘ(R₄), N(R₄)(R₅), S(O)ₘN(R₄)(R₅) or C(R₆)(R₇)(R₈); wherein m is 0, 1 or 2; such that Z is not amino or dimethylamino if X and Y are both —CH₂—;
Each R_{B} is:
   (i) halogen, hydroxy, —COOH, C₁-C₄alkyl, C₁-C₄haloalkyl, C₁-C₄aminoalkyl, C₁-C₄alkoxy, mono- or di-(C₁-C₆alkyl)aminoC₀-C₄alkyl, or mono- or di-(C₁-C₆alkyl); or
   (ii) taken together with R₃, R₄ or R₆ to form a 4- to 10-membered heterocycloalkyl that is optionally substituted, and is preferably substituted with from 0 to 3 substituents independently chosen from R_{D}; such that if R_{B} forms a heterocycloalkyl with R₃, then the heterocycloalkyl is not substituted with C₁-C₄alkoxycarbonyl;
or two R_{B} are taken together to form an optionally substituted C₃-C₈cycloalkyl or 4- to 8-membered heterocycloalkyl;
R₁ is:
   (i) hydrogen, halogen, cyano, nitro, —COOH, aminocarbonyl or aminosulfonyl; or
   (ii) C₁-C₆alkyl, C₂-C₆alkenyl, C₂-C₆alkynyl, (C₃-C₈cycloalkyl)C₀-C₄alkyl, C₁-C₆alkoxy, C₁-C₆alkoxycarbonyl, C₁-C₆alkylsulfonylC₀-C₄alkyl, C₁-C₆alkylaminosulfonylC₀-C₄alkyl, mono- or di-(C₁-C₆alkyl)aminoC₀-C₄alkyl, or mono- or di-(C₁-C₆alkyl)aminocarbonylC₀-C₄alkyl or a (4- to 8-membered heterocycle)C₀-C₄alkyl; each of which is optionally substituted, and each of which is preferably substituted with from 0 to 6 substituents independently chosen from halogen, hydroxy, cyano and amino; or
   (iii) taken together with R₂ or R₉ to form a fused 5- to 8-membered heterocycle that is optionally substituted, and is preferably substituted with from 0 to 4 substituents independently chosen from R_{E};
R₃ is:
   (i) C₁-C₈alkyl, C₂-C₈alkenyl, C₂-C₈alkynyl, C₁-C₆alkanone, C₂-C₈alkyl ether, mono- or di-(C₁-C₆alkyl)aminoC₁-C₄alkyl, (C₃-C₈cycloalkyl)C₀-C₄alkyl, mono- or di-(C₁-C₆alkyl) amino carbonyl, (5- to 7-membered heterocycle)-C(═O)—, or phenylC₀-C₄alkyl; each of which is optionally substituted, and each of which is preferably substituted with from 0 to 3 substituents independently chosen from halogen, amino, C₁-C₆alkyl or C₁-C₆alkoxy; or
   (ii) taken together with R₂, R₉ or R_{B} to form a 4- to 10-membered heterocycle that is optionally substituted, and is preferably substituted with from 0 to 3 substituents independently chosen from R_{D};
R₄ is:
   (i) hydrogen;
   (ii) C₁-C₈alkyl, C₂-C₈alkenyl, C₂-C₈alkynyl, (C₃-C₈cycloalkyl)C₀-C₄alkyl, C₁-C₆alkylsulfonyl, (C₃-C₈cycloalkyl)sulfonyl, C₂-C₈alkyl ether, mono- or di-(C₁-C₆alkyl)aminosulfonyl, mono- or di-(C₁-C₆alkyl)aminocarbonyl or (5- or 6-membered heterocycle)C₀-C₄alkyl; each of which is optionally substituted, and each of which is preferably substituted with from 0 to 6 substituents independently chosen from halogen, oxo, hydroxy, cyano, amino, aminosulfonyl, aminocarbonyl, C₁-C₆alkyl, C₁-C₆alkoxy, C₁-C₆alkylsulfonyl, mono- or di-(C₁-C₆alkyl)aminosulfonyl, mono- or di-(C₁-C₆alkyl)amino and mono- or di-(C₁-C₆alkyl)aminocarbonyl, such that R₄ is not C₁-C₈alkyl that is substituted with COOH or C₁-C₈alkoxycarbonyl;
   (iii) taken together with R₂, R₅, R₉ or R_{B} to form a 4- to 10-membered heterocycle that is optionally substituted, and is preferably substituted with from 0 to 4 substituents independently chosen from R_{D};
R₅ is:
   (i) hydrogen, cyano or aminocarbonyl;
   (ii) C₁-C₈alkyl, C₂-C₈alkenyl, C₂-C₈alkynyl, (C₃-C₈cycloalkyl)C₀-C₄alkyl, C₁-C₆alkylsulfonyl, (C₃-C₈cycloalkyl)sulfonyl, C₂-C₈alkyl ether, mono- or di-(C₁-C₆alkyl)aminosulfonyl, mono- or di-(C₁-C₆alkyl)aminocarbonyl, mono- or di-(C₁-C₆alkyl)aminoC₀-C₄alkyl, phenylC₀-C₄alkyl or (5- or 6-membered heterocycle)C₀-C₄alkyl; each of which is optionally substituted, and each of which is preferably substituted with from 0 to 6 substituents independently chosen from halogen, oxo, hydroxy, cyano, amino, aminosulfonyl, aminocarbonyl, C₁-C₆alkyl, C₁-C₆haloalkylC₁-C₆alkoxy, C₁-C₆alkylsulfonyl, mono- or di-(C₁-C₆alkyl)aminosulfonyl, mono- or di-(C₁-C₆alkyl)amino, mono- or di-(C₁-C₆alkyl)aminocarbonyl, 4- to 7-membered heterocycle, and phenyl, such that R₅ is not C₁-C₈alkyl that is substituted with COOH or C₁-C₈alkoxycarbonyl; or
   (iii) taken together with R₄ to form an optionally substituted 4- to 8-membered heterocycloalkyl;
R₆ is taken together with one or two of R₂, R₇, R₈, R₉ or R_{B} to form a 4- to 10-membered cycloalkyl or heterocycle that is substituted with from 0 to 3 substituents independently chosen from R_{D};
X, R₉, R₁₀, Ar₁, Ar₂, and each R_{A}, R_{D} and R_{E} are as described for Formula I; and
Y, R₂, R₇ and R₈ are as described for Formula II.

Within further aspects, substituted heteroaryl CB 1 antagonists of Formula I that further satisfy Formula IV are provided: 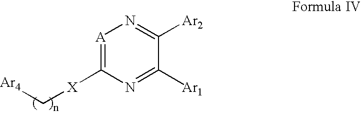
Within Formula IV:
A is CR₁ or N;
Ar₁ and Ar₂ are independently chosen from 5- to 10-membered carbocycles and heterocycles, each of which is substituted with from 1 to 6 substituents independently chosen from R_{A}; such that at least one of Ar₁ and Ar₂ is substituted aryl or substituted heteroaryl;
Ar₄ is phenyl or a 5- or 6-membered aromatic heterocycle, each of which is optionally substituted, and each of which is preferably substituted with from 0 to 4 substituents independently chosen from R_{A};
such that at least one of Ar₁, Ar₂ and Ar₄ is a heterocycle;
X is O, N(R₂) or SOₘN(R₂);
R₁ is:
   (i) hydrogen, halogen, hydroxy, cyano, amino, nitro, —COOH, aminocarbonyl or aminosulfonyl;
   (ii) C₁-C₆alkyl, C₂-C₆alkenyl, C₂-C₆alkynyl, (C₃-C₈cycloalkyl)C₀-C₄alkyl, C₁-C₆alkoxy, C₁-C₆alkoxycarbonyl, C₁-C₆alkylsulfonylC₀-C₄alkyl, mono- or di-(C₁-C₆alkyl)aminoC₀-C₄alkyl, or mono- or di-(C₁-C₆alkyl)aminocarbonylC₀-C₄alkyl or a (4- to 8-membered heterocycle)C₀-C₄alkyl; each of which is optionally substituted, and each of which is preferably substituted with from 0 to 6 substituents independently chosen from R_{E}; or
   (iii) taken together with R₂ to form a fused 5- to 8-membered heterocycle that is substituted with from 0 to 4 substituents independently chosen from R_{E};
R₂ is:
   (i) hydrogen or aminocarbonyl;
   (ii) C₁-C₆alkyl, C₂-C₆alkenyl, C₂-C₆alkynyl, (C₃-C₈cycloalkyl)C₀-C₄alkyl, C₂-C₆alkyl ether, C₁-C₆alkoxycarbonyl, mono- or di-(C₁-C₄alkyl)aminocarbonyl, mono- or di-(C₁-C₄alkyl)aminosulfonyl, mono- or di-(C₁-C₄alkyl)aminoC₀-C₄alkyl, C₁-C₆alkylsulfonyl or (4- to 8-membered heterocycle)C₀-C₄alkyl; each of which is optionally substituted, and each of which is preferably substituted with from 0 to 6 substituents independently chosen from R_{E}; or
   (iii) taken together with R₁ to form an optionally substituted fused 5- to 8-membered heterocycle;
n is 1, 2 or 3; and
Each R_{A} and R_{E} is as described for Formula I.

The present invention further provides, within other aspects, substituted heteroaryl CB1 antagonists of Formula I that further satisfy Formula V: 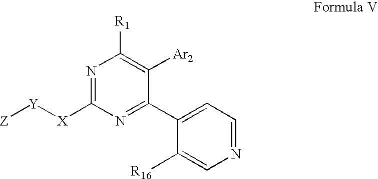
Within Formula V:
Ar₂ is a 5- to 10-membered carbocycle or heterocycle, each of which is substituted with from 0 to 6 substituents independently chosen from R_{A};
X is O, N(R₂) or SOₘN(R₂);
Y is as described for Formula II;
Z and each R_{A} and R_{E} are as described for Formula I;
Each R_{B} is:
   (i) halogen, hydroxy, —COOH, aminocarbonyl, C₁-C₄alkyl, C₁-C₄alkoxy, C₁-C₄haloalkyl, C₁-C₄aminoalkyl, mono- or di-(C₁-C₆alkyl)aminoC₀-C₄alkyl, or mono- or di-(C₁-C₆alkyl)aminocarbonyl; or
   (ii) taken together with R₃ or R₄ to form a 4- to 10-membered heterocycloalkyl that is optionally substituted, and is preferably substituted with from 0 to 3 substituents independently chosen from R_{D}; such that if R_{B} forms a heterocycloalkyl with R₃, then the heterocycloalkyl is not substituted with C₁-C₄alkoxycarbonyl;
Each R_{D} is independently chosen from:
   (i) hydroxy, halogen, cyano, amino, oxo, nitro, —COOH, aminocarbonyl and aminosulfonyl;
   (ii) C₁-C₆alkyl, C₁-C₆alkenyl, C₁-C₆alkynyl, C₃-C₈cycloalkylC₀-C₄alkyl, C₁-C₆alkoxy, C₁-C₆alkylthio, C₁-C₆alkyl ether, mono- or di-(C₁-C₆alkyl)amino, mono- or di-(C₁-C₆alkyl)aminocarbonyl, mono- or di-(C₁-C₆alkyl)aminosulfonyl, C₁-C₆alkylsulfinyl, C₁-C₆alkylsulfonyl, (4- to 8-membered heterocycle)C₀-C₄alkyl and phenylC₀-C₄alkyl, each of which is substituted with from 0 to 4 substituents independently chosen from R_{E}; and
   (iii) taken together with R₅ to form a 4- to 8-membered heterocycloalkyl;
R₁ is:
   (i) hydrogen, hydroxy, amino, halogen, cyano, nitro, —COOH, aminocarbonyl or aminosulfonyl; or
   (ii) C₁-C₆alkyl, C₂-C₆alkenyl, C₂-C₆alkynyl, (C₃-C₈cycloalkyl)C₀-C₄alkyl, C₁-C₆alkoxy, C₁-C₆alkoxycarbonyl, C₁-C₆alkylsulfonylC₀-C₄alkyl, C₁-C₆alkylaminosulfonylC₀-C₄alkyl, mono- or di-(C₁-C₆alkyl)aminoC₀-C₄alkyl, or mono- or di-(C₁-C₆alkyl)aminocarbonylC₀-C₄alkyl or a (4- to 8-membered heterocycle)C₀-C₄alkyl; each of which is optionally substituted, and each of which is preferably substituted with from 0 to 6 substituents independently chosen from R_{E}; or
R₂, if present, is taken together with R₃, R₄ or R₆ to form a 4- to 10-membered heterocycle that is optionally substituted, and is preferably substituted with from 0 to 3 substituents independently chosen from R_{D};
R₃ is:
   (i) hydrogen;
   (ii) C₁-C₈alkyl, C₁-C₈alkenyl, C₁-C₈alkynyl, C₁-C₆alkanone, C₁-C₈alkyl ether, mono- or di-(C₁-C₆alkyl)aminoC₁-C₄alkyl, (C₃-C₈cycloalkyl)C₀-C₄alkyl, mono- or di-(C₁-C₆alkyl) amino carbonyl, (5- to 7-membered heterocycle)-C(═O)—, or (4- to 8-membered heterocycloalkyl)C₀-C₄alkyl; each of which is optionally substituted, and each of which is preferably substituted with from 0 to 3 substituents independently chosen from hydroxy, halogen, C₁-C₆alkyl or C₁-C₆alkoxy; or
   (iii) taken together with R₂ or R_{B} to form a 4- to 8-membered heterocycle that is substituted with from 0 to 3 substituents independently chosen from R_{D};
R₄ is:
   (i) hydrogen or aminocarbonyl;
   (ii) C₁-C₈alkyl, C₁-C₈alkenyl, C₁-C₈alkynyl, (C₃-C₈cycloalkyl)C₀-C₄alkyl, C₁-C₆alkylsulfonyl, (C₃-C₈cycloalkyl)sulfonyl, C₁-C₆alkoxycarbonyl, C₂-C₈alkyl ether, mono- or di-(C₁-C₆alkyl)aminosulfonyl, mono- or di-(C₁-C₆alkyl)aminocarbonyl or (5- or 6-membered heterocycle)C₀-C₄alkyl; each of which is substituted with from 0 to 6 substituents independently chosen from halogen, hydroxy, oxo, cyano, amino, aminosulfonyl, aminocarbonyl, C₁-C₆alkyl, C₁-C₆alkoxy, C₁-C₆alkylsulfonyl, mono- or di-(C₁-C₆alkyl)aminosulfonyl, mono- or di-(C₁-C₆alkyl)amino and mono- or di-(C₁-C₆alkyl)aminocarbonyl; or
   (iii) taken together with R₂, R₅, or R_{B} to form a 4- to 10-membered heterocycle that is substituted with from 0 to 4 substituents independently chosen from R_{D};
R₅ is:
   (i) hydrogen or aminocarbonyl;
   (ii) C₁-C₈alkyl, C₁-C₈alkenyl, C₁-C₈alkynyl, (C₃-C₈cycloalkyl)C₀-C₄alkyl, C₁-C₆alkylsulfonyl, (C₃-C₈cycloalkyl)sulfonyl, C₁-C₆alkoxycarbonyl, C₂-C₈alkyl ether, mono- or di-(C₁-C₆alkyl)aminosulfonyl, mono- or di-(C₁-C₆alkyl)aminocarbonyl, phenylC₀-C₄alkyl or (5- or 6-membered heterocycle)C₀-C₄alkyl; each of which is optionally substituted, and each of which is preferably substituted with from 0 to 6 substituents independently chosen from halogen, hydroxy, oxo, cyano, amino, —COOH, aminosulfonyl, aminocarbonyl, C₁-C₆alkyl, C₁-C₆alkoxy, C₁-C₆alkylsulfonyl, mono- or di-(C₁-C₆alkyl)aminosulfonyl, mono- or di-(C₁-C₆alkyl)amino, mono- or di-(C₁-C₆alkyl)aminocarbonyl and phenyl; or
   (iii) taken together with R₄ or R_{D} to form an optionally substituted 4- to 8-membered heterocycle;
R₆ is:
   (i) hydrogen, hydroxy, halogen, cyano, amino, aminocarbonyl, aminosulfonyl or —COOH;
   (ii) C₁-C₈alkyl, C₂-C₈alkenyl, C₂-C₈alkynyl, (C₃-C₈cycloalkyl)C₀-C₄alkyl, C₁-C₆alkoxy, C₁-C₆alkylthio, C₁-C₆alkylsulfinyl, C₁-C₆alkylsulfonyl, (C₃-C₈cycloalkyl)sulfonyl, C₁-C₆alkoxycarbonyl, mono- or di-(C₁-C₆alkyl)aminosulfonyl, mono- or di-(C₁-C₆alkyl)aminocarbonyl, mono- or di-(C₁-C₆alkyl)aminoC₀-C₄alkyl or (5- or 6-membered heterocycle)C₀-C₄alkyl; each of which is optionally substituted, and each of which is preferably substituted with from 0 to 6 substituents independently chosen from R_{E}; or
   (iii) taken together with one or two of R₂, R₇, R₈, or R_{B} to form a 4- to 10-membered carbocycle or heterocycle that is optionally substituted, and is preferably substituted with from 0 to 3 substituents independently chosen from R_{D};
R₇ and R₈ are independently:
   (i) hydrogen, hydroxy, halogen, cyano, amino, aminocarbonyl, aminosulfonyl or —COOH;
   (ii) C₁-C₈alkyl, C₁-C₈alkenyl, C₁-C₈alkynyl, (C₃-C₈cycloalkyl)C₀-C₄alkyl, C₁-C₆alkoxy, C₁-C₆alkanoyl, C₁-C₈alkylthio, C₁-C₈alkylsulfinyl, C₁-C₆alkylsulfonyl, (C₃-C₈cycloalkyl)sulfonyl, mono- or di-(C₁-C₆alkyl)amino, mono- or di-(C₁-C₆alkyl)aminosulfonyl, or mono- or di-(C₁-C₆alkyl)aminocarbonyl, each of which is optionally substituted, and each of which is preferably substituted with from 0 to 4 substituents independently chosen from R_{E};
   (iii) taken together with R₆ to form an optionally substituted carbocycle or heterocycle; or
   (iv) R₇ and R₈ taken together form an optionally substituted carbocycle or heterocycle; and
R₁₆ is chloro, fluoro or C₁-C₃alkyl.

In still further aspects, the present invention provides substituted heteroaryl CB1 antagonists of Formula I that further satisfy Formula VI: 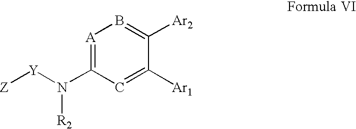
Within Formula VI:
A, B and C are independently chosen from nitrogen and CR₁, such that exactly one of A, B and C is nitrogen;
Ar₁ is a 5- to 10-membered heterocycle that is substituted with from 1 to 6 substituents independently chosen from R_{A};
Ar₂ is a 5- to 10-membered carbocycle or heterocycle, each of which is optionally substituted, and each of which is preferably substituted with from 0 to 6 substituents independently chosen from R_{A};
Y is as described for Formula II;
Z, R₂, R₅, and each R_{A}, R_{D} and R_{E} is as described for Formula I;
Each R_{B} is:
   (i) halogen, hydroxy, —COOH, aminocarbonyl, C₁-C₄alkyl, C₁-C₄alkoxy, C₁-C₄haloalkyl, C₁-C₄aminoalkyl, mono- or di-(C₁-C₆alkyl)aminoC₀-C₄alkyl, or mono- or di-(C₁-C₆alkyl)aminocarbonyl; or
   (ii) taken together with R₃, R₄ or R₆ to form a 4- to 10-membered carbocycle or heterocycle that is optionally substituted, and is preferably substituted with from 0 to 4 substituents independently chosen from R_{D};
or two R_{B} are taken together to form an optionally substituted C₃-C₈cycloalkyl or a 4- to 8-membered heterocycloalkyl;
Each R₁ is independently:
   (i) hydrogen, halogen, hydroxy, cyano, amino, nitro, —COOH, aminocarbonyl or aminosulfonyl;
   (ii) C₁-C₆alkyl, C₂-C₆alkenyl, C₂-C₆alkynyl, (C₃-C₈cycloalkyl)C₀-C₄alkyl, C₁-C₆alkoxy, C₁-C₆alkoxycarbonyl, C₁-C₆alkylsulfonyl C₀-C₄alkyl, C₁-C₆alkylaminosulfonylC₀-C₄alkyl, mono- or di-(C₁-C₆alkyl)aminoC₀-C₄alkyl, or mono- or di-(C₁-C₆alkyl)aminocarbonylC₀-C₄alkyl or a (4- to 8-membered heterocycle)C₀-C₄alkyl; each of which is optionally substituted, and each of which is preferably substituted with from 0 to 6 substituents independently chosen from R_{E}; or
   (iii) if located at the A or C position, taken together with R₂ to form a fused 5- to 8-membered carbocycle or heterocycle that is optionally substituted, and is preferably substituted with from 0 to 4 substituents independently chosen from R_{E};
R₃ is:
   (i) hydrogen;
   (ii) C₁-C₈alkyl, C₁-C₈alkenyl, C₁-C₈alkynyl, C₁-C₆alkanone, C₂-C₈alkyl ether, mono- or di-(C₁-C₆alkyl)aminoC₁-C₄alkyl, (C₃-C₈cycloalkyl)C₀-C₄alkyl, mono- or di-(C₁-C₆alkyl) amino carbonyl, (5- to 7-membered heterocycle)-C(═O)—, phenylC₀-C₄alkyl or (4- to 8-membered heterocycle)C₀-C₄alkyl; each of which is optionally substituted, and each of which is preferably substituted with from 0 to 6 substituents independently chosen from R_{E}; or
   (iii) taken together with R₂ or R_{B} to form a 4- to 10-membered heterocycle that is optionally substituted, and is preferably substituted with from 0 to 4 substituents independently chosen from R_{D};
R₄ is:
   (i) hydrogen;
   (ii) C₁-C₈alkyl, C₁-C₈alkenyl, C₁-C₈alkynyl, (C₃-C₈cycloalkyl)C₀-C₄alkyl, C₁-C₆alkylsulfonyl, (C₃-C₈cycloalkyl)sulfonyl, C₁-C₆alkoxycarbonyl, C₂-C₈alkyl ether, mono- or di-(C₁-C₆alkyl)aminosulfonyl, mono- or di-(C₁-C₆alkyl)aminocarbonyl or (5- or 6-membered heterocycle)C₀-C₄alkyl; each of which is optionally substituted, and each of which is preferably substituted with from 0 to 6 substituents independently chosen from halogen, hydroxy, oxo, cyano, amino, aminosulfonyl, aminocarbonyl, C₁-C₆alkyl, C₁-C₆alkoxy, C₁-C₆alkylsulfonyl, mono- or di-(C₁-C₆alkyl)aminosulfonyl, mono- or di-(C₁-C₆alkyl)amino and mono- or di-(C₁-C₆alkyl)aminocarbonyl; or
   (iii) taken together with one or two of R₂, R₅ or R_{B} to form a 4- to 10-membered heterocycle that is optionally substituted, and is preferably substituted with from 0 to 4 substituents independently chosen from R_{D};
R₆ is:
   (i) hydrogen, hydroxy, halogen, cyano, amino, aminocarbonyl, aminosulfonyl or —COOH;
   (ii) C₁-C₈alkyl, C₂-C₈alkenyl, C₂-C₈alkynyl, (C₃-C₈cycloalkyl)C₀-C₄alkyl, C₁-C₆alkoxy, C₁-C₆alkylthio, C₁-C₆alkylsulfinyl, C₁-C₆alkylsulfonyl, (C₃-C₈cycloalkyl)sulfonyl, C₁-C₆alkoxycarbonyl, mono- or di-(C₁-C₆alkyl)aminosulfonyl, mono- or di-(C₁-C₆alkyl)aminocarbonyl, mono- or di-(C₁-C₆alkyl)aminoC₀-C₄alkyl or (5- or 6-membered heterocycle)C₀-C₄alkyl; each of which is optionally substituted, and each of which is preferably substituted with from 0 to 6 substituents independently chosen from R_{E}; or
   (iii) taken together with one or two of R₂, R₇, R₈ or R_{B} to form a 4- to 10-membered carbocycle or heterocycle that is optionally substituted, and is preferably substituted with from 0 to 3 substituents independently chosen from R_{D}; and
R₇ and R₈, are independently:
   (i) hydrogen, hydroxy, halogen, cyano, amino, aminocarbonyl, aminosulfonyl or —COOH;
   (ii) C₁-C₈alkyl, C₁-C₈alkenyl, C₁-C₈alkynyl, (C₃-C₈cycloalkyl)C₀-C₄alkyl, C₁-C₆alkoxy, C₁-C₈alkylthio, C₁-C₈alkylsulfinyl, C₁-C₆alkylsulfonyl, (C₃-C₈cycloalkyl)sulfonyl, C₁-C₆alkoxycarbonyl, mono- or di-(C₁-C₆alkyl)amino, mono- or di-(C₁-C₆alkyl)aminosulfonyl, mono- or di-(C₁-C₆alkyl)aminocarbonyl, phenylC₀-C₄alkyl or (5- or 6-membered heterocycle)C₀-C₄alkyl; each of which is optionally substituted, and each of which is preferably substituted with from 0 to 6 substituents independently chosen from phenyl and R_{E}; or
   (iii) taken together with R₆ to form an optionally substituted carbocycle or heterocycle;
or R₇ and R₈ are taken together to form a 4- to 8-membered carbocycle or heterocycle that is substituted with from 0 to 3 substituents independently chosen from R_{D}.

Within other aspects, the present invention provides substituted heteroaryl CB1 antagonists of Formula I that further satisfy Formula VII: 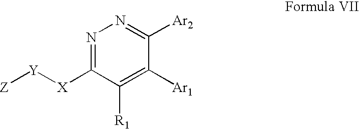
Within Formula VII:
Y, R₂, R₄, Ar₁ and Ar₂ are as described for Formula II;
X is O, N(R₂) or SOₘN(R₂);
Z, R₃, R₅, R₆, and each R_{A} and R_{D} and R_{E} are as described for Formula I;
Each R_{B} is:
   (i) halogen, hydroxy, —COOH, aminocarbonyl, C₁-C₄alkyl, C₁-C₄alkoxy, C₁-C₄haloalkyl, C₁-C₄aminoalkyl, mono- or di-(C₁-C₆alkyl)aminoC₀-C₄alkyl, or mono- or di-(C₁-C₆alkyl)aminocarbonyl; or
   (ii) taken together with R₃, R₄ or R₆ to form a 4- to 10-membered carbocycle or heterocycle that is optionally substituted, and is preferably substituted with from 0 to 4 substituents independently chosen from R_{D};
or two R_{B} are taken together to form an optionally substituted C₃-C₈cycloalkyl or a 4- to 8-membered heterocycloalkyl;
R₁ is:
   (i) hydrogen, halogen, hydroxy, cyano, amino, nitro, —COOH, aminocarbonyl or aminosulfonyl;
   (ii) C₁-C₆alkyl, C₂-C₆alkenyl, C₂-C₆alkynyl, (C₃-C₈cycloalkyl)C₀-C₄alkyl, C₁-C₆alkoxy, C₁-C₆alkoxycarbonyl, C₁-C₆alkylsulfonylC₀-C₄alkyl, mono- or di-(C₁-C₆alkyl)aminoC₀-C₄alkyl, or mono- or di-(C₁-C₆alkyl)aminocarbonylC₀-C₄alkyl or a (4- to 8-membered heterocycle)C₀-C₄alkyl; each of which is optionally substituted, and each of which is preferably substituted with from 0 to 6 substituents independently chosen from R_{E}; or
   (iii) taken together with R₂ or R₉ to form a fused 5- to 8-membered carbocycle or heterocycle that is optionally substituted, and is preferably substituted with from 0 to 4 substituents independently chosen from R_{E};
R₇ is:
   (i) hydrogen, hydroxy, halogen, cyano, amino, aminocarbonyl, aminosulfonyl or —COOH;
   (ii) C₁-C₈alkyl, C₁-C₈alkenyl, C₁-C₈alkynyl, (C₃-C₈cycloalkyl)C₀-C₄alkyl, C₁-C₆alkoxy, C₁-C₈alkylthio, C₁-C₈alkylsulfinyl, C₁-C₆alkylsulfonyl, (C₃-C₈cycloalkyl)sulfonyl, mono- or di-(C₁-C₆alkyl)amino, mono- or di-(C₁-C₆alkyl)aminosulfonyl, or mono- or di-(C₁-C₆alkyl)aminocarbonyl, each of which is substituted with from 0 to 4 substituents independently chosen from R_{E}; such that R₇ is not C₁-C₆hydroxyalkyl; or
   (iii) taken together with R₆ or R₈ to form an optionally substituted cycloalkyl or heterocycle;
R₈ is:
   (i) halogen, cyano, amino, aminosulfonyl or —COOH;
   (ii) C₁-C₈alkyl, C₁-C₈alkenyl, C₁-C₈alkynyl, (C₃-C₈cycloalkyl)C₀-C₄alkyl, C₁-C₆alkoxy, C₁-C₆alkanoyl, C₁-C₈alkylthio, C₁-C₈alkylsulfinyl, C₁-C₆alkylsulfonyl, (C₃-C₈cycloalkyl)sulfonyl, mono- or di-(C₁-C₆alkyl)amino, mono- or di-(C₁-C₆alkyl)aminosulfonyl, or mono- or di-(C₁-C₆alkyl)aminocarbonyl, each of which is substituted with from 0 to 4 substituents independently chosen from hydroxy, halogen, cyano, amino, nitro, aminocarbonyl, aminosulfonyl, —COOH, C₁-C₆alkyl, C₁-C₆haloalkyl, C₁-C₆alkoxy, C₁-C₆haloalkoxy, C₁-C₆alkylthio, C₁-C₆alkoxycarbonyl, C₁-C₆alkanoyloxy, C₃-C₆alkanone, mono- or di-(C₁-C₆alkyl)amino, C₁-C₆alkylsulfonyl, mono- or di-(C₁-C₆alkyl)aminosulfonyl, and mono- or di-(C₁-C₆alkyl)aminocarbonyl; or
   (iii) taken together with R₆ or R₇ to form an optionally substituted cycloalkyl or heterocycle.

Within certain aspects, substituted heteroaryl CB1 antagonists of Formula I, and other Formulas provided herein, exhibit a Kᵢ of no greater than 2 micromolar, 1 micromolar, 500 nanomolar, 100 nanomolar, 50 nanomolar or 10 nanomolar in a CB1 ligand binding assay and/or have an IC₅₀ value of no greater than 2 micromolar, 1 micromolar, 500 nanomolar, 100 nanomolar, 50 nanomolar or 10 nanomolar in an assay for determination of CB1 antagonist activity.

In certain embodiments, substituted heteroaryl CB1 antagonists provided herein exhibit no detectable agonist activity.

Within certain aspects, compounds as described herein are labeled with a detectable marker (e.g., radiolabeled or fluorescein conjugated).

The present invention further provides, within other aspects, pharmaceutical compositions comprising at least one substituted heteroaryl CB1 antagonist as described herein in combination with a physiologically acceptable carrier or excipient.

The present invention further provides methods for treating a condition responsive to CB1 modulation in a patient, comprising administering to the patient a therapeutically effective amount of at least one compound as described herein. Such conditions include, for example, appetite disorders, obesity, dependency disorders such as alcohol dependency and nicotine dependency, asthma, liver cirrhosis, sepsis, irritable bowel disease, Crohn's disease, depression, schizophrenia, memory disorders, cognitive disorders, movement disorders, metabolic disorders and bone loss.

In further aspects, methods are provided for suppressing appetite in a patient, comprising administering to the patient an appetite reducing amount of at least one substituted heteroaryl CB1 antagonist as described herein.

The present invention further provides pharmaceutical compositions, comprising (a) a first agent that is a substituted heteroaryl CB1 antagonist as described above, (b) a second agent that is suitable for treating an appetite disorder, obesity, an addictive disorder, asthma, liver cirrhosis, sepsis, irritable bowel disease, Crohn's disease, depression, schizophrenia, a memory disorder, a cognitive disorder, a movement disorder, a metabolic disorder or bone loss; and (c) a physiologically acceptable carrier or excipient.

The present invention also provides packaged pharmaceutical preparations, comprising: (a) a composition comprising a substituted heteroaryl CB1 antagonist as described above in a container; and (b) instructions for using the composition to treat one or more conditions responsive to CB1 modulation.

Within further aspects, the present invention provides methods for determining the presence or absence of CB1 in a sample, comprising: (a) contacting a sample with a substituted heteroaryl CB1 antagonist as described herein under conditions that permit binding of the compound to CB1; and (b) detecting a signal indicative of a level of the compound bound to CB1.

In yet another aspect, the invention provides methods of preparing the compounds disclosed herein, including the intermediates.

These and other aspects of the present invention will become apparent upon reference to the following detailed description.

### DETAILED DESCRIPTION

As noted above, the present invention provides substituted heteroaryl CB1 antagonists. Such compounds may be used in vitro or in vivo in a variety of contexts as described herein.

Terminology

Compounds are generally described herein using standard nomenclature. For compounds having asymmetric centers, it should be understood that (unless otherwise specified) all of the optical isomers and mixtures thereof are encompassed. In addition, compounds with carbon-carbon double bonds may occur in Z- and E- forms, with all isomeric forms of the compounds being included in the present invention unless otherwise specified. If a compound exists in various tautomeric forms, a recited compound is not limited to any one specific tautomer, but rather is intended to encompass all tautomeric forms. Certain compounds are described herein using a general formula that includes variables (e.g., X, A, Ar₁). Unless otherwise specified, each variable within such a formula is defined independently of any other variable, and any variable that occurs more than one time in a formula is defined independently at each occurrence.

The term “substituted heteroaryl CB1 antagonists” encompasses all compounds of Formula I, and includes pharmaceutically acceptable salts, solvates and esters of such compounds. It will be apparent that, unless otherwise specified herein, such formulas encompass compounds in which one or both of Ar₁ and Ar₂ is a heterocycle, as well as compounds in which neither Ar₁ nor Ar₂ is a heterocycle. Compounds in which neither, one or both of Ar₁ and Ar₂ is aromatic are also encompassed.

A “pharmaceutically acceptable salt” of a compound recited herein is an acid or base salt that is suitable for use in contact with the tissues of human beings or animals without excessive toxicity or carcinogenicity, and preferably without irritation, allergic response, or other problem or complication. Such salts include mineral and organic acid salts of basic residues such as amines, as well as alkali or organic salts of acidic residues such as carboxylic acids. Specific pharmaceutically acceptable salts include, but are not limited to, salts of acids such as hydrochloric, phosphoric, hydrobromic, malic, glycolic, fumaric, sulfuric, sulfamic, sulfanilic, formic, toluenesulfonic, methanesulfonic, benzene sulfonic, ethane disulfonic, 2-hydroxyethylsulfonic, nitric, benzoic, 2-acetoxybenzoic, citric, tartaric, lactic, stearic, salicylic, glutamic, ascorbic, pamoic, succinic, fumaric, maleic, propionic, hydroxymaleic, hydroiodic, phenylacetic, alkanoic such as acetic, HOOC—(CH₂)ₙ—COOH where n is 0-4, and the like. Similarly, pharmaceutically acceptable cations include, but are not limited to sodium, potassium, calcium, aluminum, lithium and ammonium. Those of ordinary skill in the art will recognize further pharmaceutically acceptable salts for the compounds provided herein, including those listed by Remington's Pharmaceutical Sciences, 17th ed., Mack Publishing Company, Easton, Pa., p. 1418 (1985). In general, a pharmaceutically acceptable acid or base salt can be synthesized from a parent compound that contains a basic or acidic moiety by any conventional chemical method. Briefly, such salts can be prepared by reacting the free acid or base forms of these compounds with a stoichiometric amount of the appropriate base or acid in water or in an organic solvent, or in a mixture of the two; generally, the use of nonaqueous media, such as ether, ethyl acetate, ethanol, isopropanol or acetonitrile, is preferred.

It will be apparent that each compound provided herein may, but need not, be formulated as a solvate (e.g., a hydrate) or non-covalent complex. In addition, the various crystal forms and polymorphs are within the scope of the present invention. Also provided herein are prodrugs of the compounds provided herein. A “prodrug” is a compound that may not fully satisfy the structural requirements of the compounds provided herein, but is modified in vivo, following administration to a patient, to produce a compound provided herein. For example, a prodrug may be an acylated derivative of a compound as provided herein. Prodrugs include compounds wherein hydroxy, amine or sulfhydryl groups are bonded to any group that, when administered to a mammalian subject, cleaves to form a free hydroxy, amino, or sulfhydryl group, respectively. Examples of prodrugs include, but are not limited to, acetate, formate, phosphate and benzoate derivatives of alcohol and amine functional groups within the compounds provided herein. Prodrugs of the compounds provided herein may be prepared by modifying functional groups present in the compounds in such a way that the modifications are cleaved in vivo to yield the parent compounds.

As used herein, the term “alkyl” refers to a straight or branched chain saturated aliphatic hydrocarbon. Alkyl groups include groups having from 1 to 8 carbon atoms (C₁-C₈alkyl), from 1 to 6 carbon atoms (C₁-C₆alkyl) and from 1 to 4 carbon atoms (C₁-C₄alkyl), such as methyl, ethyl, propyl, isopropyl, n-butyl, sec-butyl, tert-butyl, pentyl, 2-pentyl, isopentyl, neopentyl, hexyl, 2-hexyl, 3-hexyl or 3-methylpentyl. “C₀-C₄alkyl” refers to a single covalent bond (C₀) or an alkylene group having 1, 2, 3 or 4 carbon atoms; “C₀-C₆alkyl” refers to a single covalent bond or a C₁-C₆alkylene group; “C₀-C₈alkyl” refers to a single covalent bond or a C₁-C₈alkylene group. In certain instances, a substituent of an alkyl group is indicated, as in the term “C₁-C₄hydroxyalkyl,” which refers to a C₁-C₄alkyl group that is substituted with one or more hydroxy groups, and “C₁-C₄aminoalkyl,” which refers to a C₁-C₄alkyl group that is substituted with one or more —NH₂ groups.

“Alkylene” refers to a divalent alkyl group, as defined above. C₁-C₄alkylene is an alkylene group having 1, 2, 3 or 4 carbon atoms.

“Alkenyl” refers to straight or branched chain alkene groups, which comprise at least one unsaturated carbon-carbon double bond. Alkenyl groups include C₂-C₈alkenyl, C₂-C₆alkenyl and C₂-C₄alkenyl groups, which have from 2 to 8, 2 to 6 or 2 to 4 carbon atoms, respectively, such as ethenyl, allyl or isopropenyl. “Alkynyl” refers to straight or branched chain alkyne groups, which have one or more unsaturated carbon-carbon bonds, at least one of which is a triple bond. Alkynyl groups include C₂-C₈alkynyl, C₂-C₆alkynyl and C₂-C₄alkynyl groups, which have from 2 to 8, 2 to 6 or 2 to 4 carbon atoms, respectively.

A “cycloalkyl” is a saturated or partially saturated cyclic group in which all ring members are carbon, such as cyclopropyl, cyclobutyl, cyclopentyl and cyclohexyl, as well as partially saturated variants thereof. Certain cycloalkyl groups are C₃-C₈cycloalkyl, in which the ring contains from 3 to 8 ring members, all of which are carbon. A “(C₃-C₈cycloalkyl)C₀-C₄alkyl” is a C₃-C₈cycloalkyl group linked via a single covalent bond or a C₁-C₄alkylene group.

By “alkoxy,” as used herein, is meant an alkyl group attached via an oxygen bridge. Alkoxy groups include C₁-C₆alkoxy and C₁-C₄alkoxy groups, which have from 1 to 6 or 1 to 4 carbon atoms, respectively. Methoxy, ethoxy, propoxy, isopropoxy, n-butoxy, sec-butoxy, tert-butoxy, n-pentoxy, 2-pentoxy, 3-pentoxy, isopentoxy, neopentoxy, hexoxy, 2-hexoxy, 3-hexoxy, and 3-methylpentoxy are representative alkoxy groups.

“Alkylthio” refers to an alkyl group as described above attached via a sulfur bridge.

“Alkylsulfinyl” refers to groups of the formula —(SO)-alkyl, in which the sulfur atom is the point of attachment. Alkylsulfinyl groups include C₁-C₆alkylsulfinyl and C₁-C₄alkylsulfinyl groups, which have from 1 to 6 or 1 to 4 carbon atoms, respectively.

“Alkylsulfonyl” refers to groups of the formula —SO₂-alkyl, in which the sulfur atom is the point of attachment. Alkylsulfonyl groups include C₁-C₆alkylsulfonyl and C₁-C₄alkylsulfonyl groups, which have from 1 to 6 or 1 to 4 carbon atoms, respectively. “C₁-C₄haloalkylsulfonyl” is an alkylsulfonyl group of from 1 to 4 carbon atoms that is substituted with at least one halogen (e.g., trifluoromethylsulfonyl). “C₁-C₆alkylsulfonylC₀-C₄alkyl” is a C₁-C₆alkylsulfonyl group linked via a single covalent bond or a C₁-C₄alkylene group. “(C₃-C₈cycloalkyl)sulfonyl” refers to groups of the formula —(SO₂)—(C₃-C₈cycloalkyl), in which the sulfur atom is the point of attachment.

The term “alkanoyl” refers to an acyl group (e.g., —C═O)alkyl), where attachment is through the carbon of the keto group. Alkanoyl groups include C₂-C₈alkanoyl, C₂-C₆alkanoyl and C₂-C₄alkanoyl groups, which have from 2 to 8, 2 to 6 or 2 to 4 carbon atoms, respectively. “C₁alkanoyl” refers to —C═O)H, which (along with C₂-C₈alkanoyl) is encompassed by the term “C₁-C₈alkanoyl.” Ethanoyl is C₂alkanoyl. A “haloalkanoyl” group (e.g., C₁-C₂haloalkanoyl) is an alkanoyl group in which one or more hydrogens on the alkyl portion is replaced with the corresponding number of independently chosen halogens.

An “alkanone” is a ketone group in which carbon atoms are in a linear or branched alkyl arrangement. “C₃-C₈alkanone,” “C₃-C₆alkanone” and “C₃-C₄alkanone” refer to an alkanone having from 3 to 8, 6 or 4 carbon atoms, respectively. A C₃ alkanone has the structure —CH₂—C═O—CH₃.

Similarly, “alkyl ether” refers to a linear or branched ether substituent. Alkyl ether groups include C₂-C₈alkyl ether, C₂-C₆alkyl ether and C₂-C₄alkyl ether groups, which have 2 to 8, 6 or 4 carbon atoms, respectively. A C₂ alkyl ether has the structure —CH₂—O—CH₃.

The term “alkoxycarbonyl” refers to an alkoxy group linked via a carbonyl (i.e., a group having the general structure —C(═O)—O-alkyl). Alkoxycarbonyl groups include C₁-C₈, C₁-C₆ and C₁-C₄alkoxycarbonyl groups, which have from 1 to 8, 6 or 4 carbon atoms, respectively, in the alkyl portion of the group. “C₁alkoxycarbonyl” refers to —C(═O)—O—CH₃.

“Alkanoyloxy,” as used herein, refers to an alkanoyl group linked via an oxygen bridge (i.e., a group having the general structure —C(═O)-alkyl). Alkanoyloxy groups include C₂-C₈, C₂-C₆ and C₂-C₄alkanoyloxy groups, which have from 2 to 8, 6 or 4 carbon atoms, respectively. “C₂alkanoyloxy” refers to —C(═O)—O—CH₃.

“Alkylamino” refers to a secondary or tertiary amine that has the general structure —NH-alkyl or —N(alkyl)(alkyl), wherein each alkyl is selected independently from alkyl, cycloalkyl and (cycloalkyl)alkyl groups. Such groups include, for example, mono- and di-(C₁-C₈alkyl)amino groups, in which each C₁-C₈alkyl may be the same or different, as well as mono- and di-(C₁-C₆alkyl)amino groups and mono- and di-(C₁-C₄alkyl)amino groups.

“Alkylaminoalkyl” refers to an alkylamino group linked via an alkylene group (i.e., a group having the general structure -alkylene-NH-alkyl or -alkylene-N(alkyl)(alkyl)) in which each alkyl is selected independently from alkyl, cycloalkyl and (cycloalkyl)alkyl groups. Alkylaminoalkyl groups include, for example, mono- and di-(C₁-C₈alkyl)aminoC₁-C₈alkyl, mono- and di-(C₁-C₆alkyl)aminoC₁-C₆alkyl and mono- and di-(C₁-C₆alkyl)aminoC₁-C₄alkyl. “Mono- or di-(C₁-C₆alkyl)aminoC₀-C₆alkyl” refers to a mono- or di-(C₁-C₆alkyl)amino group linked via a single covalent bond or a C₁-C₆alkylene group. The following are representative alkylaminoalkyl groups: 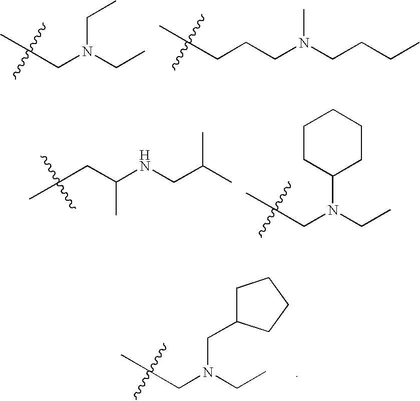
It will be apparent that the definition of “alkyl” as used in the terms “alkylamino” and “alkylaminoalkyl” differs from the definition of “alkyl” used for all other alkyl-containing groups, in the inclusion of cycloalkyl and (cycloalkyl)alkyl groups (e.g., (C₃-C₈cycloalkyl)C₀-C₄alkyl).

The term “aminocarbonyl” refers to an amide group (i.e., —C(═O)NH₂). “Mono- or di-(C₁-C₆alkyl)aminocarbonylC₀-C₄alkyl” refers to an aminocarbonyl group in which one or both hydrogens are replaced with an independently selected C₁-C₆alkyl group, and which is linked via a single covalent bond or a C₁-C₄alkylene group.

The term “aminosulfonyl” refers to a sulfonamide group (i.e., —SO₂NH₂). “Mono- or di-(C₁-C₆alkyl)aminosulfonylC₀-C₄alkyl” refers to an aminosulfonyl group in which one or both hydrogens are replaced with an independently selected C₁-C₆alkyl group, and which is linked via a single covalent bond or a C₁-C₄alkylene group.

The term “halogen” refers to fluorine, chlorine, bromine or iodine.

A “haloalkyl” is an alkyl group that is substituted with 1 or more independently chosen halogens (e.g., “C₁-C₈haloalkyl” groups have from 1 to 8 carbon atoms; “C₁-C₆haloalkyl” groups have from 1 to 6 carbon atoms). Examples of haloalkyl groups include, but are not limited to, mono-, di- or tri-fluoromethyl; mono-, di- or tri-chloromethyl; mono-, di-, tri-, tetra- or penta-fluoroethyl; mono-, di-, tri-, tetra- or penta-chloroethyl; and 1,2,2,2-tetrafluoro-1-trifluoromethyl-ethyl. Typical haloalkyl groups are trifluoromethyl and difluoromethyl. The term “haloalkoxy” refers to a haloalkyl group as defined above attached via an oxygen bridge. “C₁-C₈haloalkoxy” groups have 1 to 8 carbon atoms.

A dash (“—”) that is not between two letters or numbers is used to indicate a point of attachment for a substituent. For example, —C(═O)NH₂ is attached through the carbon atom.

A “carbocycle” has from 1 to 3 fused, pendant or spiro rings, each of which has only carbon ring members. Typically, a carbocycle that has a single ring contains from 3 to 8 ring members (i.e., C₃-C₈carbocycles); rings having from 4 or 5 to 7 ring members (i.e., C₄-C₇carbocycles or C₅-C₇carbocycles) are recited in certain embodiments. Carbocycles comprising fused, pendant or spiro rings typically contain from 9 to 14 ring members. Carbocycles may be optionally substituted with a variety of substituents, as indicated. Unless otherwise specified, a carbocycle may be a cycloalkyl group (i.e., each ring is saturated or partially saturated as described above) or an aryl group (i.e., at least one ring within the group is aromatic). Representative aromatic carbocycles are phenyl, naphthyl and biphenyl. In certain embodiments preferred carbocycles have a single ring, such as phenyl and C₃-C₈cycloalkyl groups.

A “heterocycle” (also referred to herein as a “heterocyclic group”) has from 1 to 3 fused, pendant or spiro rings, at least one of which is a heterocyclic ring (i.e., one or more ring atoms is a heteroatom independently chosen from oxygen, sulfur and nitrogen, with the remaining ring atoms being carbon). Typically, a heterocyclic ring comprises 1, 2, 3 or 4 heteroatoms; within certain embodiments each heterocyclic ring has 1 or 2 heteroatoms per ring. Each heterocyclic ring generally contains from 3 to 8 ring members (rings having from 4 or 5 to 7 ring members are recited in certain embodiments) and heterocycles comprising fused, pendant or spiro rings typically contain from 9 to 14 ring members. Certain heterocycles comprise a sulfur atom as a ring member; in certain embodiments, the sulfur atom is oxidized to SO or SO₂. Heterocycles may be optionally substituted with a variety of substituents, as indicated. Certain heterocycles are 4- to 10-membered or 5- to 10-membered, which comprise one or two rings—in certain embodiments, such heterocycles are monocyclic (e.g., 4- to 8-membered, 5- to 8-membered, 5- to 7-membered, or 5- or 6-membered); in other embodiments, such heterocycles are 9- or 10-membered bicyclic heterocycles.

Certain heterocycles are heteroaryl groups (i.e., at least one heterocyclic ring within the group is aromatic), such as a 5- to 10-membered heteroaryl (which may be monocyclic or bicyclic) or a 6-membered heteroaryl (e.g., pyridyl or pyrimidyl). Other heterocycles are heterocycloalkyl groups. Certain heterocycles may be linked by a single covalent bond or via an alkylene group, as indicated, for example, by the terms “(6-membered heteroarylC₀-C₄alkyl” and “(4- to 8-membered heterocycloalkyl)C₀-C₄alkyl.” Any heterocycle may, but need not, be bridged. A heterocycle that is “bridged” comprises an alkylene (e.g., methylene or ethylene) link between non-adjacent ring atoms (typically carbon atoms). The following are representative bridged heterocycles: 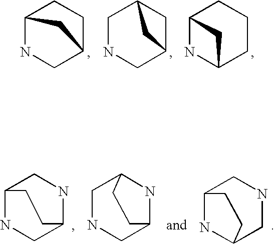
An unbridged ring lacks a link between non-adjacent ring atoms.

A “substituent,” as used herein, refers to a molecular moiety that is covalently bonded to an atom within a molecule of interest. For example, a “ring substituent” may be a moiety such as a halogen, alkyl group, haloalkyl group or other group discussed herein that is covalently bonded to an atom (such as a carbon or nitrogen atom) that is a ring member. The term “substitution” refers to replacing a hydrogen atom in a molecular structure with a substituent as described above, such that the valence on the designated atom is not exceeded, and such that a chemically stable compound (i.e., a compound that can be isolated, characterized, and tested for biological activity) results from the substitution.

Groups that are “optionally substituted” are unsubstituted or are substituted by other than hydrogen at one or more available positions, typically 1, 2, 3, 4 or 5 positions, by one or more suitable groups (which may be the same or different). Optional substitution is also indicated by the phrase “substituted with from 0 to X substituents,” where X is the maximum number of possible substituents. Certain optionally substituted groups are substituted with from 0 to 2, 3 or 4 independently selected substituents (i.e., are unsubstituted or substituted with up to the recited maximum number of substitutents).

“CB1,” as used herein, refers to the human cannabinoid receptor reported by Hoeche et al. (1991) New Biol. 3(9):880-85, as well as allelic variants thereof and homologues thereof found in other species.

A “CB1 antagonist” is a compound that detectably inhibits signal transduction mediated by CB1. Such inhibition may be determined using the representative agonist-induced GTP binding assay provided in Example 64. Preferred CB1 antagonists have an IC₅₀ of 2 μM or less in this assay, more preferably 1 μM or less, and still more preferably 500 nM or less or 100 nM or less. In certain embodiments, the CB1 antagonist is specific for CB1 (i.e., the IC₅₀ value in a similar assay performed using the predominantly peripheral cannabinoid receptor CB2 is greater than 2 μM and/or the IC₅₀ ratio (CB2/CB1) is at least 10, preferably 100, and more preferably at least 1000). CB1 antagonists preferably have minimal agonist activity (i.e., induce an increase in the basal activity of CB1 that is less than 5% of the increase that would be induced by one EC₅₀ of the agonist CP55,940, and more preferably have no detectable agonist activity within the assay described in Example 64). CB1 antagonists for use as described herein are generally non-toxic. CB1 antagonists include neutral antagonists and inverse agonists.

A “neutral antagonist” of CB1 is a compound that inhibits the activity of CB1 agonist (e.g., endocannabinoids) at CB1, but does not significantly change the basal activity of the receptor (i.e., within a GTP binding assay as described in Example 64 performed in the absence of agonist, CB1 activity is reduced by no more than 10%, more preferably by no more than 5%, and even more preferably by no more than 2%; most preferably, there is no detectable reduction in activity). Neutral antagonists may, but need not, also inhibit the binding of agonist to CB1.

An “inverse agonist” of CB1 is a compound that reduces the activity of CB1 below its basal activity level in the absence of activating concentrations of agonist. Inverse agonists may also inhibit the activity of agonist at CB1, and/or may inhibit binding of CB1 agonist to CB1. The ability of a compound to inhibit the binding of CB1 agonists to the CB1 receptor may be measured by a binding assay, such as the radioligand binding assay given in Example 63. The reduction in basal activity of CB1 produced by an inverse agonist may be determined from a GTP binding assay, such as the assay of Example 64.

A “non-competitive CB1 antagonist” is a CB1 antagonist that (1) does not detectably inhibit binding of CB1 agonist (e.g., CP55,940) to CB1 at antagonist concentrations up to 10 μM and (2) reduces the maximal functional response elicited by agonist. Compounds that satisfy these two conditions may be identified using the assays provided herein. Such compounds generally do not display detectable activity in the competition binding assay described in Example 63. In functional assays, a non-competitive antagonist concentration-dependently reduces the maximal functional response elicited by agonist without altering agonist EC₅₀. The suppression of functional activity by a non-competitive antagonist cannot be overcome by increasing agonist concentrations (i.e., the antagonist activity is insurmountable).

A “therapeutically effective amount” (or dose) is an amount that, upon administration to a patient, results in a discernible patient benefit (e.g., provides detectable relief from a condition being treated). Such relief may be detected using any appropriate criteria, including the alleviation of one or more symptoms of dependency or an appetite disorder, or the promotion of weight loss. In the case of appetite suppression, a therapeutically effective amount is sufficient to decrease patient appetite, as assessed using patient reporting or actual food intake. Such an amount is referred to herein as an “appetite reducing amount.” A therapeutically effective amount or dose generally results in a concentration of compound in a body fluid (such as blood, plasma, serum, CSF, synovial fluid, lymph, cellular interstitial fluid, tears or urine) that is sufficient to result in detectable alteration in CB1-mediated signal transduction (using an assay provided herein). The discernible patient benefit may be apparent after administration of a single dose, or may become apparent following repeated administration of the therapeutically, effective dose according to a predetermined regimen, depending upon the indication for which the compound is administered.

A “patient” is any individual treated with a compound as provided herein. Patients include humans, as well as other animals such as companion animals (e.g., dogs and cats) and livestock. Patients may be experiencing one or more symptoms of a condition responsive to CB1 modulation or may be free of such symptom(s) (i.e., treatment may be prophylactic in a patient considered to be at risk for the development of such symptoms).

Substituted Heteroaryl CB1 Antagonists

As noted above, the present invention provides substituted heteroaryl CB1 antagonists that may be used in a variety of contexts, including in the treatment of appetite disorders, obesity and addictive disorders. Such compounds may also be used within in vitro assays (e.g., assays for CB1 activity), as probes for detection and localization of CB1 and within assays to identify other CB1 antagonists, including non-competitive CB1 antagonists.

Within certain substituted heteroaryl CB1 antagonists of Formulas I-VII, variables are as follows:

A, B and C

In certain substituted heteroaryl CB1 antagonists of Formulas I and VI, the variable “C” is N. Within certain such compounds of Formula I, A is CR₁, and B is N; in other such compounds, A is N and B is CR₁; and in still further compounds, A and B are both N; or A and B are both CR₁. Accordingly, Formula I (and, unless otherwise specified, other formulas recited herein) encompasses compounds with any of the following core structures:

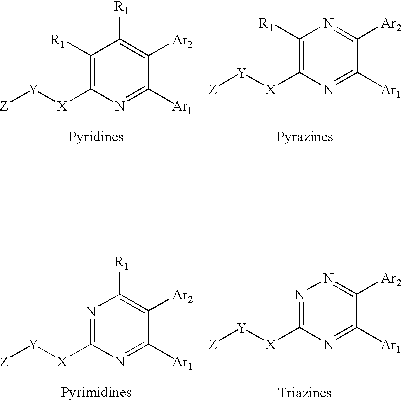

In further substituted heteroaryl CB1 antagonists of Formulas I and VI, the variable “C” is CR₁. Accordingly, Formula I (and, unless otherwise specified, other formulas provided herein) encompasses compounds with any of the following core structures: 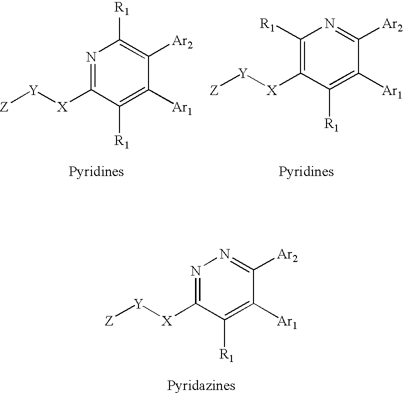
Representative R₁ groups include, for example, (i) hydrogen, chloro, bromo, fluoro, cyano, aminocarbonyl, C₁-C₄alkyl, C₁-C₄haloalkyl, C₁-C₄haloalkoxy, C₁-C₄alkoxycarbonyl, mono- and di-(C₁-C₄alkyl)aminocarbonyl and C₁-C₄alkanoyl; and (ii) C₁-C₄alkoxy that is unsubstituted or substituted with hydroxy, amino, C₁-C₄alkoxy, mono- or di-(C₁-C₄alkyl)amino or a 4- to 7-membered heterocycloalkyl; in certain embodiments, each R₁ is hydrogen, bromo, chloro, cyano, amino, methyl, ethyl, methylamino or ethylamino; in further such embodiments, each R₁ is hydrogen, bromo, chloro, cyano, methyl or ethyl.

In certain pyridines of Formula VI, C is nitrogen and B is CH or carbon substituted with halogen, hydroxy, cyano, amino, C₁-C₆alkyl, C₂-C₆alkenyl, C₂-C₆alkynyl, C₁-C₆haloalkyl, C₁-C₆alkoxy or C₁-C₆haloalkoxy.

R₁ groups at the “A” or “C” position may alternatively be taken together with R₂ or R₉ to form a fused carbocycle or heterocycle, each of which is optionally substituted. Certain such compounds satisfy Formula VIII or Formula VIIIa: 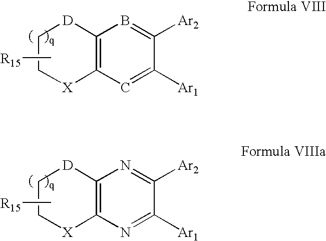
wherein:
q is 0 or 1;
D is O, S, SO, SO₂, NH or CH₂;
R₁₅ represents from 0 to 3 substituents that are preferably independently chosen from oxo, aminocarbonyl, aminosulfonyl, C₁-C₆alkyl, C₁-C₆haloalkyl, C₁-C₆alkoxy, C₁-C₆haloalkoxy, C₁-C₆alkoxycarbonyl, C₁-C₆alkanoyloxy, mono- or di-(C₁-C₆alkyl)amino, C₁-C₆alkylsulfonyl, mono- or di-(C₁-C₆alkyl)aminosulfonyl and mono- or di-(C₁-C₆alkyl)aminocarbonyl;
And the remaining variables are as described above.
   Ar₁ and Ar₂

In certain substituted heteroaryl CB1 antagonists provided herein, Ar₁ and Ar₂ are independently chosen from 5- to 7-membered carbocycles and heterocycles, each of which is substituted with from 0 to 6 substituents independently chosen from R_{A}, as described above. Within certain embodiments, at least one of Ar₁ and Ar₂ is aromatic; in further embodiments, at least one of Ar₁ and Ar₂ is a heterocycle.

Representative Ar₁ groups include phenyl and pyridyl (e.g., pyridin-4-yl), each of which is substituted with from 0 to 3 substituents (e.g., 0, 1, 2 or 3 substituents) or with 1 or 2 substitutents independently chosen from halogen (e.g., chloro, bromo or fluoro), cyano, aminocarbonyl, C₁-C₄alkyl, C₁-C₄alkoxy, C₁-C₄alkoxycarbonyl, mono- or di-(C₁-C₄alkyl)aminocarbonyl and C₁-C₄alkanoyl. Certain such Ar₁ groups are substituted with one or two halogens (e.g., 2-chloro-pyridin-4-yl, 4-fluorophenyl, 4-chlorophenyl or 2,4-dichlorophenyl).

Additional representative Ar₁ groups include cycloalkyl (e.g., cyclohexyl) and heterocycloalkyl groups (e.g., a 6-membered heterocycloalkyl group, such as piperazinyl, piperidinyl, morpholinyl or thiomorpholinyl), each of which is optionally substituted as described above, and each of which is preferably substituted with from 0 to 2 substituents independently chosen from halogen (e.g., chloro, bromo or fluoro), cyano, aminocarbonyl, C₁-C₄alkyl, C₁-C₄alkoxy, C₁-C₄alkoxycarbonyl, mono- or di-(C₁-C₄alkyl)aminocarbonyl and C₁-C₄alkanoyl. In certain such compounds, Ar₁ is morpholinyl or thiomorpholinyl, each of which is optionally substituted.

Representative Ar₂ groups include phenyl, pyrrolyl and pyridyl, each of which is substituted with from 0 to 3 substituents (e.g., 1 or 2 substituents) independently chosen from (i) chloro, bromo, fluoro, cyano, aminocarbonyl, C₁-C₄alkyl, C₁-C₄haloalkyl, C₁-C₄haloalkoxy, C₁-C₄alkoxycarbonyl, mono- or di-(C₁-C₄alkyl)aminocarbonyl and C₁-C₄alkanoyl; and (ii) C₁-C₄alkoxy that is unsubstituted or substituted with hydroxy, amino, C₁-C₄alkoxy, mono- or di-(C₁-C₄alkyl)amino or a 4- to 7-membered heterocycloalkyl. Certain such Ar₂ groups are substituted with one or more halogens (e.g., 2-chloro-pyridin-4-yl, 4-fluorophenyl, 4-chlorophenyl or 2,4-dichlorophenyl). In certain compounds, both Ar₁ and Ar₂ are both 4-fluorophenyl or 4-chlorophenyl.

Additional representative Ar₂ groups include cycloalkyl (e.g., cyclohexyl) and heterocycloalkyl groups, such as a 6-membered heterocycloalkyl (e.g., piperazinyl, piperidinyl, morpholinyl or thiomorpholinyl), each of which is optionally substituted (i) chloro, bromo, fluoro, cyano, aminocarbonyl, C₁-C₄alkyl, C₁-C₄haloalkyl, C₁-C₄haloalkoxy, C₁-C₄alkoxycarbonyl, mono- or di-(C₁-C₄alkyl)aminocarbonyl and C₁-C₄alkanoyl; and (ii) C₁-C₄alkoxy that is unsubstituted or substituted with hydroxy, amino, C₁-C₄alkoxy, mono- or di-(C₁-C₄alkyl)amino or a 4- to 7-membered heterocycloalkyl. In certain such compounds, Ar₂ is morpholinyl or thiomorpholinyl, each of which is optionally substituted.

X, Y and Z

In certain substituted heteroaryl CB1 antagonists of the Formulas described above, X is N(R₂), X is C(R₉)(R₁₀), or X is O. Certain such compounds satisfy one of the following Subformulas A-EJ:

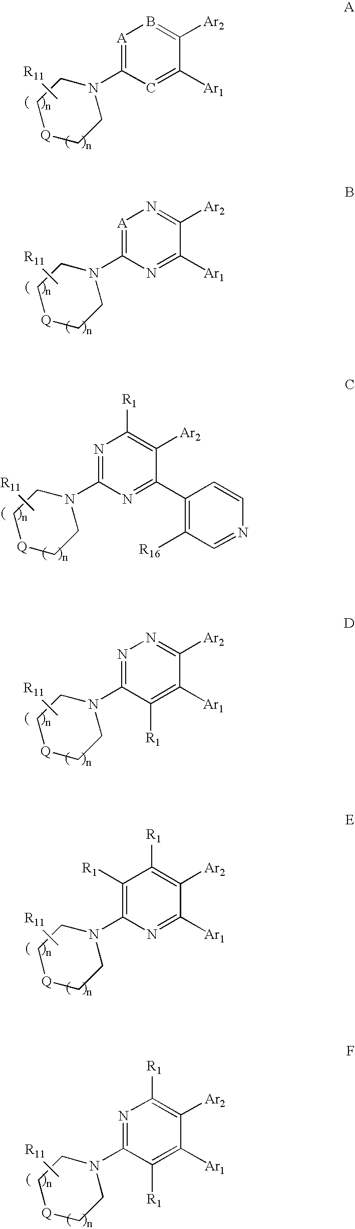

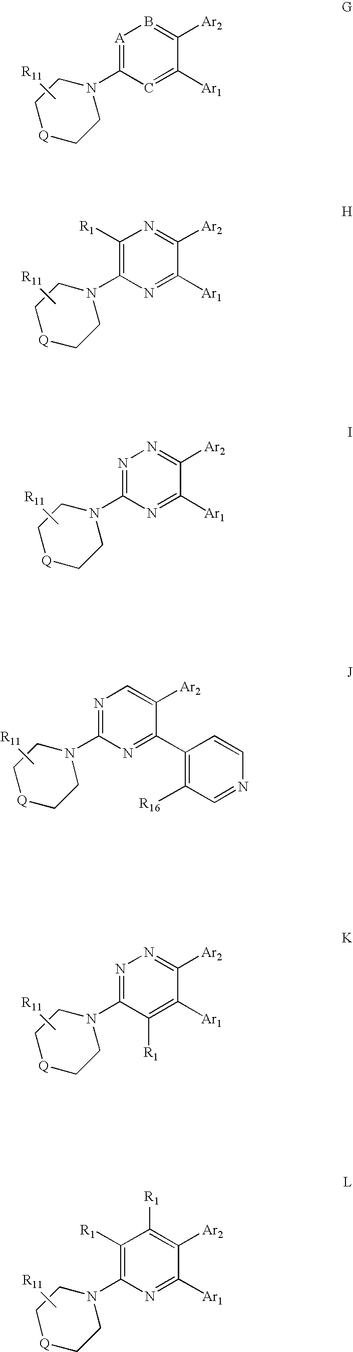

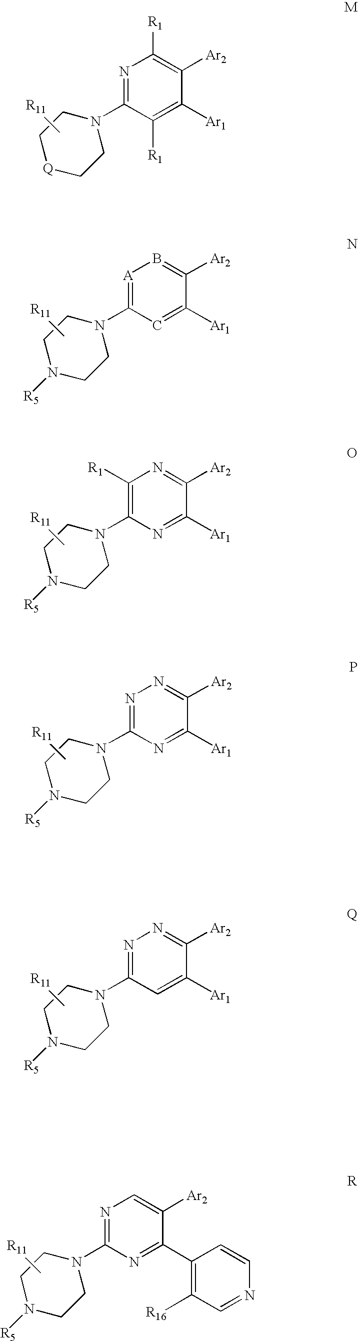

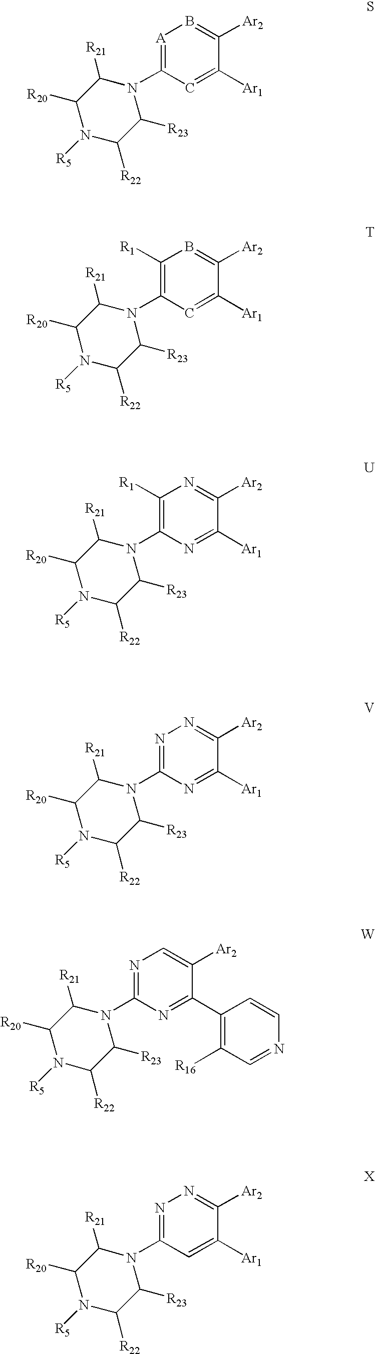

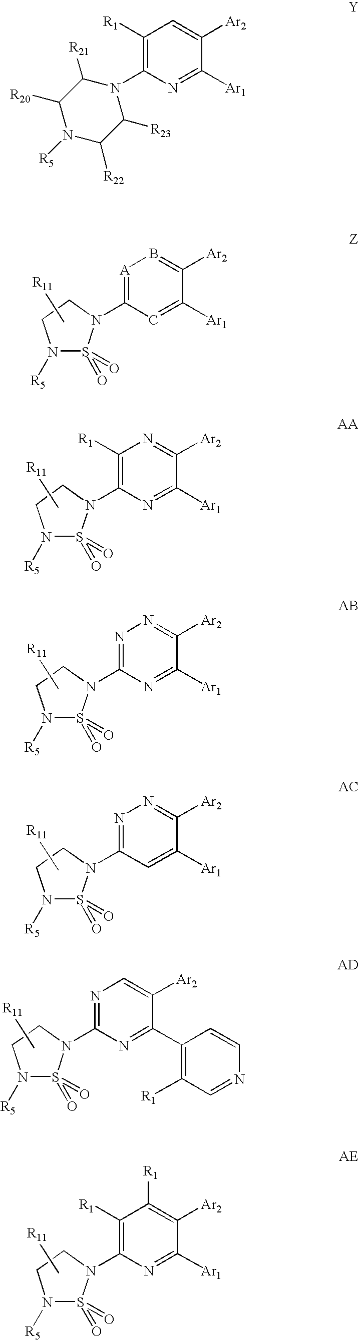

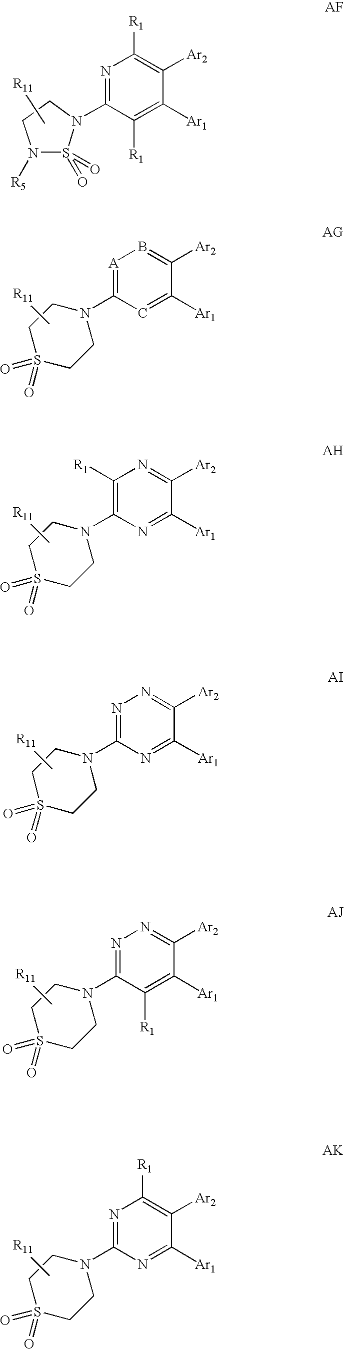

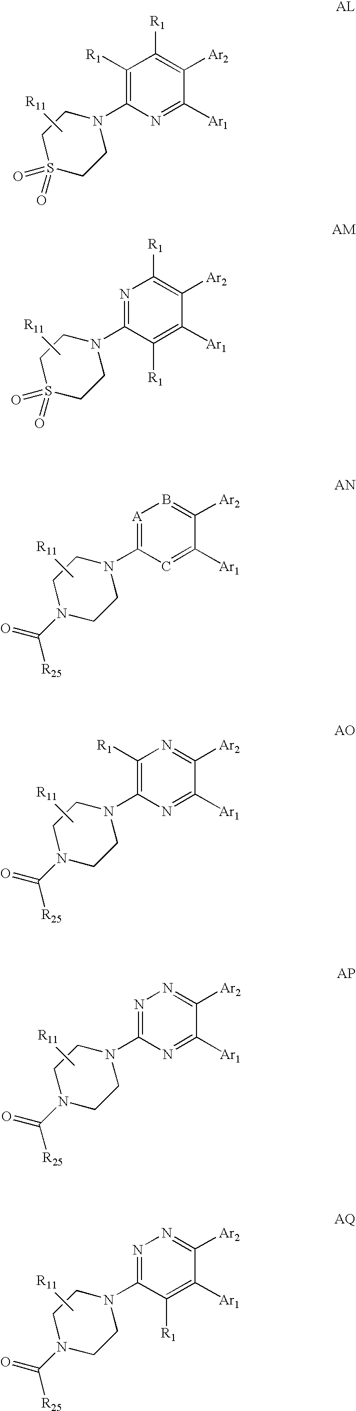

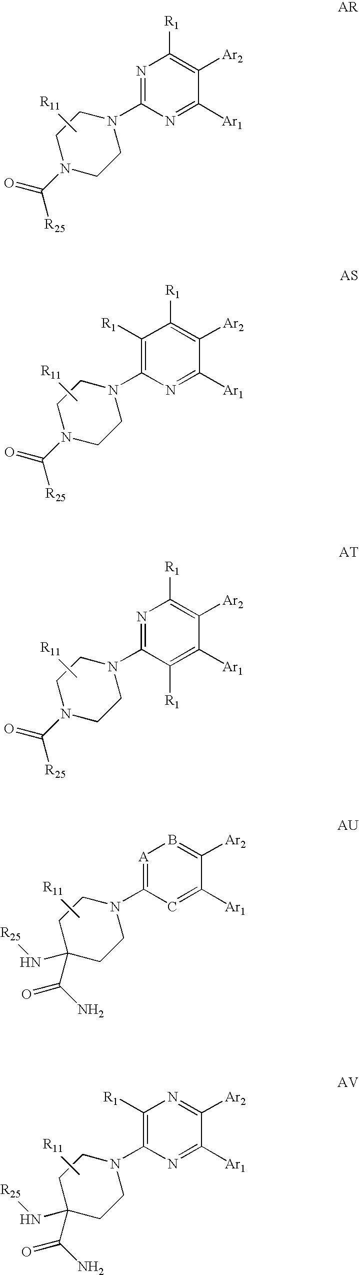

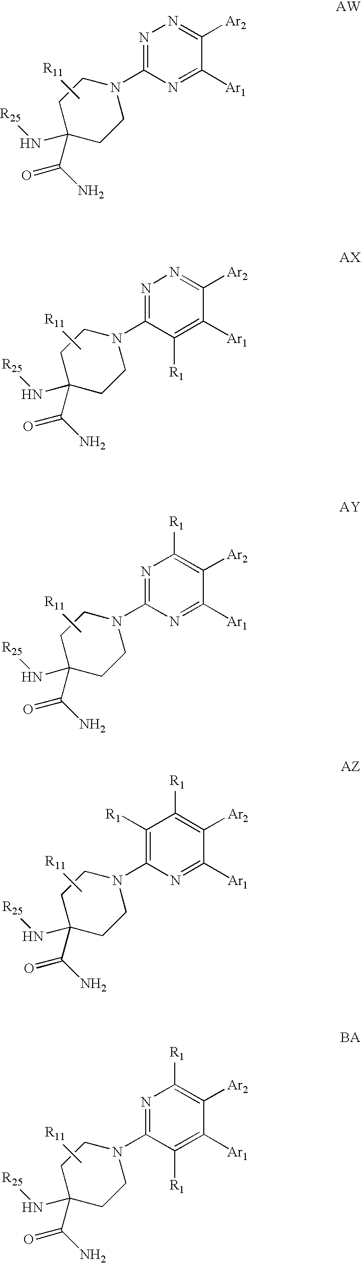

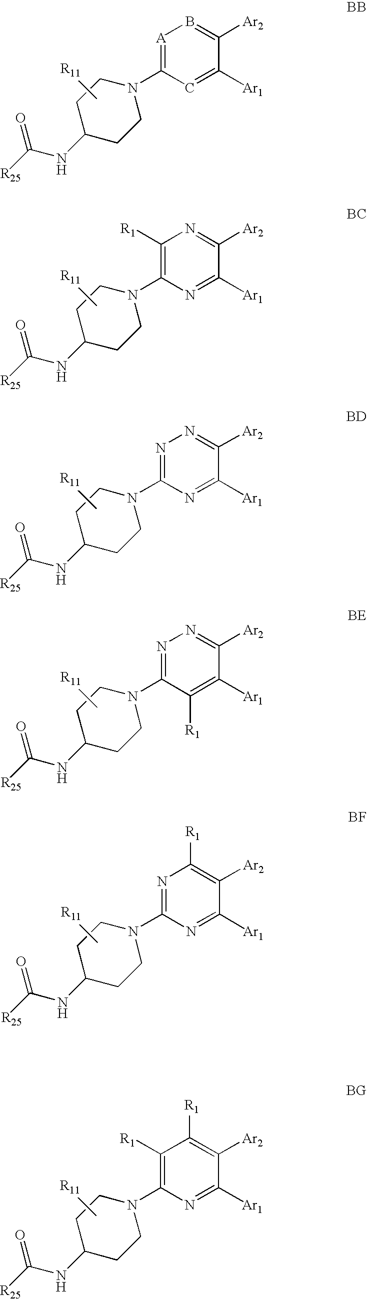

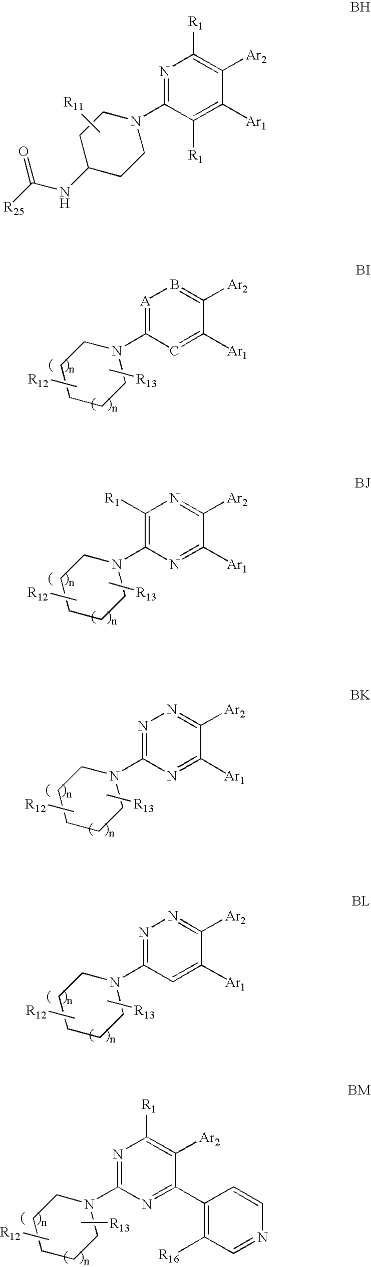

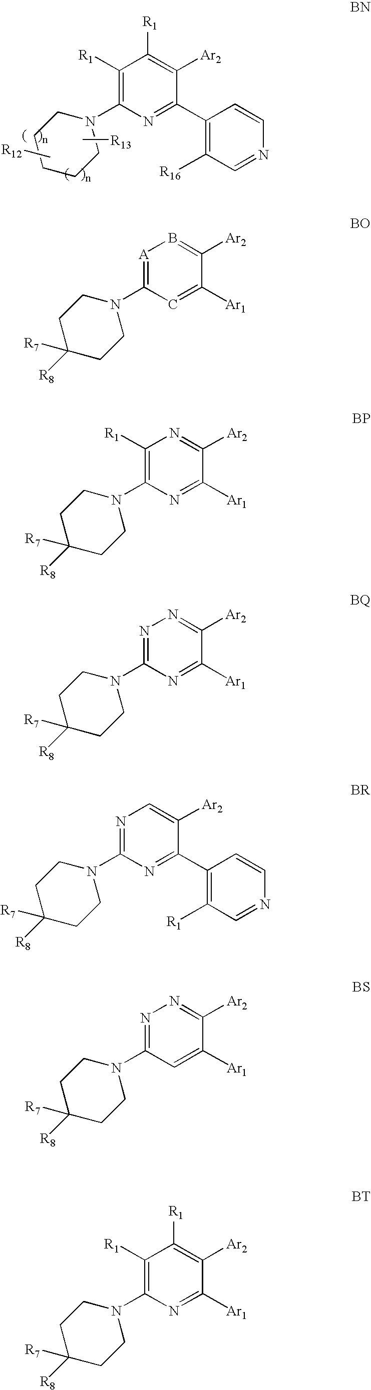

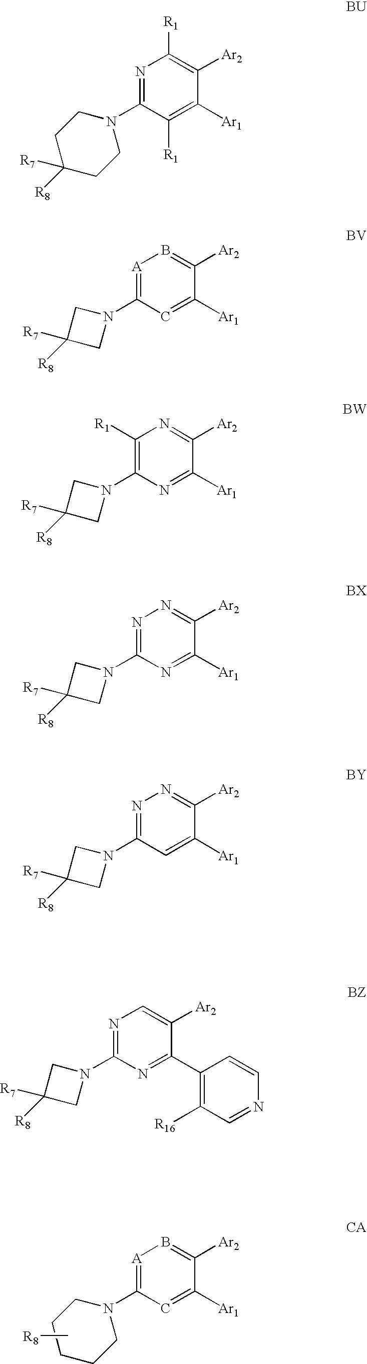

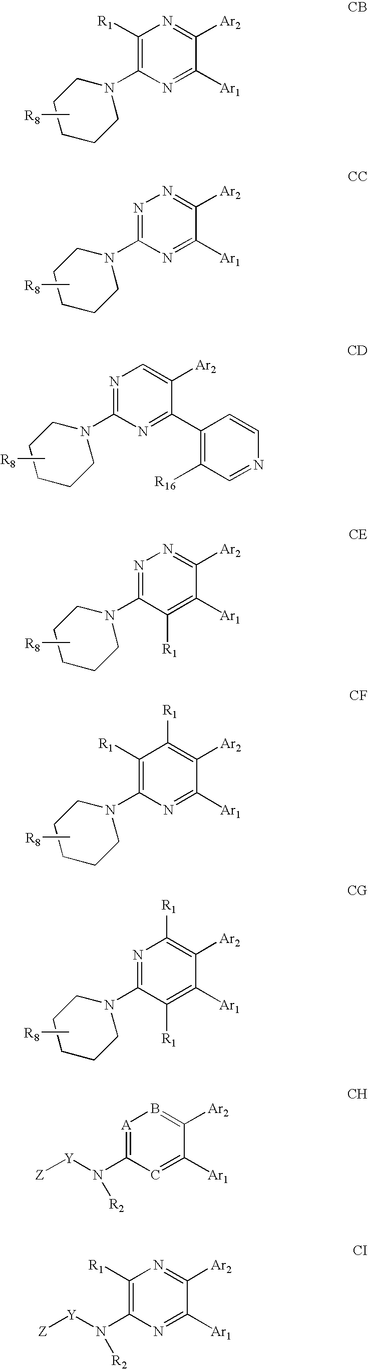

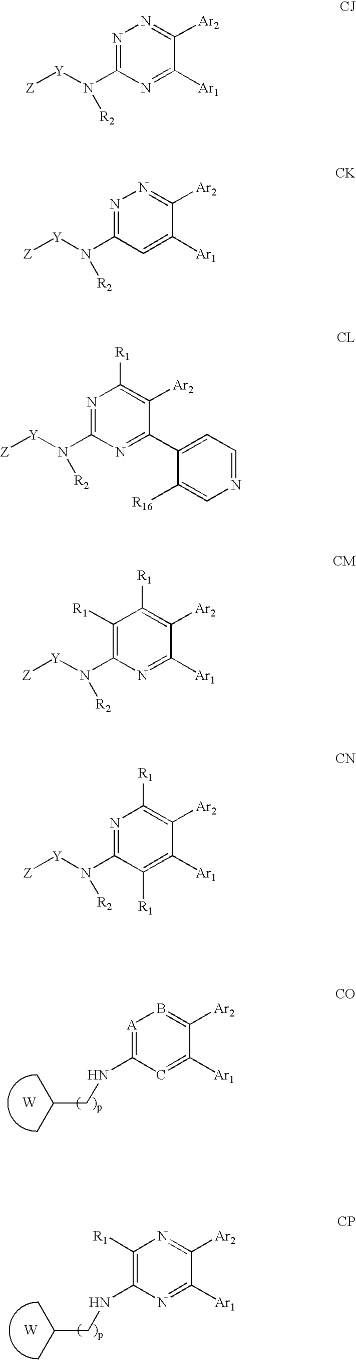

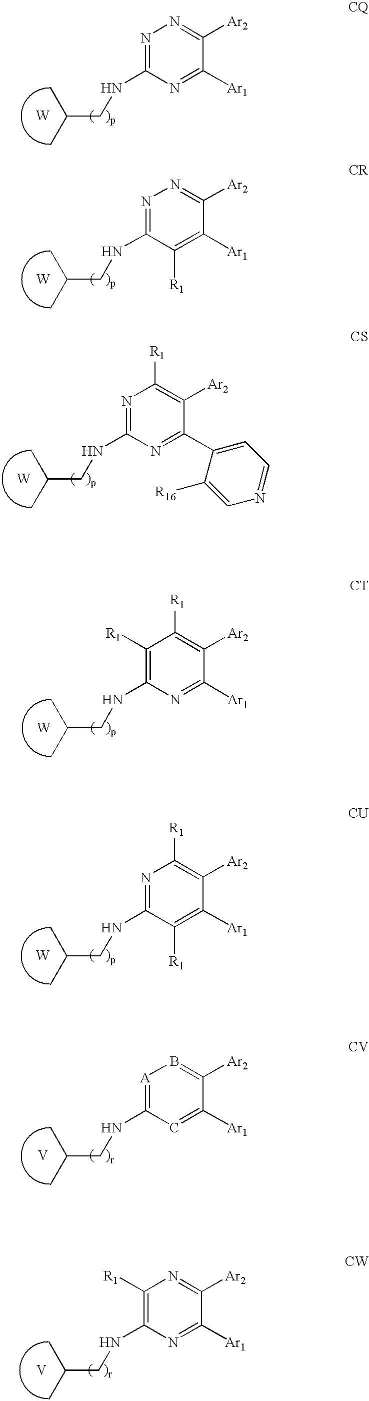

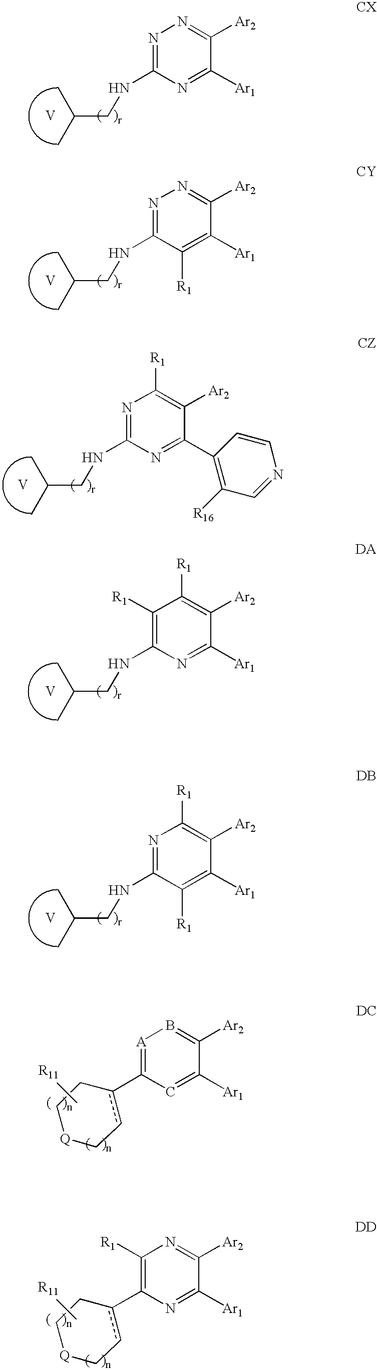

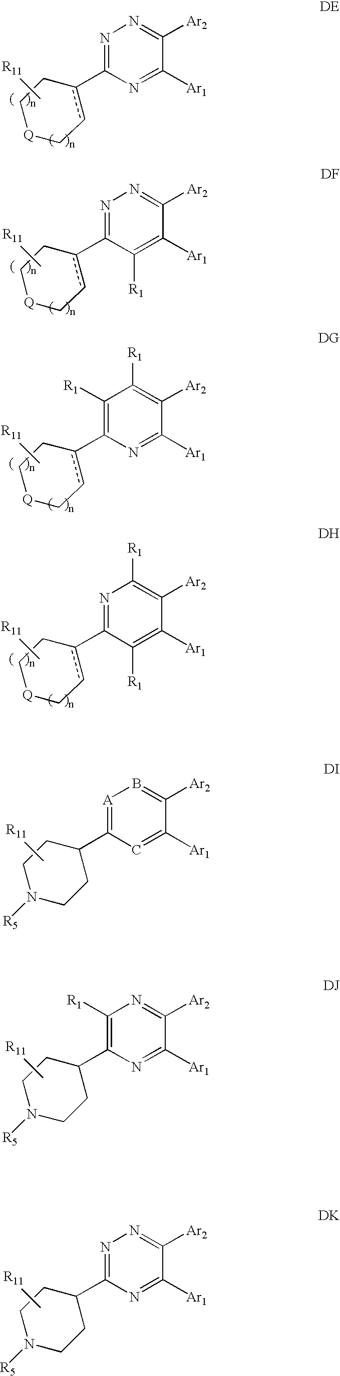

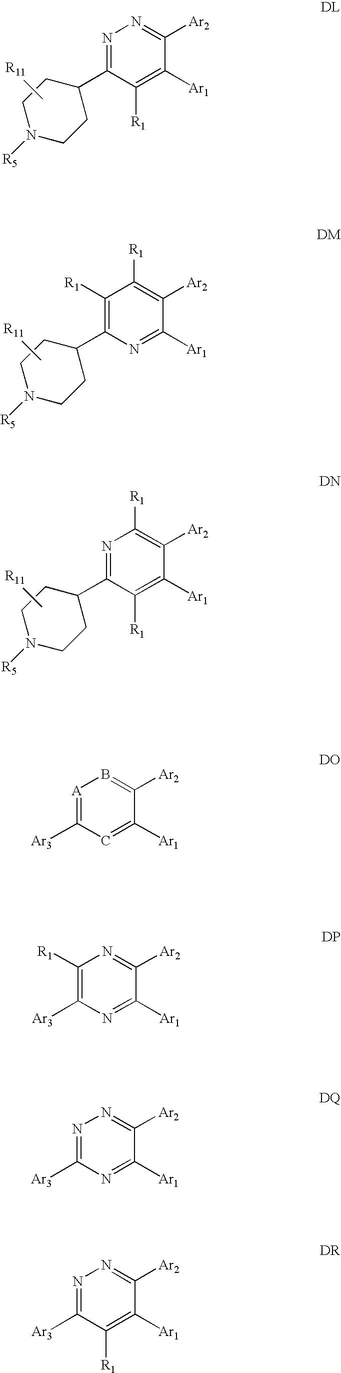

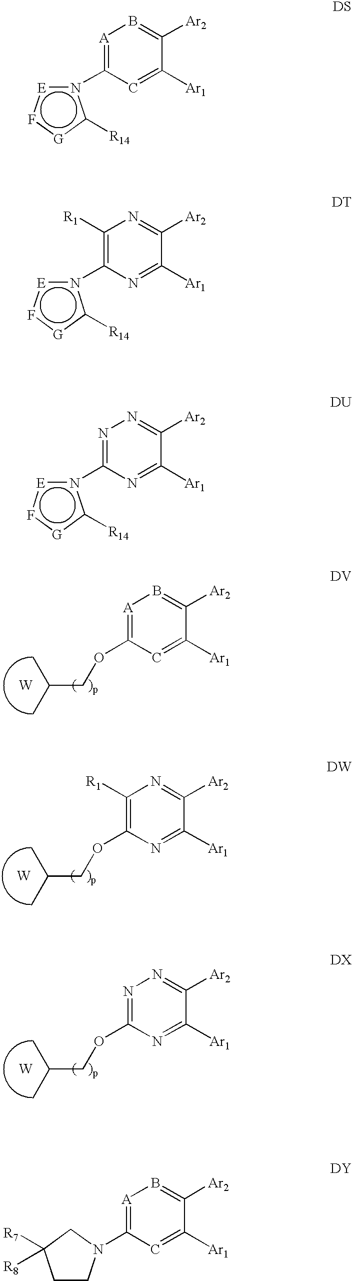

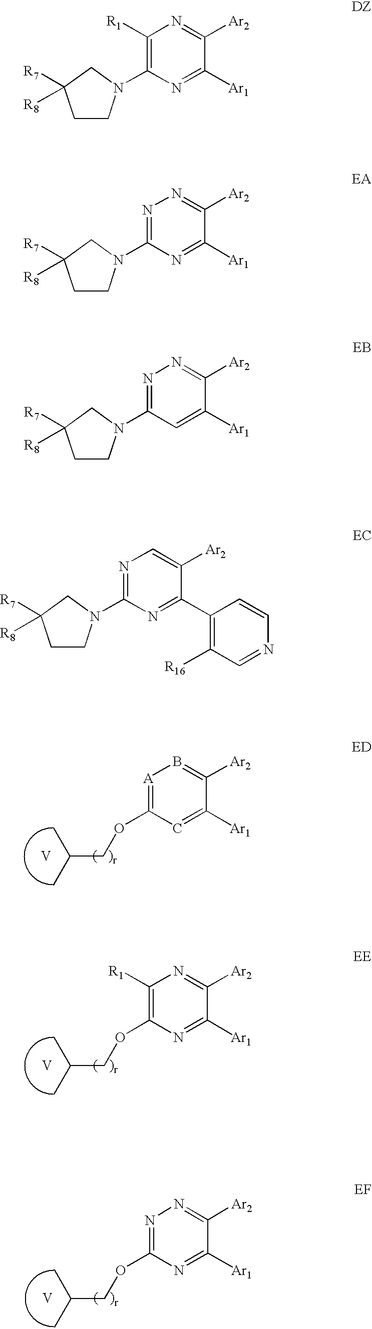

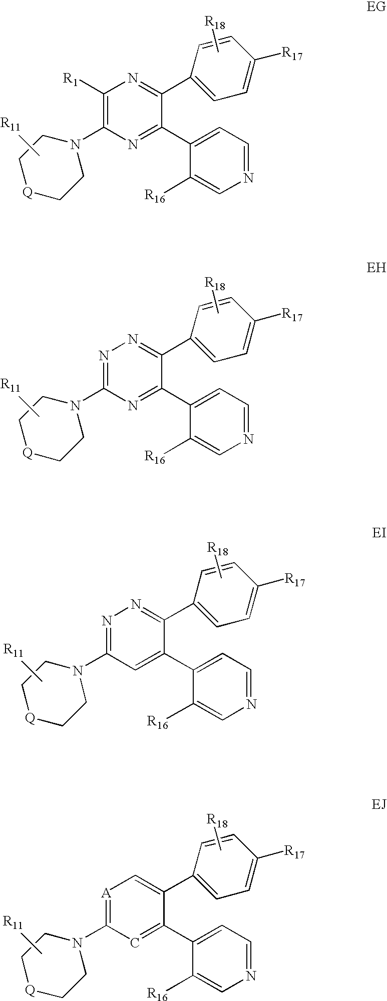

Within the above Subformulas A-EJ, variables are as defined above for any of Formulas I-VII, except as follows:
[symbol] represents a single or double bond.
E, F and G are independently chosen from N and CR₁₄.
Q is NR₅, O or SOₘ, wherein m is 0, 1 or 2; in certain embodiments Q is SO₂, SO, S, O or NH.
Z is O(R₃) or N(R₄)(R₅); in certain embodiments, Z is C₁-C₆alkoxy or (C₁-C₆alkoxy)C₁-C₆alkoxy.
Each n is independently 0, 1 or 2; in certain embodiments at least one n is not 0.
p is 0 or 1.
r is 1,2, 3 or 4.
R₂ is hydrogen, C₁-C₆alkyl, C₂-C₆alkenyl, C₂-C₆alkynyl or C₁-C₆alkanoyl. In certain compounds, R₂ is hydrogen, C₁-C₆alkyl or C₁-C₆alkanoyl. In other such compounds, R₂ is hydrogen or C₁-C₆alkyl.
R₃ is: (i) hydrogen; or (ii) C₁-C₈alkyl, C₂-C₈alkenyl, C₂-C₈alkynyl or (C₃-C₈cycloalkyl)C₀-C₄alkyl; each of which is substituted with from 0 to 3 substituents independently chosen from hydroxy, oxo, amino, C₁-C₆alkyl, C₁-C₆alkoxy, C₁-C₆alkoxycarbonyl. In certain compounds, R₃ is hydrogen or C₁-C₄alkyl.
R₄ is: (i) hydrogen; or (ii) C₁-C₈alkyl, C₂-C₈alkenyl, C₂-C₈alkynyl, (C₃-C₈cycloalkyl)C₀-C₄alkyl, C₁-C₆alkanoyl, C₁-C₆alkylsulfonyl, (C₃-C₈cycloalkyl)sulfonyl, C₁-C₆alkoxycarbonyl, mono- or di-(C₁-C₆alkyl)aminosulfonyl, mono- or di-(C₁-C₆alkyl)aminocarbonyl, phenylC₀-C₄alkyl or (5- or 6-membered heterocycle)C₀-C₄alkyl; each of which is substituted with from 0 to 3 substituents independently chosen from halogen, hydroxy, oxo, aminocarbonyl, C₁-C₆alkyl, mono- or di-(C₁-C₆alkyl)amino and mono- or di-(C₁-C₆alkyl)aminocarbonyl.
R₅ is: (i) hydrogen, cyano or aminocarbonyl; or (ii) C₁-C₈alkyl, C₂-C₈alkenyl, C₂-C₈alkynyl, (C₃-C₈cycloalkyl)C₀-C₄alkyl, C₁-C₆alkanoyl, C₁-C₆alkylsulfonyl, (C₃-C₈cycloalkyl)sulfonyl, C₁-C₆alkoxycarbonyl, mono- or di-(C₁-C₆alkyl)aminosulfonyl, mono- or di-(C₁-C₆alkyl)aminocarbonyl, mono- or di-(C₁-C₆alkyl)aminoC₁-C₄alkyl, or (5- or 6-membered heterocycle)C₀-C₄alkyl; each of which is substituted with from 0 to 3 substituents independently chosen from halogen, hydroxy, oxo, aminocarbonyl, C₁-C₆alkyl, mono- or di-(C₁-C₆alkyl)amino and mono- or di-(C₁-C₆alkyl)aminocarbonyl. In certain compounds, R₅ is (i) hydrogen or cyano; or (ii) C₁-C₆alkyl, (C₃-C₈cycloalkyl)C₀-C₄alkyl, C₂-C₆alkyl ether, C₁-C₆alkanoyl, C₁-C₆alkylsulfonyl, (C₃-C₈cycloalkyl)sulfonyl, mono- or di-(C₁-C₆alkyl)aminocarbonyl, mono- or di-(C₁-C₆alkyl)aminoC₀-C₄alkyl, mono- or di-(C₁-C₆alkyl)aminosulfonyl, or (5- or 6-membered heteroaryl)C₀-C₄alkyl, each of which is substituted with from 0 to 4 substituents independently chosen from hydroxy, halogen, cyano, amino, oxo, C₁-C₆alkyl, aminocarbonyl, mono- or di-(C₁-C₆alkyl)aminocarbonyl, or mono- or di-(C₁-C₆alkyl)amino.
R₁₁ represents 0, 1 or 2 substituents independently chosen from oxo, COOH, aminocarbonyl, C₁-C₄alkyl, C₁-C₄hydroxyalkyl, C₂-C₄alkyl ether, mono- or di-(C₁-C₄alkyl)aminoC₀-C₄alkyl, mono- or di-(C₁-C₄alkyl)aminocarbonyl and groups that are taken together with R₅ to form a 5- to 8-membered heterocycloalkyl; or R₁₁ represents two substituents that are taken together to form a C₁-C₂alkylene bridge. In certain embodiments, R₁₁ represents 0, 1 or 2 substituents independently chosen from oxo, COOH, aminocarbonyl, C₁-C₄alkyl, C₁-C₄hydroxyalkyl, C₂-C₄alkyl ether, mono- or di-(C₁-C₄alkyl)aminoC₀-C₄alkyl and mono- or di-(C₁-C₄alkyl)aminocarbonyl.
   With regard to R₁₂ and R₁₃:
R₁₂ is hydroxy, halogen, cyano, amino, C₁-C₄alkyl, (C₃-C₈cycloalkyl)C₀-C₄alkyl, C₂-C₄alkenyl, C₁-C₄hydroxyalkyl, C₁-C₄alkoxy, C₂-C₄alkyl ether, C₁-C₄alkanoyl, C₁-C₄alkylsulfonyl, mono- or di-(C₁-C₆alkyl)amino, mono- or di-(C₁-C₆alkyl)amino or (5- to 7-membered heterocycle)C₀-C₂alkyl, each of which is substituted with from 0 to 3 substituents independently chosen from oxo, amino, hydroxy, C₁-C₄alkoxy, and mono- or di-(C₁-C₆alkyl)amino; and
R₁₃ represents from 0 to 3 substituents independently chosen from halogen, cyano, amino, aminocarbonyl, C₁-C₄alkyl, C₂-C₄alkenyl, C₁-C₄hydroxyalkyl, C₁-C₄alkoxy, C₂-C₄alkyl ether and C₁-C₄alkanoyl; or
R₁₂ and R₁₃ are taken together to form a fused or spiro 5- to 7-membered heterocycle that is substituted with from 0 to 2 substituents independently chosen from hydroxy, oxo, C₁-C₄alkyl and C₁-C₄hydroxyalkyl.
Each R₁₄ is independently chosen from hydroxy, C₁-C₄alkyl, C₁-C₄hydroxyalkyl, C₁-C₄carboxyalkyl, C₂-C₄alkyl ether, C₁-C₄alkylsulfonyl, mono- or di-(C₁-C₄alkyl)aminoC₀-C₄alkyl, mono- or di-(C₁-C₄alkyl)aminocarbonylC₀-C₄alkyl and (4- to 8-membered heterocycle)C₀-C₄alkyl.
R₁₆ is chloro, fluoro or methyl.
R₁₇ is: (i) chloro, bromo, fluoro, cyano, aminocarbonyl, C₁-C₄alkyl, C₁-C₄haloalkyl, C₁-C₄haloalkoxy, C₁-C₄alkoxycarbonyl, mono- or di-(C₁-C₄alkyl)aminocarbonyl or C₁-C₄alkanoyl; or (ii) C₁-C₄alkoxy that is unsubstituted or substituted with hydroxy, amino, C₁-C₄alkoxy, mono- or di-(C₁-C₄alkyl)amino or a 4- to 7-membered heterocycloalkyl.
R₁₈ is absent or represents one substituent chosen from chloro, bromo, fluoro, cyano, aminocarbonyl, C₁-C₄alkyl, C₁-C₄alkoxy, C₁-C₄haloalkyl, or C₁-C₄haloalkoxy.
One of R₂₀ and R₂₁ is taken together with R₂₂ or R₂₃ to form a methylene or ethylene bridge, and those variables designated R₂₀, R₂₁, R₂₂ and R₂₃ that do not form bridge are hydrogen. In certain such compounds, the group designated: 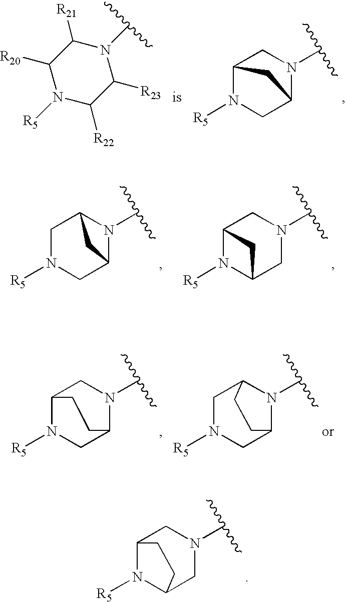
R₂₅ is: (i) hydrogen; or (ii) C₁-C₈alkyl, C₁-C₈alkenyl, C₁-C₈alkynyl, (C₃-C₈cycloalkyl)C₀-C₄alkyl, C₁-C₆alkylsulfonyl, (C₃-C₈cycloalkyl)sulfonyl, C₁-C₆alkoxycarbonyl, C₂-C₈alkyl ether, mono- or di-(C₁-C₆alkyl)aminosulfonyl, mono- or di-(C₁-C₆alkyl)aminocarbonyl or (5- or 6-membered heterocycle)C₀-C₄alkyl; each of which is substituted with from 0 to 6 substituents independently chosen from halogen, hydroxy, oxo, cyano, amino, aminosulfonyl, aminocarbonyl, C₁-C₆alkyl, C₁-C₆alkoxy, C₁-C₆alkylsulfonyl, mono- or di-(C₁-C₆alkyl)aminosulfonyl, mono- or di-(C₁-C₆alkyl)amino and mono- or di-(C₁-C₆alkyl)aminocarbonyl. In certain embodiments, R₂₅ is other than hydrogen. 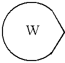
   is a 4- to 8-membered heterocycle that is substituted with from 0 to 5 substituents independently chosen from (i) oxo, cyano, C₁-C₄alkyl, C₁-C₄haloalkyl, C₁-C₄alkoxy, C₁-C₄alkoxycarbonyl, and mono- or di-(C₁-C₆alkyl)amino; and (ii) pyridyl, pyrimidyl and phenyl, each of which is substituted with from 0 to 4 substituents independently chosen from halogen, cyano, C₁-C₄alkyl and C₁-C₄haloalkyl. 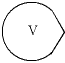
   is a 4- to 8-membered carbocycle or heterocycle that is substituted with from 0 to 4 substituents independently chosen from oxo, cyano, C₁-C₄alkyl, C₁-C₄haloalkyl, C₁-C₄alkoxy and C₁-C₄alkoxycarbonyl. In certain embodiments, 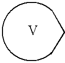
   is phenyl or pyridyl. In further embodiments, 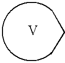
   is a heterocycle that is optionally substituted as indicated above.
Ar₃ is a 5-membered heteroaryl that is substituted with from 0 to 3 substituents independently chosen from hydroxy, C₁-C₄alkyl, C₁-C₄hydroxyalkyl, C₁-C₄carboxyalkyl, C₂-C₄alkyl ether, C₁-C₄alkylsulfonyl, mono- or di-(C₁-C₄alkyl)aminoC₀-C₄alkyl, mono- or di-(C₁-C₄alkyl)aminocarbonylC₀-C₄alkyl and (4- to 8-membered heterocycle)C₀-C₄alkyl.

Within certain substituted heteroaryl CB1 antagonists of the Formulas and Subformulas provided herein, variables satisfy one or more of the following limitations:
(i) At least one of Ar₁ and Ar₂ is phenyl, naphthyl or a 5- to 10-membered heteroaryl that is substituted with from 0 to 6 substituents independently chosen from R_{A};
(ii) Ar₁ and/or Ar₂ is not phenyl;
(iii) Ar₁ and Ar₂ are 4-fluorophenyl or Ar₁ and Ar₂ are 4-chlorophenyl;
(iv) Ar₁ is 2-chloro-pyridin-4-yl;
(v) Ar₁ and/or Ar₂ is naphthyl or a 9- or 10-membered bicyclic heterocycle;
(vi) X is O or N(R₂);
(vii) X is C(R₉)(R₁₀), Z is N(R₄)(R₅) and R₉ and R₄ are taken together to form a 4- to 7-membered heterocycloalkyl;
(viii) Z is not hydrogen;
(ix) One R_{A} is a heterocycle linked via a single bond or a methylene;
(x) R₁ is hydrogen, bromo, chloro, cyano, amino, methyl, ethyl, methylamino or ethylamino.
(xi) R₂ is hydrogen or C₁-C₆alkyl.
(xii) R₁ or R₂ is aminocarbonyl, mono or dialkylaminocarbonyl or a heterocycloalkyl linked via an amide linkage;
(xiii) R₁ and R₂ together form a 6-membered heterocycloalkyl that is optionally substituted with alkyl, oxo or alkoxy;
(xiv) R₂ is hydrogen, alkyl, hydroxyalkyl, alkyl ether, aminocarbonyl, mono or dialkylaminocarbonyl, mono or dialkylamino or taken together with R₅ or R₆ to form a carbocycle or heterocycle;
(xv) R₂ and R₄ together form a bridged heterocycle;
(xvi) R₃ is not heterocycloalkyl or phenyl;
(xvii) R₄ is taken together with R₂ and R₅ to form a fused bicyclic heterocycle;
(xviii) R₉ and R₄ together form a bridged carbocycle or heterocycle;
(xix) R₆ forms a ring with R₂ or R_{B};
(xx) at least one of R₆, R₇ and R₈ is not hydrogen;
(xxi) R₇ is hydrogen or aminocarbonyl, and R₈ is mono- or di-(C₁-C₆alkyl)amino or C₁-C₆alkanoylamino.
(xxii) R₇ and R₈ are taken together to form an optionally substituted 5- to 7-membered heterocycle.
(xxiii) Y is C₁-C₄alkylene that is optionally substituted with R_{B};
(xiv) Y is C₁-C₄alkylene that is optionally substituted with C₁-C₄alkyl; and/or
(xv) B and C are independently N, CH or carbon substituted with halogen, hydroxy, cyano, amino, C₁-C₆alkyl, C₂-C₆alkenyl, C₂-C₆alkynyl, C₁-C₆haloalkyl, C₁-C₆alkoxy or C₁-C₆haloalkoxy.

Representative compounds provided herein include, but are not limited to, those specifically described in the Examples below. It will be apparent that the specific compounds recited herein are representative only, and are not intended to limit the scope of the present invention. Further, as noted above, all compounds of the present invention may be present as a free acid or base or as a pharmaceutically acceptable salt.

As noted above, compounds provided herein are CB1 antagonists. Certain such compounds are non-competitive CB1 antagonists. In addition, or alternatively, certain compounds provided herein display CB1 specificity. CB1 antagonist activity may be confirmed using an agonist-induced GTP binding assay, such as the assay described in Example 64, herein. Such assays employ a CB1-containing cell membrane preparation (e.g., a preparation of membranes of insect cells that recombinantly express CB1) to determine the effect of a test compound on CB1 agonist-induced GTP binding to CB1. Briefly, a first cell membrane preparation comprising CB1 is contacted with: (i) labeled GTP; (ii) a CB1 agonist; and (iii) a test compound to yield a test membrane preparation. Simultaneously, or in either order, a second cell membrane preparation comprising CB1 is contacted with: (i) labeled GTP; and (ii) a CB1 agonist to yield a control membrane preparation. The labeled GTP is preferably GTPγ³⁵S; a representative CB1 agonist is CP55,940. Such contact is performed under conditions that are suitable for GTP binding to CB1, such as the conditions described in Example 64. The concentrations of labeled GTP and CB1 agonist used are generally concentrations that are sufficient to result in a detectable increase in the amount of labeled GTP bound to the membrane preparation in the presence of CB1 agonist. Such concentrations may be determined by routine experimentation; representative suitable concentrations are provided in Example 64. Generally, a range of test compound concentrations is used (e.g., ranging from 10^{-10 m} to 10⁻⁵M).

After sufficient contact (e.g., incubation) to allow GTP binding to the membrane preparations, a signal that corresponds to (represents) the amount of bound, labeled GTP is detected (typically, unbound labeled GTP is first removed via a washing step). In other words, simultaneously or in either order: (i) a test signal that represents an amount of bound, labeled GTP in the test membrane preparation is detected; and (ii) a control signal that represents an amount of bound, labeled GTP in the control membrane preparation is detected. The nature of the signal detected is determined by the type of label used. For example, if the GTP is radioactively labeled, the signal detected is radioactive decay (e.g., via liquid scintillation spectrometry). The CB1 antagonist activity of the test compound is then determined by comparing the test signal with the control signal. A test signal that is lower than the control signal indicates that the test compound is a CB1 antagonist.

In certain embodiments, preferred compounds are cannabinoid receptor-specific. This means that they only bind to, activate, or inhibit the activity of certain receptors other than cannabinoid receptors (preferably other than CB1) with affinity constants of greater than 100 nanomolar, preferably greater than 1 micromolar, more preferably greater than 4 micromolar. Alternatively, or in addition, such compounds exhibit 200-fold greater affinity for CB1 than for other cellular receptors. Such other non-cannabinoid cellular receptors include histamine receptors, bioactive peptide receptors (including NPY receptors such as NPY Y5), and hormone receptors (e.g., melanin-concentrating hormone receptors). Assays for evaluating binding to such receptors are well known, and include those disclosed in U.S. Pat. No. 6,566,367, which is incorporated herein by reference for its disclosure of NPY receptor binding assays in Example 676 columns 82-83; and in PCT International Application Publication No. WO 02/094799 which is incorporated herein by reference for its disclosure of an MCH receptor binding assay in Example 2, pages 108-109.

Utility of the compounds provided herein for the various diseases and disorders may be demonstrated in animal disease models that are known in the art, such as:
Colombo et al. (1998) Life Sciences 63:113-17 and Vickers and Kennett (2005) Curr. Drug. Targets 6:215-23—food intake and weight loss (rats)
Simiand et al. (1998) Behavioral Pharm. 9:179-181—sweet food intake (marmosets)
Rowland et al. (2001) Psychopharm. 159:111-16—food intake (rats)
Arnone et al. (1997) Psychopharm. 132:104-106—sucrose and ethanol intake (mice)
Colombo et al. (2004) Eur. J. Pharmacol. 498:119-23—alcohol motivational properties (rats)
Serra et al. (2002) Eur. J. Pharmacol. 443:95-97—alcohol deprivation effect (rats)
Rubino et al. (2000) Life Sciences 22:2213-29—opiate withdrawal syndrome (rats)
Chaperon et al. (1998) Psychopharm. 135:324-32—motor activity, place conditioning (rats)
Abraham et al. (1993) J. Clin. Invest. 93:776 and Milne and Piper (1995) Eur. J. Pharmacol. 282:243—bronchial hyperresponsiveness (sheep and guinea pigs)
Kadoi et al. (2005) British Journal of Anaesthesia 94(5):563-68—septic shock (rats)
Batkai et al. (2001) Nature Medicine 7(7):827-32—vasodilation in liver cirrhosis (rats)
Tsusumi et al. (2000) Biol. Pharm. Bull. (Japan) 23(5):657-59—constipation (monkeys)
Kapur (2001) J. Pathology 194(3):277-88—chronic intestinal pseudo-obstruction (rodents)

If desired, compounds provided herein may be evaluated for certain pharmacological properties including, but not limited to, oral bioavailability (preferred compounds are orally bioavailable to an extent allowing for therapeutically effective doses of less than 140 mg/kg, preferably less than 50 mg/kg, more preferably less than 30 mg/kg, even more preferably less than 10 mg/kg, still more preferably less than 1 mg/kg and most preferably less than 0.1 mg/kg), toxicity (a preferred compound is nontoxic when a therapeutically effective amount is administered to a subject), side effects (a preferred compound produces side effects comparable to placebo when a therapeutically effective amount of the compound is administered to a subject), serum protein binding and in vitro and in vivo half-life (a preferred compound exhibits an in vivo half-life allowing for Q.I.D. dosing, preferably T.I.D. dosing, more preferably B.I.D. dosing, and most preferably once-a-day dosing). In addition, differential penetration of the blood brain barrier may be desirable. Routine assays that are well known in the art may be used to assess these properties, and identify superior compounds for a particular use. For example, assays used to predict bioavailability include transport across human intestinal cell monolayers, including Caco-2 cell monolayers. Penetration of the blood brain barrier of a compound in humans may be predicted from the brain levels of the compound in laboratory animals given the compound (e.g., intravenously). Serum protein binding may be predicted from albumin binding assays. Compound half-life is inversely proportional to the frequency of dosage of a compound. In vitro half-lives of compounds may be predicted from assays of microsomal half-life as described herein.

As noted above, preferred compounds provided herein are nontoxic. In general, the term “nontoxic” as used herein shall be understood in a relative sense and is intended to refer to any substance that has been approved by the United States Food and Drug Administration (“FDA”) for administration to mammals (preferably humans) or, in keeping with established criteria, is susceptible to approval by the FDA for administration to mammals (preferably humans). In addition, a highly preferred nontoxic compound generally satisfies one or more of the following criteria: (1) does not substantially inhibit cellular ATP production; (2) does not significantly prolong heart QT intervals; (3) does not cause substantial liver enlargement, or (4) does not cause substantial release of liver enzymes.

As used herein, a compound that does not substantially inhibit cellular ATP production is a compound that satisfies the criteria set forth in Example 66, herein. In other words, cells treated as described in Example 66 with 100 μM of such a compound exhibit ATP levels that are at least 50% of the ATP levels detected in untreated cells. In more highly preferred embodiments, such cells exhibit ATP levels that are at least 80% of the ATP levels detected in untreated cells.

A compound that does not significantly prolong heart QT intervals is a compound that does not result in a statistically significant prolongation of heart QT intervals (as determined by electrocardiography) in guinea pigs, minipigs or dogs upon administration of a dose that yields a serum concentration equal to the EC₅₀ or IC₅₀ for the compound. In certain preferred embodiments, a dose of 0.01, 0.05, 0.1, 0.5, 1, 5, 10, 40 or 50 mg/kg administered parenterally or orally does not result in a statistically significant prolongation of heart QT intervals. By “statistically significant” is meant results varying from control at the p<0.1 level or more preferably at the p<0.05 level of significance as measured using a standard parametric assay of statistical significance such as a student's T test.

A compound does not cause substantial liver enlargement if daily treatment of laboratory rodents (e.g., mice or rats) for 5-10 days with a dose that yields a serum concentration equal to the EC₅, or IC₅₀ for the compound results in an increase in liver to body weight ratio that is no more than 100% over matched controls. In more highly preferred embodiments, such doses do not cause liver enlargement of more than 75% or 50% over matched controls. If non-rodent mammals (e.g. dogs) are used, such doses should not result in an increase of liver to body weight ratio of more than 50%, preferably not more than 25%, and more preferably not more than 10% over matched untreated controls. Preferred doses within such assays include 0.01, 0.05, 0.1, 0.5, 1, 5, 10, 40 or 50 mg/kg administered parenterally or orally.

Similarly, a compound does not promote substantial release of liver enzymes if administration of twice the minimum dose that yields a serum concentration equal to the EC₅₀ or IC₅₀ for the compound does not elevate serum levels of ALT, LDH or AST in laboratory rodents by more than 100% over matched mock-treated controls. In more highly preferred embodiments, such doses do not elevate such serum levels by more than 75% or 50% over matched controls. Alternatively, a compound does not promote substantial release of liver enzymes if, in an in vitro hepatocyte assay, concentrations (in culture media or other such solutions that are contacted and incubated with hepatocytes in vitro) that are equal to the EC₅₀ or IC₅₀ for the compound do not cause detectable release of any of such liver enzymes into culture medium above baseline levels seen in media from matched mock-treated control cells. In more highly preferred embodiments, there is no detectable release of any of such liver enzymes into culture medium above baseline levels when such compound concentrations are five-fold, and preferably ten-fold the EC₅₀ or IC₅₀ for the compound.

In other embodiments, certain preferred compounds do not inhibit or induce microsomal cytochrome P450 enzyme activities, such as CYP1A2 activity, CYP2A6 activity, CYP2C9 activity, CYP2C19 activity, CYP2D6 activity, CYP2E1 activity or CYP3A4 activity at a concentration equal to the EC₅₀ or IC₅₀ for the compound.

Certain preferred compounds are not clastogenic (e.g., as determined using a mouse erythrocyte precursor cell micronucleus assay, an Ames micronucleus assay, a spiral micronucleus assay or the like) at a concentration equal the EC₅₀ or IC₅₀ for the compound. In other embodiments, certain preferred compounds do not induce sister chromatid exchange (e.g., in Chinese hamster ovary cells) at such concentrations.

For detection purposes, as discussed in more detail below, compounds provided herein may be isotopically-labeled or radiolabeled. For example, such compounds may have one or more atoms replaced by an atom of the same element having an atomic mass or mass number different from the atomic mass or mass number usually found in nature. Examples of isotopes that can be present in the compounds provided herein include isotopes of hydrogen, carbon, nitrogen, oxygen, phosphorous, fluorine and chlorine, such as ²H, ³H, ¹¹C, ¹³C, ¹⁴C, ¹⁵N, ¹⁸O, ¹⁷O, ³¹P, ³²P, ³⁵S, ¹⁸F and ³⁶Cl. In addition, substitution with heavy isotopes such as deuterium (i.e., ²H) can afford certain therapeutic advantages resulting from greater metabolic stability, for example increased in vivo half-life or reduced dosage requirements and, hence, may be preferred in some circumstances.

Preparation of Substituted Heteroaryl CB1 Antagonists

Compounds provided herein may generally be prepared using standard synthetic methods. In general, starting materials are commercially available from suppliers such as Sigma-Aldrich Corp. (St. Louis, Mo.), or may be synthesized from commercially available precursors using established protocols. By way of example, a synthetic route similar to that shown in any of the following Schemes may be used, together with synthetic methods known in the art of synthetic organic chemistry, or variations thereon appreciated by those skilled in the art. It will be apparent that the reagents and synthetic transformations in the following Schemes can be readily modified to produce additional substituted heteroaryl CB1 antagonists. Each variable in the following Schemes refers to any group consistent with the description of the compounds provided herein.

When a protecting group is required, an optional deprotection step may be employed. Suitable protecting groups and methodology for protection and deprotection, such as those described in Protecting Groups in Organic Synthesis by T. Greene, are well known. Compounds and intermediates requiring protection/deprotection will be readily apparent.

Certain definitions used in the following Schemes and in the Examples include:

| | |
|---|---|
| AcOH | acetic acid |
| BINAP | 2,2′-bis(diphenylphosphino)-1,1′-binaphthyl |
| BOC | t-butoxycarbonyl |
| CDCl₃ | deuterated chloroform |
| DCM | dichloromethane |
| DMA | N,N-dimethylacetamide |
| DEAD | diethyl azodicarboxylate |
| DIEA | diisopropylethylamine |
| EtOH | ethanol |
| EtOAc | ethyl acetate |
| h | hour(s) |
| ¹H NMR | proton nuclear magnetic resonance |
| Hz | hertz |
| LC-MS | liquid chromatography/mass spectrometry |
| m-CPBA | m-chloroperoxybenzoic acid |
| MeOH | methanol |
| MHz | megahertz |
| MH⁺ or (M + 1) | mass + 1 |
| Min | minute(s) |
| MS | mass spectrometry |
| n-BuLi | N-butyl lithium |
| NBS | N-bromosuccinimide |
| NCS | N-chlorosuccinimide |
| NMP | N-methyl-2-pyrrolidone |
| δ | chemical shift |
| Pd(PPh₃)₄ | tetrakis(triphenylphosphine) palladium (0) |
| Pd₂(dba)₃ | tris(dibenzylideneacetone)dipalladium(0) |
| PTLC | preparative thin layer chromatography |
| rt | room temperature |
| Rt | retention time |
| TEA | triethylamine |
| TFA | trifluoroacetic acid |
| THF | tetrahydrofuran |
| TLC | thin layer chromatography |

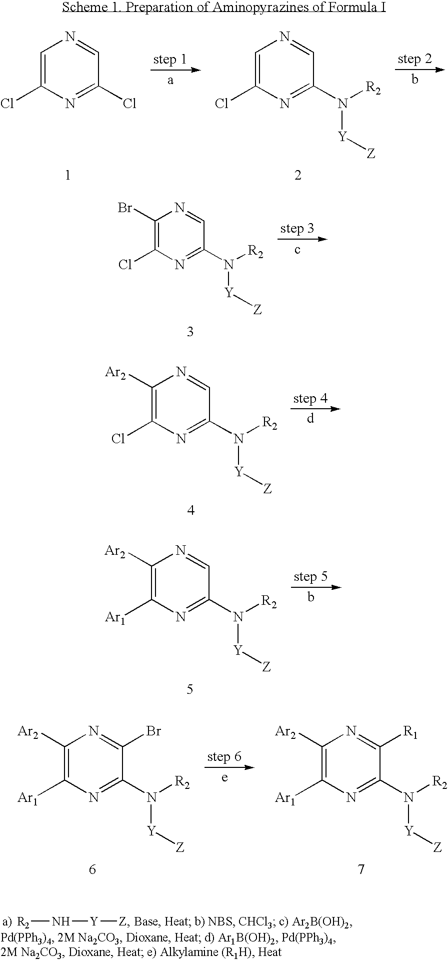

Scheme 1 illustrates a method for preparing compounds of Formula I wherein X is NR₂. In step 1, dichloropyrazine 1 is reacted with primary or secondary amine in the presence of base and heat to provide 2-amino-6-chloropyrazine 2. Bromination of 2 in step 2 provides 2-amino-5-bromo-6-chloropyrazine 3. Step 3 entails Suzuki cross-coupling with aryl or heteroaryl boronic acid to provide 4. Further reaction of 4 in step 4 with aryl boronic acid under Suzuki cross-coupling conditions provides diarylpyrazine 5 (i.e., compound of Formula I wherein R₁ is hydrogen). Diarylpyrazine 5 may be further elaborated in step 5 by bromination to produce tetrasubstituted pyrazine 6. Bromo derivative 6 may be reacted in step 6 with an alkylamine to produce compounds of Formula I wherein R₁ is alkylamino (7). Those skilled in the art will recognize that bromine in compound 6 can readily be converted to a variety of other substituents including aryl, alkyl and alkoxy by standard procedures. If desired, a variety of well-known alternative cross-coupling strategies can be employed to introduce Ar₁ and Ar₂. For example aryl or heteroaryl tin compounds may be employed in the coupling reactions. Alternatively, palladium-catalyzed amination can be conducted in step 3 or step 4 to introduce a saturated heterocycle such as morpholine as Ar₁ or Ar₂.

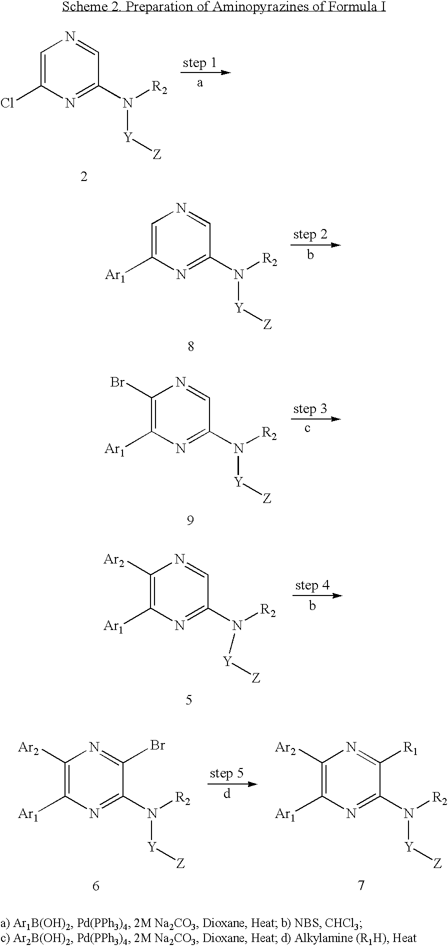

Scheme 2 illustrates a variation of Scheme 1 wherein the order of steps is modified to introduce Ar₁ prior to bromination.

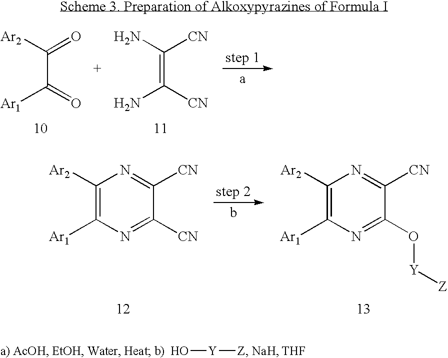

Scheme 3 illustrates a method of preparing compounds of Formula I wherein X is oxygen and R₁ is cyano. In step 1, diketone 10 is condensed with diaminomaleonitrile 11 under acidic conditions to provide dicyanopyrazine 12. Step 2 entails displacement of one of the cyano groups with alkoxide to produce 13. If Ar₁ and Ar₂ are not equivalent, step 2 yields a mixture of compounds that must be separated by chromatography. Those skilled in the art will recognize that the cyano group in 13 can be further elaborated via a variety of standard methods.

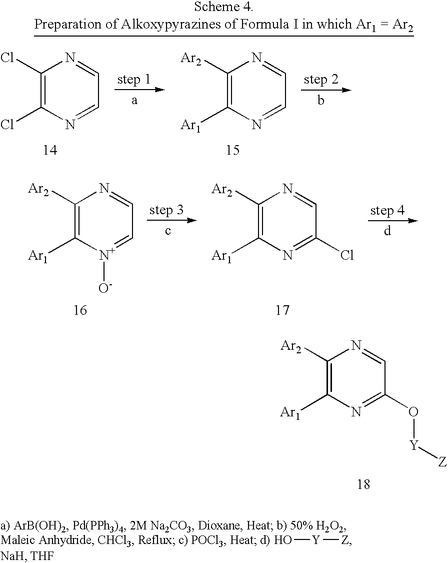

Scheme 4 provides a method for preparing compounds of Formula I wherein X is oxygen, R₁ is hydrogen and Ar₁ is equivalent to Ar₂. Step 1 entails Suzuki coupling reaction of dichloropyazine 14 to obtain diarylpyrazine 15. In step 2, the N-oxide of diarylpyrazine 15 is prepared by standard methodology. Treatment of N-oxide 16 with phosphorous oxychloride in step 3 provides chloropyrazine 17. Reaction of chloropyrazine 17 with alkoxide in step 4 provides alkoxypyrazine 18.

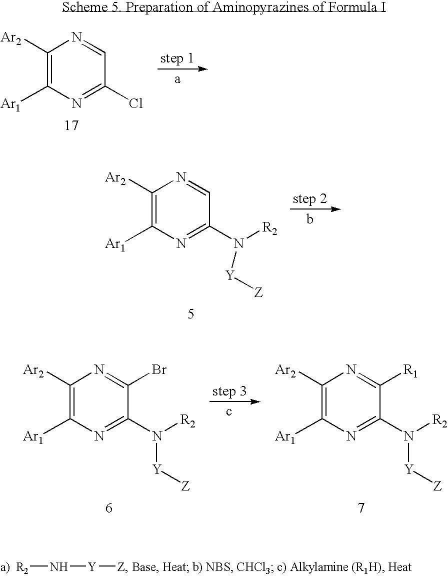

In Scheme 5, chloropyrazine 17 from Scheme 4 is elaborated according to Scheme 1.

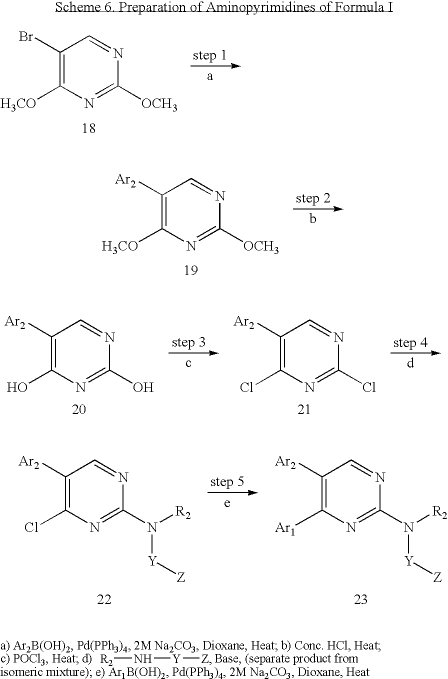

Scheme 6 illustrates a method for preparing pyrimidines of Formula I wherein X is nitrogen. In Step 1, dialkoxybromopyrimidine 18 is reacted with aryl boronic acid under Suzuki coupling conditions to obtain arylpyrimidine 19. Demethylation of 19 in step 2 under acidic conditions provides dihydroxypyrimidine 20. Treatment of 20 with phosphorous oxychloride in step 3 provides dichloropyrimidine 21. In step 4, dichloropyrimidine 21 is treated with alkylamine in the presence of base to provide, after separation from the resulting isomeric mixture of displacement products, 2-aminopyrimidine 22. Arylation in Step 5 provides 23. Those skilled in the art will recognize that Scheme 5 can readily be modified to produce compounds of Formula I wherein Ar₁ or Ar₂ is a saturated heterocycle such as morpholine.

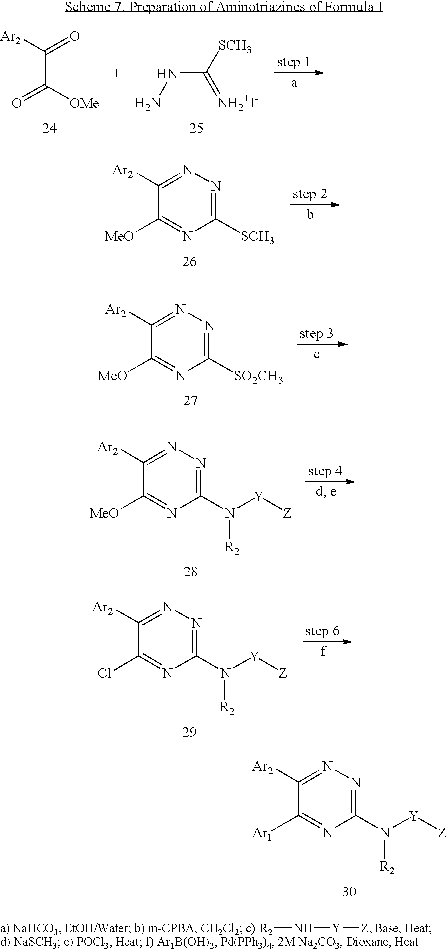

Scheme 7 provides a method for preparing certain triazines of Formula I. In step 1, an aqueous solution of S-methyl-thio-semicarbazide hydrogen iodide 25 is reacted with oxo-aryl-acetic acid methyl ester 24 to obtain 3-thiomethyltriazine 26. Oxidation of 26 to the corresponding sulfone 27 is accomplished in step 2. In step 3, the sulfone group is displaced with substituted amine to obtain 3-aminotriazine 28. Treatment of 28 with sodium methanethiolate followed by reaction with phosphorous oxychloride in step 4 provides chlorotriazine 29. Arylation of 29 under standard Suzuki coupling conditions provides 30.

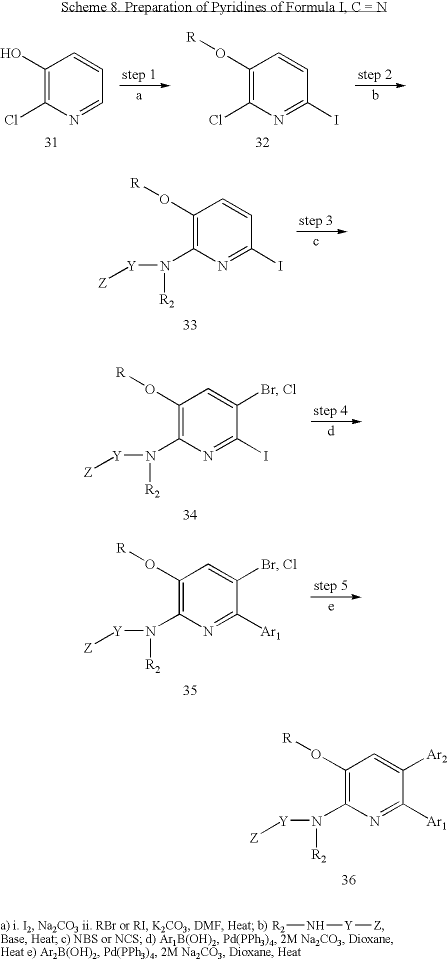

Scheme 8 illustrates a method for preparing diaryl pyridine compounds of Formula I wherein C is nitrogen. In step 1, 2-chloro-3-hydroxypyridine 31 is alkylated with alkyl halide (RBr or RI) and converted to the corresponding iodopyridine 32 wherein the substituent OR is an alkoxy substituent. Step 2 entails selective displacement of the chloro substituent in 32 to obtain aminopyridine 33. In step 3, 33 is brominated or chlorinated to obtain dihalopyridine 34. Selective reaction of the iodo substitutent in 34 in step 4 under Suzuki coupling conditions provides aryl pyridine 35. Further Suzuki reaction of 35 in step 5 provides diaryl pyridine 36.

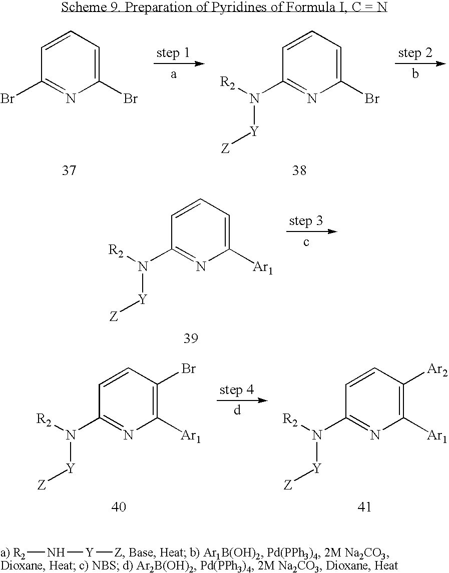

Scheme 9 provides a method for the preparation of certain pyridines of formula I. In step 1 2,6-dibromopyridine 37 is heated with an amine in the presence of a base and optionally a solvent to afford the 2-bromo-6-aminopyridine 38 which is reacted with an aryl boronic acid under Suzuki coupling conditions to obtain arylpyridine 39. Bromination of 39 in step 3 provides the bromo-pyridine 40 which is further reacted with an aryl boronic acid under Suzuki coupling conditions to give the diaryl pyridine 41.

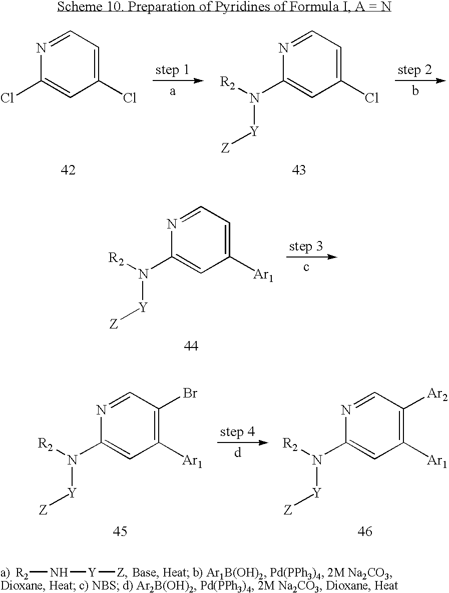

Scheme 10 illustrates a method for the preparation of certain pyridines of formula I. 2,4-Dichloropyridine 42 is heated with an amine in the presence of a base and optionally a solvent to afford the 4-chloro-2-aminopyridine 43. Reacting 43 with an arylboronic acid under Suzuki reaction conditions results in the 4-arylpyridine 44. Pyridine 44 is brominated and the resulting bromo-pyridine 45 is reacted with an arylboronic acid under Suzuki reaction conditions results in the diaryl pyridine 46.

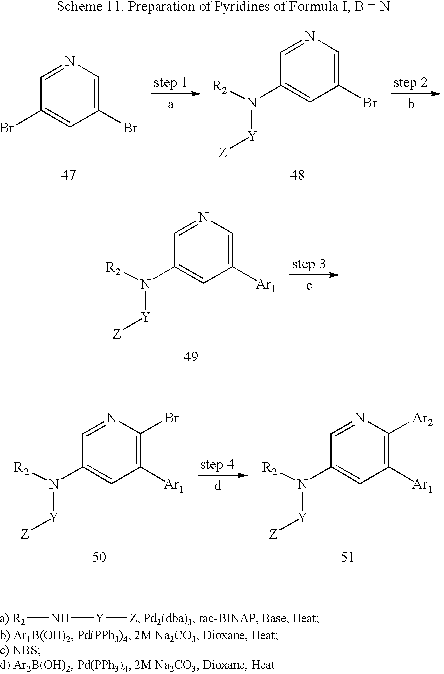

Scheme 11 provides a method for the preparation of certain pyridines of formula I. In step 1 3,6-dibromopyridine 47 is heated with an amine in under Buchwald type conditions to afford the 3-bromo-5-aminopyridine 48. This pyridine 48 is reacted with an aryl boronic acid under Suzuki coupling conditions to obtain aryl pyridine 49. Bromination of 49 in step 3 provides the bromo-pyridine 50 which is further reacted with an aryl boronic acid under Suzuki coupling conditions to give the diaryl pyridine 51.

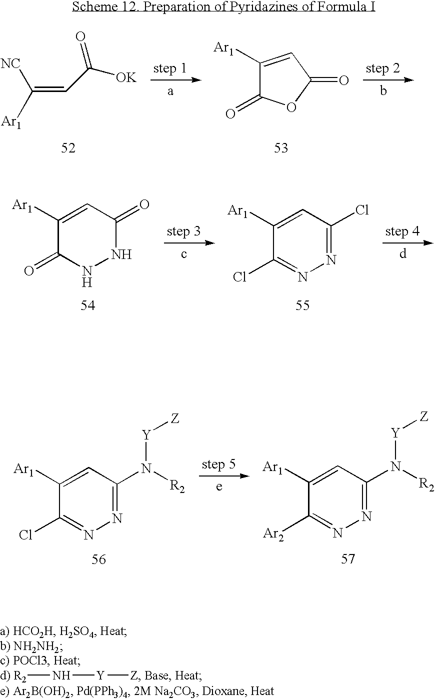

Scheme 12 provides a method for preparing diaryl pyridazines of Formula I. In step 1, 52 is cyclized under acidic conditions to provide aryl maleic anhydride 53. Reaction of aryl maleic anhydride 53 with hydrazine in step 2 provides 54. In step 3, 54 is converted to dichloropyridazine 55 by heating with phosphorous oxychloride. Treatment of dichloropyridazine 55 with alkylamine and base in step 4 provides a mixture of products. In some cases, the yield for this reaction can be improved by use of palladium (0) catalyst (e.g. Buchwald coupling conditions). Separation of the desired aminopyridazine product 56 followed by reaction under Suzuki conditions provides diaryl pyridazine 57.

In certain embodiments, a compound provided herein may contain one or more asymmetric carbon atoms, so that the compound can exist in different stereoisomeric forms. Such forms can be, for example, racemates or optically active forms. As noted above, all stereoisomers are encompassed by the present invention. Nonetheless, it may be desirable to obtain single enantiomers (i.e., optically active forms). Standard methods for preparing single enantiomers include asymmetric synthesis and resolution of the racemates. Resolution of the racemates can be accomplished, for example, by conventional methods such as crystallization in the presence of a resolving agent, or chromatography using, for example a chiral HPLC column.

Compounds may be radiolabeled by carrying out their synthesis using precursors comprising at least one atom that is a radioisotope. Each radioisotope is preferably carbon (e.g., ¹⁴C), hydrogen (e.g., ³H), sulfur (e.g., ³⁵S) or iodine (e.g., ¹²⁵I). Tritium labeled compounds may also be prepared catalytically via platinum-catalyzed exchange in tritiated acetic acid, acid-catalyzed exchange in tritiated trifluoroacetic acid, or heterogeneous-catalyzed exchange with tritium gas using the compound as substrate. In addition, certain precursors may be subjected to tritium-halogen exchange with tritium gas, tritium gas reduction of unsaturated bonds, or reduction using sodium borotritide, as appropriate. Preparation of radiolabeled compounds may be conveniently performed by a radioisotope supplier specializing in custom synthesis of radiolabeled probe compounds.

Pharmaceutical Compositions

The present invention also provides pharmaceutical compositions comprising one or more compounds provided herein, together with at least one physiologically acceptable carrier or excipient. Pharmaceutical compositions may comprise, for example, one or more of water, buffers (e.g., neutral buffered saline or phosphate buffered saline), ethanol, mineral oil, vegetable oil, dimethylsulfoxide, carbohydrates (e.g., glucose, mannose, sucrose or dextrans), mannitol, proteins, adjuvants, polypeptides or amino acids such as glycine, antioxidants, chelating agents such as EDTA or glutathione and/or preservatives. In addition, other active ingredients may (but need not) be included in the pharmaceutical compositions provided herein.

Pharmaceutical compositions may be formulated for any appropriate manner of administration, including, for example, topical, oral (including, but not limited to, sublingual), nasal, rectal or parenteral administration. The term parenteral as used herein includes subcutaneous, intradermal, intravascular (e.g., intravenous), intramuscular, spinal, intracranial, intrathecal and intraperitoneal injection, as well as any similar injection or infusion technique. In certain embodiments, compositions suitable for oral use are preferred. Such compositions include, for example, tablets, troches, lozenges, aqueous or oily suspensions, dispersible powders or granules, emulsion, hard or soft capsules, or syrups or elixirs. Within yet other embodiments, compositions of the present invention may be formulated as a lyophilizate.

Compositions intended for oral use may further comprise one or more components such as sweetening agents, flavoring agents, coloring agents and/or preserving agents in order to provide appealing and palatable preparations. Tablets contain the active ingredient in admixture with physiologically acceptable excipients that are suitable for the manufacture of tablets. Such excipients include, for example, inert diluents (e.g., calcium carbonate, sodium carbonate, lactose, calcium phosphate or sodium phosphate), granulating and disintegrating agents (e.g., corn starch or alginic acid), binding agents (e.g., starch, gelatin or acacia) and lubricating agents (e.g., magnesium stearate, stearic acid or talc). The tablets may be uncoated or they may be coated by known techniques to delay disintegration and absorption in the gastrointestinal tract and thereby provide a sustained action over a longer period. For example, a time delay material such as glyceryl monosterate or glyceryl distearate may be employed.

Formulations for oral use may also be presented as hard gelatin capsules wherein the active ingredient is mixed with an inert solid diluent (e.g., calcium carbonate, calcium phosphate or kaolin), or as soft gelatin capsules wherein the active ingredient is mixed with water or an oil medium (e.g., peanut oil, liquid paraffin or olive oil).

Aqueous suspensions contain the active material(s) in admixture with excipients suitable for the manufacture of aqueous suspensions. Such excipients include suspending agents (e.g., sodium carboxymethylcellulose, methylcellulose, hydropropylmethylcellulose, sodium alginate, polyvinylpyrrolidone, gum tragacanth and gum acacia); and dispersing or wetting agents (e.g., naturally-occurring phosphatides such as lecithin, condensation products of an alkylene oxide with fatty acids such as polyoxyethylene stearate, condensation products of ethylene oxide with long chain aliphatic alcohols such as heptadecaethyleneoxycetanol, condensation products of ethylene oxide with partial esters derived from fatty acids and a hexitol such as polyoxyethylene sorbitol monooleate, or condensation products of ethylene oxide with partial esters derived from fatty acids and hexitol anhydrides such as polyethylene sorbitan monooleate). Aqueous suspensions may also comprise one or more preservatives, such as ethyl or n-propyl p-hydroxybenzoate, one or more coloring agents, one or more flavoring agents, and one or more sweetening agents, such as sucrose or saccharin.

Oily suspensions may be formulated by suspending the active ingredient(s) in a vegetable oil (e.g., arachis oil, olive oil, sesame oil or coconut oil) or in a mineral oil such as liquid paraffin. The oily suspensions may contain a thickening agent such as beeswax, hard paraffin or cetyl alcohol. Sweetening agents such as those set forth above, and/or flavoring agents may be added to provide palatable oral preparations. Such suspensions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, such as sweetening, flavoring and coloring agents, may also be present.

Pharmaceutical compositions may also be formulated as oil-in-water emulsions. The oily phase may be a vegetable oil (e.g., olive oil or arachis oil), a mineral oil (e.g., liquid paraffin) or a mixture thereof. Suitable emulsifying agents include naturally-occurring gums (e.g., gum acacia or gum tragacanth), naturally-occurring phosphatides (e.g., soy bean lecithin, and esters or partial esters derived from fatty acids and hexitol), anhydrides (e.g., sorbitan monoleate) and condensation products of partial esters derived from fatty acids and hexitol with ethylene oxide (e.g., polyoxyethylene sorbitan monoleate). An emulsion may also comprise one or more sweetening and/or flavoring agents.

Syrups and elixirs may be formulated with sweetening agents, such as glycerol, propylene glycol, sorbitol or sucrose. Such formulations may also comprise one or more demulcents, preservatives, flavoring agents and/or coloring agents.

Formulations for topical administration typically comprise a topical vehicle combined with active agent(s), with or without additional optional components. Suitable topical vehicles and additional components are well known in the art, and it will be apparent that the choice of a vehicle will depend on the particular physical form and mode of delivery. Topical vehicles include water; organic solvents such as alcohols (e.g., ethanol or isopropyl alcohol) or glycerin; glycols (e.g., butylene, isoprene or propylene glycol); aliphatic alcohols (e.g., lanolin); mixtures of water and organic solvents and mixtures of organic solvents such as alcohol and glycerin; lipid-based materials such as fatty acids, acylglycerols (including oils, such as mineral oil, and fats of natural or synthetic origin), phosphoglycerides, sphingolipids and waxes; protein-based materials such as collagen and gelatin; silicone-based materials (both non-volatile and volatile); and hydrocarbon-based materials such as microsponges and polymer matrices. A composition may further include one or more components adapted to improve the stability or effectiveness of the applied formulation, such as stabilizing agents, suspending agents, emulsifying agents, viscosity adjusters, gelling agents, preservatives, antioxidants, skin penetration enhancers, moisturizers and sustained release materials. Examples of such components are described in Martindale—The Extra Pharmacopoeia (Pharmaceutical Press, London 1993) and Martin (ed.), Remington's Pharmaceutical Sciences. Formulations may comprise microcapsules, such as hydroxymethylcellulose or gelatin-microcapsules, liposomes, albumin microspheres, microemulsions, nanoparticles or nanocapsules.

A topical formulation may be prepared in a variety of physical forms including, for example, solids, pastes, creams, foams, lotions, gels, powders, aqueous liquids and emulsions. Typical modes of delivery for topical compositions include application using the fingers; application using a physical applicator such as a cloth, tissue, swab, stick or brush; spraying (including mist, aerosol or foam spraying); dropper application; sprinkling; soaking; and rinsing. Controlled release vehicles can also be used.

A pharmaceutical composition may be prepared as a sterile indictable aqueous or oleaginous suspension. The compound(s) provided herein, depending on the vehicle and concentration used, can either be suspended or dissolved in the vehicle. Such a composition may be formulated according to the known art using suitable dispersing, wetting and/or suspending agents such as those mentioned above. Among the acceptable vehicles and solvents that may be employed are water, 1,3-butanediol, Ringer's solution and isotonic sodium chloride solution. In addition, sterile, fixed oils may be employed as a solvent or suspending medium. For this purpose any bland fixed oil may be employed, including synthetic mono- or diglycerides. In addition, fatty acids such as oleic acid find use in the preparation of indictable compositions, and adjuvants such as local anesthetics, preservatives and/or buffering agents can be dissolved in the vehicle.

Compounds may also be formulated as suppositories (e.g., for rectal administration). Such compositions can be prepared by mixing the drug with a suitable non-irritating excipient that is solid at ordinary temperatures but liquid at the rectal temperature and will therefore melt in the rectum to release the drug. Suitable excipients include, for example, cocoa butter and polyethylene glycols.

Compositions for inhalation typically can be provided in the form of a solution, suspension or emulsion that can be administered as a dry powder or in the form of an aerosol using a conventional propellant (e.g., dichlorodifluoromethane or trichlorofluoromethane).

Pharmaceutical compositions may be formulated for release at a pre-determined rate. Instantaneous release may be achieved, for example, via sublingual administration (i.e., administration by mouth in such a way that the active ingredient(s) are rapidly absorbed via the blood vessels under the tongue rather than via the digestive tract). Controlled release formulations (i.e., formulations such as a capsule, tablet or coated tablet that slows and/or delays release of active ingredient(s) following administration) may be administered by, for example, oral, rectal or subcutaneous implantation, or by implantation at a target site. In general, a controlled release formulation comprises a matrix and/or coating that delays disintegration and absorption in the gastrointestinal tract (or implantation site) and thereby provides a delayed action or a sustained action over a longer period. One type of controlled-release formulation is a sustained-release formulation, in which at least one active ingredient is continuously released over a period of time at a constant rate. Preferably, the therapeutic agent is released at such a rate that blood (e.g., plasma) concentrations are maintained within the therapeutic range, but below toxic levels, over a period of time that is at least 4 hours, preferably at least 8 hours, and more preferably at least 12 hours. Such formulations may generally be prepared using well known technology and administered by, for example, oral, rectal or subcutaneous implantation, or by implantation at the desired target site. Carriers for use within such formulations are biocompatible, and may also be biodegradable; preferably the formulation provides a relatively constant level of modulator release. The amount of modulator contained within a sustained release formulation depends upon, for example, the site of implantation, the rate and expected duration of release and the nature of the condition to be treated or prevented.

Controlled release may be achieved by combining the active ingredient(s) with a matrix material that itself alters release rate and/or through the use of a controlled-release coating. The release rate can be varied using methods well known in the art, including (a) varying the thickness or composition of coating, (b) altering the amount or manner of addition of plasticizer in a coating, (c) including additional ingredients, such as release-modifying agents, (d) altering the composition, particle size or particle shape of the matrix, and (e) providing one or more passageways through the coating. The amount of modulator contained within a sustained release formulation depends upon, for example, the method of administration (e.g., the site of implantation), the rate and expected duration of release and the nature of the condition to be treated or prevented.

The matrix material, which itself may or may not serve a controlled-release function, is generally any material that supports the active ingredient(s). For example, a time delay material such as glyceryl monosterate or glyceryl distearate may be employed. Active ingredient(s) may be combined with matrix material prior to formation of the dosage form (e.g., a tablet). Alternatively, or in addition, active ingredient(s) may be coated on the surface of a particle, granule, sphere, microsphere, bead or pellet that comprises the matrix material. Such coating may be achieved by conventional means, such as by dissolving the active ingredient(s) in water or other suitable solvent and spraying. Optionally, additional ingredients are added prior to coating (e.g., to assist binding of the active ingredient(s) to the matrix material or to color the solution). The matrix may then be coated with a barrier agent prior to application of controlled-release coating. Multiple coated matrix units may, if desired, be encapsulated to generate the final dosage form.

In certain embodiments, a controlled release is achieved through the use of a controlled release coating (i.e., a coating that permits release of active ingredient(s) at a controlled rate in aqueous medium). The controlled release coating should be a strong, continuous film that is smooth, capable of supporting pigments and other additives, non-toxic, inert and tack-free. Coatings that regulate release of the modulator include pH-independent coatings, pH-dependent coatings (which may be used to release modulator in the stomach) and enteric coatings (which allow the formulation to pass intact through the stomach and into the small intestine, where the coating dissolves and the contents are absorbed by the body). It will be apparent that multiple coatings may be employed (e.g., to allow release of a portion of the dose in the stomach and a portion further along the gastrointestinal tract). For example, a portion of active ingredient(s) may be coated over an enteric coating, and thereby released in the stomach, while the remainder of active ingredient(s) in the matrix core is protected by the enteric coating and released further down the GI tract. pH dependent coatings include, for example, shellac, cellulose acetate phthalate, polyvinyl acetate phthalate, hydroxypropylmethylcellulose phthalate, methacrylic acid ester copolymers and zein.

In certain embodiments, the coating is a hydrophobic material, preferably used in an amount effective to slow the hydration of the gelling agent following administration. Suitable hydrophobic materials include alkyl celluloses (e.g., ethylcellulose or carboxymethylcellulose), cellulose ethers, cellulose esters, acrylic polymers (e.g., poly(acrylic acid), poly(methacrylic acid), acrylic acid and methacrylic acid copolymers, methyl methacrylate copolymers, ethoxy ethyl methacrylates, cyanoethyl methacrylate, methacrylic acid alkamide copolymer, poly(methyl methacrylate), polyacrylamide, ammonio methacrylate copolymers, aminoalkyl methacrylate copolymer, poly(methacrylic acid anhydride) and glycidyl methacrylate copolymers) and mixtures of the foregoing. Representative aqueous dispersions of ethylcellulose include, for example, AQUACOAT® (FMC Corp., Philadelphia, Pa.) and SURELEASE® (Colorcon, Inc., West Point, Pa.), both of which can be applied to the substrate according to the manufacturer's instructions. Representative acrylic polymers include, for example, the various EUDRAGIT® (Rohm America, Piscataway, N.J.) polymers, which may be used singly or in combination depending on the desired release profile, according to the manufacturer's instructions.

The physical properties of coatings that comprise an aqueous dispersion of a hydrophobic material may be improved by the addition or one or more plasticizers. Suitable plasticizers for alkyl celluloses include, for example, dibutyl sebacate, diethyl phthalate, triethyl citrate, tributyl citrate and triacetin. Suitable plasticizers for acrylic polymers include, for example, citric acid esters such as triethyl citrate and tributyl citrate, dibutyl phthalate, polyethylene glycols, propylene glycol, diethyl phthalate, castor oil and triacetin.

Controlled-release coatings are generally applied using conventional techniques, such as by spraying in the form of an aqueous dispersion. If desired, the coating may comprise pores or channels or to facilitate release of active ingredient. Pores and channels may be generated by well known methods, including the addition of organic or inorganic material that is dissolved, extracted or leached from the coating in the environment of use. Certain such pore-forming materials include hydrophilic polymers, such as hydroxyalkylcelluloses (e.g., hydroxypropylmethylcellulose), cellulose ethers, synthetic water-soluble polymers (e.g., polyvinylpyrrolidone, cross-linked polyvinylpyrrolidone and polyethylene oxide), water-soluble polydextrose, saccharides and polysaccharides and alkali metal salts. Alternatively, or in addition, a controlled release coating may include one or more orifices, which may be formed my methods such as those described in U.S. Pat. Nos. 3,845,770; 4,034,758; 4,077,407; 4,088,864; 4,783,337 and 5,071,607. Controlled-release may also be achieved through the use of transdermal patches, using conventional technology (see, e.g., U.S. Pat. No. 4,668,232).

Further examples of controlled release formulations, and components thereof, may be found, for example, in U.S. Pat. Nos. 5,524,060; 4,572,833; 4,587,117; 4,606,909; 4,610,870; 4,684,516; 4,777,049; 4,994,276; 4,996,058; 5,128,143; 5,202,128; 5,376,384; 5,384,133; 5,445,829; 5,510,119; 5,618,560; 5,643,604; 5,891,474; 5,958,456; 6,039,980; 6,143,353; 6,126,969; 6,156,342; 6,197,347; 6,387,394; 6,399,096; 6,437,000; 6,447,796; 6,475,493; 6,491,950; 6,524,615; 6,838,094; 6,905,709; 6,923,984; 6,923,988; and 6,911,217; each of which is hereby incorporated by reference for its teaching of the preparation of controlled release dosage forms.

In addition to or together with the above modes of administration, a compound provided herein may be conveniently added to food or drinking water (e.g., for administration to non-human animals including companion animals (such as dogs and cats) and livestock). Animal feed and drinking water compositions may be formulated so that the animal takes in an appropriate quantity of the composition along with its diet. It may also be convenient to present the composition as a premix for addition to feed or drinking water.

Compound(s) provided herein are generally administered in a therapeutically effective amount. Preferred systemic doses are no higher than 50 mg per kilogram of body weight per day (e.g., ranging from about 0.001 mg to about 50 mg per kilogram of body weight per day), with oral doses generally being about 5-20 fold higher than intravenous doses (e.g., ranging from 0.01 to 40 mg per kilogram of body weight per day).

The amount of active ingredient that may be combined with the carrier materials to produce a single dosage unit will vary depending, for example, upon the patient being treated and the particular mode of administration. Dosage units will generally contain from about 10 μg to about 500 mg of an active ingredient. In certain embodiments, the dosage unit contains an amount of the compound that is sufficient to effect a decrease in the patient's caloric intake (i.e., an appetite-suppressing amount) following single dose administration or repeated administration according to a predetermined regimen. Optimal dosages may be established using routine testing, and procedures that are well known in the art.

Pharmaceutical compositions may be used for treating a condition responsive to CB1 modulation. Such conditions include, for example:
appetite disorders (e.g., binge eating disorder, bulimia, anorexia);
obesity and complications associated therewith, including left ventricular hypertrophy;
weight loss or control (e.g., reducing calorie or food intake and/or appetite suppression); and
addictive disorders such as:
   alcohol dependency (e.g., alcohol abuse, addiction and/or dependency including treatment for abstinence, craving reduction and relapse prevention of alcohol intake);
   nicotine dependency (e.g., smoking addiction, cessation and/or dependency including treatment for craving reduction and relapse prevention of tobacco smoking); and
   drug dependency (e.g., chronic treatment with or abuse of drugs such as opioids, barbiturates, cannabis, cocaine, amphetamines, phencyclide, hallucinogens, and/or benzodiazepines).
metabolic disorders (e.g., type 2 diabetes, dyslipidemia and metabolic syndrome);
bone loss (e.g., resulting from estrogen deficiency).

Other conditions responsive to CB1 modulation include CNS disorders (e.g., anxiety, depression, panic disorder, bipolar disorder, psychosis, schizophrenia, behavioral addiction, dementia (including memory loss, Alzheimer's disease, dementia of aging, vascular dementia, mild cognitive impairment, age-related cognitive decline, and mild neurocognitive disorder), attention deficit disorder (ADD/ADHD), stress, amnesia, cognitive disorders, memory disorders, neurodegeneration, cerebellar and spinocerebellar disorder, cranial trauma, cerebral vascular accidents, obsessive-compulsive disorder, senile dementia, impulsivity), thymic disorders, septic shock, Tourette's syndrome, Huntington's chorea, Raynaud's syndrome, peripheral neuropathy, diabetes (type II or non insulin dependent), glaucoma, migraine, seizure disorders, epilepsy, locomotor disorders (movement disorders induced by medicaments, dyskinesias or Parkinson's disease), respiratory disorders (such as asthma), gastrointestinal disorders (e.g., dysfunction of gastrointestinal motility or intestinal propulsion, constipation, chronic intestinal pseudo-obstruction, irritable bowel syndrome, Crohn's disease), liver cirrhosis, vomiting, diarrhea, ulcer, multiple sclerosis, cardiovascular disorder, dystonia, endotoxemic shocks, hemorrhagic shocks, hypotension, insomnia, a disorder of the endocrine system, urinary or bladder disorders, cancer, infectious disease, inflammation, infection, cancer, neuroinflammation (such as atherosclerosis), Guillain-Barre syndrome, viral encephalitis, cranial trauma, sepsis, hair loss or a reproductive disorder. In certain embodiments, the condition responsive to CB1 modulation is an appetite disorder, obesity, an addictive disorder, asthma, liver cirrhosis, sepsis, irritable bowel disease, Crohn's disease, depression, schizophrenia, a memory disorder, a cognitive disorder, a movement disorder, a metabolic disorder and/or bone loss.

Certain pharmaceutical compositions provided herein comprise a first agent that is a compound as provided herein in combination with a second agent that differs in structure from the first agent and is suitable for treating the condition of interest. In certain embodiments, the second agent is not a CB1 antagonist as provided herein. In certain embodiments, the second agent is suitable for treating an appetite disorder, obesity, an addictive disorder, asthma, liver cirrhosis, sepsis, irritable bowel disease, Crohn's disease, depression, schizophrenia, a memory disorder, a cognitive disorder, a movement disorder and/or bone loss. Representative second agents for use within such pharmaceutical compositions include anti-obesity agents such as MCH receptor antagonists, apo-B/MTP inhibitors, 11β-hydroxy steroid dehydrogenase-1 inhibitors, peptide YY₃-36 or an analog thereof, MCR-4 agonists, CCK-A agonists, monoamine reuptake inhibitors, sympathomimetic agents, β₃ adrenergic receptor agonists, dopamine agonists, melanocyte-stimulating hormone receptor analogues, 5-HT2c receptor agonists, leptin or an analog thereof, leptin receptor agonists, galanin antagonists, lipase inhibitors, bombesin agonists, neuropeptide-Y receptor antagonists, thyromimetic agents, dehydroepiandrosterone or analog thereof, glucocorticoid receptor antagonists, orexin receptor antagonists, glucagon-like peptide-1 receptor agonists, ciliary neurotrophic factors, human agouti-related protein antagonists, ghrelin receptor antagonists, histamine 3 receptor antagonists, and neuromedin U receptor agonists. Such agents include, for example, phentermine, orlistat and sibutramine (e.g., sibutramine HCl monohydrate, sold as Meridia® (Abbott Laboratories)).

Representative second agents suitable for treating an addictive disorder include, for example, Methadone, LAAM (levo-alpha-acetyl-methadol), naltrexone (e.g., ReVia™), ondansetron (e.g., Zofran®), sertraline (e.g., Zoloft®), fluoxetine (e.g., Prozac®), diazepam (e.g., Valium®) and chlordiazepoxide (e.g., Librium), varenicline and buproprion (e.g., Zyban® or Wellbutrin®). Other representative second agents for use within the pharmaceutical compositions provided herein include nicotine receptor partial agonists, opioid antagonists and/or dopaminergic agents.

Pharmaceutical compositions may be packaged for treating conditions responsive to CB1 modulation (e.g., treatment of appetite disorder, obesity and/or addictive disorder, or other disorder indicated above). Packaged pharmaceutical preparations generally comprise a container holding a pharmaceutical composition as described above and instructions (e.g., labeling) indicating that the composition is to be used for treating a condition responsive to CB1 modulation in a patient. In certain embodiments, a packaged pharmaceutical preparation comprises one or more compounds provided herein and one or more additional agents in the same package, either in separate containers within the package or in the same container (i.e., as a mixture). Preferred mixtures are formulated for oral administration (e.g., as pills, capsules, tablets or the like). In certain embodiments, the package comprises a label bearing indicia indicating that the components are to be taken together for the treatment of an appetite disorder, obesity, an addictive disorder, asthma, liver cirrhosis, sepsis, irritable bowel disease, Crohn's disease, depression, schizophrenia, a memory disorder, a cognitive disorder, a movement disorder, a metabolic disorder and/or bone loss.

Methods of Use

Within certain aspects, the present invention provides methods for treating a condition responsive to CB1 modulation in a patient and/or for appetite suppression. The patient may be afflicted with such a condition, or may be free of symptoms but considered at risk for developing such a condition. A condition is “responsive to CB1 modulation” if the condition or symptom(s) thereof are alleviated, attenuated, delayed or otherwise improved by modulation of CB1 activity. Such conditions include, for example, appetite disorders, obesity, addictive disorders, asthma, liver cirrhosis, sepsis, irritable bowel disease, Crohn's disease, depression, schizophrenia, memory disorders, cognitive disorders, movement disorders, metabolic disorders and bone loss, as well as other disorders indicated above. In general, such methods comprise administering to the patient a therapeutically effective amount of at least one compound as provided herein.

It will be apparent that compounds provided herein may be administered alone or in combination with one or more additional agents that are suitable for treating the disorder of interest. Within combination therapy, the compound(s) and additional agent(s) may be present in the same pharmaceutical composition, or may be administered separately in either order. Representative additional agents for use in such methods include the second agents described above.

Suitable dosages for compounds provided herein (either alone or within such combination therapy) are generally as described above. Dosages and methods of administration of any additional agent(s) can be found, for example, in the manufacturer's instructions or in the Physician's Desk Reference. In certain embodiments, combination administration results in a reduction of the dosage of the additional agent required to produce a therapeutic effect (i.e., a decrease in the minimum therapeutically effective amount). Thus, preferably, the dosage of additional agent in a combination or combination treatment method of the invention is less than the maximum dose advised by the manufacturer for administration of the agent without combination with a compound of Formula I. More preferably this dose is less than ¾, even more preferably less than ½, and highly preferably less than 1/4 of the maximum dose, while most preferably the dose is less than 10% of the maximum dose advised by the manufacturer for administration of the agent(s) when administered without combination administration as described herein. It will be apparent that the dose of compound as provided herein needed to achieve the desired effect may similarly be affected by the dose and potency of the additional agent.

Administration to the patient can be by way of any means discussed above, including oral, topical, nasal or transdermal administration, or intravenous, intramuscular, subcutaneous, intrathecal, epidural, intracerebroventrilcular or like injection. Oral administration is preferred in certain embodiments (e.g., formulated as pills, capsules, tablets or the like).

Treatment regimens may vary depending on the compound used and the particular condition to be treated. In general, a dosage regimen of 4 times daily or less is preferred, with 1 or 2 times daily particularly preferred. It will be understood, however, that the specific dose level and treatment regimen for any particular patient will depend upon a variety of factors including the activity of the specific compound employed, the age, body weight, general health, sex, diet, time of administration, route of administration, and rate of excretion, drug combination and the severity of the particular disease undergoing therapy. Dosages are generally as described above; in general, the use of the minimum dose sufficient to provide effective therapy is preferred. Patients may generally be monitored for therapeutic effectiveness using medical or veterinary criteria suitable for the condition being treated or prevented. For example, treatment of obesity is considered to be effective if it results in a statistically significant decrease in weight or BMI.

Within separate aspects, the present invention provides a variety of non-pharmaceutical in vitro and in vivo uses for the compounds provided herein. For example, such compounds may be labeled and used as probes for the detection and localization of CB1 (in samples such as cell preparations or tissue sections, preparations or fractions thereof). In addition, compounds provided herein that comprise a suitable reactive group (such as an aryl carbonyl, nitro or azide group) may be used in photoaffinity labeling studies of receptor binding sites. In addition, compounds provided herein may be used as positive controls in assays for receptor activity, as standards for determining the ability of a candidate agent to bind to CB1, or as radiotracers for positron emission tomography (PET) imaging or for single photon emission computerized tomography (SPECT). Such methods can be used to characterize CB1 receptors in living subjects. For example, a compound may be labeled using any of a variety of well known techniques (e.g., radiolabeled with a radionuclide such as tritium, as described herein), and incubated with a sample for a suitable incubation time (e.g., determined by first assaying a time course of binding). Following incubation, unbound compound is removed (e.g., by washing), and bound compound detected using any method suitable for the label employed (e.g., autoradiography or scintillation counting for radiolabeled compounds; spectroscopic methods may be used to detect luminescent groups and fluorescent groups). As a control, a matched sample containing labeled compound and a greater (e.g., 10-fold greater) amount of unlabeled compound may be processed in the same manner. A greater amount of detectable label remaining in the test sample than in the control indicates the presence of CB1 in the sample. Detection assays, including receptor autoradiography (receptor mapping) of CB1 in cultured cells or tissue samples may be performed as described by Kuhar in sections 8.1.1 to 8.1.9 of Current Protocols in Pharmacology (1998) John Wiley & Sons, New York.

Compounds provided herein may further be used within assays for the identification of other non-competitive antagonists of CB1. In general, such assays are standard competition binding assays, in which a labeled compound as provided herein is displaced by a test compound. Briefly, such assays are performed by: (a) contacting CB1 with a labeled (e.g., radiolabeled) compound and a test compound, under conditions that permit binding to CB1 (b) removing unbound labeled compound and unbound test compound; (c) detecting a signal that corresponds to the amount of bound, labeled compound; and (d) comparing the signal to a reference signal that corresponds to the amount of bound labeled compound in a similar assay performed in the absence of test compound. In practice, the reference signal and the signal described in step (c) are generally obtained simultaneously (e.g., the assays are performed in different wells of the same plate); in addition, multiple concentrations of test compound are generally assayed. Non-competitive antagonist activity can be confirmed for test compounds that decrease the amount of bound, labeled compound using procedures described herein.

The following Examples are offered by way of illustration and not by way of limitation. Unless otherwise specified all reagents and solvent are of standard commercial grade (available, for example, from Sigma-Aldrich, St. Louis, Mo.)) and are used without further purification. Using routine modifications, the starting materials may be varied and additional steps employed to produce other compounds provided herein.

### EXAMPLES

Mass spectroscopy data in the following Examples is Electrospray MS, obtained in positive ion mode using a Micromass Time-of-Flight LCT (Micromass, Beverly Mass.), equipped with a Waters 600 pump (Waters Corp.; Milford, Mass.), Waters 996 photodiode array detector, and a Gilson 215 autosampler (Gilson, Inc.; Middleton, Wis.). MassLynx (Advanced Chemistry Development, Inc; Toronto, Canada) version 4.0 software with OpenLynx Global Server™, OpenLynx™ and AutoLynx™ processing is used for data collection and analysis. MS conditions are as follows: capillary voltage=3.5 kV; cone voltage=30 V, desolvation and source temperature=350° C. and 120° C., respectively; mass range=181-750 with a scan time of 0.22 seconds and an interscan delay of 0.05 min.

Sample volume of 1 microliter is injected onto a 50×4.6 mm Chromolith SpeedROD RP-18e column (Merck KGaA, Darmstadt, Germany), and eluted using a 2-phase linear gradient at a flow rate of 6 ml/min. Sample is detected using total absorbance count over the 220-340 nm UV range. The elution conditions are: Mobile Phase A—95% water, 5% MeOH with 0.05% TFA; Mobile Phase B—5% water, 95% MeOH with 0.025% TFA. The following gradient is used: 0-0.5 min 10-100% B, hold at 100% B to 1.2 min, return to 10% B at 1.21 min. Inject to inject cycle is 2.15 min.

### Example 1

### SYNTHESIS OF 1-[5-(4-CHLOROPHENYL)-6-(2-CHLOROPHENYL)-PYRAZIN-2-YL]-4-ETHYLAMINO-PIPERIDINE-4-CARBOXYLIC ACID AMIDE

### STEP 1. 1-(6-CHLORO-PYRAZIN-2-YL)-4-ETHYLAMINO-PIPERIDINE-4-CARBOXYLIC ACID AMIDE

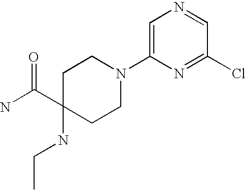

A mixture of 2,6-dichloro-pyrazine (3.3 g, 22 mmol), 4-ethylamino-piperidine-4-carboxylic acid amide (3.85 g, 22.5 mmol) and K₂CO₃ (3.7 g, 26 mmol)- in CH₃CN (30 mL) is heated at 100° C. for 1 h. The reaction mixture is cooled and evaporated under reduced pressure. The residue is mixed with water and filtered to collect a white solid. ¹H NMR (CDCl₃): 7.99 (s, 1H), 7.78 (s, 1H), 7.08 (br, 1H), 5.37 (br, 1H), 3.91 (m, 2H), 3.46 (m, 2H), 2.56 (q, 2H), 2.17 (m, 2H), 1.70 (m, 2H), 1.12 (t, 3H).

### STEP 2. 1-(5-BROMO-6-CHLORO-PYRAZIN-2-YL)-4-ETHYLAMINO-PIPERIDINE-4-CARBOXYLIC ACID AMIDE

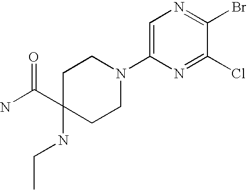

A mixture of 1-(6-chloro-pyrazin-2-yl)-4-ethylamino-piperidine-4-carboxylic acid amide (449 mg, 1.58 mmol) and NBS (300 mg, 1.68 mmol) in CHCl₃ (5 mL) is stirred at rt overnight. The reaction mixture is diluted with CH₂Cl₂ (20 mL), washed with aqueous Na₂CO₃ solution and water, and concentrated under vacuum. The residue is purified on silica gel column with 5% MeOH in CH₂Cl₂ to give the title compound. ¹H NMR (CDCl₃): 7.77 (s, 1H), 7.06 (br, 1H), 5.34 (br, 1H), 3.85 (m, 2H), 3.48 (m, 2H), 2.56 (q, 2H), 2.15 (m, 2H), 1.71 (m, 2H), 1.11 (t, 3H).

### STEP 3. 1-[5-(4-CHLOROPHENYL)-6-(2-CHLOROPHENYL)-PYRAZIN-2-YL]-4-ETHYLAMINO-PIPERIDINE-4-CARBOXYLIC ACID AMIDE

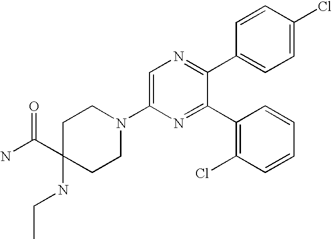

A mixture of 1-(5-bromo-6-chloro-pyrazin-2-yl)-4-ethylamino-piperidine-4-carboxylic acid amide (29 mg, 0.08 mmol), 4-chlorophenylboronic acid (13 mg, 0.08 mmol), Na₂CO₃ (34 mg, 0.32 mmol), Pd(PPh₃)₄ (5 mg), water (0.3 mL) and dioxane (0.8 mL) is degassed with argon for 10 min, then sealed and heated at 105° C. for 1 h. The reaction mixture is cooled, and 2-chlorophenylboronic acid (13 mg, 0.08 mmol) is added. The reaction mixture is sealed again and heated at 105° C. for 1 h. The mixture is cooled, diluted with water (1 mL) and 1 N NaOH (0.5 mL), and extracted with EtOAc (2 mL). The extract is washed once with water and concentrated under vacuum. The residue is purified by PTLC (5% MeOH in CH₂Cl₂) to produce the title compound. ¹H NMR (CDCl₃): 8.23 (s, 1H), 7.13-7.35 (m, 8H), 7.12 (br, 1H), 5.58 (br, 1H), 4.08 (m, 2H), 3.46 (m, 2H), 2.56 (q, 2H), 2.20 (m, 2H), 1.74 (m, 2H), 1.11 (t, 3H).

### Example 2

### SYNTHESIS OF 1-[5-(4-CHLOROPHENYL)-6-(2,4-DICHLOROPHENYL)-PYRAZIN-2-YL-4-ETHYLAMINO-PIPERIDINE-4-CARBOXYLIC ACID AMIDE

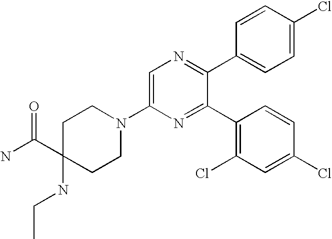

This compound is prepared as described in Example 1. LC-MS: m/z expected 504.8; found 505.0 (MH⁺).

### Example 3

### SYNTHESIS OF 1-[5-(4-CHLOROPHENYL)-6-(2,4-DICHLOROPHENYL)-3-(METHYLAMINO)-PYRAZIN-2-YL]-4-ETHYLAMINO-PIPERIDINE-4-CARBOXAMIDE

### STEP 1.1-[3-BROMO-5-(4-CHLOROPHENYL)-6-(2,4-DICHLOROPHENYL)-PYRAZIN-2-YL]-4-ETHYLAMINO-PIPERIDINE-4-CARBOXYLIC ACID AMIDE

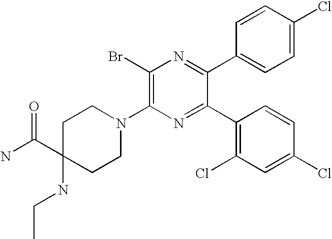

A mixture of 1-[5-(4-chlorophenyl)-6-(2,4-dichlorophenyl)-pyrazin-2-yl]-4-ethylamino-piperidine-4-carboxylic acid amide (20 mg, 0.04 mmol) and NBS (11 mg, 0.06 mmol) in CHCl₃ (1 mL) is stirred at rt for 3 h. The reaction mixture is diluted with CH₂Cl₂ (1 mL), washed with aqueous Na₂CO₃ and water, and concentrated to give the title compound, which is used in the next step without further purification.

### STEP 2. 1-[5-(4-CHLOROPHENYL)-6-(2,4-DICHLOROPHENYL)-3-METHYLAMINO-PYRAZIN-2-YL]-4-ETHYLAMINO-PIPERIDINE-4-CARBOXYLIC ACID AMIDE

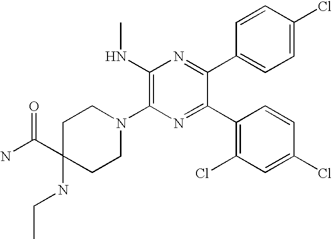

A mixture of the product from Step 1 and methylamine (4M in NMP, 1 mL) in a sealed tube is heated at 130° C. overnight. The mixture is diluted with CH₂Cl₂, and washed with water (5 times) and concentrated. The residue is purified by PTLC (5% MeOH in CH₂Cl₂) to produce the title compound. LC-MS: m/z expected 533.9; found 534.05 (MH⁺), Rt=1.60 min.

### Example 4

### SYNTHESIS OF 1-[5-(4-CHLOROPHENYL)-6-(3-CHLORO-PYRIDIN-4-YL)-PYRAZIN-2-YL]-4-ETHYLAMINO-PIPERIDINE-4-CARBOXYLIC ACID AMIDE

### STEP 1. 1-[6-CHLORO-5-(4-CHLOROPHENYL)-PYRAZIN-2-YL]-4-ETHYLAMINO-PIPERIDINE-4-CARBOXYLIC ACID AMIDE

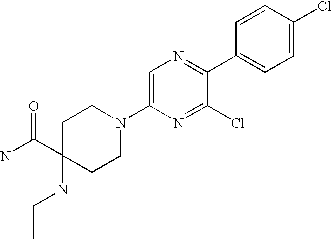

A mixture of 1-(5-bromo-6-chloro-pyrazin-2-yl)-4-ethylamino-piperidine-4-carboxylic acid amide (Example 1; 580 mg, 1.6 mmol), 4-chlorophenylboronic acid (263 mg, 1.6 mmol), Na₂CO₃ (353 mg, 3.2 mmol), Pd(PPh₃)₄ (80 mg), water (3.4 mL) and dioxane (17 mL) is degassed with argon for 10 min, then sealed and heated at 105° C. for 6 h. The mixture is cooled, diluted with water and I N NaOH, and extracted with EtOAc. The extract is washed once with water and concentrated under vacuum. The residue is purified by PTLC (5% MeOH in CH₂Cl₂) to produce the title compound.

### STEP 2. 1-[5-(4-CHLOROPHENYL)-6-(3-CHLORO-PYRIDIN-4-YL)-PYRAZIN-2-YL]-4-ETHYLAMINO-PIPERIDINE-4-CARBOXYLIC ACID AMIDE

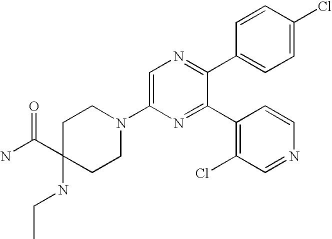

A mixture of 1-[6-chloro-5-(4-chlorophenyl)-pyrazin-2-yl]-4-ethylamino-piperidine-4-carboxylic acid amide (26 mg, 0.065 mmol), 3-chloro-pyridine-4-yl boronic acid (31 mg, 0.2 mmol), Na₂CO₃ (43 mg, 0.41 mmol), Pd(PPh₃)₄ (8 mg), water (0.4 mL) and dioxane (1.0 mL) is degassed with argon for 10 min, then sealed and heated at 130° C. for overnight. The mixture is cooled, diluted with water (1 mL) and 1 N NaOH (0.5 mL), and extracted with EtOAc (2 mL). The extract is washed once with water and concentrated under vacuum. The residue is purified by PTLC (5% MeOH in CH₂Cl₂) to produce the title compound. LC-MS: m/z expected 471.4; found 472.2 (MH⁺), Rt=1.43 min.

### Example 5

### SYNTHESIS OF TERT-BUTYL 4-[5-(4-CHLOROPHENYL)-6-(3-CHLORO-PYRIDIN-4-YL)-2-PYRAZINYL]-1-PIPERAZINECARBOXYLATE

### STEP 1. tert-BUTYL-4-(6-CHLORO-PYRAZIN-2-YL)-PIPERAZINE-1-CARBOXYATE

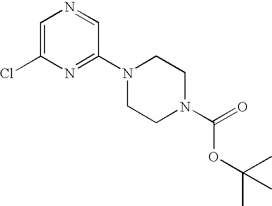

A mixture of 2,6-dichloro-pyrazine (15 g, 0.1 mol), piperazine-1-carboxylic acid tert-butyl ester (19.7 g, 0.1 mmol) and K₂CO₃ (47 g, 0.3 mol) in CH₃CN (150 mL) is stirred at rt for 18 h and heated at 70° C. for 2 h. The reaction mixture is evaporated under vacuum, diluted with water (150 mL), and extracted with EtOAc (3×75 mL). The combined extracts are washed with brine (100 mL), dried over MgSO₄ and concentrated under vacuum to give the title compound as a colorless viscous oil.

### STEP 2. TERT-BUTYL-4-(5-BROMO-6-CHLORO-PYRAZIN-2-YL)-PIPERAZINE-1-CARBOXYLATE

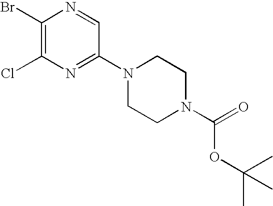

A mixture of tert-butyl-4-(6-chloro-pyrazin-2-yl)-piperazine-1-carboxylate (29.8 g, 0.1 mol) and NBS (17.8 g, 0.1 mol) in CHCl₃ (250 mL) is stirred at rt overnight. The reaction mixture is washed with diluted aqueous Na₂CO₃ solution and brine, dried and concentrated under vacuum and purified by flash column chromatography using 5 to 40% EtOAc in hexane to afford the title compound as a white solid. ¹H NMR (CDCl₃): 7.76 (s, 1H), 3.56 (m, 8H), 1.48 (s, 9H).

### STEP 3. tert-BUTYL-4-[6-CHLORO-5-(4-CHLOROPHENYL)-PYRAZIN-2-YL]-PIPERAZINE-1-CARBOXYATE

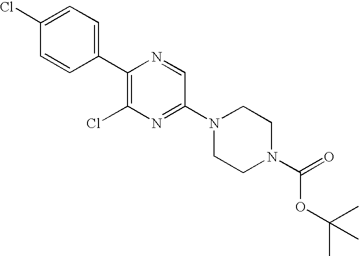

The product of step 2 is converted to the title compound via the Suzuki coupling procedure described in Example 4, Step 1. ¹H NMR (CDCl₃): 8.08 (s, 1H), 7.67 (d, 2H), 7.42 (d, 2H), 3.64 (m, 4H), 3.58 (m, 4H), 1.49 (s, 9H).

### STEP 4. tert-BUTYL-4-[5-(4-CHLOROPHENYL)-6-(3-CHLORO-PYRIDIN-4-YL)-2-PYRAZINYL]-1-PIPERAZINECARBOXYLATE

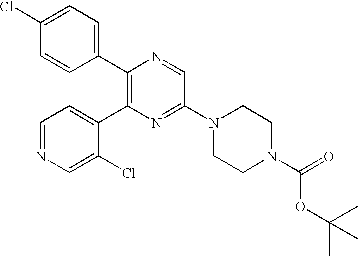

The product of step 3 is converted to the title compound via the Suzuki coupling procedure described in Example 4, Step 2. ¹H NMR (CDCl₃) 8.58 (s, 1H), 8.51 (d, 1H), 8.26 (s, 1H), 7.26 (m, 5H), 3.67 (m, 4H), 3.60 (m, 4H), 1.48 (s, 9H).

### Example 6

### SYNTHESIS OF 2-(4-CHLOROPHENYL)-3-(3-CHLORO-PYRIDIN-4-YL)-5-(1-PIPERAZINYL)-PYRAZINE

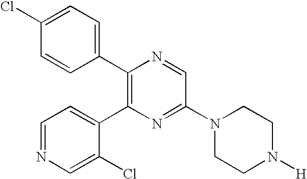

A solution of tert-butyl-4-[5-(4-chlorophenyl)-6-(3-chloro-pyridin-4-yl)-2-pyrazinyl]-1-piperazinecarboxylate (475 mg, 0.98 mmol) and TFA (2 mL) in CH₂Cl₂ is stirred at rt overnight. The reaction mixture is evaporated under vacuum, basified with aqueous Na₂CO₃ solution, and extracted with EtOAc. The extract is washed with brine, dried and concentrated to give the title compound.

### Example 7

### SYNTHESIS OF 2-(4-CHLOROPHENYL)-3-(3-CHLORO-PYRIDIN-4-YL)-5-[4-(ISOPROPYLSULFONYL)-1-PIPERAZINYL]-PYRAZINE

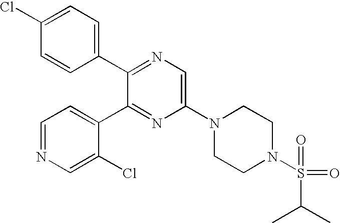

To solution of 2-(4-chlorophenyl)-3-(3-chloro-pyridin-4-yl)-5-(1-piperazinyl)-pyrazine (2 mg, 0.057 mmol) and ethyl-diisopropyl-amine (22 mg, 0.17 mmol) in CH₂Cl₂ (1 mL) is added isopropylsulfonyl chloride (8.5 mg, 0.06 mmol). The reaction mixture is stirred at rt for 15 min, washed with water, concentrated and purified by PTLC (5% MeOH in CH₂Cl₂) to produce the title compound. LC-MS: m/z expected 492.4; found 493.0 (MH⁺), Rt=1.69 min.

### Example 8

### SYNTHESIS OF 2-(4-CHLOROPHENYL)-3-(3-CHLORO-PYRIDIN-4-YL)-5-(4-ISOBUTYRYL-1-PIPERAZINYL)-PYRAZINE

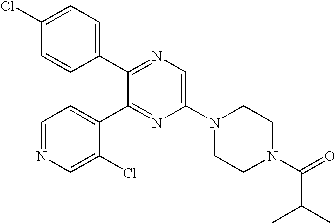

This compound is prepared by reaction of 2-(4-chlorophenyl)-3-(3-chloro-pyridin-4-yl)-5-(1-piperazinyl)-pyrazine with isobutyryl chloride following the procedure given in the previous example. LC-MS: m/z expected 456.4; found 457.2 (MH⁺), Rt=1.66 min.

### Example 9

### SYNTHESIS OF 2-(4-CHLOROPHENYL)-3-(3-CHLORO-PYRIDIN-4-YL)-5-(4-ISOBUTYL-1-PIPERAZINYL)-PYRAZINE

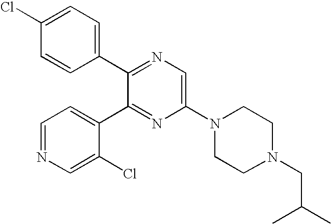

A mixture of 2-(4-chlorophenyl)-3-(3-chloro-pyridin-4-yl)-5-(1-piperazinyl)-pyrazine (14 mg, 0.036 mmol), 2-methyl-propionaldehyde (4 mg, 0.056 mmol) and NaBH(OAc)₃ (22 mg, 0.1 mmol) in CH₂Cl₂ (1 mL) is stirred at rt for 2 h. The mixture is washed with aqueous Na₂CO₃ solution, concentrated under vacuum, and purified by PTLC (5% MeOH in CH₂Cl₂) to produce the title compound. LC-MS: m/z expected 442.4; found 443.2 (MH⁺), Rt=1.42 min.

### Example 10

### SYNTHESIS OF 2-(4-CHLOROPHENYL)-3-(3-CHLORO-PYRIDIN-4-YL)-5-(4-ISOBUTYL-1-PIPERAZINYL)-PYRAZINE

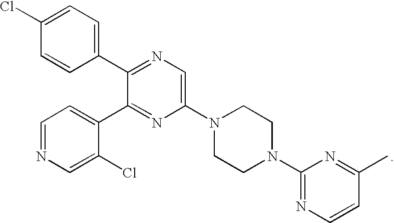

A mixture of 2-(4-chlorophenyl)-3-(3-chloro-pyridin-4-yl)-5-(1-piperazinyl)-pyrazine (14 mg, 0.036 mmol), 2-chloro-4-methyl-pyrimidine (18 mg, 0.14 mmol) and K₂CO₃ (30 mg, 0.22 mmol) in CH₃CN (1 mL) is stirred at reflux overnight. The mixture is washed with aqueous Na₂CO₃ solution, concentrated under vacuum, and purified by PTLC (5% MeOH in CH₂Cl₂) to produce the title compound. LC-MS: m/z expected 478.4; found 479.2 (MH⁺), Rt=1.84 min.

### Example 11

### SYNTHESIS OF 4-[3-(3-CHLORO-PYRIDIN-4-YL)-5-(1,1-DIOXIDO-4-THIOMORPHOLINYL)-PYRAZIN-2-YL]-BENZONITRILE

### STEP 1. 4-(6-CHLORO-PYRAZIN-2-YL)-THIOMORPHOLINE

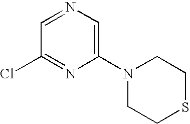

A mixture of 2,6-dichloro-pyrazine (6.0 g, 0.4 mol), thiomorpholine (4.2 g, 0.4 mmol) and K₂CO₃ (11.2 g, 0.8 mol) in CH₃CN (50 mL) is heated at 80° C. overnight. The reaction mixture is evaporated under vacuum, diluted with water (150 mL), filtered and dried to give the title compound as a tan solid.

### STEP 2. 4-(5-BROMO-6-CHLORO-PYRAZIN-2-YL)-THIOMORPHOLINE

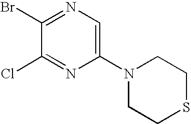

A mixture of 4-(6-chloro-pyrazin-2-yl)-thiomorpholine (4.2 g, 0.02 mol) and NBS (5.2 g, 0.029 mol) in CHCl₃ (100 mL) is stirred at rt overnight. The reaction mixture is washed with aqueous Na₂CO₃ and brine, dried, concentrated under vacuum, and purified by flash column chromatography using 100% CH₂Cl₂ to afford the title compound.

### STEP 3. 4-(5-BROMO-6-CHLORO-PYRAZIN-2-YL)-THIOMORPHOLINE 1,1-DIOXIDE

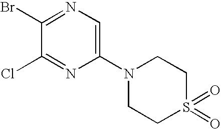

A mixture of 4-(5-bromo-6-chloro-pyrazin-2-yl)-thiomorpholine (2.47 g, 8.4 mmol) and m-CPBA (77%, 3.95 g, 17.6 mmol) in CH₂Cl₂ (50 mL) is stirred at rt overnight. The reaction mixture is washed with aqueous Na₂CO₃ and water, concentrated and purified by flash column chromatography using 2.5% MeOH in CH₂Cl₂ to afford the title compound. ¹H NMR (CDCl₃): 7.90 (s, 1H), 4.16 (m, 4H), 3.09 (m, 4H).

### STEP 4. 4-[3-CHLORO-5-(1,1-DIOXIDOTHIOMORPHOLIN4-YL)- PYRAZIN-2-YL]BENZONITRILE

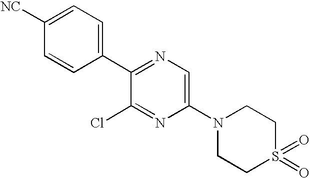

The product of step 3 is converted to the title compound with 4-cyanophenyl boronic acid via the Suzuki coupling procedure used in Example 4, Step 1. LC-MS: m/z expected 348.8; found 350.2 (MH⁺), Rt=1.38 min.

### STEP 5. 4-[3-(3-CHLORO-PYRIDIN-4-YL)-5-(1,1-DIOXIDOTHIOMORPHOLIN4-YL)-PYRAZIN-2-YL]BENZONITRILE

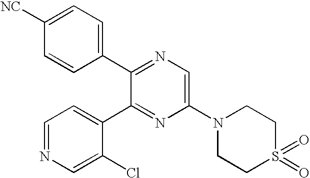

The product of step 4 is converted to the title compound via the Suzuki coupling procedure used in Example 4, Step 2. LC-MS: m/z expected 425.9; found 427.2 (MH⁺), Rt=1.47 min.

### Example 12

### SYNTHESIS OF tert-BUTYL-4-[5-BROMO-6-(4-CHLOROPHENYL)-PYRAZIN-2-YL]-PIPERAZINE-1-CARBOXYLATE

### STEP 1. tert-BUTYL-4-(6-CHLORO-PYRAZIN-2-YL)-PIPERAZINE-1-CARBOXYATE

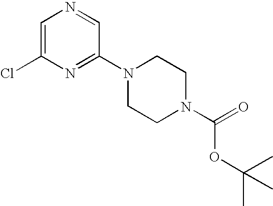

tert-Butyl-1-piperazine carboxylate (8.8 g, 47.5 mmol) is added to a suspension of 2,6-dichloro-pyrazine (6.4 g, 43.2 mmol) and potassium carbonate (18.0 g, 130 mmol) in anhydrous acetonitrile (100 mL). After addition, the reaction mixture is stirred at 90° C. for 3 h, and the reaction is monitored by TLC. The reaction mixture is cooled to rt and then diluted with ether (100 mL) and water (50 mL). The organic layer is separated and the aqueous layer is extracted with ether (2×100 mL). The combined organic layers are dried over anhydrous MgSO₄ and the solvents are removed in vacuo to give the title compound as a white solid.

### STEP 2. tert-BUTYL-4-[6-(4-CHLOROPHENYL)-PYRAZIN-2-YL)-PIPERAZINE-1-CARBOXYATE

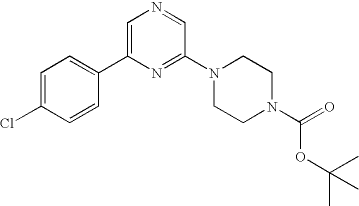

4-Chlorophenylboronic acid (2.1 g, 13.5 mmol) is added to a solution of 2 tert-butyl-4-(6-chloro-pyrazin-2-yl)-piperazine-1-carboxylate (4.0 g, 13.5 mmol) in a mixture of dioxane (60 mL), water (10 mL) and 2N sodium carbonate (13.5 mL, 27 mmol). The solution is degassed by bubbling nitrogen through the solution for 10 min and is then charged with Pd(PPh₃)₄ (780 mg, 5 mol %). After addition, the reaction mixture is stirred at 100° C. for 6 h, and the reaction is monitored by TLC. The reaction mixture is cooled to rt and then diluted with ether (100 mL) and water (50 mL). The organic layer is separated and the aqueous layer is extracted with ether (2×50 mL). The combined organic layers are dried over anhydrous MgSO₄ and the solvents are removed in vacuo to give the title compound as an off-white solid.

### STEP 3. tert-BUTYL-4-[5-BROMO-6-(4-CHLOROPHENYL)-PYRAZIN-2-YL]-PIPERAZINE-1-CARBOXYATE

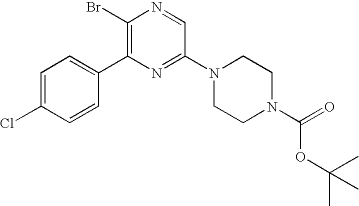

NBS (1.94 g, 10.88 mmol) is added portionwise to a solution of tert-butyl-4-[6-(4-chloro-phenyl)-pyrazin-2-yl)-piperazine-1-carboxylate (4.08 g, 10.88 mmol) in CHCL₃ (50 mL) at 0° C. The reaction is stirred at 0° C. for 1 h, and then allowed to warm to rt and stirred at rt for 2 h. The reaction mixture is diluted with ether (200 mL), washed with water (2×50 mL) and then with brine, and dried over anhydrous MgSO₄. The solvents are removed in vacuo. Purification using flash column chromatography (silica gel) with 25% EtOAc in hexane as eluent gives the title compound as a pale yellow solid.

### Example 13

### GENERAL PROCEDURE FOR THE SUZUKI REACTION OF ARYL BORONIC ACIDS WITH tert-BUTYL-4-[5-BROMO-6-(4-CHLOROPHENYL)-PYRAZIN-2-YL]-PIPERAZINE-1-CARBOXYLATE

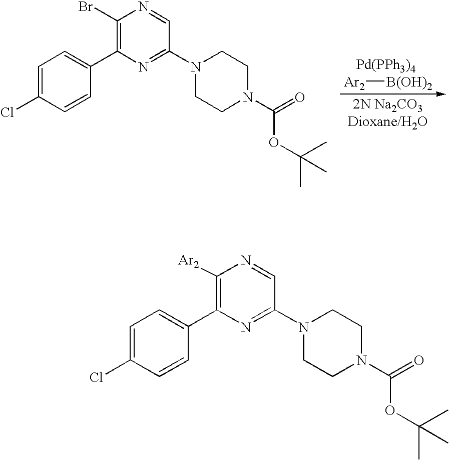

Arylboronic acid (0.21 mmol) is added to a solution of tert-butyl-4-[5-bromo-(6-chlorophenyl)-pyrazin-2-yl]-piperazine-1-carboxylate (80 mg, 0.18 mmol) in a mixture of dioxane (3 mL), water (0.5 mL) and 2N sodium carbonate solution (0.18 mL, 0.36 mmol). The solution is degassed by bubbling nitrogen through the solution for 2 min and is then charged with Pd(PPh₃)₄ (10 mg, 5 mol %). After addition, the reaction mixture is stirred at 100° C. for 16 h. The reaction mixture is cooled to rt and then diluted with ether (10 mL) and water (5 mL). The organic layer is separated and the aqueous layer is extracted with ether (2×5 mL). The combined organic layers are dried over anhydrous MgSO₄ and the solvents are removed in vacuo. Purification by PTLC gives the corresponding arylation product.

### Example 14

### SYNTHESIS OF tert-BUTYL-4-[6-(4-CHLOROPHENYL)-5-(3-FLUOROPYRIDIN-4-YL)-PYRAZIN-2-YL]-PIPERAZINE-1-CARBOXYLATE

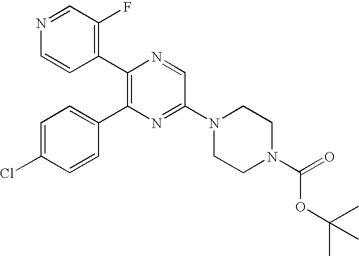

This compound is prepared as described in Example 13, using 3-fluoropyridine-4-boronic acid, and is obtained as a pale yellow solid; ¹H NMR (CDCl₃, 300 MHz): δ 8.47 (1H, d, J=5 Hz), 8.35 (1H, d, J=2 Hz), 8.20 (1H, s), 7.66 (1H, m), 7.50 (2H, m), 7.34 (1H, d, J=8 Hz), 7.28 (1H, d, J=8 Hz), 3.75 (4H, m), 3.61 (4H, m), 1.50 (9H, s). LC-MS: m/z for C₂₄H₂₅ClFN₅O₂ expected 469.2 (³⁵Cl), 471.2 (³⁷Cl); found 470.3 (MH⁺), 472.3 (MH⁺).

### Example 15

### SYNTHESIS OF tert-BUTYL-4-[5-{4-(AMINOCARBONYL)-PHENYL}-6-(4-CHLOROPHENYL)-PYRAZIN-2-YL]-PIPERAZINE-1-CARBOXYLATE

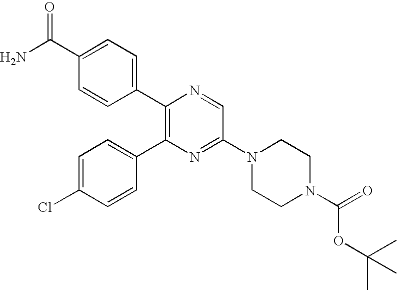

This compound is prepared as described in Example 13, using 4-aminocarbonylphenylboronic acid, and is obtained as an off-white solid. LC-MS: m/z for C₂₆H₂₈ClN₅O₃ expected 493.2 (³⁵Cl), 495.2 (³⁷Cl); found 494.4 (MH⁺), 496.2 (MH⁺).

### Example 16

### SYNTHESIS OF tert-BUTYL-4-[6-(4-CHLOROPHENYL)-5-(4-CYANOPHENYL)-PYRAZIN-2-YL]-PIPERAZINE-1-CARBOXYLATE

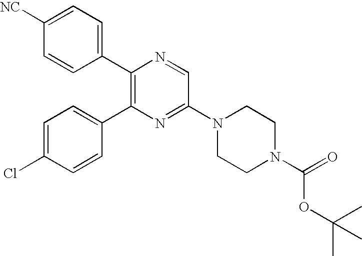

This compound is prepared as described in Example 13, using 4-cyanophenylboronic acid, and is obtained as a white solid. LC-MS: m/z for C₂₆H₂₆ClN₅O₂ expected 475.2 (³⁵Cl), 477.2 (³⁷Cl); found 476.2 (MH⁺), 477.8 (MH⁺).

### Example 17

### SYNTHESIS OF tert-BUTYL-4-[5-{4-(ACETYLPHENYL}-6-(4-CHLOROPHENYL)-PYRAZIN-2-YL]-PIPERAZINE-1-CARBOXYLATE

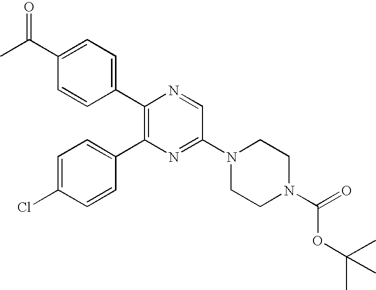

This compound is prepared as described in Example 13, using 4-acetylphenylboronic acid, and is obtained as a white solid. LC-MS: m/z for C₂₇H₂₉ClN₄O₃ expected 492.2 (³⁵Cl), 494.2 (³⁷Cl); found 493.4 (MH⁺), 495.2 (MH⁺).

### Example 18

### SYNTHESIS OF TERT-BUTYL-4-[5-{1-(TERT-BUTOXYCARBONYL)-1H-PYRROL-2-YL}-6-(4-CHLOROPHENYL)-PYRAZIN-2-YL]-PIPERAZINE-1-CARBOXYLATE

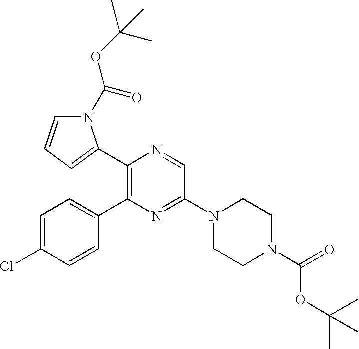

This compound is prepared as described in Example 13, using 1-(tert-butoxycarbonyl)pyrrol-2-boronic acid, and is obtained as a pale yellow solid. LC-MS: m/z for C₂₈H₃₄ClN₅O₄ expected 539.2 (³⁵Cl), 541.2 (³⁷Cl); found 540.3 (MH⁺), 542.3 (MH⁺).

### Example 19

### GENERAL PROCEDURE FOR THE CATALYTIC AMINATION REACTION OF tert-BUTYL-4-[5-BROMO-6-(4-CHLOROPHENYL)PYRAZIN-2-YL]PIPERAZINE-1-CARBOXYLATE WITH PRIMARY AND SECONDARY AMINES

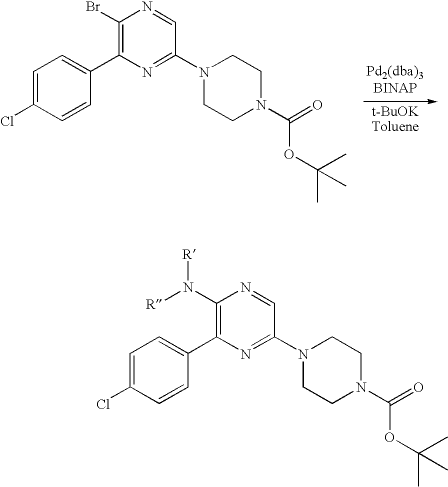

The corresponding primary or secondary amine (R′NHR″) (0.24 mmol) is added to a solution of tert-butyl-4-[5-bromo-(6-chlorophenyl)pyrazin-2-yl]piperazine-1-carboxylate (100 mg, 0.22 mmol), sodium tert-butoxide (30 mg, 0.31 mmol), tris(dibenzylideneacetone)dipalladium(0) (6 mg, 3 mol %) and rac-BINAP (12 mg, 9 mol %) in toluene (3 mL). The resulting solution is stirred at 100° C. for 6-16 h (TLC control). The reaction mixture is cooled to rt and then diluted with EtOAc (10 mL) and water (5 mL). The organic layer is separated and the aqueous layer is extracted with EtOAc (2×5 mL). The combined organic layers are dried over anhydrous MgSO₄ and the solvents are removed in vacuo. Purification by PTLC gives the corresponding amination product.

### Example 20

### tert-BUTYL-4-[6-(4-CHLOROPHENYL)-5-(4-METHYLPIPERAZIN-1-YL)PYRAZIN-2-YL]PIPERAZINE-1-CARBOXYLATE

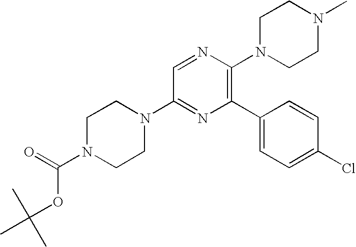

This compound is prepared as described above, using 1-methylpiperazine as the corresponding amine, and is obtained as a yellow solid; ¹H NMR (CDCl₃, 300 MHz): δ 8.21 (2H, d, J=9 Hz), 7.73 (1H, s), 7.38 (2H, d, J=9 Hz), 3.58 (4H, m), 3.49 (4H, m), 3.11 (4H, m), 2.57 (4H, m), 2.38 (3H, s), 1.48 (9H, s); LC-MS: m/z for C₂₄H₃₃ClN₆O₂ expected 472.2 (³⁵Cl), 474.2 (³⁷Cl); found 473.4 (MH⁺), 475.2 (MH⁺).

### Example 21

### tert-BUTYL-4-[6-(4-CHLOROPHENYL)-5-MORPHOLIN-4-YLPYRAZIN-2-YL]PIPERAZINE-1-CARBOXYLATE

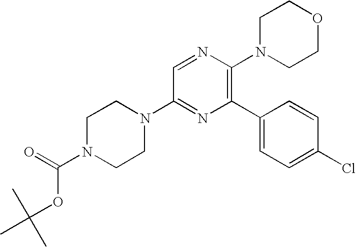

This compound is prepared as described above, using morpholine as the corresponding amine, and is obtained as a yellow solid; LC-MS: m/z for C₂₃H₃₀ClN₅O₃ expected 459.2 (³⁵Cl), 461.2 (³⁷Cl); found 460.4 (MH⁺), 462.4 (MH⁺).

### Example 22

### GENERAL PROCEDURE FOR PREPARATION OF 2-ALKOXYL-5,6-BIS(4-CHLOROPHENYL)-PYRAZINE-3-CARBONITRILE

### STEP 1. 1,2-BIS(4-CHLOROPHENYL)-2-HYDROXYETHANONE

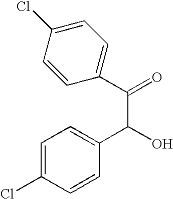

To 4-chlorobenzaldehyde (14.06 g, 100 mmol) in EtOH (20 ml) is added a solution of sodium cyanide (1.06 g, 21.6 mmol) in water (10 ml). The mixture is heated to reflux for 2.5 h and then extracted with DCM. The organic phase is washed with sodium bisulfite solution, dried with MgSO₄ and concentrated in vacuo. The compound is isolated by crystallization from ether/heptane. ¹H NMR (CDCl₃): 7.82 (d, 2H), 7.38 (d, 2H), 7.30 (d, 2H), 7.24 (d, 2H), 5.87 (s, 1H), 4.47 (s, 1H).

### STEP 2. 1,2-BIS(4-CHLOROPHENYL)ETHAN-1,2-DIONE

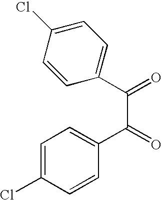

A mixture of 1,2-bis(4-chlorophenyl)-2-hydroxyethanone (5.6 g, 20 mmol) and NBS (10.68 g, 60 mmol) in dry carbon tetrachloride (200 ml) is refluxed in an oil bath. The reaction is monitored by TLC. After the reaction is completed and quenched by water (100 ml). The aqueous layer is extracted with carbon tetrachloride (2×100 ml). The combined organic layers are dried over anhydrous MgSO₄ and concentrated in vacuo. The residue is recrystallized in EtOH to give the title compound as light yellow crystals. ¹H NMR (CDCl₃): 7.94 (d, 4H), 7.53 (d, 4H).

### STEP 3. 5, 6-BIS(4-CHLOROPHENYL)-PYRAZINE-2,3-DICARBONITRILE

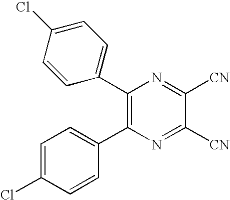

1,2-bis(4-chlorophenyl)ethan-1,2-dione, (5.1 g, 18.3 mmol), diaminomaleonitrile (2.18, 20.2 mmol) and AcOH (1.5 ml) in EtOH (40 ml) and water (25 ml) are heated at 75° C. overnight. The reaction mixture is cooled and diluted with water (50 ml). The precipitate is filtered and washed with EtOH and ether to give the title compound as a pale yellow solid. ¹H NMR (CDCl₃): 7.49 (d, 4H), 7.33 (d, 4H).

### STEP 4. GENERAL PROCEDURE FOR THE SYNTHESIS OF 5,6-BIS-(4-CHLOROPHENYL)-3-ALKOXYL-PYRAZINE-2-CARBONITRILE

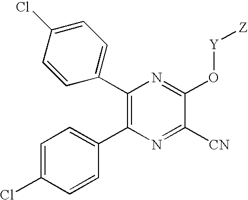

60% Sodium hydride (18 mg, 0.45 mmol) is added to a stirred mixture of 5,6-bis(4-chlorophenyl)-pyrazine-2,3-dicarbonitrile (105 mg, 0.3 mmol) and an alcohol (0.36 mmol) in THF (2.5 ml). Stirring is continued overnight, followed by evaporation. PTLC purification is used to isolate the product.

### Example 23

### SYNTHESIS OF 5,6-BIS-(4-CHLOROPHENYL)-3-(2-METHOXY-ETHOXY)-PYRAZINE-2-CARBONITRILE

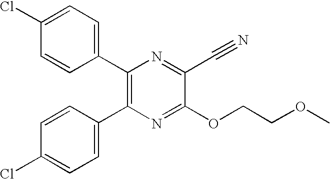

This compound is prepared from 5,6-bis(4-chlorophenyl)-pyrazine-2,3-dicarbonitrile and 2-methoxyethanol as described in Example 22, Step 4. ¹H NMR (CDCl₃): 7.30-7.41 (m, 8H), 4.70 (dd, 2H), 3.83 (dd, 2H), 3.47 (s, 3H).

### Example 24

### SYNTHESIS OF 5,6-BIS(4-CHLOROPHENYL)-3-[2-(2-ETHOXY-ETHOXY)-ETHOXY]-2-PYRAZINECARBONITRILE

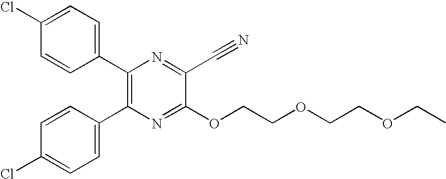

This compound is prepared from 5,6-bis(4-chlorophenyl)-pyrazine-2,3-dicarbonitrile and 2-(2-methoxyethoxy)ethanol as described in Example 22, Step 4. ¹H NMR (CDCl₃): 7.30-7.43 (m, 8H), 4.71 (dd, 2H), 3.96 (dd, 2H), 3.75 (dd, 2H), 3.61 (dd, 2H), 3.54 (q, 2H), 1.22 (t, 3H).

### Example 25

### SYNTHESIS OF 4-[5,6-BIS-(4-CHLOROPHENYL)-3-CYANO-PYRAZIN-2-YLOXY]-PIPERIDINE-1-CARBOXYLIC ACID TERT-BUTYL ESTER

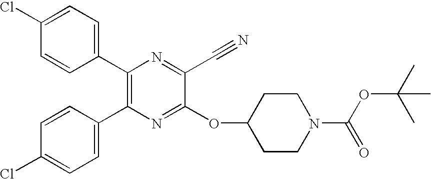

This compound is prepared from 5,6-bis(4-chlorophenyl)-pyrazine-2,3-dicarbonitrile and 1-(tert-butyloxycarbonyl)-4-hydroxypiperidine as described in Example 22, Step 4. ¹H NMR (CDCl₃): ¹H NMR (CDCl₃): 7.30-7.4 (m, 8H), 5.54 (m, 1H), 3.74 (m, 2H), 3.47 (m, 2H), 1.48 (s, 9H).

### Example 26

### SYNTHESIS OF 5,6-BIS(4-CHLOROPHENYL)-3-[(3R)-TETRAHYDRO-3-FURANYLOXY]-2-PYRAZINECARBONITRILE

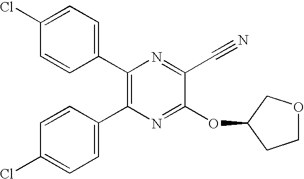

This compound is prepared from 5,6-bis(4-chlorophenyl)-pyrazine-2,3-dicarbonitrile and R-(−)3-hydroxytetrahydrofuran as described in Example 22, Step 4. ¹H NMR (CDCl₃): 7.30-7.41 (m, 8H), 5.69 (m, 1H), 3.96-4.18 (m, 4H), 2.29-2.37 (m, 2H).

### Example 27

### SYNTHESIS OF 5,6-BIS(4-CHLOROPHENYL)-3-(TETRAHYDRO-2-FuRANYLMETHOXY)-2-PYRAZINECARBONITRILE

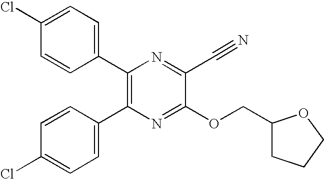

This compound is prepared from 5,6-bis(4-chlorophenyl)-pyrazine-2,3-dicarbonitrile and 2-hydroxymethyl-tetrahydrofuran as described in Example 22, Step 4. ¹H NMR (CDCl₃): 7.29-7.41(m, 8H), 4.56 (d, 2H), 4.37 (m, 1H), 3.95 (m, 1H), 3.84 (m, 1H), 1.87-2.16 (m, 4H).

### Example 28

### SYNTHESIS OF TERT-BUTYL (3S)-3-{[5,6-BIS(4-CHLOROPHENYL)-3-CYANO-2-PYRAZINYL]OXY}-1-PYRROLIDINECARBOXYLATE

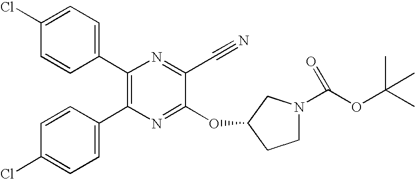

This compound is prepared from 5,6-bis(4-chlorophenyl)-pyrazine-2,3-dicarbonitrile and S-1-(tertbutyloxycarbonyl)-3-hydroxypyrrolidine as described in Example 22, Step 4. ¹H NMR (CDCl₃): 7.30-7.40 (m, 8H), 5.67 (m, 1H), 3.75 (d, 2H), 3.62 (m, 2H), 2.82 (m, 2H), 1.48 (s, 9H).

### Example 29

### SYNTHESIS OF TERT-BUTYL (3R)-3-{[5,6-BIS(4-CHLOROPHENYL)-3-CYANO-2-PYRAZINYL]OXY}-1-PYRROLIDINECARBOXYLATE

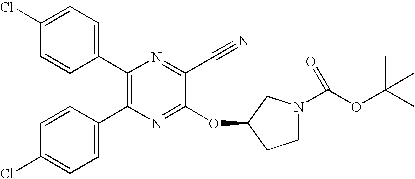

This compound is prepared from 5,6-bis(4-chlorophenyl)-pyrazine-2,3-dicarbonitrile and R-1-(tertbutyloxycarbonyl)-3-hydroxypyrrolidine as described in Example 22, Step 4. ¹H NMR (CDCl₃): 7.30-7.40 (m, 8H), 5.67 (m, 1H), 3.75 (d, 2H), 3.62 (m, 2H), 2.82 (m, 2H), 1.48 (s, 9H).

### Example 30

### SYNTHESIS OF 5,6-BIS(4-CHLOROPHENYL)-3-[2-(2-OXO-1-PYRROLIDINYL) ETHOXY]-2-PYRAZINECARBONITRILE

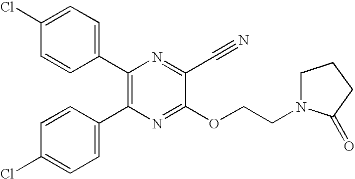

This compound is prepared from 5,6-bis(4-chlorophenyl)-pyrazine-2,3-dicarbonitrile and 1-(2-hydroxyethyl)-2-pyrrolidinone as described in Example 22, Step 4. ¹H NMR (CDCl₃): 7.31-7.40 (m, 8H), 4.68 (t, 2H), 3.77 (t, 2H), 3.68 (t, 2H), 2.39 (t, 2H), 2.07 (t, 2H).

### Example 31

### SYNTHESIS OF 5,6-BIS(4-CHLOROPHENYL)-3-[2-(1-PYRROLIDINYL)-ETHOXY]-2-PYRAZINECARBONITRILE

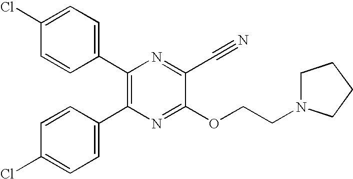

This compound is prepared from 5,6-bis(4-chlorophenyl)-pyrazine-2,3-dicarbonitrile and 1-(2-hydroxyethyl)-2-pyrrolidine as described in Example 22, Step 4. ¹H NMR (CDCl₃): 7.30-7.42 (m, 8H), 4.68 (t, 2H), 3.01 (t, 2H), 2.68 (m, 4H), 1.87 (m, 4H).

### Example 32

### SYNTHESIS OF 5,6-BIS(4-CHLOROPHENYL)-3-[2-(2,5-DIOXO-1-PYRROLIDINYL)-ETHOXY]-2-PYRAZINECARBONITRILE

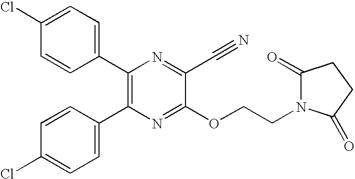

This compound is prepared from 5,6-bis(4-chlorophenyl)-pyrazine-2,3-dicarbonitrile and 1-(2-hydroxyethyl)-2-pyrrolidine-2,5-dione as described in Example 22, Step 4. ¹H NMR (CDCl₃): 7.31-7.42 (m, 8H), 4.76 (d, 2H), 3.99 (d, 2H), 2.71 (s, 4H).

### Example 33

### SYNTHESIS OF 5,6-BIS(4-CHLOROPHENYL)-3-[2-(4-MORPHOLINYL)-ETHOXY]-2-PYRAZINECARBONITRILE

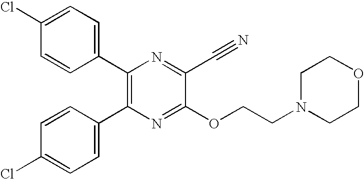

This compound is prepared from 5,6-bis(4-chlorophenyl)-pyrazine-2,3-dicarbonitrile and 4-(2-hydroxyethyl)morpholine as described in Example 22, Step 4. ¹H NMR (CDCl₃): 7.30-7.42 (m, 8H), 4.68 (t, 2H), 3.68 (t, 4H), 2.88 (t, 2H), 2.60 (t, 4H).

### Example 34

### SYNTHESIS OF 5,6-BIS(4-FLUOROPHENYL)-3-[2-(4-PYRIDINYL)-ETHOXY]-2-PYRAZINECARBONITRILE

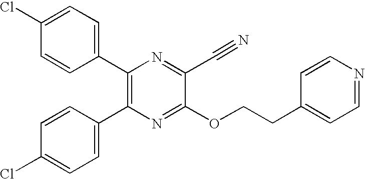

This compound is prepared from 5,6-bis(4-chlorophenyl)-pyrazine-2,3-dicarbonitrile and 4-(2-hydroxyethyl)pyridine as described in Example 22, Step 4. ¹H NMR (CDCl₃): 8.57 (d, 2H), 7.26-7.38 (m, 10H), 4.76 (t, 2H), 3.20 (t, 2H).

### Example 35

### SYNTHESIS OF 5,6-BIS(4-CHLOROPHENYL)-3-[2-(2-PYRIDINYL)-ETHOXY]-2-PYRAZINECARBONITRILE

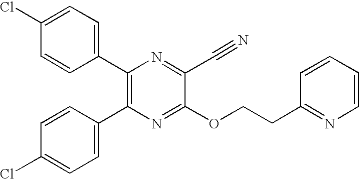

This compound is prepared from 5,6-bis(4-chlorophenyl)-pyrazine-2,3-dicarbonitrile and 2′-(2-hydroxyethyl)pyridine as described in Example 22, Step 4. ¹H NMR (CDCl₃): 8.55 (d, 1H), 7.65 (dt, 1H), 7.26-7.41 (m, 9H), 7.16 (ddd, 1H), 4.92 (t, 2H), 3.36 (t, 2H).

### Example 36

### SYNTHESIS OF 2,3-BIS(4-CHLOROPHENYL)-5-(2-METHOXYETHOXY)-PYRAZINE

### STEP 1. 2,3-BIS(4-CHLOROPHENYL)-PYRAZINE

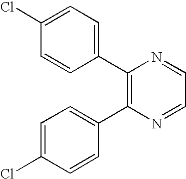

To a sealed tube containing 5,6-dichloropyrazine (9.66 g, 64.6 mmol) is added 4-chlorophenylboronic acid (21.2 g, 129 mmol), potassium carbonate (26.4 g, 194 mmol) and bis(triphenylphosphine)palladium(II) dichloride (600 mg). MeCN (150 mL) and water (150 mL) are added, and nitrogen bubbled through the mixture for 15 min. The mixture is then heated at 70° C. under nitrogen for 18 h. After cooling to rt, the organic phase is separated and the aqueous phase is extracted with EtOAc (2×200 mL). The combined organic phase is dried over anhydrous MgSO₄, concentrated and purified by silica gel column chromatography to give the title compound as a white solid. MS: m/z expected 301.2; found 302.2 (MH⁺).

### STEP 2. 2,3-BIS(4-CHLOROPHENYL)-PYRAZINE-N-OXIDE

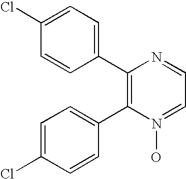

A solution of 2,3-bis(4-chlorophenyl)-pyrazine (21.3 g, 71 mmol), 50% H₂O₂ (9.66 mL, 142 mmol), and maleic anhydride (12.1 g, 124 mmol) in CHCL₃ (300 mL) is refluxed for 2 h. After cooling to rt, the mixture is washed with water, 10% potassium carbonate and water, successively. The organic phase is dried over anhydrous MgSO₄, concentrated and purified by silica gel column chromatography to give the title compound as a white solid.

### STEP 3. 5-CHLORO-2,3-BIS(4-CHLOROPHENYL)-PYRAZINE

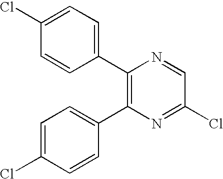

A solution of 2,3-bis(4-chlorophenyl)-pyrazine N-oxide (12.7 g, 40 mmol) in POCl₃ (80 mL) is refluxed for 20 min. After most of the POCl₃ is removed under reduced pressure, the residue is poured into ice water, and made alkaline with potassium carbonate. The product is extracted with DCM, dried over anhydrous MgSO₄, concentrated and purified by silica gel column chromatography to give the title compound as a light yellow solid. MS: m/z expected 335.6; found 336.1 (MH+).

### STEP 4. 2,3-BIS(4-CHLOROPHENYL)-5-(2-METHOXYETHOXY)-PYRAZINE

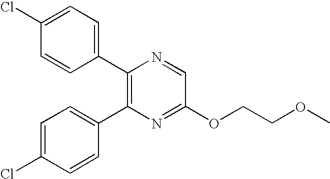

A solution of 2-methoxyethanol (10 mg, 0.15 mmol) in anhydrous THF (0.5 mL) is treated with 60% NaH (6 mg) under nitrogen at rt for 30 min, followed by addition of a solution of 5-chloro-2,3-bis(4-chlorophenyl)-pyrazine (33 mg, 1 mmol) in anhydrous THF (1 mL). The mixture is stirred at 50° C. overnight, and quenched by addition of water. THF is evaporated and the residue is purified by silica gel column chromatography to give the title compound as a white foam. MS: m/z expected 375.2; found 376.1 (MH+). ¹H NMR (CDCl₃): 3.46 (s, 3H), 3.79 (t, J=4.5 Hz, 2H), 4.59 (t, J=4.5 Hz, 2H), 7.26-7.39 (m, 8H), 8.31 (s, 1H).

### Example 37

### SYNTHESIS OF 5-(AZETIDIN-3-YLOXY)-2,3-BIS(4-CHLOROPHENYL)-PYRAZINE

### STEP 1. TERT-BUTYL 3-(5,6-BIS(4-CHLOROPHENYL)-PYRAZIN-2-YLOXY)AZETIDINE-1-CARBOXYLATE

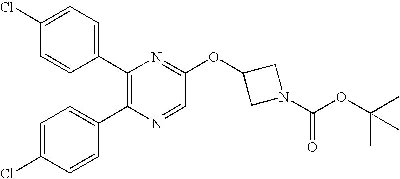

This compound is prepared as described in Example 36 (Step 4) using tert-butyl 3-hydroxyazetidine-1-carboxylate as starting material, and is obtained as a white solid. MS: m/z expected 472.0; found 473.0 (MH+). ¹H NMR (CDCl₃): 1.45 (s, 9H), 4.05 (dd, J=10.2, 4.5 Hz, 2H), 4.36 (dd, J=10.2, 4.5 Hz, 2H), 5.37-5.44 (m, 1H), 7.26-7.34 (m, 8H), 8.29 (s, 1H).

### STEP 2. 5-(AZETIDIN-3-YLOXY)-2,3-BIS(4-CHLOROPHENYL)-PYRAZINE

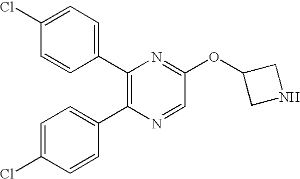

A solution of tert-butyl 3-(5,6-bis(4-chlorophenyl)-pyrazin-2-yloxy)azetidine-1-carboxylate (236 mg, 0.5 mmol) in anhydrous DCM (2 mL) is treated with TFA (1 mL) for 2 h at rt. After concentration, the residue is neutralized with saturated sodium bicarbonate and the product is extracted with DCM. The extracts are dried over anhydrous MgSO₄, concentrated and purified by silica gel column chromatography to give the title compound as a white solid. MS: m/Z expected 372.2; found 373.2 (MH+).

### Example 38

### SYNTHESIS OF 1-(3-(5,6-BIS(4-CHLOROPHENYL)-PYRAZIN-2-YLOXY)-AZETIDIN-1-YL)-2-METHYLPROPAN-1-ONE

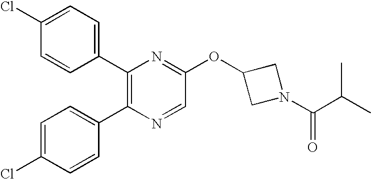

This compound is prepared as described in Example 7 by the reaction of 5-(azetidin-3-yloxy)-2,3-bis(4-chlorophenyl)-pyrazine with isobutyryl chloride.

### Example 39

### SYNTHESIS OF 2,3-BIS(4-CHLOROPHENYL)-5-(1-(ISOPROPYLSULFONYL)-AZETIDIN-3-YLOXY)-PYRAZINE

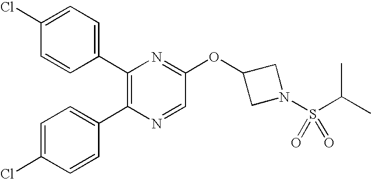

This compound is prepared as described in Example 7 by the reaction of 5-(azetidin-3-yloxy)-2,3-bis(4-chlorophenyl)-pyrazine with isopropylsulfonyl chloride.

### Example 40

### SYNTHESIS OF 2,3-BIS(4-CHLOROPHENYL)-5-(1-(PYRIMIDIN-2-YL)-AZETIDIN-3-YLOXY)-PYRAZINE

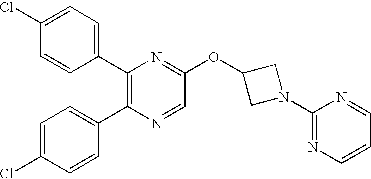

This compound is prepared as described in Example 10 by the reaction of 5-(azetidin-3-yloxy)-2,3-bis(4-chlorophenyl)-pyrazine with 2-chloropyrimidine.

### Example 41

### SYNTHESIS OF 4-[5,6-BIS-(4-CHLOROPHENYL)-PYRAZIN-2-YL]-3,6-DIHYDRO-2H-PYRIDINE-1-CARBOXYLIC ACID TERT-BUTYL ESTER

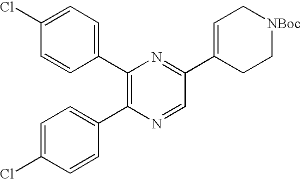

5-Chloro-2,3-bis(4-chlorophenyl)-pyrazine (Example 36; 5 g, 14.9 mmol), 4-(4,4,5,5-tetramethyl-[1,3,2]dioxaborolan-2-yl)-3,6-dihydropyridine-1(2H)-carboxylic acid tert-butyl ester (6.9 g, 22.3 mmol), potassium carbonate (6.2 g, 45 mmol) and DMF (20 mL) are charged into a flask. The mixture is degassed, and [1,1′-bis(diphenylphosphino)ferrocene]-dichloropalladium (II) DCM complex (1:1) (0.6 g, 0.73 mmol) is added to the flask. The resulting mixture is stirred at 90° C. overnight, cooled to rt and poured into cold water (60 mL). The aqueous mixture is extracted with EtOAc/hexanes (1:1, 3×40 mL). The combined organic phase is washed with water and dried over MgSO₄. Removal of solvents gives a residue which is purified by column chromatography (4:1 hexanes/EtOAc) to afford the title product as an off-white solid. MS: m/z expected 481.1; found 482.3 (MH+). ¹H NMR (CDCl₃) 2.1-2.3 (m, 3H), 3.6-3.8 (m, 3H), 5.00 (m, 1H), 7.26-7.37 (m, 8H), 8.53 (s, 1H).

### Example 42

### SYNTHESIS OF 4-[5,6-BIS-(4-CHLOROPHENYL)-PYRAZIN-2-YL]-PIPERIDINE-1-CARBOXYLIC ACID TERT-BUTYL ESTER

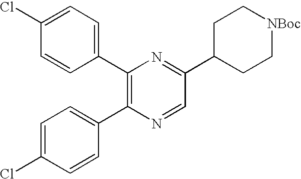

To a solution of 4-[5,6-bis-(4-chlorophenyl)-pyrazin-2-yl]-3,6-dihydro-2H-pyridine-1-carboxylic acid tert-butyl ester (0.8 g, 1.66 mmol) in EtOH (5 mL) is added PtO₂ (38 mg, 0.167 mmol). The mixture is stirred at rt under H₂ for 1 h. The catalyst is removed by filtration, and flash column chromatography (hexanes/EtOAc 4:1) gives the title product. MS: m/z expected 483.2; found 506.3 (MNa+). ¹H NMR (CDCl₃): 1.8-2.0 (m, 4H), 2.8-3.0 (m, 3H), 4.3 (br, 2H), 7.26-7.37 (m, 8H), 8.47 (s, 1H).

### Example 43

### SYNTHESIS OF 2,3-BIS-(4-CHLOROPHENYL)-5-PIPERIDIN-4-YL-PYRAZINE

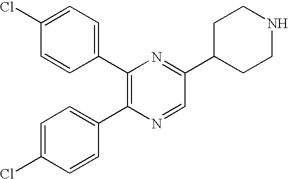

To a solution of 4-[5,6-bis-(4-chlorophenyl)-pyrazin-2-yl]-piperidine-1-carboxylic acid tert-butyl ester in DCM (5 mL) is added TFA (1 mL). The mixture is stirred at rt for 2 h. Removal of volatiles gives a residue which is purified by column chromatography (20% MeOH in EtOAc) to give the title product. MS: expected 383.1; found 384.2 (MH+).

### Example 44

### SYNTHESIS OF 1-{4-[5,6-BIS-(4-CHLOROPHENYL)-PYRAZIN-2-YL]-PIPERIDIN-1-YL}-PROPAN-1-ONE

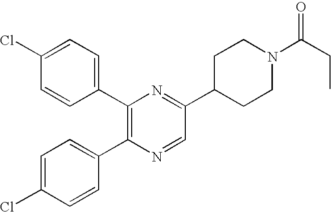

To a solution of 2,3-bis-(4-chlorophenyl)-5-piperidin-4-yl-pyrazine (38 mg, 0.1 mmol) in anhydrous DCM (2 mL) is added TEA (20 mg, 0.2 mmol), followed by propionyl chloride (9.2; mg, 0.1 mmol). The mixture is allowed to stand at rt overnight. PTLC purification gives a white solid product. MS: m/z expected 439.1; found 440.2 (MH+). ¹H NMR (CDCl₃): 1.18 (t, J=7.5 Hz, 3H), 1.8-2.1 (m, 4H), 2.40 (q, J=7.5 Hz, 2H), 2.7 (m, 1H), 3.1 (m, 1H), 3.2 (m, 1H), 4.03 (d, J=13.8 Hz, 1H), 4.83 (d, J=12.9 Hz, 1H), 7.20-7.39 (m, 8H), 8.47 (s, 1H).

### Example 45

### SYNTHESIS OF 1-{4-[5,6-BIS-(4-CHLOROPHENYL)-PYRAZIN-2-YL]-PIPERIDIN-1-YL}-2-METHYL-PROPAN-1-ONE

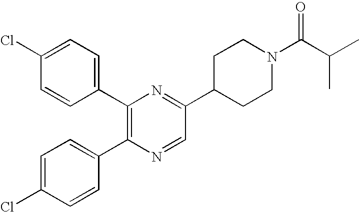

This compound is prepared following the procedure given in Example 44 by reaction of 2,3-bis-(4-chlorophenyl)-5-piperidin-4-yl-pyrazine with isobutyryl chloride. MS: m/z expected 453.1; found 454.3 (MH+).

### Example 46

### SYNTHESIS OF 3-BIS-(4-CHLOROPHENYL)-5-(1-ETHANESULFONYL-PIPERIDIN-4-YL)-PYRAZINE

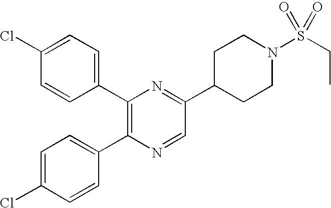

This compound is prepared following the procedure given in Example 44 by reaction of 2,3-bis-(4-chlorophenyl)-5-piperidin-4-yl-pyrazine with ethylsulfonyl chloride. MS: m/z expected 475.1; found 476.2 (MH+). ¹H NMR (CDCl₃): 1.40 (t, J=7.2 Hz, 3H), 2.0-2.20 (m, 4H), 2.9-3.1 (m, 4H), 3.99 (d, J=12.3 Hz, 2H), 7.26-7.40 (m, 8H), 8.48 (s, 1H).

### Example 47

### SYNTHESIS OF 2,3-BIS-(4-CHLOROPHENYL)-5-[1-(PPROPANE-2-SULFONYL)-PIPERIDIN-4-YL]-PYRAZINE

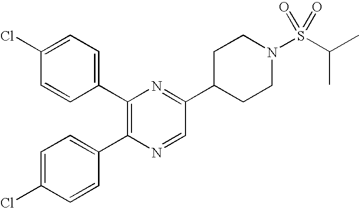

This compound is prepared following the procedure given in Example 44 by reaction of 2,3-bis-(4-chlorophenyl)-5-piperidin-4-yl-pyrazine with isopropylsulfonyl chloride. MS: m/z expected 489.1; found 490.1 (MH+).

### Example 48

### SYNTHESIS OF 4-[5,6-BIS-(4-CHLOROPHENYL)-PYRAZIN-2-YL]-PIPERIDINE-1-SULFONIC ACID DIMETHYLAMIDE

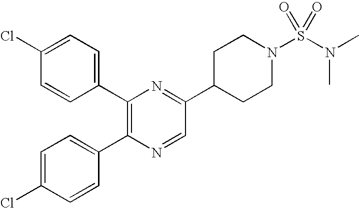

This compound is prepared following the procedure given in Example 44 by reaction of 2,3-bis-(4-chlorophenyl)-5-piperidin-4-yl-pyrazine with dimethylsulfamoyl chloride. MS: m/z expected 490.1; found 491.3 (MH+).

### Example 49

### SYNTHESIS OF 4,5-BIS-(4-CHLOROPHENYL)-2-(4,4-DIMETHYL-1,1-DIOXIDO-1,2,5-THIADIAZOLIDIN-2-YL)-PYRIMIDINE

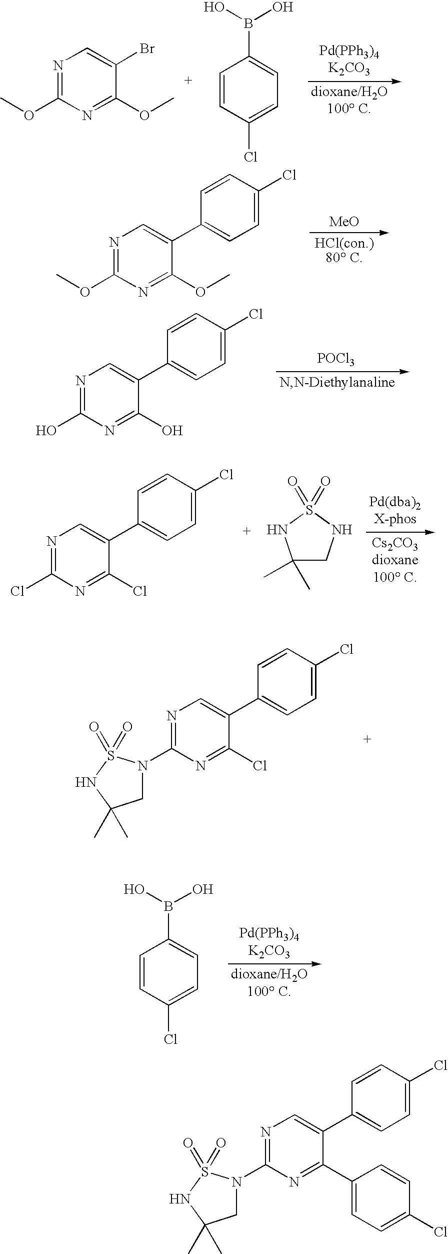

### STEP 1. 5-(4-CHLOROPHENY)2,4-DIMETHOXY-PYRIMIDINE

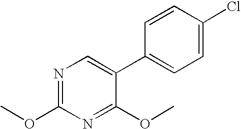

A mixture of 5-bromo-2,6-dimethoxy-pyrimidine (10.0 g, 45.7 mmol), 4-chlorophenyl-boroic acid (8.6 g, 54.8 mmol), K₂CO₃ (15.2 g, 110 mmol) and Pd(PPh₃)₄ (1 g, 3 mmol %) in degassed dioxane (30 mL) and H₂O (5 mL) is heated at 100° C. for 14 h. The reaction mixture is cooled, and EtOAc is added. The organic layer is dried over Na₂SO₄ and evaporated under reduced pressure. The residue is purified by flash column and eluted with 10% EtOAc in hexane to give the title compound. m/z: 250.9.

### STEP 2. 5-(4-CHLOROPHENYL)PYRIMIDINE-2,4-DIOL

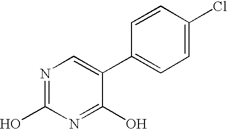

5-(4-Chloropheny)2,4-dimethoxy-pyrimidine (12.0 g, 47.9 mmol) is dissolved in MeOH (250 mL) and HCl (con., 30 mL) and heated to 80° C. for 14 h. The reaction mixture is cooled, and the solvent is removed under reduced pressure. The solid is washed with H₂O and dried under vacuum (70° C.) to give the title compound.

### STEP 3. 2,4-DICHLORO-5-(4-CHLOROPHENY)PYRIMIDINE

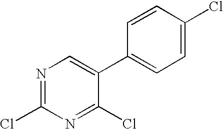

5-(4-Chlorophenyl)pyrimidine-2,4-diol (11.0 g, 49.4 mmol) is mixed in POCl₃ 100 mL at rt. N,N-Diethylaniline is added, and the reaction mixture is heated to 100° C. for 12 h. The reaction mixture is cooled and evaporated under reduced pressure. The residue is poured into ice-H₂O to form a solid. The solid is filtered and washed with H₂O and dried under vacuum (70° C.) to give the title compound.

### STEP 4. 4-CHLORO-5-(4-CHLOROPHENYL)-2-(4,4-DIMETHYL-1,1-DIOXIDO-1,2,5-THIADIAZOLIDIN-2-YL)-PYRIMIDINE

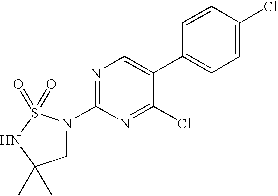

3,3-Dimethyl-[1,2,5]thiadiazolidine 1,1-dioxide (570 mg, 3.8 mmol) and NaH (270 mg, 6.7 mmol, 60%), is dissolved in THF and heated to 45° C. for 1 h and cooled to rt. 2,4-Dichloro-5-(4-chloro-pheny)pyrimidine (1 g, 3.8 mmol) is added, and the reaction mixture is heated to 35° C. for 14 h. The reaction mixture is cooled, and saturated NH₄Cl is added. The aqueous layer is extracted with EtOAc, and the combined organic layer is dried and evaporated under reduced pressure. The residue is purified by PTLC to give the title compound. ¹H NMR (CDCl₃): 8.47 (s, 1H), 7.47-7.35. (d, 2H), 7.32-7.26 (d, 2H), 4.48 (b, 1H), 4.05 (s, 2H), 1.57 (s, 6H). m/z: 372.9.

### STEP 5. 4,5-BIS-(4-CHLOROPHENYL)-2-(4,4-DIMETHYL-1,1-DIOXIDO-1,2,5]-THIADIAZOLIDIN-2-YL)-PYRIMIDINE

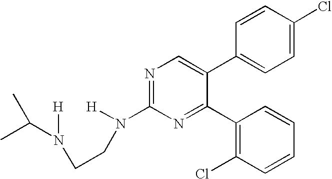

4-Chloro-5-(4-chlorophenyl)-2-(4,4-dimethyl-1,1-dioxido-1,2,5-thiadiazolidin-2-yl)-pyrimidine (50 mg, 0.13 mmol), 4-chlorophenylboroic acid (24 mg, 0.15 mmol), K₂CO₃ (28 mg, 0.2 mmol) and Pd(PPh₃)₄ (2 mg) are dissolved in degassed dioxane (1.5 mL) and H₂O (0.3 mL). The reaction mixture is heated to 100° C. for 14 h. EtOAc is added to the reaction mixture. The organic layer is evaporated under reduced pressure. The residue is purified by PTLC to give the title compound. ¹H NMR (CDCl₃): 8.52 (s, 1H), 7.48-7.28. (m, 6H), 7.10-7.07 (d, 2H), 4.54 (b, 1H), 4.10 (s, 2H), 1.58 (s, 6H).

### Example 50

### SYNTHESIS OF 1-[5-(4-CHLORO-PHENYL)-4-(2-CHLORO-PHENYL)-PYRIMIDIN-2-YL]-3-ISOPROPYL-IMIDAZOLIDIN-2-ONE

### STEP 1. N-[5-(4-CHLORO-PHENYL)-4-(2-CHLORO-PHENYL)-PYRIMIDIN-2-YL]-N′-ISOPROPYL-ETHANE-1,2-DIAMINE

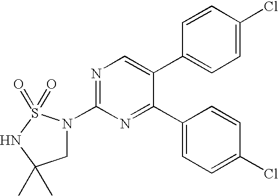

A mixture of 2,4-dichloro-5-(4-chlorophenyl)pyrimidine (300 mg, 1.15 mmol), N′1′-isopropyl-ethane-1,2-diamine (118 mg, 1.15 mmol) and K₂CO₃ (482 mg, 3.47 mmol) in CH₃CN (10 mL) is stirred at 30° C. overnight. The mixture is diluted with EtOAc, washed with water, and concentrated under vacuum. The residue is purified by silica gel column with EtOAc/Hexane (1:1) to give the title compound as the more polar product.

### STEP 2. 1-[5-(4-CHLORO-PHENYL)-4-(2-CHLORO-PHENYL)-PYRIMIDIN-2-YL]-3-ISOPROPYL-IMIDAZOLIDIN-2-ONE

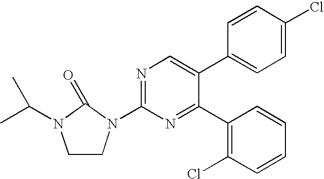

A mixture of N-[5-(4-chloro-phenyl)-4-(2-chloro-phenyl)-pyrimidin-2-yl]-N′-isopropyl-ethane-1,2-diamine (12.5 mg, 0.03 mmol), 1,1′-carbonyldiimidazole (25 mg, 0.15 mmol) and CsCO₃ (20 mg, 0.06 mmol) in CH₃CN (1 mL) is heated at 80° C. overnight. The mixture is cooled, diluted with water (1 mL) and 1 N NaOH (0.5 mL), and extracted with EtOAc (2 mL). The extract is washed once with water and concentrated under vacuum. The residue is purified by PTLC (5% MeOH in CH₂Cl₂) to produce the title compound. ¹H NMR (CDCl₃): 8.68 (s, 1H), 7.27-7.31 (m, 4H), 7.21 (d, 2H), 7.00 (d, 2H), 4.36 (m, 1H), 4.07 (t, 2H), 3.43 (t, 2H), and 1.21 (d, 6H).

### Example 51

### SYNTHESIS OF [6-(2-CHLORO-4-METHOXY-PHENYL)-3-(1-ETHYL-PROPOXY)-5-(4-FLUORO-PHENYL)-PYRIDIN-2-YL]-METHYL-AMINE

### STEP 1. 2-CHLORO-3-(1-ETHYL-PROPOXY)-6-IODO-PYRIDINE

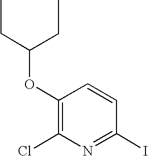

To a solution of 2-chloro-3-pyridinol (23.4 g, 0.18 mol) in Na₂CO₃ (225 mL, 1.0 m aqueous solution, 0.225 mol) is added 12 (45.8 g, 0.18 mol) and the resulting mixture is stirred overnight to dissolve all I₂ and form a white solid precipitate. The mixture is then diluted with EtOAc and acidified with concentrated HCl to pH 2-3. The mixture is extracted with EtOAc (100 mL×3) and the combined extracts are washed with H₂O, dried, evaporated to give a yellow solid. The solid is then dissolved in DMF (300 ml). To this solution is added solid K₂CO₃ (40 g) and 3-bromopentane (44.8 ml, 2 eq). The resulting mixture is heated to 90° C. (gentle reflux) for 24 h, then cooled to rt, poured into 5% EtOAc/hexane (500 mL), washed with H₂O (100 mL) several times, and dried over Na₂SO₄. Solvent is removed to give the product as an oil, which is used in the next step.

### STEP 2. [3-(1-ETHYL-PROPOXY)-6-IODO-PYRIDIN-2-YL]-METHYL-AMINE

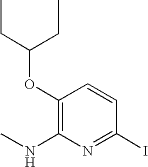

Crude 2-chloro-3-(1-ethyl-propoxy)-6-iodo-pyridine (40 g) is dissolved in CH₃NH₂ (4N in NMP, 85 ml, 3 eq) and sealed and heated to 100° C. for 2 days. The mixture is then diluted with 5% EtOAc in hexane, washed with H₂O several times and dried. Solvent is removed to give a dark green oil. Crystals form on cooling. The mixture of oil and crystals is filtered. The solid is washed with hexane, dried to give the title compound as a light green crystalline solid. The filtrate is collected to give an oil which is purified by column (3% EtOAc/hexane) to give additional solid title compound.

### STEP 3. [5-BROMO-3-(1-ETHYL-PROPOXY)-6-IODO-PYRIDIN-2-YL]-METHYL-AMINE

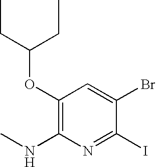

To a solution of the solid from step 2, (3.47 g, 10.83 mmol) in CHCl₃ (40 ml) is added NBS (2.12 g, 11.9 mmol) at 0° C., warmed to rt, stirred for 20 min, and then evaporated to remove CHCl₃. 6% EtOAc in hexane is added into the residue and washed with sat. NaHCO₃ and H₂O, dried and evaporated. The formed crystals are collected by filtration. The solid is washed with hexane, and dried to give the title compound, which is then purified by column (1% EtOAc in hexane).

### STEP 4. [5-BROMO-6-(2-CHLORO-4-METHOXY-PHENYL)-3-(1-ETHYL-PROPOXY)-PYRIDIN-2-YL]-METHYL-AMINE

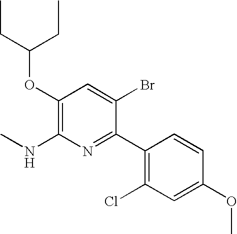

This compound is prepared from 2-chloro-4-methoxy-phenylboronic acid and [5-bromo-3-(1-ethyl-propoxy)-6-iodo-pyridin-2-yl]-methyl-amine according to the procedure given in Example 4, step 1.

### STEP 5. [6-(2-CHLORO-4-METHOXY-PHENYL)-3-(1-ETHYL-PROPOXY)-5-(4-FLUORO-PHENYL)-PYRIDIN-2-YL]-METHYL-AMINE

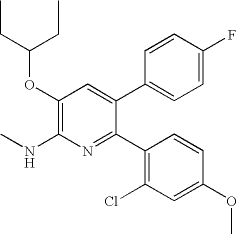

This compound is prepared from 4-fluoro-phenylboronic acid and [5-bromo-6-(2-chloro-4-methoxy-phenyl)-3-(1-ethyl-propoxy)-pyridin-2-yl]-methyl-amine according to the procedure given in Example 4, step 2.

### Example 52

### SYNTHESIS OF 3-(ETHYLAMINO)-1-{5-(3-CHLOROPYRIDIN-4-YL)-6-[4-(TRIFLUOROMETHYL)PHENYL]-1,2,4-TRIAZIN-3-YL}AZETIDINE-3-CARBOXAMIDE

### STEP 1. 1-(3-CHLOROPYRIDIN-4-YL)-2-[4-(TRIFLUOROMETHYL)PHENYL]ETHANOL

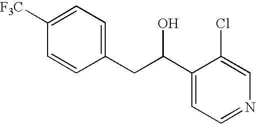

An oven-dried round bottom flask is charged with zinc (4.29 g, 65.6 mmol) and 20 mL of anhydrous THF. To this flask is added 0.2 mL of 1,2-dibromoethane, and the reaction mixture is heated at 66° C. A solution of 4-trifluoromethylbenzyl bromide (10.8 g, 45 mmol) and 1,2-dibromoethane (0.2 mL) in THF (25 mL) is added slowly via cannula. After stirring for 1 h at 66° C., the reaction mixture is cooled to rt, and then is added into a solution of 3-chloro-4-pyridinecarboxaldehyde (5.1 g, 36 mmol) in THF (60 mL) at 0° C. The reaction mixture is allowed to warm to rt and stirred for 5 h at rt before it is quenched with the addition of saturated aqueous ammonium chloride. The organic layer is separated, and the aqueous layer is back extracted with EtOAc (150 mL×2). The combined organic layers are washed with water and brine, dried over sodium sulfate, and concentrated. Purification of the residue by flash column chromatography (50% EtOAc in hexanes) affords the title compound as a yellow solid.

### STEP 2. 1-(3-CHLOROPYRIDIN-4-YL)-2-[4-(TRIFLUOROMETHYL)PHENYL]ETHANE-1,2-DIONE

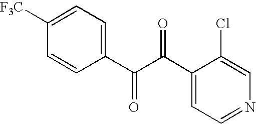

To a solution of 5.89 g of 1-(3-chloropyridin-4-yl)-2-[4-(trifluoromethyl)phenyl]ethanol (8.0 mmol) in DCM (80 mL), is added Dess-Martin Periodinane (4.13 g, 19.5 mmol). After stirring for 1 h at rt, the reaction is quenched with saturated aqueous NaHCO₃ and Na₂S₂O₃ solution. The organic layer is separated, and the aqueous layer is back extracted with DCM (60 mL×2). The combined organic layers are washed with water and brine, dried over sodium sulfate, and concentrated. Purification of the residue by flash column chromatography (30% EtOAc in hexanes) affords 1-(3-chloropyridin-4-yl)-2-[4-(trifluoromethyl)phenyl]ethanone as a yellow solid.

To a solution of 3.4 g of 1-(3-chloropyridin-4-yl)-2-[4-(trifluoromethyl)phenyl]ethanone (11.34 mmol) in 1,4-dioxine (80 ml) and water (6 mL), is added 3.1 g of selenium dioxide (28.35 mmol). The reaction mixture is heated under reflux for 16 h. After being cooled to rt, the reaction mixture is diluted with EtOAc and water. The organic layer is separated, and the aqueous layer is back extracted with EtOAc (50 mL×2). The combined organic layers are washed with water and brine, dried over sodium sulfate, and concentrated. Purification of the residue by flash column chromatography (30% EtOAc in hexanes) affords the title compound as a yellow solid.

### STEP 3. 3-CHLORO-5-(3-CHLOROPYRIDIN-4-YL)-6-[4-(TRIFLUOROMETHYL)PHENYL]-1,2,4-TRIAZINE

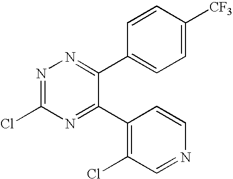

To a solution of 117 mg of 1-(3-chloropyridin-4-yl)-2-[4-(trifluoromethyl)phenyl]ethane-1,2-dione (0.373 mmol) in 3 mL of DMA, is added 43.7 mg of semicarbazide hydrochloride (0.392 mmol) and 276.4 mg of potassium carbonate (2 mmol). The reaction mixture is heated at 160° C. for 16 h. After cooling to rt, the reaction mixture is diluted with EtOAc and water. The aqueous layer is separated, and then neutralized with aqueous ammonium chloride. The aqueous layer is extracted with EtOAc (25 mL×3). The combined organic layers are washed with water and brine, dried over sodium sulfate, and concentrated to give essentially pure cyclized products as a mixture of two regio-isomers. The cyclized products are dissolved in 3 mL of phosphorus oxychloride, and 21.5 mg of N,N-diethylaniline is added. The resulting mixture is heated at 105° C. for 16 h. After cooling to rt, the reaction mixture is carefully poured into ice-water. The aqueous solution is neutralized with 1 N aqueous NaOH and extracted with EtOAc. The combined organic layers are washed with water and brine, dried over sodium sulfate, and concentrated. Purification of the residue by PTLC (20% EtOAc in hexanes) affords 3-chloro-5-(3-chloropyridin-4-yl)-6-[4-(trifluoromethyl)phenyl]-1,2,4-triazine and 3-chloro-6-(3-chloropyridin-4-yl)-5-[4-(trifluoromethyl)phenyl]-1,2,4-triazine.

3-chloro-5-(3-chloropyridin-4-yl)-6-[4-(trifluoromethyl)phenyl]-1,2,4-triazine: LC-MS: m/z for C₁₅H₇Cl₂F₃N₄ expected 370.0 (³⁵Cl), 372.0 (³⁷Cl), 374.0 (³⁷Cl×2); found 371.00 (MH⁺), 372.99 (MH⁺), 374.99 (MH⁺). ¹H-NMR ( δ, ppm, CDCl₃ as internal standard): 8.70 (d, J=4.8 Hz, 1H), 8.64 (s, 1H), 7.64 (d, J=8.7 Hz, 2H), 7.60 (d, J=8.7 Hz, 2H), 7.48 (d, J=5.1 Hz, 1H).

3-chloro-6-(3-chloropyridin-4-yl)-5-[4-(trifluoromethyl)phenyl]-1,2,4-triazine: LC-MS: m/z for C₁₅H₇Cl₂F₃N₄ expected 370.0 (³⁵Cl), 372.0 (³⁷Cl), 374.0 (³⁷Cl×2); found 371.00 (MH⁺), 373.00 (MH⁺), 375.00 (MH⁺). ¹H-NMR (δ, ppm, CDCl₃ as internal standard): 8.75 (d, J=5.1 Hz, 1H), 8.65 (s, 1H), 7.67 (d, J=9.3 Hz, 2H), 7.64 (d, J=5.1 Hz, 1H), 7.63 (d, J=9.3 Hz, 2H).

### STEP 4. 3-(ETHYLAMINO)-1-{5-(3-CHLOROPYRIDIN-4-YL)-6-[4-(TRIFLUOROMETHYL)PHENYL]-1,2,4-TRIAZIN-3-YL}AZETIDINE-3-CARBOXAMIDE

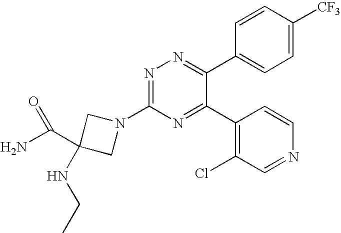

To a solution of 15 mg of 3-chloro-5-(3-chloropyridin-4-yl)-6-[4-(trifluoromethyl)phenyl]-1,2,4-triazine (0.04 mmol) in 1 mL of acetonitrile, is added 10.8 mg of 3-(ethylamino)azetidine-3-carboxamide (acetic acid salt, 0.06 mmol) and 33.2 mg of potassium carbonate (0.24 mmol). The resulting mixture is heated at 80° C. for 16 h. After being cooled to rt, the mixture is diluted with EtOAc and water. The organic layer is separated, and the aqueous layer is back extracted with EtOAc. The combined organic layers are washed with water and brine, dried over sodium sulfate, and concentrated. Purification of the residue by PTLC (5% MeOH in DCM) affords the title compound. LC-MS: m/z for C₂₁H₁₉ClF₃N₇O expected 477.13 (³⁵Cl), 479.13 (³⁷Cl); found 478.19 (MH⁺), 480.16 (MH⁺). ¹H-NMR (δ, ppm, CDCl₃ as internal standard): 8.60 (d, J=4.8 Hz, 1H), 8.59 (s, 1H), 7.53 (d, J=8.7 Hz, 2H), 7.45 (d, J=8.4 Hz, 2H), 7.32 (d, J=5.1 Hz, 1H), 7.09 (m, 1H), 5.54 (m, 1H), 4.75-4.73 (m, 2H), 4.18-4.11 (m, 2H), 2.67 (q, J=7.2 Hz, 2H), 1.20 (t, J=7.2 Hz, 3H).

### Example 53

### SYNTHESIS OF 4-[6-(3-CHLORO-PYRIDIN-4-YL)-5-(4-TRIFLUOROMETHYL-PHENYL)-PYRAZIN-2-YLOXY]-PIPERIDINE-1-CARBOXYLIC ACID TERT-BUTYL ESTER AND DERIVATIVES THEREOF

### STEP 1. 5-BROMO-6-CHLORO-PYRAZIN-2-YLAMINE

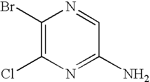

6-Chloro-2-aminopyrazine (21.6 g, 166.7 mmol) in 200 ml of CHCl₃ is treated with NBS (30 g, 168.4 mmol) at 0° C. in portions over 1.5 h. The reaction mixture is stirred for another 2 h and quenched by the addition of 100 ml water. The aqueous layer is extracted with CHCl₃ (3×100 ml). The combined organic layer is washed with sat. brine, dried over MgSO₄, concentrated and purified by column to give the title compound. NMR (CDCl₃, 400 MHz): δ 7.68 (s, 1H), 4.78 (2H, b); LC-MS: expected 208.44 (³⁵Cl), found 209.3 (MH⁺).

### STEP 2. 5-BROMO-6-CHLORO-PYRAZIN-2-OL

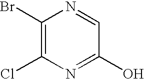

5-Bromo-6-chloro-2-aminopyrazine (2.08 g, 10 mmol) is dissolved in 16 ml concentrated H₂SO₄ and cooled to 0° C. Sodium nitrite (700 mg, 10 mmol) is added to the reaction mixture portionwise over 30 min, and stirring is continued for another 15 min. The sticky reaction mixture is slowly added to 100 g ice. The precipitate is collected by filtration and washed to acid-free to give the title compound as an off white solid. NMR (CDCl₃, 400 MHz): δ 11.8 (b, 1H), 7.80 (1H, s); LC-MS: expected 209.43 (³⁵Cl), found 210.3 (MH⁺).

### STEP 3. 4-(5-BROMO-6-CHLORO-PYRAZIN-2-YLOXY)-PIPERIDINE-1-CARBOXYLIC ACID TERT-BUTYL ESTER

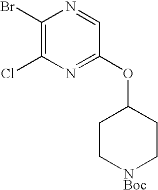

A mixture of 5-bromo-6-chloro-pyrazin-2-ol (1.4 g, 7 mmol), triphenylphosphine (2.2 g, 8.4 mmol) and 1-N-Boc-4-hydroxypiperdine (1.7 g, 8.4 mmol) in 20 ml THF is treated with DEAD (3.66 ml, 8.4 mmol, 40% in toluene) dropwise over 15 min. The reaction mixture is stirred overnight, concentrated, and purified by column chromatography to give the title compound as white solid. NMR (CDCl₃, 400 MHz): δ 7.80 (1H, s), 5.15 (1H, m), 3.74(2H, m), 3.31(2H, m), 1.97 (2H, m), 1.73 (2H, m), 1.47 (9H, s); LC-MS: expected 392.68, found 293.7 (MH⁺).

### STEP 4. 4-[6-CHLORO-5-(4-TRIFLUOROMETHYL-PHENYL)-PYRAZIN-2-YLOXY]-PIPERIDINE-1-CARBOXYLIC ACID TERT-BUTYL ESTER

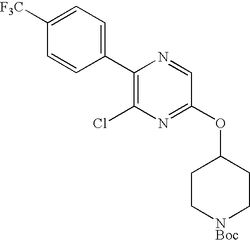

4-(5-Bromo-6-chloro-pyrazin-2-yloxy)-piperidine-1-carboxylic acid tert-butyl ester (1.1 g, 2.8 mmol), 4-trifluoromethylphenylboronic acid (0.47 g, 3.36 mmol) and Pd(PPh₃)₄ (64 mg, 2 mol %) in 25 ml 1,4-dioxone is treated with 4.2 ml of 2M K₂CO₃ (8.4 mmol). The reaction mixture is heated to reflux for 2 h. After cooling, the reaction mixture is diluted with 100 ml EtOAc. The aqueous layer is extracted with EtOAc (2×20 ml). The combined organic layer is washed with sat. brine, dried over MgSO₄, concentrated and purified by column chromatography to give the title compound. NMR (CDCl₃, 400 MHz): δ 7.80 (1H, s), 5.15 (1H, m), 3.74(2H, m), 3.31(2H, m), 1.97 (2H, m), 1.73 (2H, m), 1.47 (9H, s); LC-MS: expected 392.68, found 293.7 (MH⁺).

### STEP 5. 4-[6-(3-CHLORO-PYRIDIN-4-YL)-5-(4-TRIFLUOROMETHYL-PHENYL)-PYRAZIN-2-YLOXY]-PIPERIDINE-1-CARBOXYLIC ACID TERT-BUTYL ESTER

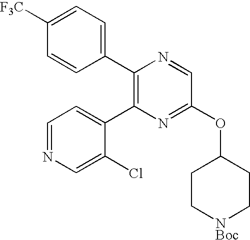

4-[6-Chloro-5-(4-trifluoromethyl-phenyl)-pyrazin-2-yloxy]-piperidine-1-carboxylic acid tert-butyl ester (1.1 g, 2.5 mmol), 3-chloro-pyridyl-4-boronic acid (1.3 g, 7.5 mmol) and Pd(PPh₃)₄ (64 mg, 2 mol %) in 25 ml 1,4-dioxone is treated with 6.5 ml of 2M K₂CO₃ (12.5 mmol). The reaction mixture is heated to 135° C. in a sealed tube overnight. After cooling, the reaction mixture is diluted with 100 ml EtOAc. The aqueous layer is extracted with EtOAc (2×20 ml). The combined organic layer is washed with sat. brine, dried over MgSO₄, concentrated and purified by column chromatography to give the title compound. LC-MS: expected 534.96 (³⁵Cl), found 535.3 (MH⁺).

The Boc group may be replaced with a variety of moieties using, for example, the methods described in Examples 6 and 7.

### Example 54

### SYNTHESIS OF 3-[6-(3-CHLORO-PYRIDIN-4-YL)-5-(4-TRIFLUOROMETHYL-PHENYL)-PYRAZIN-2-YLOXY]-AZETIDINE-1-CARBOXYLIC ACID TERT-BUTYL ESTER AND DERIVATIVES THEREOF

### STEP 1. 6-(3-CHLORO-PYRIDIN-4-YL)-PYRAZIN-2-YLAMINE

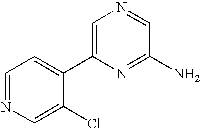

6-Chloro-2-aminopyrazine (7.8 g, 60 mmol), 3-chloro-pyridyl-4-boronic acid (14 g, 0.18 mol) and Pd(PPh₃)₄ (2 g, 5 mol %) in 180 ml of 1,4-dioxone is treated with 150 ml of 2M K₂CO₃ (0.3 mol). The reaction mixture is heated to 135° C. in a sealed tube overnight. After cooling, the reaction mixture is diluted with 250 ml EtOAc. The aqueous layer is extracted with EtOAc (2×100 ml). The combined organic layer is washed with sat. brine, dried over MgSO₄, concentrated and purified by column chromatography to give the title compound. NMR (CDCl₃, 400 MHz): δ 8.70 (s, 1H), 8.58 (1H, d, J=4 Hz)), 8.34 (1H, s), 8.04 (1H, s), 7.53 (1H, m), 4.72 (2H, b); LC-MS: expected 206.63 (³⁵Cl), found 207.3 (MH⁺).

### STEP 2. 5-BROMO-6-(3-CHLORO-PYRIDIN-4-YL)-PYRAZIN-2-YLAMINE

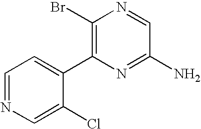

6-(3-Chloro-pyridin-4-yl)-pyrazin-2-ylamine (4.5 g, 21.8 mmol) in 100 ml of CHCl₃ is treated with NBS (3.9 g, 22 mmol) at 0° C. portionwise over 1.5 h. The reaction mixture is stirred for another 2 h and quenched by the addition of 100 ml water. The aqueous layer is extracted with CHCl₃ (3×100 ml). The combined organic layer is washed with sat. brine, dried over MgSO₄, concentrated and purified by column to give the title compound. NMR (CDCl₃, 400 MHz): δ 8.70 (s, 1H), 8.58 (1H, d, J=4 Hz)), 8.04 (1H, s), 7.53 (1H, m), 4.72 (2H, b); LC-MS: expected 285.53 (³⁵Cl), found 286.3 (MH⁺).

### STEP 3. 6-(3-CHLORO-PYRIDIN-4-YL)-5-(4-TRIFLUOROMETHYL-PHENYL)-PYRAZIN-2-YLAMINE

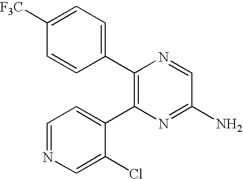

5-Bromo-6-(3-chloro-pyridin-4-yl)-pyrazin-2-ylamine (1.2 g, 4.2 mmol), 4-trifluoromethylphenylboronic acid (0.77 g, 4.62 mmol) and Pd(PPh₃)₄ (97 mg, 2 mol %) in 25 ml 1,4-dioxone is treated with 5 ml of 2M K₂CO₃ (10 mmol). The reaction mixture is heated to reflux for 2 h. After cooling, the reaction mixture is diluted with 100 ml EtOAc. The aqueous layer is extracted with EtOAc (2×20 ml). The combined organic layer is washed with sat. brine, dried over MgSO₄, concentrated and purified by column chromatography to give the title compound. NMR (CDCl₃, 400 MHz): δ 8.56 (s, 1H), 8.51 (1H, d, J=4 Hz), 8.15 (1H, s), 7.26 (2H, m), 6.92 (2H, m), 4.71 (2H, b); LC-MS: expected 350.73 (³⁵Cl), found 351.3 (MH⁺).

### STEP 4. 5-CHLORO-3-(3-CHLORO-PYRIDIN-4-YL)-2-(4-TRIFLUOROMETHYL-PHENYL)-PYRAZINE

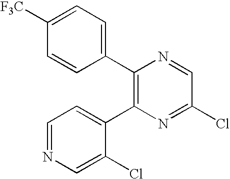

6-(3-Chloro-pyridin-4-yl)-5-(4-trifluoromethyl-phenyl)-pyrazin-2-ylamine (1 g, 2.85 mmol) and copper chloride (I) in 8 ml of concentrated HCl is treated with sodium nitrite (0.24 g, 3.42 mmol) at 0° C. portionwise over 30 min. The reaction mixture is stirred for another 1 h at 0° C. and 2 h at rt. The reaction is quenched by the addition of 25 ml of ice water and the pH is adjusted to 8 by 2N NaOH. The aqueous layer is extracted with EtOAc (3×60 ml). The combined organic layer is dried over MgSO₄, concentrated and purified by column chromatography to give the title compound. NMR (CDCl₃, 400 MHz): δ 8.74 (s, 1H), 8.58 (2H, t, J=4 Hz), 7.36 (2H, m), 7.0 (2H, m); LC-MS: expected 370.16 (³⁵Cl), found 371.3 (MH⁺).

### STEP 5. 3-[6-(3-CHLORO-PYRIDIN-4-YL)-5-(4-TRIFLUOROMETHYL-PHENYL)-PYRAZIN-2-YLOXY]-AZETIDINE-1-CARBOXYLIC ACID TERT-BUTYL ESTER

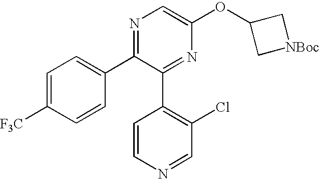

1-N-Boc-3-Hydroxyazetedine (0.21 g, 1.2 mmol) in 5 ml THF is treated with NaH (60 mg, 1.5 mmol, 60%). The reaction mixture is stirred for 30 min at rt, followed by the addition of 5-chloro-3-(3-chloro-pyridin-4-yl)-2-(4-trifluoromethyl-phenyl)-pyrazine (0.37, 1 mmol). The reaction mixture is stirred overnight and quenched by the addition of 10 ml 1M sat. NH₄Cl. The aqueous layer is extracted with EtOAc (3×25 ml). The combined organic layer is dried over MgSO₄, concentrated and purified by column to give the title compound. LC-MS: expected 506.93 (³⁵Cl), found 506.9 (MH⁺).

The Boc group may be replaced with a variety of moieties using, for example, the methods described in Examples 6 and 7.

### Example 55

### SYNTHESIS OF 3-[5-(3-CHLORO-PYRIDIN-4-YL)-6-(4-TRIFLUOROMETHYL-PHENYL)-PYRIDAZIN-3-YLOXY]-AZETIDINE-1-CARBOXYLIC ACID TERT-BUTYL ESTER AND RELATED COMPOUNDS

### STEP 1. 3-CHLORO-4-METHYLPYRIDINE

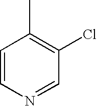

Diisopropylamine (25 ml, 185 mmol) in 200 ml THF is treated with 78 ml of n-BuLi (2.5M, 202.4 mmol) at −78° C. The reaction mixture is stirred for another 30 min. 3-Chloropyridine (20 g, 176 mmol) in 100 ml THF is added to the reaction mixture over 1.5 h, followed by the addition of 25 ml iodomethane in 80 ml of THF. The reaction mixture is stirred for another 1 h before quenching by the addition of 200 ml 1M HCl. The aqueous layer is extracted with EtOAc (3×150 ml). The combined organic layer is dried over MgSO₄, concentrated and purified by column to give the title compound as an oil. NMR (CDCl₃, 400 MHz): δ 8.50 (s, 1H), 8.35 (1H, d, J=2 Hz), 7.14 (1H, d J=2 Hz), 2.27 (3H, s); LC-MS: expected 127.57 (³⁵Cl), 129.57 (³⁷Cl); found 128.3 (MH⁺), 130.3 (MH⁺).

### STEP 2. 1-(3-CHLORO-PYRIDIN-4-YL)-2-(4-TRIFLUOROMETHYL-PHENYL)-ETHANONE

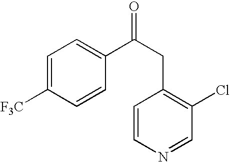

To a solution of 3-chloro-4-methylpyridine (8 g, 62.7 mmol) in 100 ml THF is added 0.5M potassium hexamethyldisilizane (69 mmol, 138 ml) at −50° C. over 10 min. The reaction mixture is stirred for another 30 min. Trifluoromethylbenzoate ethyl ester (14 g, 69 mmol) in 60 ml of THF is added to the reaction mixture. The reaction mixture is stirred overnight and quenched by the addition of 100 ml 1M HCl. The aqueous layer is extracted with EtOAc (3×100 ml). The combined organic layer is dried over MgSO₄, concentrated and purified by column to give the title compound as an oil. NMR (CDCl₃, 400 MHz): δ 8.63 (s, 1H), 8.48 (1H, d, J=2 Hz), 8.14 (2H, d, J=8 Hz), 7.80 (2H, d, J=8 Hz), 7.21 (1H, d, J=2 Hz), 4.46 (2H, s); LC-MS: expected 299.68 (³⁵Cl), found 300.3 (MH⁺).

### STEP 3. 4-(3-CHLORO-PYRIDIN-4-YL)-4-OXO-3-(4-TRIFLUOROMETHYL-PHENYL)-BUTYRIC ACID ETHYL ESTER

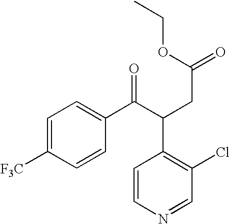

1-(3-Chloro-pyridin-4-yl)-2-(4-trifluoromethyl-phenyl)-ethanone (4.7 g, 15.72 mmol) in 80 ml of DMSO is treated with NaH (880 mg, 60%, 22.5 mmol) at 0° C. The reaction is stirred for 1 h at rt and BrCH₂CO₂Et (2.26 ml, 20 mmol) is slowly added. The reaction is stirred for another 5 h and quenched by the addition of 100 ml of 1M HCl. The aqueous layer is extracted with EtOAc (3×100 ml). The combined organic layer is dried over MgSO₄, concentrated and purified by column to give the title compound as an oil. NMR (CDCl₃, 400 MHz): δ 8.63 (s, 1H), 8.38 (1H, d, J=4 Hz), 8.04 (2H, d, J=8 Hz), 7.70 (2H, d, J=8 Hz), 7.09 (1H, d, J=4 Hz), 5.52 (1H, dd, J=4, 12 Hz), 4.14 (2H, m), 3.28 (1H, dd, J=12, 20 HZ), 2.69 (1H, dd, J=4, 12 Hz); LC-MS: expected 385.76 (³⁵Cl), found 386.3 (MH⁺).

### STEP 4. 5-(3-CHLORO-PYRIDIN-4-YL)-6-(4-TRIFLUOROMETHYL-PHENYL)-4,5-DIHYDRO-2H-PYRIDAZIN-3-ONE

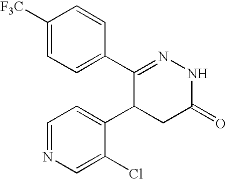

4-(3-Chloro-pyridin-4-yl)-4-oxo-3-(4-trifluoromethyl-phenyl)-butyric acid ethyl ester (3.6 g, 9.35 mmol) in 10 ml of tert-amyl alcohol is treated with anhydrous hydrazine (0.3 ml, 9.35 mmol) at 90° C. The reaction mixture is stirred for 24 h and concentrated at 180° C. under vacuum. The residue is dissolved in EtOAc (100 ml), washed with sat. NH₄Cl, followed by sat. brine, dried over MgSO₄, concentrated and purified by column chromatography to give the title compound as an oil. NMR (CDCl₃, 400 MHz): δ 8.87 (s, 1H), 8.69 (1H, s), 8.04 (1H, d, J=4 Hz), 7.70 (2H, d, J=8 Hz), 7.62 (2H, d, J=8 Hz), 6.68 (1H, d, J=4 Hz), 4.48 (1H, m), 3.04 (1H, m), 2.88 (1H, m); LC-MS: expected 353.73 (³⁵Cl), found 354.3 (MH⁺).

### STEP 5. 5-(3-CHLORO-PYRIDIN-4-YL)-6-(4-TRIFLUOROMETHYL-PHENYL)-PYRIDAZIN-3-OL

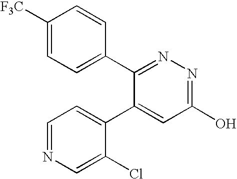

5-(3-Chloro-pyridin-4-yl)-6-(4-trifluoromethyl-phenyl)-4,5-dihydro-2H-pyridazin-3-one (1.6 g, 4.52 mmol) in 20 ml acetic acid is heated to 70° C. and treated with bromine (0.465 ml, 9.04 mmol). The reaction is stirred for 24 h and concentrated under vacuum. The residue is dissolved in EtOAc (100 ml), washed with sat. Na₂CO₃, followed by sat. brine, dried over MgSO₄, concentrated and purified by column chromatography to give the title compound as a white solid. LC-MS: expected 353.73 (³⁵Cl), found 354.3 (MH⁺).

### STEP 6. 6-CHLORO-4-(3-CHLORO-PYRIDIN-4-YL)-3-(4-TRIFLUOROMETHYL-PHENYL)-PYRIDAZINE

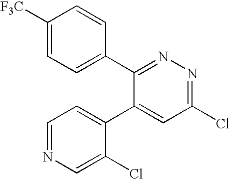

5-(3-Chloro-pyridin-4-yl)-6-(4-trifluoromethyl-phenyl)-pyridazin-3-ol (1.5 g, 4.26 mmol) is dissolved in 15 ml trichlorophosphine oxide and heated to reflux overnight. The reaction mixture is concentrated and the residue is dissolved in water. pH is adjusted to 7 by the addition of 5N NaOH. The aqueous layer is extracted with EtOAc (3×60 ml). The combined organic layer is dried over MgSO₄, concentrated and purified by column chromatography to give the title compound. LC-MS: expected 370.16 (³⁵Cl), found 371.3 (MH⁺).

### STEP 7A. 4-[5-(3-CHLORO-PYRIDIN-4-YL)-6-(4-TRIFLUOROMETHYL-PHENYL)-PYRIDAZIN-3-YL]-PIPERAZINE-1-CARBOXYLIC ACID TERT-BUTYL ESTER

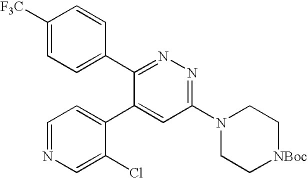

6-Chloro-4-(3-chloro-pyridin-4-yl)-3-(4-trifluoromethyl-phenyl)-pyridazine (0.37 g, 1 mmol) and N-bocpiperazine (0.19 g, 1.02 mmol), in 2 ml DMSO and 2 ml of DMA, is treated with KF (0.11, 2 mmol) at 85° C. The reaction is stirred for 16 h. The mixture is then diluted with 60 ml EtOAc, washed with sat. NH₄Cl, followed by sat. brine, dried over MgSO₄, concentrated and purified by column chromatography to give the title compound. LC-MS: expected 519.93, found 521.3 (MH⁺).

### STEP 7B. {1-[5-(3-CHLORO-PYRIDIN-4-YL)-6-(4-TRIFLUOROMETHYL-PHENYL)-PYRIDAZIN-3-YL]-PIPERIDIN-4-YL}-CARBAMIC ACID TERT-BUTYL ESTER

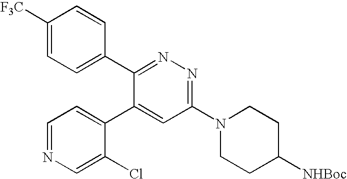

This compound is prepared from 6-Chloro-4-(3-chloro-pyridin-4-yl)-3-(4-trifluoromethyl-phenyl)-pyridazine and tert-butyl piperidin-4-ylcarbamate, as described in Step 7A. LC-MS: expected 533.93, found 535.1 (MH⁺).

### STEP 7C. 3-[5-(3-CHLORO-PYRIDIN-4-YL)-6-(4-TRIFLUOROMETHYL-PHENYL)-PYRIDAZIN-3-YLOXY]-AZETIDINE-1-CARBOXYLIC ACID TERT-BUTYL ESTER

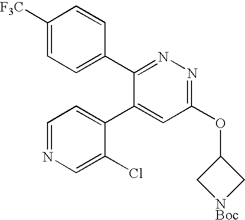

1-N-Boc-3-Hydroxyazetedine (0.21 g, 1.2 mmol) in 5 ml THF is treated with NaH (60 mg, 1.5 mmol, 60%). The reaction mixture is stirred for 30 min at rt, followed by the addition of 6-chloro-4-(3-chloro-pyridin-4-yl)-3-(4-trifluoromethyl-phenyl)-pyridazine (0.37, 1 mmol). The reaction mixture is stirred overnight and quenched by the addition of 10 ml 1M sat. NH₄Cl. The aqueous layer is extracted with EtOAc (3×25 ml). The combined organic layer is dried over MgSO₄, concentrated and purified by column to give the title compound. LC-MS: expected 506.9, found 507.3 (MH⁺).

### Example 56

### SYNTHESIS OF 3′-CHLORO-6-(4-PROPIONYPIPERAZIN-1-YL)-3-[4-(TRIFLOUROMETHYL)PHENYL]-[2,4′]-BIPYRIDINE

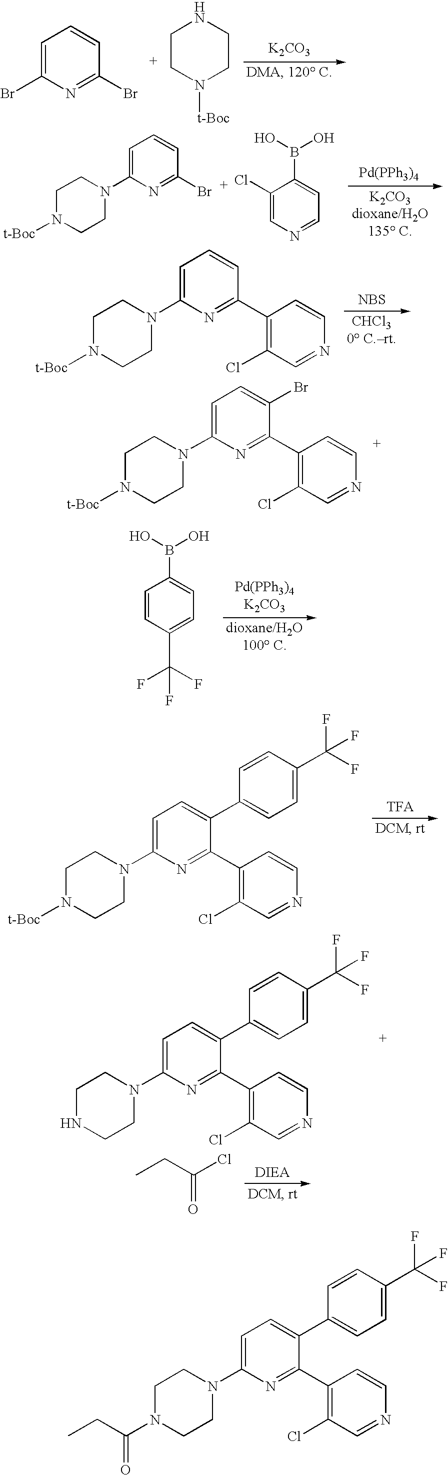

### STEP 1. PREPARATION OF 4-(6-BROMO-PYRIDIN-2-YL)-PIPERAZINE-1-CARBOXYLIC ACID TERT-BUTYL ESTER

A mixture of 2,6-dibromo-pyridine (1.0 g, 4.2 mmol), 4-tert-butylcarboxylate-piperazine (0.78 g, 4.2 mmol) and K₂CO₃ (0.69 g, 5.0 mmol) in DMA (20 mL) is heated at 120° C. for 14 h. The reaction mixture is cooled. H₂O and EtOAc are added. The organic layer is separated. EtOAc is extracted (2×30 mL). The combined organic layer is dried over Na₂SO₄ and evaporated under reduced pressure. The crude product is used without further purification in the next step.

### STEP 2. PREPARATION OF 4-(3′-CHLORO-[2,4]-BIYRIDINYL-6-YL)-PIPERAZINE-CARBOXYLIC ACID TERT-BUTYL ESTER

A mixture of 4-(6-bromo-pyridin-2-yl)-piperazine-1-carboxylic acid tert-butyl ester (1.44 g, 4.2 mmol), 3-chloro-4-pyridynboronic acid (2.0 g, 12.6 mmol), K₂CO₃ (3.5 g, 25.2 mmol) and Pd(PPh₃)₄ (500 mg, 2% mmol) is dissolved in degassed dioxane (25 mL)/H₂O (12 mL) and heated to 135° C. for 16 h. The reaction mixture is cooled. The crude product is purified by flash column and eluted with 1% MeOH/DCM to give the title compound.

### STEP 3. PREPARATION OF 4-(3-BROMO-3′-CHLORO-[2,4′]-BIPYRIDINYL-6-YL)-PIPERAZINE-1-CARBOXYIC ACID TERT-BUTYL ESTER

4-(3′-chloro-[2,4]-bipyridinyl-6-yl)-piperazine-carboxylic acid tert-butyl ester (0.98 g, 2.61 mmol) is dissolved in CHCl₃ (15 mL) and cooled to 0° C. NBS (445 mg, 2.5 mmol) is added portionwise and the mixture is warmed to rt and stirred for 14 h. The crude product is purified by a flash column and eluted with 1% MeOH/DCM to give the title compound.

### STEP 4. PREPARATION OF 4-[3′-CHLORO-3-(4-TRIFLUOROMETHYL-PHENYL)-[2,4′]-BIPYRIDINY-6-YL]-PIPERAZINE-1-CARBOXYLIC ACID TERT-BUTYL ESTER

A mixture of 4-(3-bromo-3′-chloro-[2,4′]bipyridinyl-6-yl)-piperazine-1-carboxyic acid tert-butyl ester (500 mg, 1.1 mmol), 4-(trifluoromethyl)phenylboronic acid (228 mg, 1.2 mmol), K₂CO3 (346 mg, 2.5 mmol) and Pd(PPh₃)₄ (50 mg, 5% mmol) is dissolved in degassed dioxane (10 mL)/H₂O (1.3 mL) and heated to 100° C. for 14 h. The reaction mixture is cooled. The crude product is purified by PTLC and eluted with 1% MeOH/DCM to give the title compound.

### STEP 5. PREPARATION OF 3′-CHLORO-6-PIPERAZIN-1-YL-3-(4-TRIFLOUROMETHYL-PHENYL)-[2,4′]-BIPYRIDINE

4-[3′-Chloro-3-(4-triflouromethyl-phenyl)-[2,4′]bipyridiny-6-yl]-piperazine-1-carboxylic acid tert-butyl ester (470 mg. 0.9 mmol) is dissolved in DCM (7.5 mL) at rt. TFA (1 mL) is added dropwise and the mixture is stirred for 6 h. The solvent is removed under reduced pressure. EtOAc is added and the solution is washed with NaHCO₃ (2×10 mL) and dried over Na₂SO₄. The solvent is removed under reduced pressure to give the title compound, which is used in the next step without further purification.

### STEP 6. PREPARATION OF 3′-CHLORO-6-(4-PROPIONYPIPERAZIN-1-YL)-3-[4-(TRIFLOUROMETHYL)PHENYL]-[2,4′]-BIPYRIDINE

3′-Chloro-6-piperazin-1-yl-3-(4-triflouromethyl-phenyl)-[2,4′]-bipyridine (10 mg, 0.024 mmol) is dissolved in DCM at rt. DIEA (0.1 mL, excess) is added and the reaction is stirred for 5 min. Propionyl chloride (2 drops) is added and the reaction is stirred for 1 h at rt, purified by PTLC and eluted with 0.5% MeOH/DCM to give the title compound. ¹H NMR (CDCl₃): 8.54 (s, 1H), 8.42-8.40 (d, 1H), 7.65-6.62 (d, 1H), 7.47-7.44 (d, 2H), 7.18-7.16 (m, 3H), 6.83-6.80 (d, 1H), 3.79-3.71 (m, 4H), 3.62-3.57 (m, 4H), 2.41-2.39 (q, 2H), 1.21-1.15 (t, 3H). m/z: 474.0.

### Example 57

### SYNTHESIS OF 3′-CHLORO-2-(1,1-DIOXIDOTHIOMORPHOLIN-4-YL)-5-[4-(TRIFLUOROMETHYL)PHENYL]-4,4′-BIPYRIDINE

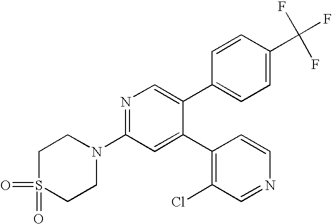

### STEP 1. PREPARATION OF 2,3′-DICHLORO 4,4′-BIPYRIDINE

A mixture of 2-chloro-4-iodo-pyridine (1.0 g, 4.2 mmol), 3-chloropyridine-4-boronic acid (2.0 g, 12.7 mmol), Na₂CO₃ (2.7 g, 25 mmol), Pd(PPh₃)₄ (0.2 g), dioxane (30 mL) and water (15 mL) in a sealed tube is purged with argon gas for 15 min, and stirred at 130° C. overnight. The mixture is cooled, diluted with water and 1 N NaOH, and extracted with EtOAc. The extract is washed once with water and concentrated under vacuum. The residue is purified by silica gel column (5% MeOH in CH₂Cl₂) to produce the title compound. LC-MS; Rt=1.38 minute. mass expected (225.08), mass found (226.93, M+1).

### STEP 2. PREPARATION OF 3′-CHLORO-2-THIOMORPHOLIN-4-YL-4,4′-BIPYRIDINE

A mixture of 2,3′-dichloro-4,4′-bipyridine (1.2 g, 5.3 mmol), thiomorpholine (1.1 g, 10.6 mmol) and K₂CO₃ (1.5 g, 10.6 mmol) in DMA (15 mL) is stirred at 140° C. overnight. The mixture is cooled, and diluted with water. The product is collected by filtration and dried. LC-MS; Rt=1.49 minute. mass expected (291.80), mass found (292.43, M+1).

### STEP 3. PREPARATION OF 5-BROMO-3′-CHLORO-2-THIOMORPHOLIN-4-YL-4,4′-BIPYRIDINE

A mixture of 3′-chloro-2-thiomorpholin-4-yl-4,4′-bipyridine (1.3 g, 4.5 mmol) and NBS (478 mg, 2.7 mmol) in CHCl₃ (25 mL) is stirred at rt overnight. The reaction mixture is washed with diluted aqueous Na₂CO₃ solution and brine, dried, concentrated under vacuum, and purified by flash column chromatography using Hexane/EtOAc (4:1) to afford the title compound as a white solid. ¹H NMR (CDCl₃): 8.71 (s, 1H), 8.57 (d, 1H), 8.33 (s, 1H), 7.20 (d, 1H), 6.45 (s, 1H), 3.95 (m, 4H), 2.67 (m, 4H).

### STEP 4. PREPARATION OF 5-BROMO-3′-CHLORO-2-(1,1-DIOXOTHIOMORPHOLIN-4-YL)-4,4′-BIPYRIDINE

A mixture of 5-bromo-3′-chloro-2-thiomorpholin-4-yl-4,4′-bipyridine (320 mg, 0.86 mmol) and m-CPBA (77%, 386 mg, 1.72 mmol) in CH₂Cl₂ (10 mL) is stirred at rt overnight. The reaction mixture is diluted with CH₂Cl₂ and washed with aqueous Na₂CO₃ and water, and concentrated to give the title compound. ¹H NMR (CDCl₃): 8.73 (s, 1H), 8.60 (d, 1H), 8.41 (s, 1H), 7.18 (d, 1H), 6.60 (s, 1H), 4.17 (m, 4H), 3.07 (m, 4H).

### STEP 5. PREPARATION OF 3′-CHLORO-2-(1,1-DIOXIDOTHIOMORPHOLIN4-YL)-5-[4-(TRIFLUOROMETHYL)PHENYL]-4,4′-BIPYRIDINE

A mixture of 5-bromo-3′-chloro-2-(1,1-dioxothiomorpholin-4-yl)-4,4′-bipyridine (30 mg, 0.74 mmol), 4-(trifluoromethyl)benzeneboronic acid (14 mg, 0.74 mmol), Na₂CO₃ (16 mg, 0.15 mmol), Pd(PPh₃)₄ (3 mg), dioxane (0.5 mL) and water (0.1 mL) in a sealed tube is purged with argon gas for 10 min, and stirred at 90° C. overnight. The mixture is cooled, diluted with water and 1 N NaOH, and extracted with EtOAc. The extract is washed once with water and concentrated under vacuum. The residue is purified by PTLC (5% MeOH in CH₂Cl₂) to produce the title compound. ¹H NMR (CDCl₃): 8.59 (s, 1H), 8.43 (d, 1H), 8.32 (s, 1H), 7.49 (d, 2H), 7.18 (d, 2H), 7.03 (d, 1H), 6.67 (s, 1H), 4.25 (m, 4H), 3.12 (m, 4H). LC-MS; Rt=1.32 minute. Mass expected (467.07); mass found (468.29, M+1).

### Example 58

### SYNTHESIS OF N-{1-[6-(3-CHLORO-PYRIDIN-4-YL)-5-(4-TRIFLUOROMETHYL-PHENYL)-PYRAZIN-2-YL]-PIPERIDIN-4-YL}-N-METHYL-ISOBUTYRAMIDE AND RELATED COMPOUNDS

### STEP 1. SYNTHESIS OF 1-(6-CHLORO-PYRAZIN-2-YL)-PIPERIDIN-4-OL

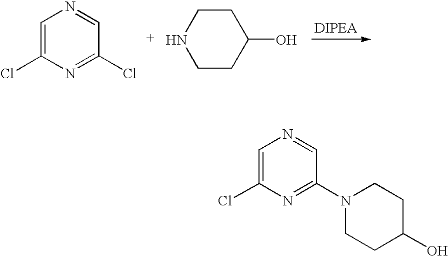

A mixture of 2,6-dichloro-pyrazine (5 g, 0.0336 mol), 4-hydroxypiperazine (3.4 g, 0.0336 mol) and N,N-diisopropylethyl amine (DIPEA) (8.67 g, 0.0672 mol) in CH₃CN (80 mL) is stirred under N₂ for 5 h and concentrated to dryness. The residue is dissolved in EtOAc (200 mL). The solution is washed with sat. Na₂CO₃, and then with brine, and dried over Na₂SO₄. Removal of solvent gives the title compound as a white solid. ¹H NMR (CDCl₃, 400 MHz): δ 7.989 (1H, s), 7.769 (1H, s), 3.9984.057 (3H, m), 3.278-3.343 (2H, m), 1.955-2.039 (2H, m), 1.581-1.646 (3H, m). LC-MS: m/z expected 213.07, found 213.97 (MH⁺).

### STEP 2. SYNTHESIS OF 2-[1-(6-CHLORO-PYRAZIN-2-YL)-PIPERIDIN-4-YL]-ISOINDOLE-1,3-DIONE

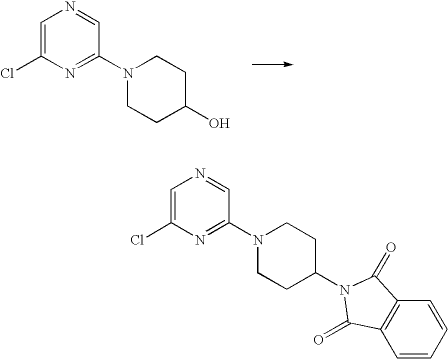

To a solution of 1-(6-chloro-pyrazin-2-yl)-piperidin-4-ol (14.5 g, 0.0679 mol), phthalimide (10 g, 0.0679 mol) and triphenylphosphine (21.3 g, 0.0814 mol) in THF (200 mL) is added diisopropyl azodicarboxylate over a period of 10 min with an ice/water bath cooling. The mixture is stirred at 0° C. to rt for 2 h. The solid is filtered to give the first yield of product as a white solid. Mother liquor is concentrated to a half of original volume and stirred in an ice/water bath for 30 min. The precipitate is collected to afford a second yield of product. ¹H NMR (CDCl₃, 400 MHz): δ 8.031 (1H, s), 7.812-7.843(2H, m), 7.710-7.731 (2H, m), 7.258 (1H, s), 4.4934.532 (2H, m), 4.40 (1H, m), 3.013 (2H, m), 2.50-2.55 (2H, m), 1.826-1.856(2H, m).

### STEP 3. SYNTHESIS OF 2-{1-[6-(3-CHLORO-PYRIDIN-4-YL)-PYRAZIN-2-YL]-PIPERIDIN-4-YL}-ISOINDOLE-1,3-DIONE

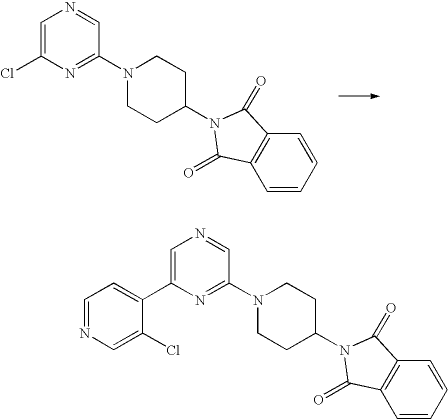

To a solution of 3-chloropyridine (2.62 g, 0.023 mol) in THF (250 mL) is added lithium diisopropylamide (14 mL, 1.8 M in THF, 0.0252 mol) at −78° C. The solution is stirred at the same temperature for 1 h. A solution of zinc chloride in THF (50.4 mL, 0.5 M, 0.0252 mol) is added to the flask. The cooling bath is removed and the temperature is allowed to warn to rt. 2-[1-(6-Chloro-pyrazin-2-yl)-piperidin-4-yl]-isoindole-1,3-dione (3.2 g, 0.0093 mol) and Pd(PPh₃)₄ (1.2 g, 0.00105 mol) are added to the flask. The resulting mixture is stirred under reflux overnight, and then quenched with NH₄Cl. The two layers are separated, and the aqueous layer is extracted with EtOAc. The combined organic phase is washed with brine, dried over Na₂SO₄. Column purification gives the product as an off-white solid. ¹H NMR (CDCl₃, 400 MHz): δ 8.70 (1H, s), 8.571-8.584 (1H, m), 8.293 (1H, s), 8.248 (1H, s), 7.824-7.848 (2H, m), 7.710-7.732 (2H, m), 7.577 (1H, d), 4.605-4.639(2H, m), 4.440 (1H, m), 3.0-3.05(2H, m), 2.50-2.60 (2H, m), 1.84-1.87 (2H, d, J=10.4 Hz).

### STEP 4. SYNTHESIS OF 2-{1-[5-BROMO-6-(3-CHLORO-PYRIDIN-4-YL)-PYRAZIN-2-YL]-PIPERIDIN-4-YL}-ISOINDOLE-1,3-DIONE

A mixture of 2-{1-[6-(3-chloro-pyridin-4-yl)-pyrazin-2-yl]-piperidin-4-yl}-isoindole-1,3-dione (3.2 g, 7.62 mmol) and NBS (1.42 g, 8.0 mmol) in CHCl₃ (250 mL) is stirred at −10° C. to 0° C. for 3 h. The reaction mixture is washed with diluted aqueous Na₂CO₃ solution and brine, dried and concentrated under vacuum, and purified by flash column chromatography using 10 to 25% EtOAc in hexane to afford the title compound as a white solid. LC-MS: m/z expected 499.02, found 499.86 (MH⁺).

### STEP 5. SYNTHESIS OF 2-{1-[6-(3-CHLORO-PYRIDIN-4-YL)-5-(5-TRIFLUOROMETHYL-PYRIDIN-2-YL)-PYRAZIN-2-YL]-PIPERIDIN-4-YL}-ISOINDOLE-1,3-DIONE

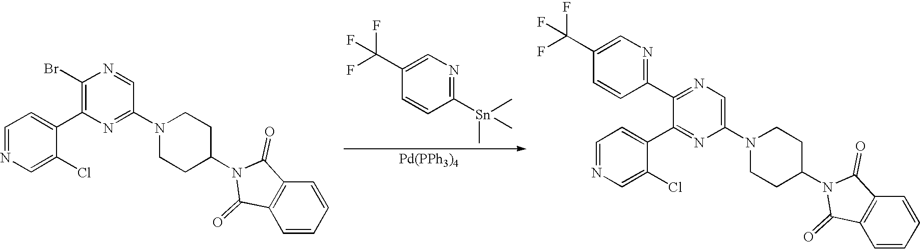

A solution of 2-chloro-5-trifluoromethylpyridine (1.60 g, 8.8 mmol) hexamethylditin (2.88 g, 8.8 mmol) and Pd(PPh₃)₄ (0.255 g, 0.22 mmol) in mesitylene (80 mL) is heated to 110° C. under nitrogen for 3 h, and then cooled. 2-{1-[5-bromo-6-(3-chloro-pyridin-4-yl)-pyrazin-2-yl]-piperidin-4-yl}-isoindole-1,3-dione (2.2 g, 4.4 mmol) and Pd(PPh₃)₄ (0.51 g, 0.44 mmol) are added. The resulting mixture is stirred at 140° C. overnight. Removal of the volatiles under high vacuum gives a residue, which is column purified to yield the title compound as a pale yellow solid. ¹H NMR (CDCl₃, 400 MHz): δ 8.53-8.544 (2H, m), 8.44 (1H, s), 8.31 (1H, s), 8.067-8.088 (1H, d,) 7.890-7.917 (1H, m), 7.819-7.839 (2H, m), 7.703-7.725 (2H, m), 7.352-7.363(1H, d), 4.25(2H, d, J=14 Hz), 4.450 (1H, m), 3.054-3.115 (2H, m), 2.543-2.586 (2H, m), 1.858-1.884 (2H, d, J=10.4 Hz). LC-MS: m/z expected 564.13, found 564.98 (MH⁺).

### STEP 6. SYNTHESIS OF 1-[6-(3-CHLORO-PYRIDIN-4-YL)-5-(5-TRIFLUOROMETHYL-PYRIDIN-2-YL)-PYRAZIN-2-YL]-PIPERIDIN-4-YLAMINE

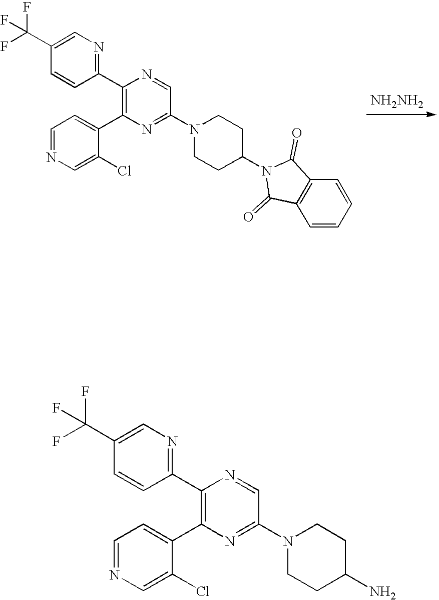

To a solution of 2-{1-[6-(3-chloro-pyridin-4-yl)-5-(5-trifluoromethyl-pyridin-2-yl)-pyrazin-2-yl]-piperidin-4-yl}-isoindole-1,3-dione (0.6 g, 1.06 mmol) in EtOH (20 mL) is added NH₂NH₂ (0.068 g, 2.12 mmol) in one portion. The resulting mixture is stirred at rt for 1.5 h. The solution is concentrated to half volume. EtOAc (20 mL) is added and the mixture is cooled to rt and filtered. Mother liquor is concentrated to almost dryness, and EtOAc (20 mL) is added again. The cloudy mixture is filtered again. Mother liquor is concentrated to dryness to afford the title compound as an off-white solid. LC-MS: m/z expected 434.12; found 435.02 (MH⁺).

### STEP 7. SYNTHESIS OF N-{1-[6-(3-CHLORO-PYRIDIN-4-YL)-5-(5-TRIFLUOROMETHYL-PYRIDIN-2-YL)-PYRAZIN-2-YL]-PIPERIDIN-4-YL}-ACETAMIDE

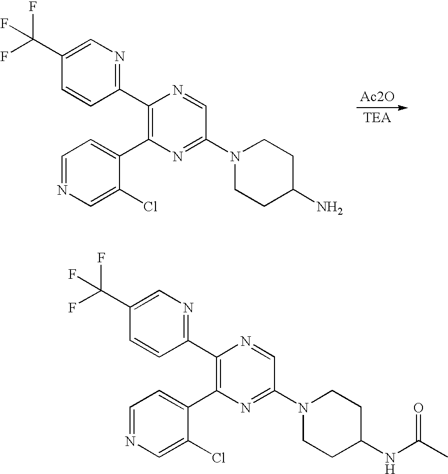

To a solution of 1-[6-(3-Chloro-pyridin-4-yl)-5-(5-trifluoromethyl-pyridin-2-yl)-pyrazin-2-yl]-piperidin-4-ylamine (44 mg, 0.1 mmol) in anhydrous DCM (2 mL) is added TEA (20 mg, 0.2 mmol), followed by acetic anhydride (15.3 mg, 0.15 mmol). The mixture is allowed to stand at rt overnight. PTLC purification gives a white solid product. MS: m/z expected 476.13; found 477.03 (MH+).

### SYNTHESIS OF N-{1-[6-(3-CHLORO-PYRIDIN-4-YL)-5-(4-TRIFLUOROMETHYL-PHENYL)-PYRAZIN-2-YL]-PIPERIDIN-4-YL}-N-METHYL-ISOBUTYRAMIDE

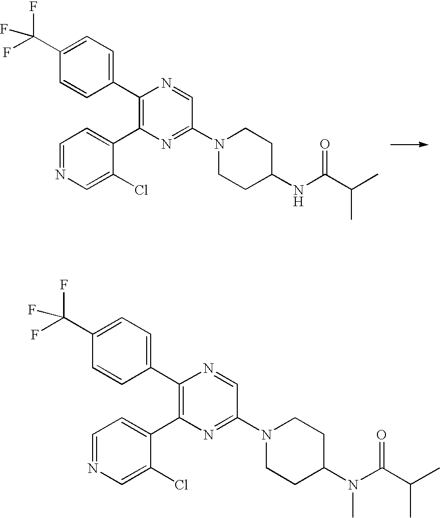

To a solution of N-{1-[6-(3-Chloro-pyridin-4-yl)-5-(4-trifluoromethyl-phenyl)-pyrazin-2-yl]-piperidin-4-yl}-isobutyramide (15 mg, 0.03 mmol—prepared essentially as described in steps 1-7 above, employing readily apparent starting materials) in anhydrous THF (1 mL) is added MeI (6.4 mg, 0.045 mmol) and t-BuOK (0.045 mL, 0.045 mmol, 1.0 M in THF). The mixture is stirred at rt for 1 h. PTLC purification gives a white solid product. MS: m/z expected 517.97; found 518.10 (MH+).

### Example 59

### Additional Representative CB1 Antagonists

Using routine modifications, the starting materials may be varied and additional steps employed to produce other compounds provided herein. Compounds listed in Tables I-IV are prepared using such methods. In Tables I-III, a “*” in the column headed “IC₅₀” indicates that the IC₅₀ at CB1, determined as described in Example 64, herein, is 2 micromolar or less. “Ret.” is the retention time in min and mass spectroscopy data generated as described above is presented as in the column headed “MS”. All mass spectroscopy data is presented as M+1 unless otherwise noted.

LENGTHY TABLE REFERENCED HERE
US20070078135A1-20070405-T00001
Please refer to the end of the specification for access instructions.

LENGTHY TABLE REFERENCED HERE
US20070078135A1-20070405-T00002
Please refer to the end of the specification for access instructions.

LENGTHY TABLE REFERENCED HERE
US20070078135A1-20070405-T00003
Please refer to the end of the specification for access instructions.

LENGTHY TABLE REFERENCED HERE
US20070078135A1-20070405-T00004
Please refer to the end of the specification for access instructions.

### Example 60

### Baculoviral Preparations for CB1 Expression

This Example illustrates the preparation of recombinant baculovirus for use in generating CB1-expressing insect cells.

The human CB1 sequence has GenBank Accession Number HSU73304, and was reported by Hoehe et al. (1991) New Biol. 3(9):880-85. Human CB1 (hCB1) cDNA is amplified from a human brain cDNA library (Gibco BRL, Gaithersburg, Md.) using PCR, in which the 5′ primer includes the optimal Kozak sequence CCACC. The resulting PCR product is cloned into pcDNA3.1/V5-His-TOPO (Invitrogen Corp, Carlsbad, Calif.) using the multiple cloning site, and then subcloned into pBACPAK₈ (BD Biosciences, Palo Alto, Calif.) at the Bam/Xho site to yield a hCB1 baculoviral expression vector.

The hCB1 baculoviral expression vector is co-transfected along with BACULOGOLD DNA (BD PharMingen, San Diego, Calif.) into Sf9 cells. The Sf9 cell culture supernatant is harvested three days post-transfection. The recombinant virus-containing supernatant is serially diluted in Hink's TNM-FH insect medium (JRH Biosciences, Kansas City, Mo.) supplemented with Grace's salts and with 4.1 mM L-Gln, 3.3 g/L LAH, 3.3 g/L ultrafiltered yeastolate and 10% heat-inactivated fetal bovine serum (hereinafter “insect medium”) and plaque assayed for recombinant plaques. After four days, recombinant plaques are selected and harvested into 1 ml of insect medium for amplification. Each 1 ml volume of recombinant baculovirus (at passage 0) is used to infect a separate T25 flask containing 2×10⁶ Sf9 cells in 5 ml of insect medium. After five days of incubation at 27° C., supernatant medium is harvested from each of the T25 infections for use as passage 1 inoculum.

Two of seven recombinant baculoviral clones are then chosen for a second round of amplification, using 1 ml of passage 1 stock to infect 1×10⁸ cells in 100 ml of insect medium divided into 2 T175 flasks. Forty-eight hours post infection, passage 2 medium from each 100 ml preparation is harvested and plaque assayed for titer. The cell pellets from the second round of amplification are assayed by affinity binding as described below to verify recombinant receptor expression. A third round of amplification is then initiated using a multiplicity of infection of 0.1 to infect a liter of Sf9 cells. Seventy-two hours post-infection the supernatant medium is harvested to yield passage 3 baculoviral stock.

The remaining cell pellet is assayed for affinity binding. Radioligand is 25 pM-5.0 nM [³H]CP55,940 for saturation binding and 0.5 nM for competition binding (New England Nuclear Corp., Boston, Mass.); the hCB1-expressing baculoviral cells are used; the assay buffer contains 50 mM Tris pH 7.4, 120 mM NaCl, 5 mM MgCl₂, 0.5% BSA and 0.2 mg/ml bacitracin; filtration is carried out using GF/C WHATMAN filters (presoaked in 0.3% non-fat dry milk (H₂O) for 2 hours prior to use); and the filters are washed twice with 5 mL cold 50 mM Tris pH. 7.4.

Titer of the passage 3 baculoviral stock is determined by plaque assay and a multiplicity of infection, incubation time course, binding assay experiment is carried out to determine conditions for optimal receptor expression.

### Example 61

### Baculoviral Infections

Log-phase Sf9 cells (Invitrogen Corp., Carlsbad, Calif.), are infected with one or more stocks of recombinant baculovirus followed by culturing in insect medium at 27° C. Infections are carried out either only with virus directing the expression of hCB1 or with this virus in combination with three G-protein subunit-expression virus stocks: 1) rat Gαₜ₂ G-protein-encoding virus stock, 2) bovine β1 G-protein-encoding virus stock, and 3) human γ2 G-protein-encoding virus stock, all of which are obtained from Biosignal Inc., Montreal, Canada.

Typical hCB1 infections are conducted using Sf9 cells that are cultured in insect medium supplemented with 10% heat-inactivated fetal bovine serum (FBS) as discussed above. Higher receptor and G-protein (Gα, Gβ, Gγ) expression can be obtained if the Sf9 cells are cultured in insect medium with 5% FBS and 5% Gibco serum-free medium (Invitrogen Corp.; Carlsbad, Calif.). Maximal CB1 expression and functional activity is achieved if the Sf9 cells are cultured in insect medium without FBS and with 10% Gibco serum-free medium. The infections are carried out at a multiplicity of infection of 0.1:1.0:0.5:0.5. At 72 hours post-infection, a sample of cell suspension is analyzed for viability by trypan blue dye exclusion, and the remaining Sf9 cells are harvested via centrifugation (3000 rpm/10 min/4° C.).

### Example 62

### Purified Recombinant Insect Cell Membranes

Sf9 cell pellets are resuspended in homogenization buffer (10 mM HEPES, 250 mM sucrose, 0.5 μg/ml leupeptin, 2 μg/ml Aprotinin, 200 μM PMSF, and 2.5 mM EDTA, pH 7.4) and homogenized using a POLYTRON homogenizer (setting 5 for 30 seconds). The homogenate is centrifuged (536×g/10 min/4° C.) to pellet the nuclei. The supernatant containing isolated membranes is decanted to a clean centrifuge tube, centrifuged (48,000×g/30 min, 4° C.) and the resulting pellet resuspended in 30 ml homogenization buffer. This centrifugation and resuspension step is repeated twice. The final pellet is resuspended in ice cold Dulbecco's PBS containing 5 mM EDTA and stored in frozen aliquots at −80° C. until needed. The protein concentration of the resulting membrane preparation (hereinafter “P2 membranes”) is measured using a Bradford protein assay (Bio-Rad Laboratories, Hercules, Calif.). By this measure, a 1-liter culture of cells typically yields 100-150 mg of total membrane protein.

### Example 63

### Radioligand Binding Assays

P2 membranes are resuspended by Dounce homogenization (tight pestle) in binding buffer (50 mM Tris pH. 7.4, 120 mM NaCl, 5 mM MgCl₂, 0.5% BSA and 0.2 mg/ml bacitracin).

For saturation binding analysis, membranes (10 μg) are added to polypropylene tubes containing 25 pM-0.5 nM [³H]CP55,940 (New England Nuclear Corp., Boston, Mass.). Nonspecific binding is determined in the presence of 10 μM CP55,940 (Tocris Cookson Inc., Ellisville, Mo.) and accounted for less than 10% of total binding. For evaluation of guanine nucleotide effects on receptor affinity, GTPγS is added to duplicate tubes at the final concentration of 50 μM.

For competition analysis, membranes (10 μg) are added to polypropylene tubes containing 0.5 nM [³H]CP55,940. Non-radiolabeled displacers are added to separate assays at concentrations ranging from 10⁻¹⁰-M to 10⁻⁵ M to yield a final volume of 0.250 mL. Nonspecific binding is determined in the presence of 10 μM CP55,940 and accounted for less than 10% of total binding. Following a one-hour incubation at rt, the reaction is terminated by rapid vacuum filtration. Samples are filtered over presoaked (0.3% non-fat dry milk for 2 hours prior to use) GF/C WHATMAN filters and rinsed 2 times with 5 mL cold 50 mM Tris pH 7.4. Remaining bound radioactivity is quantified by gamma counting. Kᵢ and Hill coefficient (“nH”) are determined by fitting the Hill equation to the measured values with the aid of SIGMAPLOT software (SPSS Inc., Chicago, Ill.).

### Example 64

### Agonist-Induced GTP Binding

This Example illustrates the use of agonist-stimulated GTPγ³⁵S binding (“GTP binding”) activity to identify CB1 agonists and antagonists, and to differentiate neutral antagonists from those that possess inverse agonist activity. This assay can also be used to detect partial agonism mediated by antagonist compounds. A compound being analyzed in this assay is referred to herein as a “test compound.” Agonist-stimulated GTP binding activity is measured as follows: Four independent baculoviral stocks (one directing the expression of hCB1 and three directing the expression of each of the three subunits of a heterotrimeric G-protein) are used to infect a culture of Sf9 cells as described in Example 61.

Agonist-stimulated GTP binding on purified membranes (prepared as described in Example 62) is initially assessed using the cannabinoid agonist CP55,940 to ascertain that the receptor/G-protein-alpha-beta-gamma combination(s) yield a functional response as measured by GTP binding.

P2 membranes are resuspended by Dounce homogenization (tight pestle) in GTP binding assay buffer (50 mM Tris pH 7.4, 120 mM NaCl, 5 mM MgCl₂, 2 mM EGTA, 0.1% BSA, 0.1 mM bacitracin, 100 KIU/mL aprotinin, 5 μM GDP) and added to reaction tubes at a concentration of 10 μg protein/reaction tube. After adding increasing doses of the agonist CP55,940 at concentrations ranging from 10⁻¹² M to 10⁻⁶ M, reactions are initiated by the addition of 100 pM GTPγ³⁵S. In competition experiments, non-radiolabeled test compounds are added to separate assays at concentrations ranging from 10⁻¹⁰M to 10⁻⁵ M along with 1 nM CP55,940 to yield a final volume of 0.25 mL.

Following a 60-minute incubation at room temperature, the reactions are terminated by vacuum filtration over GF/C filters (pre-soaked in wash buffer, 0.1% BSA) followed by washing with ice-cold wash buffer (50 mM Tris pH 7.0, 120 mM NaCl). The amount of receptor-bound (and thereby membrane-bound) GTPγ³⁵S is determined by measuring the bound radioactivity, preferably by liquid scintillation spectrometry of the washed filters. Non-specific binding is determined using 10 mM GTPγ³⁵S and typically represents less than 5 percent of total binding. Data is expressed as percent above basal (baseline). The results of these GTP binding experiments are analyzed using SIGMAPLOT software and IC₅₀ determined. The IC₅₀ may then be used to generate K; as described by Cheng and Prusoff (1973) Biochem Pharmacol 22(23):3099-108.

Neutral antagonists are those test compounds that reduce the CP55,940-stimulated GTP binding activity towards, but not below, baseline (the level of GTP bound by membranes in this assay in the absence of added CP55,940 or other agonist and in the further absence of any test compound).

In contrast, in the absence of added CP55,940, CB1 inverse agonists reduce the GTP binding activity of the receptor-containing membranes below baseline. If a test compound that displays antagonist activity does not reduce the GTP binding activity below baseline in the absence of the CB1 agonist, it is characterized as a neutral antagonist.

An antagonist test compound that elevates GTP binding activity above baseline in the absence of added CP55,940 in this GTP binding assay is characterized as having partial agonist activity. Preferred CB1 antagonists do not elevate GTP binding activity under such conditions more than 10%, more preferably less than 5% and most preferably less than 2% of the maximal response elicited by the agonist, CP55,940.

The GTP binding assay can also be used to determine antagonist selectivity towards CB1 over CB2. Agonist-stimulated GTP binding activity at CB2 is measured as described above for CB1 except that the Sf9 cells are infected with one baculoviral stock directing the expression of hCB2 and three directing the expression of each of the three subunits of a heterotrimeric G-protein. The IC₅₀ and Kᵢ are generated as described above for CB1.

### Example 65

### Surmountability Assays

Certain CB1 antagonists are insurmountable with regard to the agonist induced GTPγ³⁵s binding effect. To assess surmountability, P2 membranes are resuspended by Dounce homogenization (tight pestle) in GTP binding assay buffer (50 mM Tris pH 7.4, 120 mM NaCl, 5 mM MgCl₂, 2 mM EGTA, 10 μg/ml saponin, 0.1% BSA, 0.1 mM bacitracin, 100 KIU/mL aprotinin, 5 μM GDP) and added to reaction tubes at a concentration of 10 μg protein/reaction tube. Agonist dose-response curves (typically CP55,940) at concentrations ranging from 10⁻¹² M to 10⁻⁵ M, are run either in the absence or in the presence of a test compound at one of several doses up to 100× the IC₅₀ of the test compound as measured in the competition GTPγ³⁵S binding. The reactions are initiated by the addition of 100 pM GTPγ³⁵S to yield a final volume of 0.25 mL. Following a 90-minute incubation at room temperature, the reactions are terminated by vacuum filtration over GF/C filters (pre-soaked in wash buffer, 0.1% BSA) followed by washing with ice-cold wash buffer (50 mM Tris pH 7.0, 120 mM NaCl). The amount of receptor-bound (and thereby membrane-bound) GTPγ³⁵S is determined by measuring the bound radioactivity, preferably by liquid scintillation spectrometry of the washed filters. Non-specific binding is determined using 10 μM GTPγS and typically represents less than 5 percent of total binding. Data is expressed as percent above basal (baseline). The results of these GTP binding experiments may be conveniently analyzed using SIGMAPLOT software. A surmountable test compound is one which shifts the EC₅₀ of the agonist to the right (weaker) without affecting the maximum functional response of the agonist. Insurmountable antagonist test compounds have no significant effect on the hCB1 agonist EC₅₀ at concentrations roughly 100× the IC₅₀, but significantly reduce or eliminate the agonist stimulated GTPγ³⁵S binding response of the receptor.

### Example 66

### MDCK Cytotoxicity Assay

This Example illustrates the evaluation of compound toxicity using a Madin Darby canine kidney (MDCK) cell cytotoxicity assay.

1 μL of test compound is added to each well of a clear bottom 96-well plate (Packard, Meriden, Conn.) to give final concentration of compound in the assay of 10 μM, 100 μM or 200 μM. Solvent without test compound is added to control wells.

MDCK cells, ATCC no. CCL-34 (American Type Culture Collection, Manassas, Va.), are maintained in sterile conditions following the instructions in the ATCC production information sheet. Confluent MDCK cells are trypsinized, harvested, and diluted to a concentration of 0.1×10⁶ cells/mL with warm (37° C.) medium (VITACELL Minimum Essential Medium Eagle, ATCC catalog # 30-2003). 100 μL of diluted cells is added to each well, except for five standard curve control wells that contain 100 μL of warm medium without cells. The plate is then incubated at 37° C. under 95% O₂, 5% CO₂ for 2 hours with constant shaking. After incubation, 50 μL of mammalian cell lysis solution (from the Packard (Meriden, Conn.) ATP-LITE-M Luminescent ATP detection kit) is added per well, the wells are covered with PACKARD TOPSEAL stickers, and plates are shaken at approximately 700 rpm on a suitable shaker for 2 min.

Compounds causing toxicity will decrease ATP production, relative to untreated cells. The ATP-LITE-M Luminescent ATP detection kit is generally used according to the manufacturer's instructions to measure ATP production in treated and untreated MDCK cells. PACKARD ATP LITE-M reagents are allowed to equilibrate to room temperature. Once equilibrated, the lyophilized substrate solution is reconstituted in 5.5 mL of substrate buffer solution (from kit). Lyophilized ATP standard solution is reconstituted in deionized water to give a 10 mM stock. For the five control wells, 10 μL of serially diluted PACKARD standard is added to each of the standard curve control wells to yield a final concentration in each subsequent well of 200 nM, 100 nM, 50 nM, 25 nM, and 12.5 nM. PACKARD substrate solution (50 μL) is added to all wells, which are then covered, and the plates are shaken at approximately 700 rpm on a suitable shaker for 2 min. A white PACKARD sticker is attached to the bottom of each plate and samples are dark adapted by wrapping plates in foil and placing in the dark for 10 min. Luminescence is then measured at 22° C. using a luminescence counter (e.g., PACKARD TOPCOUNT Microplate Scintillation and Luminescence Counter or TECAN SPECTRAFLUOR PLUS), and ATP levels calculated from the standard curve. ATP levels in cells treated with test compound(s) are compared to the levels determined for untreated cells. Cells treated with 10 μM of a preferred test compound exhibit ATP levels that are at least 80%, preferably at least 90%, of the untreated cells. When a 100 μM concentration of the test compound is used, cells treated with preferred test compounds exhibit ATP levels that are at least 50%, preferably at least 80%, of the ATP levels detected in untreated cells.

LENGTHY TABLE
The patent application contains a lengthy table section. A copy of the table is available in electronic form from the USPTO web site (http://seqdata.uspto.gov/?pageRequest=docDetail&DocID=US20070078135A1). An electronic copy of the table will also be available from the USPTO upon request and payment of the fee set forth in 37 CFR 1.19(b)(3).

1. A compound of the formula: 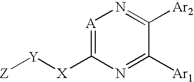
or a pharmaceutically acceptable salt thereof, wherein:
A is CR₁, or N;
Ar₁ and Ar₂ are independently chosen from 5- to 10-membered carbocycles and heterocycles, each of which is substituted with from 0 to 6 substituents independently chosen from R_{A}; such that at least one of Ar₁ and Ar₂ is an optionally substituted 5- or 6-membered heterocycle;
X is C(R₉)(R₁₀), O, N(R₂) or SOₘN(R₂)
Y is C₁-C₄alkylene that is unsubstituted or substituted with one or two substituents independently chosen from R_{B};
Z is O(R₃), S(O)ₘ(R₄), N(R₄)(R₅), S(O)ₘN(R₄)(R₅), C(R₆)(R₇) or C(R₆)(R₇)(R₈); wherein m is 0, 1 or 2;
Each R_{A} is independently chosen from:
(i) halogen, hydroxy, cyano, amino, nitro, aminocarbonyl, aminosulfonyl and —COOH; and
(ii) C₁-C₆alkyl, C₂-C₆alkenyl, C₂-C₆alkynyl, (C₃-C₈cycloalkyl)C₀-C₄alkyl, C₁-C₆alkoxy, C₁-C₆alkylthio, C₁-C₆alkylsulfinyl, C₁-C₆alkoxycarbonyl, C₁-C₆alkylsulfonylC₀-C₄alkyl, mono- or di-(C₁-C₆alkyl)aminoC₀-C₄alkyl, mono- or di-(C₁-C₆alkyl)aminosulfonylC₀-C₄alkyl, mono- or di-(C₁-C₆alkyl)aminocarbonylC₀-C₄alkyl, phenylC₀-C₄alkyl, (4- to 8-membered heterocycle)C₀-C₄alkyl and (4- to 8-membered heterocycle)C₁-C₄alkoxy; each of which is substituted with from 0 to 6 substituents independently chosen from R_{E};
or two R_{A} groups attached to adjacent ring carbon atoms are taken together to form a fused 5- to 7-membered carbocycle or heterocycle that is substituted with from 0 to 6 substituents independently chosen from R_{E};
Each R_{B} is:
(i) halogen, hydroxy, —COOH, aminocarbonyl, C₁-C₄alkyl, C₁-C₄alkoxy, C₁-C₄haloalkyl, C₁-C₄aminoalkyl, mono- or di-(C₁-C₆alkyl)aminoC₀-C₄alkyl, or mono- or di-(C₁-C₆alkyl)aminocarbonyl; or
(ii) taken together with R₃, R₄ or R₆ to form a 4- to 10-membered heterocycloalkyl that is substituted with from 0 to 3 substituents independently chosen from R_{D}; such that if R_{B} forms a heterocycloalkyl with R₃, then the heterocycloalkyl is not substituted with C₁-C₄alkoxycarbonyl;
or two R_{B} are taken together to form a C₃-C₈cycloalkyl or a 4- to 8-membered heterocycloalkyl;
Each R_{D} is independently chosen from:
(i) hydroxy, halogen, cyano, amino, oxo, nitro, —COOH, aminocarbonyl and aminosulfonyl; and
(ii) C₁-C₆alkyl, C₂-C₆alkenyl, C₂-C₆alkynyl, (C₃-C₈cycloalkyl)C₀-C₄alkyl, C₁-C₆alkoxy, C₁-C₆alkylthio, C₂-C₆alkyl ether, mono- or di-(C₁-C₆alkyl)amino, mono- or di-(C₁-C₆alkyl)aminocarbonyl, mono- or di-(C₁-C₆alkyl)aminosulfonyl, C₁-C₆alkylsulfinyl, C₁-C₆alkylsulfonyl, (4- to 8-membered heterocycle)C₀-C₄alkyl and phenylC₀-C₄alkyl, each of which is substituted with from 0 to 4 substituents independently chosen from R_{E};
Each R_{E} is independently chosen from oxo, halogen, hydroxy, cyano, amino, nitro, aminocarbonyl, aminosulfonyl, —COOH, C₁-C₆alkyl, C₁-C₆haloalkyl, C₁-C₆alkoxy, C₁-C₆haloalkoxy, C₁-C₆alkylthio, C₁-C₆alkoxycarbonyl, C₁-C₆alkanoyloxy, C₃-C₆alkanone, mono- or di-(C₁-C₆alkyl)amino, C₁-C₆alkylsulfonyl, mono- or di-(C₁-C₆alkyl)aminosulfonyl, and mono- or di-(C₁-C₆alkyl)aminocarbonyl;
R₁ is:
(i) hydrogen, halogen, cyano, nitro, —COOH or aminosulfonyl; or
(ii) C₁-C₆alkyl, C₂-C₆alkenyl, C₂-C₆alkynyl, (C₃-C₈cycloalkyl)C₀-C₄alkyl, C₁-C₆alkoxy, C₁-C₆alkoxycarbonyl, C₁-C₆alkylsulfonylC₀-C₄alkyl, C₁-C₆alkylaminosulfonylC₀-C₄alkyl, mono- or di-(C₁-C₆alkyl)aminoC₀-C₄alkyl, or mono- or di-(C₁-C₆alkyl)aminocarbonylC₀-C₄alkyl or a (4- to 8-membered heterocycle)C₀-C₄alkyl; each of which is substituted with from 0 to 6 substituents independently chosen from R_{E}; or
(iii) taken together with R₂ or R₉ to form a fused 5- to 8-membered heterocycle that is substituted with from 0 to 4 substituents independently chosen from R_{E};
R₂ is:
(i) hydrogen or aminocarbonyl;
(ii) C₁-C₆alkyl, C₂-C₆alkenyl, C₂-C₆alkynyl, (C₃-C₈cycloalkyl)C₀-C₄alkyl, C₂-C₆alkyl ether, C₁-C₆alkoxycarbonyl, mono- or di-(C₁-C₄alkyl)aminocarbonyl, mono- or di-(C₁-C₄alkyl)aminosulfonyl, mono- or di-(C₁-C₄alkyl)aminoC₀-C₄alkyl, C₁-C₆alkylsulfonyl or (4- to 8-membered heterocycle)C₀-C₄alkyl; each of which is substituted with from 0 to 6 substituents independently chosen from R_{E};
(iii) taken together with R₃, R₄ or R₆ to form a 4- to 10-membered heterocycle that is substituted with from 0 to 3 substituents independently chosen from R_{D}; or
(iv) taken together with R₁ to form an optionally substituted fused 5- to 8-membered heterocycle;
R₃ is:
(i) hydrogen;
(ii) C₁-C₈alkyl, C₂-C₈alkenyl, C₂-C₈alkynyl, C₁-C₆alkanone, C₂-C₈alkyl ether, mono- or di-(C₁-C₆alkyl)aminoC₁-C₄alkyl, (C₃-C₈cycloalkyl)C₀-C₄alkyl, mono- or di-(C₁-C₆alkyl) amino carbonyl, (5- to 7-membered heterocycle)-C(═O)—, phenylC₀-C₄alkyl or (4- to 8-membered heterocycle)C₀-C₄alkyl; each of which is substituted with from 0 to 3 substituents independently chosen from hydroxy, halogen, amino, C₁-C₆alkyl or C₁-C₆alkoxy; or
(iii) taken together with R₂, R₉ or R_{B} to form a 4- to 10-membered heterocycle that is substituted with from 0 to 3 substituents independently chosen from R_{D};
R₄ is:
(i) hydrogen;
(ii) C₁-C₈alkyl, C₂-C₈alkenyl, C₂-C₈alkynyl, (C₃-C₈cycloalkyl)C₀-C₄alkyl, C₁-C₆alkylsulfonyl, (C₃-C₈cycloalkyl)sulfonyl, C₁-C₆alkoxycarbonyl, C₂-C₈alkyl ether, mono- or di-(C₁-C₆alkyl)aminosulfonyl, mono- or di-(C₁-C₆alkyl)aminocarbonyl or (5- or 6-membered heterocycle)C₀-C₄alkyl; each of which is substituted with from 0 to 6 substituents independently chosen from halogen, hydroxy, oxo, cyano, amino, aminosulfonyl, aminocarbonyl, C₁-C₆alkyl, C₁-C₆alkoxy, C₁-C₆alkylsulfonyl, mono- or di-(C₁-C₆alkyl)aminosulfonyl, mono- or di-(C₁-C₆alkyl)amino and mono- or di-(C₁-C₆alkyl)aminocarbonyl; or
(iii) taken together with R₂, R₅, R₉ or R_{B} to form a 4- to 10-membered heterocycle that is substituted with from 0 to 4 substituents independently chosen from R_{D};
R₅ is:
(i) hydrogen, cyano or aminocarbonyl;
(ii) C₁-C₈alkyl, C₂-C₈alkenyl, C₂-C₈alkynyl, (C₃-C₈cycloalkyl)C₀-C₄alkyl, C₁-C₆alkylsulfonyl, (C₃-C₈cycloalkyl)sulfonyl, C₁-C₆alkoxycarbonyl, C₂-C₈alkyl ether, mono- or di-(C₁-C₆alkyl)aminosulfonyl, mono- or di-(C₁-C₆alkyl)aminocarbonyl, mono- or di-(C₁-C₆alkyl)aminoC₀-C₄alkyl, phenylC₀-C₄alkyl or (5- or 6-membered heterocycle)C₀-C₄alkyl; each of which is substituted with from 0 to 6 substituents independently chosen from halogen, hydroxy, oxo, cyano, amino, —COOH, aminosulfonyl, aminocarbonyl, C₁-C₆alkyl, C₁-C₆haloalkyl, C₁-C₆alkoxy, C₁-C₆alkylsulfonyl, mono- or di-(C₁-C₆alkyl)aminosulfonyl, mono- or di-(C₁-C₆alkyl)amino, mono- or di-(C₁-C₆alkyl)aminocarbonyl, 4- to 7-membered heterocycle, and phenyl; or
(iii) taken together with R₄ to form an optionally substituted 4- to 8-membered heterocycloalkyl;
R₆ is:
(i) hydrogen, hydroxy, halogen, cyano, amino, aminocarbonyl, aminosulfonyl or —COOH;
(ii) C₁-C₈alkyl, C₂-C₈alkenyl, C₂-C₈alkynyl, (C₃-C₈cycloalkyl)C₀-C₄alkyl, C₁-C₆alkoxy, C₁-C₆alkylthio, C₁-C₆alkylsulfinyl, C₁-C₆alkylsulfonyl, (C₃-C₈cycloalkyl)sulfonyl, C₁-C₆alkoxycarbonyl, mono- or di-(C₁-C₆alkyl)aminosulfonyl, mono- or di-(C₁-C₆alkyl)aminocarbonyl, mono- or di-(C₁-C₆alkyl)aminoC₀-C₄alkyl or (5- or 6-membered heterocycle)C₀-C₄alkyl; each of which is substituted with from 0 to 6 substituents independently chosen from R_{E}; or
(iii) taken together with one or two of R₂, R₇, R₈, R₉ or R_{B} to form a 4- to 10-membered carbocycle or heterocycle that is substituted with from 0 to 3 substituents independently chosen from R_{D};
R₇ is:
(i) hydrogen, hydroxy, halogen, cyano, amino, aminocarbonyl, aminosulfonyl or —COOH;
(ii) C₁-C₈alkyl, C₂-C₈alkenyl, C₂-C₈alkynyl, (C₃-C₈cycloalkyl)C₀-C₄alkyl, C₁-C₆alkoxy, C₁-C₈alkylthio, C₁-C₈alkylsulfinyl, C₁-C₆alkylsulfonyl, (C₃-C₈cycloalkyl)sulfonyl, mono- or di-(C₁-C₆alkyl)amino, mono- or di-(C₁-C₆alkyl)aminosulfonyl, or mono- or di-(C₁-C₆alkyl)aminocarbonyl, each of which is substituted with from 0 to 4 substituents independently chosen from R_{E}; such that R₇ is not C₁-C₆hydroxyalkyl; or
(iii) taken together with R₆ or R₈ to form an optionally substituted carbocycle or heterocycle;
R₈ is:
(i) halogen, cyano, amino, aminosulfonyl or —COOH;
(ii) C₁-C₈alkyl, C₂-C₈alkenyl, C₂-C₈alkynyl, (C₃-C₈cycloalkyl)C₀-C₄alkyl, C₁-C₆alkoxy, C₁-C₆alkanoyl, C₁-C₈alkylthio, C₁-C₈alkylsulfinyl, C₁-C₆alkylsulfonyl, (C₃-C₈cycloalkyl)sulfonyl, mono- or di-(C₁-C₆alkyl)amino, mono- or di-(C₁-C₆alkyl)aminosulfonyl, or mono- or di-(C₁-C₆alkyl)aminocarbonyl, each of which is substituted with from 0 to 4 substituents independently chosen from hydroxy, halogen, cyano, amino, nitro, aminocarbonyl, aminosulfonyl, —COOH, C₁-C₆alkyl, C₁-C₆haloalkyl, C₁-C₆alkoxy, C₁-C₆haloalkoxy, C₁-C₆alkylthio, C₁-C₆alkoxycarbonyl, C₁-C₆alkanoyloxy, C₃-C₆alkanone, mono- or di-(C₁-C₆alkyl)amino, C₁-C₆alkylsulfonyl, mono- or di-(C₁-C₆alkyl)aminosulfonyl, and mono- or di-(C₁-C₆alkyl)aminocarbonyl; or
(iii) taken together with R₆ or R₇ to form an optionally substituted carbocycle or heterocycle;
R₉ is:
(i) hydrogen, hydroxy, halogen, cyano or amino;
(ii) C₁-C₈alkyl, C₂-C₈alkenyl, C₂-C₈alkynyl, C₁-C₈alkoxy, C₁-C₈alkylthio, C₁-C₈alkylsulfinyl, C₁-C₈alkylamino, (C₃-C₈cycloalkyl)C₀-C₄alkyl or C₁-C₆alkylsulfonyl; each of which is substituted with from 0 to 6 substituents independently chosen from R_{E};
(iii) taken together with R₃, R₄ or R₆ to form an optionally substituted 4- to 10-membered carbocycle or heterocycle; or
(iv) taken together with R₁ to form an optionally substituted, fused 5- to 8-membered carbocycle or heterocycle; and
R₁₀ is:
(i) hydrogen, hydroxy, halogen, cyano or amino; or
(ii) C₁-C₈alkyl, C₂-C₈alkenyl, C₂-C₈alkynyl, C₁-C₈alkoxy, C₁-C₈alkylthio, C₁-C₈alkylsulfinyl, C₁-C₈alkylamino, (C₃-C₈cycloalkyl)C₀-C₄alkyl or C₁-C₆alkylsulfonyl; each of which is substituted with from 0 to 6 substituents independently chosen from R_{E}.

2. A compound or salt according to claim 1, wherein A is CR₁.

3. A compound or salt according to claim 1, wherein A is N.

4. A compound or salt according to claim 1, wherein X is N(R₂).

5. A compound or salt according to claim 4, wherein the compound has the formula: 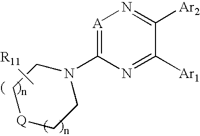
wherein:
Each n is independently 0, 1 or 2, such that at least one n is not 0;
Q is NR₅, O or SOₘ, wherein m is 0, 1 or 2; and
R₁₁ represents 0, 1 or 2 substituents independently chosen from oxo, COOH, aminocarbonyl, C₁-C₄alkyl, C₁-C₄hydroxyalkyl, C₂-C₄alkyl ether, mono- or di-(C₁-C₄alkyl)aminoC₀-C₄alkyl, mono- or di-(C₁-C₄alkyl)aminocarbonyl and groups that are taken together with R₅ to form a 5- to 8-membered heterocycloalkyl; or R₁₁ represents two substituents that are taken together to form a C₁-C₂alkylene bridge.

6. A compound or salt according to claim 5, wherein the compound has the formula: 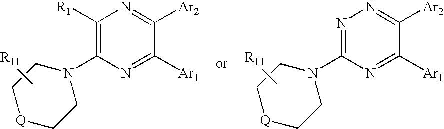
wherein:
R₁₁ represents 0, 1 or 2 substituents independently chosen from oxo, COOH, aminocarbonyl, C₁-C₄alkyl, C₁-C₄hydroxyalkyl, C₂-C₄alkyl ether, mono- or di-(C₁-C₄alkyl)aminoC₀-C₄alkyl and mono- or di-(C₁-C₄alkyl)aminocarbonyl; and
Q is SO₂, SO, S, O or NH.

7. A compound or salt according to claim 5, wherein the compound has the formula: 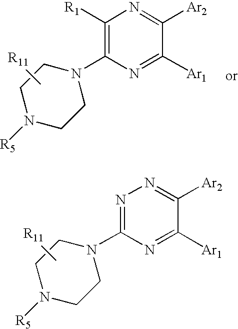
wherein R₅ is:
(i) hydrogen or cyano; or
(ii) C₁-C₆alkyl, (C₃-C₈cycloalkyl)C₀-C₄alkyl, C₂-C₆alkyl ether, C₁-C₆alkanoyl, C₁-C₆alkylsulfonyl, C₃-C₈cycloalkylsulfonyl, mono- or di-(C₁-C₆alkyl)aminocarbonyl, mono- or di-(C₁-C₆alkyl)aminoC₀-C₄alkyl, mono- or di-(C₁-C₆alkyl)aminosulfonyl, or (5- or 6-membered heteroaryl)C₀-C₄alkyl, each of which is substituted with from 0 to 4 substituents independently chosen from hydroxy, halogen, cyano, amino, oxo, C₁-C₆alkyl, aminocarbonyl, mono- or di-(C₁-C₆alkyl)aminocarbonyl, or mono- or di-(C₁-C₆alkyl)amino.

8. A compound or salt according to claim 7, wherein the compound has the formula: 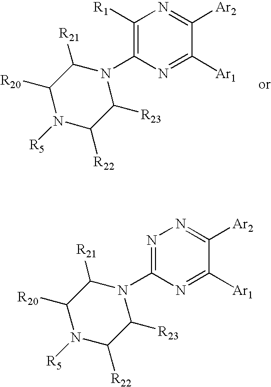
wherein one of R₂₀ and R₂₁ is taken together with R₂₂ or R₂₃ to form a methylene or ethylene bridge, and those of R₂₀, R₂₁, R₂₂ and R₂₃ that do not form bridge are hydrogen.

9. (canceled)

10. A compound or salt according to claim 4, wherein the compound has the formula: 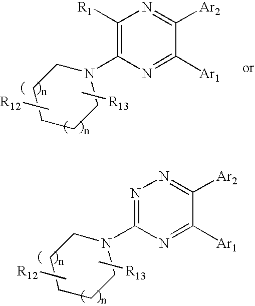
wherein:
each n is independently 0, 1 or 2;
R₁₂ is hydroxy, halogen, cyano, amino, C₁-C₄alkyl, (C₃-C₈cycloalkyl)C₀-C₄alkyl, C₂-C₄alkenyl, C₁-C₄hydroxyalkyl, C₁-C₄alkoxy, C₂-C₄alkyl ether, C₁-C₄alkanoyl, C₁-C₄alkylsulfonyl, mono- or di-(C₁-C₆alkyl)amino, mono- or di-(C₁-C₆alkyl)amino or (5- to 7-membered heterocycle)C₀-C₂alkyl, each of which is substituted with from 0 to 3 substituents independently chosen from oxo, amino, hydroxy, C₁-C₄alkoxy, and mono- or di-(C₁-C₆alkyl)amino; and
R₁₃ represents from 0 to 3 substituents independently chosen from halogen, cyano, amino, aminocarbonyl, C₁-C₄alkyl, C₂-C₄alkenyl, C₁-C₄hydroxyalkyl, C₁-C₄alkoxy, C₂-C₄alkyl ether and C₁-C₄alkanoyl; or
R₁₂ and R₁₃ are taken together to form a fused or spiro 5- to 7-membered heterocycle that is substituted with from 0 to 2 substituents independently chosen from hydroxy, oxo, C₁-C₄alkyl and C₁-C₄hydroxyalkyl.

11. A compound or salt according to claim 4, wherein the compound has the formula:

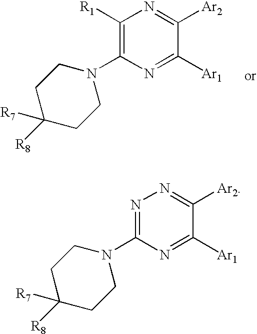

12. A compound or salt according to claim 11, wherein R₇ is hydrogen or aminocarbonyl, and R₈ is mono- or di-(C₁-C₆alkyl)amino or C₁-C₆alkanoylamino.

13. (canceled)

14. A compound or salt according to claim 4, wherein the compound has the formula:

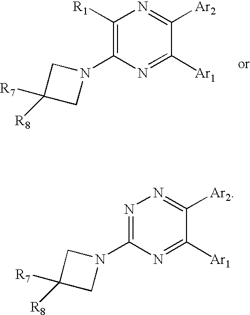

15. A compound or salt according to claim 4, wherein the compound has the formula:

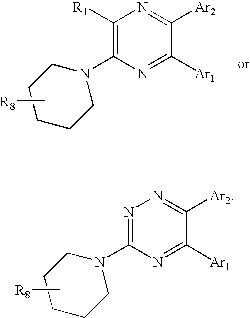

16.-19. (canceled)

20. A compound or salt according to claim 1, wherein R₁ is hydrogen, bromo, chloro, cyano, methyl, ethyl, methylamino or ethylamino.

21. A compound or salt according to claim 1, wherein Ar₁ is phenyl or pyridyl, each of which is substituted with from 0 to 3 substituents independently chosen from chloro, bromo, fluoro, cyano, aminocarbonyl, C₁-C₄alkyl, C₁-C₄alkoxy, C₁-C₄alkoxycarbonyl, mono- or di-(C₁-C₄alkyl)aminocarbonyl and C₁-C₄alkanoyl.

22. A compound or salt according to claim 21, wherein Ar₁ is pyridin-4-yl that is substituted with 1 or 2 substituents.

23. A compound or salt according to claim 21, wherein Ar₁ is substituted with 1 or 2 halogens.

24. A compound or salt according to claim 23, wherein Ar₁ is 2-chloro-pyridin-4-yl, 4-fluorophenyl, 4-chlorophenyl or 2,4-dichlorophenyl.

25. A compound or salt according to claim 1, wherein Ar₁ is cyclohexyl or a 6-membered heterocycloalkyl, each of which is substituted with from 0 to 2 substituents independently chosen from chloro, bromo, fluoro, cyano, aminocarbonyl, C₁-C₄alkyl, C₁-C₄alkoxy, C₁-C₄alkoxycarbonyl, mono- or di-(C₁-C₄alkyl)aminocarbonyl and C₁-C₄alkanoyl.

26. A compound or salt according to claim 25, wherein Ar₁ is morpholinyl or thiomorpholinyl.

27. A compound or salt according to claim 1, wherein Ar₂ is phenyl, pyrrolyl or pyridyl, each of which is substituted with from 0 to 3 substituents independently chosen from:
(i) chloro, bromo, fluoro, cyano, aminocarbonyl, C₁-C₄alkyl, C₁-C₄haloalkyl, C₁-C₄haloalkoxy, C₁-C₄alkoxycarbonyl, mono- or di-(C₁-C₄alkyl)aminocarbonyl and C₁-C₄alkanoyl; and
(ii) C₁-C₄alkoxy that is unsubstituted or substituted with hydroxy, amino, C₁-C₄alkoxy, mono- or di-(C₁-C₄alkyl)amino or a 4- to 7-membered heterocycloalkyl.

28. A compound or salt according to claim 27, wherein Ar₂ is substituted with 1 or 2 substituents.

29. A compound or salt according to claim 1, wherein Ar₂ is cyclohexyl or a 6-membered heterocycloalkyl, each of which is substituted with from 0 to 2 substituents independently chosen from:
(i) chloro, bromo, fluoro, cyano, aminocarbonyl, C₁-C₄alkyl, C₁-C₄haloalkyl, C₁-C₄haloalkoxy, C₁-C₄alkoxycarbonyl, mono- or di-(C₁-C₄alkyl)aminocarbonyl and C₁-C₄alkanoyl; and
(ii) C₁-C₄alkoxy that is unsubstituted or substituted with hydroxy, amino, C₁-C₄alkoxy, mono- or di-(C₁-C₄alkyl)amino or a 4- to 7-membered heterocycloalkyl.

30. A compound or salt according to claim 29, wherein Ar₂ is morpholinyl or thiomorpholinyl.

31. A compound or salt according to claim 5, wherein the compound has the formula: 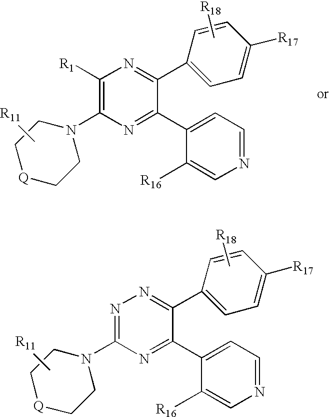
wherein:
R₁₆ is chloro, fluoro or methyl;
R₁₇ is:
(i) chloro, bromo, fluoro, cyano, aminocarbonyl, C₁-C₄alkyl, C₁-C₄haloalkyl, C₁-C₄haloalkoxy, C₁-C₄alkoxycarbonyl, mono- or di-(C₁-C₄alkyl)aminocarbonyl or C₁-C₄alkanoyl; or
(ii) C₁-C₄alkoxy that is unsubstituted or substituted with hydroxy, amino, C₁-C₄alkoxy, mono- or di-(C₁-C₄alkyl)amino or a 4- to 7-membered heterocycloalkyl; and
R₁₈ is absent or represents one substituent chosen from chloro, bromo, fluoro, cyano, aminocarbonyl, C₁-C₄alkyl, C₁-C₄alkoxy, C₁-C₄haloalkyl, or C₁-C₄haloalkoxy.

32. A compound of the formula: 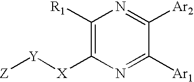
or a pharmaceutically acceptable salt thereof, wherein:
Ar₁ and Ar₂ are independently chosen from 5- to 10-membered carbocycles and heterocycles, each of which is substituted with from 0 to 6 substituents independently chosen from R_{A};
X is C(R₉)(R₁₀), O, N(R₂) or SOₘN(R₂);
Y is C₁-C₄alkylene that is unsubstituted or substituted with one or two substituents independently chosen from R_{B};
Z is O(R₃), S(O)ₘ(R₄), N(R₄)(R₅), S(O)ₘN(R₄)(R₅), C(R₆)(R₇) or C(R₆)(R₇)(R₈); wherein m is 0, 1 or 2; such that Z is not amino or dimethylamino if X and Y are both —CH₂—;
Each R_{A} is independently chosen from:
(i) halogen, hydroxy, cyano, amino, nitro, aminocarbonyl, aminosulfonyl and —COOH;
(ii) C₁-C₆alkyl, C₂-C₆alkenyl, C₂-C₆alkynyl, (C₃-C₈cycloalkyl)C₀-C₄alkyl, C₁-C₆alkoxy, C₁-C₆alkylthio, C₁-C₆alkylsulfinyl, C₁-C₆alkoxycarbonyl, C₁-C₆alkylsulfonylC₀-C₄alkyl, mono- or di-(C₁-C₆alkyl)aminoC₀-C₄alkyl, mono- or di-(C₁-C₆alkyl)aminosulfonylC₀-C₄alkyl, mono- or di-(C₁-C₆alkyl)aminocarbonylC₀-C₄alkyl, phenylC₀-C₄alkyl, (4- to 8-membered heterocycle)C₀-C₄alkyl and (4- to 8-membered heterocycle)C₁-C₄alkoxy; each of which is substituted with from 0 to 6 substituents independently chosen from R_{E};
or two R_{A} groups attached to adjacent ring carbon atoms are taken together to form a fused 5- to 7-membered carbocycle or heterocycle that is substituted with from 0 to 6 substituents independently chosen from R_{E};
Each R_{B} is:
(i) halogen, hydroxy, —COOH, C₁-C₄alkyl, C₁-C₄haloalkyl, C₁-C₄aminoalkyl, C₁-C₄alkoxy, mono- or di-(C₁-C₆alkyl)aminoC₀-C₄alkyl, or mono- or di-(C₁-C₆alkyl)aminocarbonyl; or
(ii) taken together with R₃, R₄ or R₆ to form a 4- to 10-membered heterocycloalkyl that is substituted with from 0 to 3 substituents independently chosen from R_{D}; such that if R_{B} forms a heterocycloalkyl with R₃, then the heterocycloalkyl is not substituted with C₁-C₄alkoxycarbonyl;
or two R_{B} are taken together to form a C₃-C₈cycloalkyl or a 4- to 8-membered heterocycloalkyl substituted with from 0 to 3 substituents independently chosen from R_{E};
Each R_{D} is independently chosen from:
(i) hydroxy, halogen, cyano, amino, oxo, nitro, —COOH, aminocarbonyl and aminosulfonyl; and
(ii) C₁-C₆alkyl, C₂-C₆alkenyl, C₂-C₆alkynyl, (C₃-C₈cycloalkyl)C₀-C₄alkyl, C₁-C₆alkoxy, C₁-C₆alkylthio, C₂-C₆alkyl ether, mono- or di-(C₁-C₆alkyl)amino, mono- or di-(C₁-C₆alkyl)aminocarbonyl, mono- or di-(C₁-C₆alkyl)aminosulfonyl, C₁-C₆alkylsulfinyl, C₁-C₆alkylsulfonyl, (4- to 8-membered heterocycle)C₀-C₄alkyl and phenylC₀-C₄alkyl, each of which is preferably substituted with from 0 to 4 substituents independently chosen from R_{E};
Each R_{E} is independently chosen from oxo, halogen, hydroxy, cyano, amino, nitro, aminocarbonyl, aminosulfonyl, —COOH, C₁-C₆alkyl, C₁-C₆haloalkyl, C₁-C₆alkoxy, C₁-C₆haloalkoxy, C₁-C₆alkylthio, C₁-C₆alkoxycarbonyl, C₁-C₆alkanoyloxy, C₃-C₆alkanone, mono- or di-(C₁-C₆alkyl)amino, C₁-C₆alkylsulfonyl, mono- or di-(C₁-C₆alkyl)aminosulfonyl, and mono- or di-(C₁-C₆alkyl)aminocarbonyl;
R₁ is:
(i) hydrogen, halogen, cyano, nitro, —COOH, aminocarbonyl or aminosulfonyl; or
(ii) C₁-C₆alkyl, C₂-C₆alkenyl, C₂-C₆alkynyl, (C₃-C₈cycloalkyl)C₀-C₄alkyl, C₁-C₆alkoxy, C₁-C₆alkoxycarbonyl, C₁-C₆alkylsulfonylC₀-C₄alkyl, C₁-C₆alkylaminosulfonylC₀-C₄alkyl, mono- or di-(C₁-C₆alkyl)aminoC₀-C₄alkyl, or mono- or di-(C₁-C₆alkyl)aminocarbonylC₀-C₄alkyl or a (4- to 8-membered heterocycle)C₀-C₄alkyl; each of which is substituted with from 0 to 6 substituents independently chosen from halogen, hydroxy, cyano and amino; or
(iii) taken together with R₂ or R₉ to form a fused 5- to 8-membered heterocycle that is substituted with from 0 to 4 substituents independently chosen from R_{E};
R₂ is:
(i) hydrogen or aminocarbonyl;
(ii) C₁-C₆alkyl, C₂-C₆alkenyl, C₂-C₆alkynyl, (C₃-C₈cycloalkyl)C₀-C₄alkyl, C₂-C₆alkyl ether, C₁-C₆alkoxycarbonyl, mono- or di-(C₁-C₄alkyl)aminocarbonyl, mono- or di-(C₁-C₄alkyl)aminosulfonyl, mono- or di-(C₁-C₄alkyl)aminoC₀-C₄alkyl, C₁-C₆alkylsulfonyl or (4- to 8-membered heterocycle)C₀-C₄alkyl; each of which is optionally substituted, and each of which is preferably substituted with from 0 to 6 substituents independently chosen from R_{E};
(iii) taken together with R₃, R₄ or R₆ to form a 4- to 10-membered heterocycle that is substituted with from 0 to 3 substituents independently chosen from R_{D}; or
(iv) taken together with R₁, to form an optionally substituted fused 5- to 8-membered heterocycle;
R₃ is:
(i) C₁-C₈alkyl, C₂-C₈alkenyl, C₂-C₈alkynyl, C₁-C₆alkanone, C₂-C₈alkyl ether, mono- or di-(C₁-C₆alkyl)aminoC₁-C₄alkyl, (C₃-C₈cycloalkyl)C₀-C₄alkyl, mono- or di-(C₁-C₆alkyl) amino carbonyl, (5- to 7-membered heterocycle)-C(═O)—, or phenylC₀-C₄alkyl; each of which is substituted with from 0 to 3 substituents independently chosen from halogen, amino, C₁-C₆alkyl or C₁-C₆alkoxy; or
(ii) taken together with R₂, R₉ or R_{B} to form a 4- to 10-membered heterocycle that is substituted with from 0 to 3 substituents independently chosen from R_{D};
R₄ is:
(i) hydrogen;
(ii) C₁-C₈alkyl, C₂-C₈alkenyl, C₂-C₈alkynyl, (C₃-C₈cycloalkyl)C₀-C₄alkyl, C₁-C₆alkylsulfonyl, (C₃-C₈cycloalkyl)sulfonyl, C₂-C₈alkyl ether, mono- or di-(C₁-C₆alkyl)aminosulfonyl, mono- or di-(C₁-C₆alkyl)aminocarbonyl or (5- or 6-membered heterocycle)C₀-C₄alkyl; each of which is substituted with from 0 to 6 substituents independently chosen from halogen, oxo, hydroxy, cyano, amino, aminosulfonyl, aminocarbonyl, C₁-C₆alkyl, C₁-C₆alkoxy, C₁-C₆alkylsulfonyl, mono- or di-(C₁-C₆alkyl)aminosulfonyl, mono- or di-(C₁-C₆alkyl)amino and mono- or di-(C₁-C₆alkyl)aminocarbonyl, such that R₄ is not C₁-C₈alkyl that is substituted with COOH or C₁-C₈alkoxycarbonyl;
(iii) taken together with R₂, R₅, R₉ or R_{B} to form a 4- to 10-membered heterocycle that is substituted with from 0 to 4 substituents independently chosen from R_{D};
R₅ is:
(i) hydrogen, cyano or aminocarbonyl;
(ii) C₁-C₈alkyl, C₂-C₈alkenyl, C₂-C₈alkynyl, (C₃-C₈cycloalkyl)C₀-C₄alkyl, C₁-C₆alkylsulfonyl, (C₃-C₈cycloalkyl)sulfonyl, C₂-C₈alkyl ether, mono- or di-(C₁-C₆alkyl)aminosulfonyl, mono- or di-(C₁-C₆alkyl)aminocarbonyl, mono- or di-(C₁-C₆alkyl)aminoC₀-C₄alkyl, phenylC₀-C₄alkyl or (5- or 6-membered heterocycle)C₀-C₄alkyl; each of which is substituted with from 0 to 6 substituents independently chosen from halogen, oxo, hydroxy, cyano, amino, aminosulfonyl, aminocarbonyl, C₁-C₆alkyl, C₁-C₆haloalkylC₁-C₆alkoxy, C₁-C₆alkylsulfonyl, mono- or di-(C₁-C₆alkyl)aminosulfonyl, mono- or di-(C₁-C₆alkyl)amino, mono- or di-(C₁-C₆alkyl)aminocarbonyl, 4- to 7-membered heterocycle, and phenyl, such that R₅ is not C₁-C₈alkyl that is substituted with COOH or C₁-C₈alkoxycarbonyl; or
(iii) taken together with R₄ or R_{B} to form an optionally substituted 4- to 8-membered heterocycloalkyl;
R₆ is taken together with one or two of R₂, R₇, R₈, R₉ or R_{B} to form a 4- to 10-membered cycloalkyl or heterocycle that is substituted with from 0 to 3 substituents independently chosen from R_{D};
R₇ is:
(i) hydrogen, hydroxy, halogen, cyano, amino, aminocarbonyl, aminosulfonyl or —COOH;
(ii) C₁-C₈alkyl, C₂-C₈alkenyl, C₂-C₈alkynyl, (C₃-C₈cycloalkyl)C₀-C₄alkyl, C₁-C₆alkoxy, C₁-C₈alkylthio, C₁-C₈alkylsulfinyl, C₁-C₆alkylsulfonyl, (C₃-C₈cycloalkyl)sulfonyl, mono- or di-(C₁-C₆alkyl)amino, mono- or di-(C₁-C₆alkyl)aminosulfonyl, or mono- or di-(C₁-C₆alkyl)aminocarbonyl, each of which is substituted with from 0 to 4 substituents independently chosen from R_{E}; such that R₇ is not C₁-C₆hydroxyalkyl; or
(iii) taken together with R₆ or R₈ to form an optionally substituted cycloalkyl or heterocycle;
R₈ is:
(i) halogen, cyano, amino, aminosulfonyl or —COOH;
(ii) C₁-C₈alkyl, C₂-C₈alkenyl, C₂-C₈alkynyl, (C₃-C₈cycloalkyl)C₀-C₄alkyl, C₁-C₆alkoxy, C₁-C₆alkanoyl, C₁-C₈alkylthio, C₁-C₈alkylsulfinyl, C₁-C₆alkylsulfonyl, (C₃-C₈cycloalkyl)sulfonyl, mono- or di-(C₁-C₆alkyl)amino, mono- or di-(C₁-C₆alkyl)aminosulfonyl, mono- or di-(C₁-C₆alkyl)aminocarbonyl or (5 to 7 membered heterocycle)C₀-C₂alkyl, each of which is substituted with from 0 to 4 substituents independently chosen from hydroxy, oxo, halogen, cyano, amino, nitro, aminocarbonyl, aminosulfonyl, —COOH, C₁-C₆alkyl, C₁-C₆haloalkyl, C₁-C₆alkoxy, C₁-C₆haloalkoxy, C₁-C₆alkylthio, C₁-C₆alkoxycarbonyl, C₁-C₆alkanoyloxy, C₃-C₆alkanone, mono- or di-(C₁-C₆alkyl)amino, C₁-C₆alkylsulfonyl, mono- or di-(C₁-C₆alkyl)aminosulfonyl, and mono- or di-(C₁-C₆alkyl)aminocarbonyl; or
(iii) taken together with R₆ or R₇ to form an optionally substituted cycloalkyl or heterocycle;
R₉ is:
(i) hydrogen, hydroxy, halogen, cyano or amino;
(ii) C₁-C₈alkyl, C₂-C₈alkenyl, C₂-C₈alkynyl, C₁-C₈alkoxy, C₁-C₈alkylthio, C₁-C₈alkylsulfinyl, C₁-C₈alkylamino, (C₃-C₈cycloalkyl)C₀-C₄alkyl or C₁-C₆alkylsulfonyl; each of which is substituted with from 0 to 6 substituents independently chosen from R_{E};
(iii) taken together with R₃, R₄ or R₆ to form an optionally substituted 4- to 10-membered carbocycle or heterocycle; or
(iv) taken together with R₁ to form an optionally substituted, fused 5- to 8-membered carbocycle or heterocycle; and
R₁₀ is:
(i) hydrogen, hydroxy, halogen, cyano or amino; or
(ii) C₁-C₈alkyl, C₂-C₈alkenyl, C₂-C₈alkynyl, C₁-C₈alkoxy, C₁-C₈alkylthio, C₁-C₈alkylsulfinyl, C₁-C₈alkylamino, (C₃-C₈cycloalkyl)C₀-C₄alkyl or C₁-C₆alkylsulfonyl; each of which is substituted with from 0 to 6 substituents independently chosen from R_{E}.

33. A compound or salt according to claim 32, wherein X is N(R₂).

34. A compound or salt according to claim 33, wherein the compound has the formula: 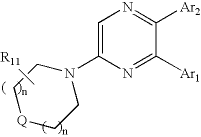
wherein:
Each n is independently 0, 1 or 2, such that at least one n is not 0;
Q is NR₅, O or SOₘ, wherein m is 0, 1 or 2; and
R₁₁ represents 0, 1 or 2 substituents independently chosen from oxo, COOH, aminocarbonyl, C₁-C₄alkyl, C₁-C₄hydroxyalkyl, C₂-C₄alkyl ether, mono- or di-(C₁-C₄alkyl)aminoC₀-C₄alkyl, mono- or di-(C₁-C₄alkyl)aminocarbonyl and groups that are taken together with R₅ to form a 5- to 8-membered heterocycloalkyl; or R₁₁ represents two substituents that are taken together to form a C₁-C₂alkylene bridge.

35. A compound or salt according to claim 34, wherein the compound has the formula: 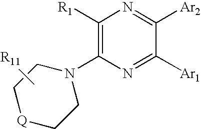
wherein:
R₁₁ represents 0, 1 or 2 substituents independently chosen from oxo, COOH, aminocarbonyl, C₁-C₄alkyl, C₁-C₄hydroxyalkyl, C₂-C₄alkyl ether, mono- or di-(C₁-C₄alkyl)aminoC₀-C₄alkyl and mono- or di-(C₁-C₄alkyl)aminocarbonyl; and
Q is SO₂, SO, S, O or NH.

36. A compound or salt according to claim 34, wherein the compound has the formula: 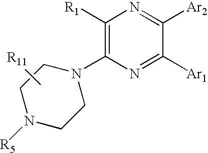
wherein R₅ is:
(i) hydrogen or cyano; or
(ii) C₁-C₆alkyl, (C₃-C₈cycloalkyl)C₀-C₄alkyl, C₁-C₆alkanoyl, C₁-C₆alkylsulfonyl, C₃-C₈cycloalkylsulfonyl, mono- or di-(C₁-C₆alkyl)aminocarbonyl, mono- or di-(C₁-C₆alkyl)aminosulfonyl, mono- or di-(C₁-C₆alkyl)aminoC₁-C₄alkyl, or (5- or 6-membered heteroaryl)C₀-C₄alkyl, each of which is substituted with from 0 to 4 substituents independently chosen from hydroxy, halogen, cyano, oxo, C₁-C₆alkyl, aminocarbonyl, mono- or di-(C₁-C₆alkyl)aminocarbonyl, or mono- or di-(C₁-C₆alkyl)amino.

37. A compound or salt according to claim 36, wherein the compound has the formula: 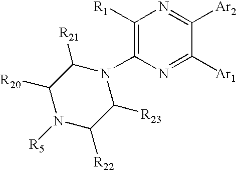
wherein one of R₂₀ and R₂₁ is taken together with R₂₂ or R₂₃ to form a methylene or ethylene bridge, and those of R₂₀, R₂₁, R₂₂ and R₂₃ that do not form bridge are hydrogen.

38. (canceled)

39. A compound or salt according to claim 33, wherein the compound has the formula: 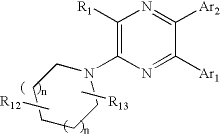
wherein:
each n is independently 0, 1 or 2;
R₁₂ is halogen, cyano, amino, C₁-C₄alkyl, (C₃-C₈cycloalkyl)C₀-C₄alkyl, C₂-C₄alkenyl, C₁-C₄hydroxyalkyl, C₁-C₄alkoxy, C₂-C₄alkyl ether, C₁-C₄alkanoyl, C₁-C₄alkylsulfonyl, mono- or di-(C₁-C₆alkyl)amino, or (5- to 7-membered heterocycle)C₀-C₂alkyl, each of which is substituted with from 0 to 3 substituents independently chosen from oxo, amino, hydroxy, C₁-C₄alkoxy, and mono- or di-(C₁-C₆alkyl)amino; and
R₁₃ represents from 0 to 3 substituents independently chosen from hydroxyl, halogen, cyano, amino, aminocarbonyl, C₁-C₄alkyl, C₂-C₄alkenyl, C₁-C₄hydroxyalkyl, C₁-C₄alkoxy, C₂-C₄alkyl ether and C₁-C₄alkanoyl; or
R₁₂ and R₁₃ are taken together to form a fused or spiro 5- to 7-membered heterocycle that is substituted with from 0 to 2 substituents independently chosen from hydroxy, oxo, C₁-C₄alkyl and C₁-C₄hydroxyalkyl.

40. A compound or salt according to claim 33, wherein the compound has the formula:

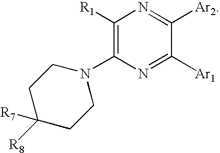

41. A compound or salt according to claim 40, wherein R₇ is hydrogen or aminocarbonyl and R₈ is mono- or di-(C₁-C₆alkyl)amino or C₁-C₆alkanoylamino.

42. (canceled)

43. A compound or salt according to claim 33, wherein the compound has the formula:

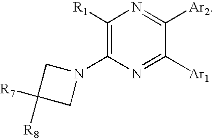

44. A compound or salt according to claim 33, wherein the compound has the formula:

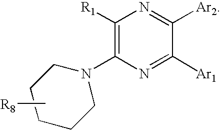

45-48. (canceled)

49. A compound of the formula: 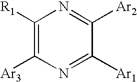
or a pharmaceutically acceptable salt thereof, wherein:
Ar₁ and Ar₂ are independently chosen from 5- to 10-membered carbocycles and heterocycles, each of which is substituted with from 0 to 6 substituents independently chosen from R_{A};
Ar₃ is a 5-membered heteroaryl that is substituted with from 0 to 3 substituents independently chosen from hydroxy, C₁-C₄alkyl, C₁-C₄hydroxyalkyl, C₁-C₄carboxyalkyl, C₂-C₄alkyl ether, C₁-C₄alkylsulfonyl, mono- or di-(C₁-C₄alkyl)aminoC₀-C₄alkyl, mono- or di-(C₁-C₄alkyl)aminocarbonylC₀-C₄alkyl and (4- to 8-membered heterocycle)C₀-C₄alkyl;
Each R_{A} is independently chosen from:
(i) halogen, hydroxy, cyano, amino, nitro, aminocarbonyl, aminosulfonyl and —COOH;
(ii) C₁-C₆alkyl, C₂-C₆alkenyl, C₂-C₆alkynyl, (C₃-C₈cycloalkyl)C₀-C₄alkyl, C₁-C₆alkoxy, C₁-C₆alkylthio, C₁-C₆alkylsulfinyl, C₁-C₆alkoxycarbonyl, C₁-C₆alkylsulfonylC₀-C₄alkyl, mono- or di-(C₁-C₆alkyl)aminoC₀-C₄alkyl, mono- or di-(C₁-C₆alkyl)aminosulfonylC₀-C₄alkyl, mono- or di-(C₁-C₆alkyl)aminocarbonylC₀-C₄alkyl, phenylC₀-C₄alkyl, (4- to 8-membered heterocycle)C₀-C₄alkyl and (4- to 8-membered heterocycle)C₁-C₄alkoxy; each of which is substituted with from 0 to 6 substituents independently chosen from R_{E};
or two R_{A} groups attached to adjacent ring carbon atoms are taken together to form a fused 5- to 7-membered carbocycle or heterocycle that is substituted with from 0 to 6 substituents independently chosen from R_{E};
Each R_{E} is independently chosen from oxo, halogen, hydroxy, cyano, amino, nitro, aminocarbonyl, aminosulfonyl, —COOH, C₁-C₆alkyl, C₁-C₆haloalkyl, C₁-C₆alkoxy, C₁-C₆haloalkoxy, C₁-C₆alkylthio, C₁-C₆alkoxycarbonyl, C₁-C₆alkanoyloxy, C₃-C₆alkanone, mono- or di-(C₁-C₆alkyl)amino, C₁-C₆alkylsulfonyl, mono- or di-(C₁-C₆alkyl)aminosulfonyl, and mono- or di-(C₁-C₆alkyl)aminocarbonyl; and
R₁ is:
(i) hydrogen, halogen, cyano, nitro, —COOH, aminocarbonyl or aminosulfonyl; or
(ii) C₁-C₆alkyl, C₂-C₆alkenyl, C₂-C₆alkynyl, (C₃-C₈cycloalkyl)C₀-C₄alkyl, C₁-C₆alkoxy, C₁-C₆alkoxycarbonyl, C₁-C₆alkylsulfonylC₀-C₄alkyl, C₁-C₆alkylaminosulfonylC₀-C₄alkyl, mono- or di-(C₁-C₆alkyl)aminoC₀-C₄alkyl, or mono- or di-(C₁-C₆alkyl)aminocarbonylC₀-C₄alkyl or a (4- to 8-membered heterocycle)C₀-C₄alkyl; each of which is substituted with from 0 to 6 substituents independently chosen from halogen, hydroxy, cyano and amino.

50. A compound or salt according to claim 32, wherein R₁ is hydrogen, bromo, chloro, cyano, methyl, ethyl, methylamino or ethylamino.

51. A compound or salt according to claim 32, wherein Ar₁ is phenyl or pyridyl, each of which is substituted with from 0 to 3 substituents independently chosen from chloro, bromo, fluoro, cyano, aminocarbonyl, C₁-C₄alkyl, C₁-C₄alkoxy, C₁-C₄alkoxycarbonyl, mono- or di-(C₁-C₄alkyl)aminocarbonyl and C₁-C₄alkanoyl.

52. A compound or salt according to claim 51, wherein Ar₁ is pyridin-4-yl that is substituted with 1 or 2 substituents.

53. A compound or salt according to claim 51, wherein Ar₁ is substituted with 1 or 2 halogens.

54. A compound or salt according to claim 53, wherein Ar₁ is 2-chloro-pyridin-4-yl, 4-fluorophenyl, 4-chlorophenyl or 2,4-dichlorophenyl.

55. A compound or salt according to claim 32, wherein Ar₁ is cyclohexyl or a 6-membered heterocycloalkyl, each of which is substituted with from 0 to 2 substituents independently chosen from chloro, bromo, fluoro, cyano, aminocarbonyl, C₁-C₄alkyl, C₁-C₄alkoxy, C₁-C₄alkoxycarbonyl, mono- or di-(C₁-C₄alkyl)aminocarbonyl and C₁-C₄alkanoyl.

56. A compound or salt according to claim 55, wherein Ar₁ is morpholinyl or thiomorpholinyl.

57. A compound or salt according to claim 32, wherein Ar₂ is phenyl, pyrrolyl or pyridyl, each of which is substituted with from 0 to 3 substituents independently chosen from:
(i) chloro, bromo, fluoro, cyano, aminocarbonyl, C₁-C₄alkyl, C₁-C₄haloalkyl, C₁-C₄haloalkoxy, C₁-C₄alkoxycarbonyl, mono- or di-(C₁-C₄alkyl)aminocarbonyl and C₁-C₄alkanoyl; and
(ii) C₁-C₄alkoxy that is unsubstituted or substituted with hydroxy, amino, C₁-C₄alkoxy, mono- or di-(C₁-C₄alkyl)amino or a 4- to 7-membered heterocycloalkyl.

58. A compound or salt according to claim 57, wherein Ar₂ is substituted with 1 or 2 substituents.

59. A compound or salt according to claim 57, wherein Ar₁ and Ar₂ are both 4-fluorophenyl or 4-chlorophenyl.

60. A compound or salt according to claim 32, wherein Ar₂ is cyclohexyl or a 6-membered heterocycloalkyl, each of which is substituted with from 0 to 2 substituents independently chosen from:
(i) chloro, bromo, fluoro, cyano, aminocarbonyl, C₁-C₄alkyl, C₁-C₄haloalkyl, C₁-C₄haloalkoxy, C₁-C₄alkoxycarbonyl, mono- or di-(C₁-C₄alkyl)aminocarbonyl and C₁-C₄alkanoyl; and
(ii) C₁-C₄alkoxy that is unsubstituted or substituted with hydroxy, amino, C₁-C₄alkoxy, mono- or di-(C₁-C₄alkyl)amino or a 4- to 7-membered heterocycloalkyl.

61. A compound or salt according to claim 60, wherein Ar₂ is morpholinyl or thiomorpholinyl.

62. A compound of the formula: 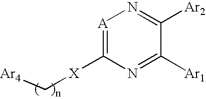
or a pharmaceutically acceptable salt thereof, wherein:
A is CR₁, or N;
Ar₁ and Ar₂ are independently chosen from 5- to 10-membered carbocycles and heterocycles, each of which is substituted with from 1 to 6 substituents independently chosen from R_{A}; such that at least one of Ar₁ and Ar₂ is substituted aryl or substituted heteroaryl;
Ar₄ is phenyl or a 5- or 6-membered aromatic heterocycle, each of which is substituted with from 0 to 4 substituents independently chosen from R_{A};
such that at least one of Ar₁, Ar₂ and Ar₄ is a heterocycle;
X is O, N(R₂) or SOₘN(R₂);
Each R_{A} is independently chosen from:
(i) halogen, hydroxy, cyano, amino, nitro, aminocarbonyl, aminosulfonyl and —COOH; and
(ii) C₁-C₆alkyl, C₂-C₆alkenyl, C₂-C₆alkynyl, (C₃-C₈cycloalkyl)C₀-C₄alkyl, C₁-C₆alkoxy, C₁-C₆alkylthio, C₁-C₆alkylsulfinyl, C₁-C₆alkoxycarbonyl, C₁-C₆alkylsulfonylC₀-C₄alkyl, mono- or di-(C₁-C₆alkyl)aminoC₀-C₄alkyl, mono- or di-(C₁-C₆alkyl)aminosulfonylC₀-C₄alkyl, mono- or di-(C₁-C₆alkyl)aminocarbonylC₀-C₄alkyl, phenylC₀-C₄alkyl, (4- to 8-membered heterocycle)C₀-C₄alkyl and (4- to 8-membered heterocycle)C₁-C₄alkoxy; each of which is substituted with from 0 to 6 substituents independently chosen from R_{E};
or two R_{A} groups attached to adjacent ring carbon atoms are taken together to form a fused 5- to 7-membered carbocycle or heterocycle that is substituted with from 0 to 6 substituents independently chosen from R_{E};
Each R_{E} is independently chosen from oxo, halogen, hydroxy, cyano, amino, nitro, aminocarbonyl, aminosulfonyl, —COOH, C₁-C₆alkyl, C₁-C₆haloalkyl, C₁-C₆alkoxy, C₁-C₆haloalkoxy, C₁-C₆alkylthio, C₁-C₆alkoxycarbonyl, C₁-C₆alkanoyloxy, C₃-C₆alkanone, mono- or di-(C₁-C₆alkyl)amino, C₁-C₆alkylsulfonyl, mono- or di-(C₁-C₆alkyl)aminosulfonyl and mono- or di-(C₁-C₆alkyl)aminocarbonyl;
R₁ is:
(i) hydrogen, halogen, hydroxy, cyano, amino, nitro, —COOH, aminocarbonyl or aminosulfonyl;
(ii) C₁-C₆alkyl, C₂-C₆alkenyl, C₂-C₆alkynyl, (C₃-C₈cycloalkyl)C₀-C₄alkyl, C₁-C₆alkoxy, C₁-C₆alkoxycarbonyl, C₁-C₆alkylsulfonylC₀-C₄alkyl, mono- or di-(C₁-C₆alkyl)aminoC₀-C₄alkyl, or mono- or di-(C₁-C₆alkyl)aminocarbonylC₀-C₄alkyl or a (4- to 8-membered heterocycle)C₀-C₄alkyl; each of which is substituted with from 0 to 6 substituents independently chosen from R_{E}; or
(iii) taken together with R₂ to form a fused 5- to 8-membered heterocycle that is substituted with from 0 to 4 substituents independently chosen from R_{E};
R₂ is:
(i) hydrogen or aminocarbonyl;
(ii) C₁-C₆alkyl, C₂-C₆alkenyl, C₂-C₆alkynyl, (C₃-C₈cycloalkyl)C₀-C₄alkyl, C₂-C₆alkyl ether, C₁-C₆alkoxycarbonyl, mono- or di-(C₁-C₄alkyl)aminocarbonyl, mono- or di-(C₁-C₄alkyl)aminosulfonyl, mono- or di-(C₁-C₄alkyl)aminoC₀-C₄alkyl, C₁-C₆alkylsulfonyl or (4- to 8-membered heterocycle)C₀-C₄alkyl; each of which is optionally substituted, and each of which is preferably substituted with from 0 to 6 substituents independently chosen from R_{E}; or
(iii) taken together with R₁ to form an optionally substituted fused 5- to 8-membered heterocycle; and
n is 1, 2 or 3.

63. A compound of the formula: 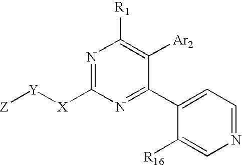
or a pharmaceutically acceptable salt thereof, wherein:
Ar₂ is a 5- to 10-membered carbocycle or heterocycle, each of which is substituted with from 0 to 6 substituents independently chosen from R_{A};
X is O, N(R₂) or SOₘN(R₂);
Y is C₁-C₄alkylene that is unsubstituted or substituted with one or two substituents independently chosen from R_{B};
Z is O(R₃), S(O)ₘ(R₄), N(R₄)(R₅), S(O)ₘN(R₄)(R₅) or C(R₆)(R₇)(R₈); wherein m is 0, 1 or 2;
Each R_{A} is independently chosen from:
(i) halogen, hydroxy, cyano, amino, nitro, oxo, aminocarbonyl, aminosulfonyl and —COOH; and
(ii) C₁-C₆alkyl, C₂-C₆alkenyl, C₂-C₆alkynyl, (C₃-C₈cycloalkyl)C₀-C₄alkyl, C₁-C₆alkoxy, C₁-C₆alkylthio, C₁-C₆alkylsulfinyl, C₁-C₆alkoxycarbonyl, C₁-C₆alkylsulfonylC₀-C₄alkyl, mono- or di-(C₁-C₆alkyl)aminoC₀-C₄alkyl, mono- or di-(C₁-C₆alkyl)aminosulfonylC₀-C₄alkyl, mono- or di-(C₁-C₆alkyl)aminocarbonylC₀-C₄alkyl, phenylC₀-C₄alkyl, (4- to 8-membered heterocycle)C₀-C₄alkyl and (4- to 8-membered heterocycle)C₁-C₄alkoxy; each of which is substituted with from 0 to 6 substituents independently chosen from R_{E};
or two R_{A} groups attached to adjacent ring carbon atoms are taken together to form a fused 5- to 7-membered carbocycle or heterocycle that is substituted with from 0 to 6 substituents independently chosen from R_{E};
Each R_{B} is:
(i) halogen, hydroxy, —COOH, aminocarbonyl, C₁-C₄alkyl, C₁-C₄alkoxy, C₁-C₄haloalkyl, C₁-C₄aminoalkyl, mono- or di-(C₁-C₆alkyl)aminoC₀-C₄alkyl, or mono- or di-(C₁-C₆alkyl)aminocarbonyl; or
(ii) taken together with R₃ or R₄ to form a 4- to 10-membered heterocycloalkyl that is substituted with from 0 to 3 substituents independently chosen from R_{D}; such that if R_{B} forms a heterocycloalkyl with R₃, then the heterocycloalkyl is not substituted with C₁-C₄alkoxycarbonyl;
Each R_{D} is independently chosen from:
(i) hydroxy, halogen, cyano, amino, oxo, nitro, —COOH, aminocarbonyl and aminosulfonyl;
(ii) C₁-C₆alkyl, C₁-C₆alkenyl, C₁-C₆alkynyl, C₃-C₈cycloalkylC₀-C₄alkyl, C₁-C₆alkoxy, C₁-C₆alkylthio, C₁-C₆alkyl ether, mono- or di-(C₁-C₆alkyl)amino, mono- or di-(C₁-C₆alkyl)aminocarbonyl, mono- or di-(C₁-C₆alkyl)aminosulfonyl, C₁-C₆alkylsulfinyl, C₁-C₆alkylsulfonyl, (4- to 8-membered heterocycle)C₀-C₄alkyl and phenylC₀-C₄alkyl, each of which is substituted with from 0 to 4 substituents independently chosen from R_{E}; and
(iii) taken together with R₅ to form a 4- to 8-membered heterocycloalkyl;
Each R_{E} is independently chosen from oxo, halogen, hydroxy, cyano, amino, nitro, aminocarbonyl, aminosulfonyl, —COOH, C₁-C₆alkyl, C₁-C₆haloalkyl, C₁-C₆alkoxy, C₁-C₆haloalkoxy, C₁-C₆alkylthio, C₁-C₆alkoxycarbonyl, C₁-C₆alkanoyloxy, C₃-C₆alkanone, mono- or di-(C₁-C₆alkyl)amino, C₁-C₆alkylsulfonyl, mono- or di-(C₁-C₆alkyl)aminosulfonyl, and mono- or di-(C₁-C₆alkyl)aminocarbonyl;
R₁ is:
(i) hydrogen, hydroxy, amino, halogen, cyano, nitro, —COOH, aminocarbonyl or aminosulfonyl; or
(ii) C₁-C₆alkyl, C₂-C₆alkenyl, C₂-C₆alkynyl, (C₃-C₈cycloalkyl)C₀-C₄alkyl, C₁-C₆alkoxy, C₁-C₆alkoxycarbonyl, C₁-C₆alkylsulfonylC₀-C₄alkyl, C₁-C₆alkylaminosulfonylC₀-C₄alkyl, mono- or di-(C₁-C₆alkyl)aminoC₀-C₄alkyl, or mono- or di-(C₁-C₆alkyl)aminocarbonylC₀-C₄alkyl or a (4- to 8-membered heterocycle)C₀-C₄alkyl; each of which is substituted with from 0 to 6 substituents independently chosen from R_{E};
R₂, if present, is taken together with R₃, R₄ or R₆ to form a 4- to 10-membered heterocycle that is substituted with from 0 to 3 substituents independently chosen from R_{D};
R₃ is:
(i) hydrogen;
(ii) C₁-C₈alkyl, C₁-C₈alkenyl, C₁-C₈alkynyl, C₁-C₆alkanone, C₁-C₈alkyl ether, mono- or di-(C₁-C₆alkyl)aminoC₁-C₄alkyl, (C₃-C₈cycloalkyl)C₀-C₄alkyl or (4- to 8-membered heterocycloalkyl)C₀-C₄alkyl; each of which is substituted with from 0 to 3 substituents independently chosen from hydroxy, halogen, C₁-C₆alkyl or C₁-C₆alkoxy; or
(iii) taken together with R₂ or R_{B} to form a 4- to 8-membered heterocycle that is substituted with from 0 to 3 substituents independently chosen from R_{D};
R₄ is:
(i) hydrogen or aminocarbonyl;
(ii) C₁-C₈alkyl, C₁-C₈alkenyl, C₁-C₈alkynyl, (C₃-C₈cycloalkyl)C₀-C₄alkyl, C₁-C₆alkylsulfonyl, (C₃-C₈cycloalkyl)sulfonyl, C₁-C₆alkoxycarbonyl, C₂-C₈alkyl ether, mono- or di-(C₁-C₆alkyl)aminosulfonyl, mono- or di-(C₁-C₆alkyl)aminocarbonyl or (5- or 6-membered heterocycle)C₀-C₄alkyl; each of which is substituted with from 0 to 6 substituents independently chosen from halogen, hydroxy, oxo, cyano, amino, aminosulfonyl, aminocarbonyl, C₁-C₆alkyl, C₁-C₆alkoxy, C₁-C₆alkylsulfonyl, mono- or di-(C₁-C₆alkyl)aminosulfonyl, mono- or di-(C₁-C₆alkyl)amino and mono- or di-(C₁-C₆alkyl)aminocarbonyl; or
(iii) taken together with R₂, R₅, or R_{B} to form a 4- to 10-membered heterocycle that is substituted with from 0 to 4 substituents independently chosen from R_{D};
R₅ is:
(i) hydrogen or aminocarbonyl;
(ii) C₁-C₈alkyl, C₁-C₈alkenyl, C₁-C₈alkynyl, (C₃-C₈cycloalkyl)C₀-C₄alkyl, C₁-C₆alkylsulfonyl, (C₃-C₈cycloalkyl)sulfonyl, C₁-C₆alkoxycarbonyl, C₂-C₈alkyl ether, mono- or di-(C₁-C₆alkyl)aminosulfonyl, mono- or di-(C₁-C₆alkyl)aminocarbonyl, phenylC₀-C₄alkyl or (5- or 6-membered heterocycle)C₀-C₄alkyl; each of which is substituted with from 0 to 6 substituents independently chosen from halogen, hydroxy, oxo, cyano, amino, —COOH, aminosulfonyl, aminocarbonyl, C₁-C₆alkyl, C₁-C₆alkoxy, C₁-C₆alkylsulfonyl, mono- or di-(C₁-C₆alkyl)aminosulfonyl, mono- or di-(C₁-C₆alkyl)amino, mono- or di-(C₁-C₆alkyl)aminocarbonyl and phenyl; or
(iii) taken together with R₄ or R_{D} to form an optionally substituted 4- to 8-membered heterocycle;
R₆ is:
(i) hydrogen, hydroxy, halogen, cyano, amino, aminocarbonyl, aminosulfonyl or —COOH;
(ii) C₁-C₈alkyl, C₂-C₈alkenyl, C₂-C₈alkynyl, (C₃-C₈cycloalkyl)C₀-C₄alkyl, C₁-C₆alkoxy, C₁-C₆alkylthio, C₁-C₆alkylsulfinyl, C₁-C₆alkylsulfonyl, (C₃-C₈cycloalkyl)sulfonyl, C₁-C₆alkoxycarbonyl, mono- or di-(C₁-C₆alkyl)aminosulfonyl, mono- or di-(C₁-C₆alkyl)aminocarbonyl, mono- or di-(C₁-C₆alkyl)aminoC₀-C₄alkyl or (5- or 6-membered heterocycle)C₀-C₄alkyl; each of which is optionally substituted, and each of which is preferably substituted with from 0 to 6 substituents independently chosen from R_{E}; or
(iii) taken together with one or two of R₂, R₇, R₈, or R_{B} to form a 4- to 10-membered carbocycle or heterocycle that is substituted with from 0 to 3 substituents independently chosen from R_{D};
R₇ and R₈ are independently:
(i) hydrogen, hydroxy, halogen, cyano, amino, aminocarbonyl, aminosulfonyl or —COOH;
(ii) C₁-C₈alkyl, C₁-C₈alkenyl, C₁-C₈alkynyl, (C₃-C₈cycloalkyl)C₀-C₄alkyl, C₁-C₆alkoxy, C₁-C₆alkanoyl, C₁-C₈alkylthio, C₁-C₈alkylsulfinyl, C₁-C₆alkylsulfonyl, (C₃-C₈cycloalkyl)sulfonyl, mono- or di-(C₁-C₆alkyl)amino, mono- or di-(C₁-C₆alkyl)aminosulfonyl, or mono- or di-(C₁-C₆alkyl)aminocarbonyl, each of which is substituted with from 0 to 4 substituents independently chosen from R_{E};
(iii) taken together with R₆ to form an optionally substituted carbocycle or heterocycle; or
(iv) R₇ and R₈ taken together form an optionally substituted carbocycle or heterocycle; and
R₁₆ is chloro, fluoro or C₁-C₃alkyl.

64.-82. (canceled)

83. A compound of the Formula: 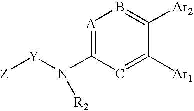
or a pharmaceutically acceptable salt thereof, wherein:
A, B and C are independently chosen from nitrogen and CR₁, such that exactly one of A, B and C is nitrogen;
Ar₁ is a 5- to 10-membered heterocycle that is substituted with from 1 to 6 substituents independently chosen from R_{A};
Ar₂ is a 5- to 10-membered carbocycle or heterocycle, each of which is substituted with from 0 to 6 substituents independently chosen from R_{A};
Y is C₁-C₄alkylene that is unsubstituted or substituted with one or two substituents independently chosen from R_{B};
Z is hydrogen, O(R₃), S(O)ₘ(R₄), N(R₄)(R₅), S(O)ₘN(R₄)(R₅) or C(R₆)(R₇)(R₈); wherein
m is 0, 1 or 2;
Each R_{A} is independently chosen from:
(i) halogen, hydroxy, cyano, amino, nitro, aminocarbonyl, aminosulfonyl and —COOH; and
(ii) C₁-C₆alkyl, C₂-C₆alkenyl, C₂-C₆alkynyl, (C₃-C₈cycloalkyl)C₀-C₄alkyl, C₁-C₆alkoxy, C₁-C₆alkylthio, C₁-C₆alkylsulfinyl, C₁-C₆alkoxycarbonyl, C₁-C₆alkylsulfonylC₀-C₄alkyl, mono- or di-(C₁-C₆alkyl)aminoC₀-C₄alkyl, mono- or di-(C₁-C₆alkyl)aminosulfonylC₀-C₄alkyl, mono- or di-(C₁-C₆alkyl)aminocarbonylC₀-C₄alkyl, phenylC₀-C₄alkyl, (4- to 8-membered heterocycle)C₀-C₄alkyl and (4- to 8-membered heterocycle)C₁-C₄alkoxy; each of which is substituted with from 0 to 6 substituents independently chosen from R_{E};
or two R_{A} groups attached to adjacent ring carbon atoms are taken together to form a fused 5- to 7-membered carbocycle or heterocycle that is substituted with from 0 to 6 substituents independently chosen from R_{E};
Each R_{B} is:
(i) halogen, hydroxy, —COOH, aminocarbonyl, C₁-C₄alkyl, C₁-C₄alkoxy, C₁-C₄haloalkyl, C₁-C₄aminoalkyl, mono- or di-(C₁-C₆alkyl)aminoC₀-C₄alkyl, or mono- or di-(C₁-C₆alkyl)aminocarbonyl; or
(ii) taken together with R₃, R₄ or R₆ to form a 4- to 10-membered carbocycle or heterocycle that is substituted with from 0 to 4 substituents independently chosen from R_{D};
or two R_{B} are taken together to form a C₃-C₈cycloalkyl or a 4- to 8-membered heterocycloalkyl;
Each R_{D} is independently chosen from:
(i) hydroxy, halogen, cyano, amino, oxo, nitro, —COOH, aminocarbonyl and aminosulfonyl; and
(ii) C₁-C₆alkyl, C₁-C₆alkenyl, C₁-C₆alkynyl, C₃-C₈cycloalkylC₀-C₄alkyl, C₁-C₆alkoxy, C₁-C₆alkylthio, C₁-C₆alkyl ether, mono- or di-(C₁-C₆alkyl)amino, mono- or di-(C₁-C₆alkyl)aminocarbonyl, mono- or di-(C₁-C₆alkyl)aminosulfonyl, C₁-C₆alkylsulfinyl, C₁-C₆alkylsulfonyl, (4- to 8-membered heterocycle)C₀-C₄alkyl and phenylC₀-C₄alkyl, each of which is substituted with from 0 to 4 substituents independently chosen from R_{E};
Each R_{E} is independently chosen from oxo, halogen, hydroxy, cyano, amino, nitro, aminocarbonyl, aminosulfonyl, —COOH, C₁-C₆alkyl, C₁-C₆haloalkyl, C₁-C₆alkoxy, C₁-C₆haloalkoxy, C₁-C₆alkylthio, C₁-C₆alkoxycarbonyl, C₁-C₆alkanoyloxy, C₃-C₆alkanone, mono- or di-(C₁-C₆alkyl)amino, C₁-C₆alkylsulfonyl, mono- or di-(C₁-C₆alkyl)aminosulfonyl and mono- or di-(C₁-C₆alkyl)aminocarbonyl;
Each R₁ is independently:
(i) hydrogen, halogen, hydroxy, cyano, amino, nitro, —COOH, aminocarbonyl or aminosulfonyl;
(ii) C₁-C₆alkyl, C₂-C₆alkenyl, C₂-C₆alkynyl, (C₃-C₈cycloalkyl)C₀-C₄alkyl, C₁-C₆alkoxy, C₁-C₆alkoxycarbonyl, C₁-C₆alkylsulfonylC₀-C₄alkyl, C₁-C₆alkylaminosulfonylC₀-C₄alkyl, mono- or di-(C₁-C₆alkyl)aminoC₀-C₄alkyl, or mono- or di-(C₁-C₆alkyl)aminocarbonylC₀-C₄alkyl or a (4- to 8-membered heterocycle)C₀-C₄alkyl; each of which is substituted with from 0 to 6 substituents independently chosen from R_{E}; or
(iii) if located at the A or C position, taken together with R₂ to form a fused 5- to 8-membered carbocycle or heterocycle that is substituted with from 0 to 4 substituents independently chosen from R_{E};
R₂ is:
(i) hydrogen or aminocarbonyl;
(ii) C₁-C₆alkyl, C₂-C₆alkenyl, C₂-C₆alkynyl, (C₃-C₈cycloalkyl)C₀-C₄alkyl, C₁-C₆alkyl ether, C₁-C₆alkoxycarbonyl, mono- or di-(C₁-C₄alkyl)aminocarbonyl, mono- or di-(C₁-C₄alkyl)aminosulfonyl, mono- or di-(C₁-C₄alkyl)aminoC₀-C₄alkyl, C₁-C₆alkylsulfonyl or (4- to 8-membered heterocycle)C₀-C₄alkyl; each of which is substituted with from 0 to 6 substituents independently chosen from R_{E};
(iii) taken together with R₃, R₄ or R₆ to form a 4- to 10-membered heterocycle that is substituted with from 0 to 3 substituents independently chosen from R_{D}; or
(iv) taken together with R₁ at the A position to form an optionally substituted fused 5- to 8-membered heterocycle;
R₃ is:
(i) hydrogen;
(ii) C₁-C₈alkyl, C₁-C₈alkenyl, C₁-C₈alkynyl, C₁-C₆alkanone, C₂-C₈alkyl ether, mono- or di-(C₁-C₆alkyl)aminoC₁-C₄alkyl, (C₃-C₈cycloalkyl)C₀-C₄alkyl, mono- or di-(C₁-C₆alkyl) amino carbonyl, (5- to 7-membered heterocycle)-C(═O)—, phenylC₀-C₄alkyl or (4- to 8-membered heterocycle)C₀-C₄alkyl; each of which is substituted with from 0 to 6 substituents independently chosen from R_{E}; or
(iii) taken together with R₂ or R_{B} to form a 4- to 10-membered heterocycle that is substituted with from 0 to 4 substituents independently chosen from R_{D};
R₄ is:
(i) hydrogen;
(ii) C₁-C₈alkyl, C₁-C₈alkenyl, C₁-C₈alkynyl, (C₃-C₈cycloalkyl)C₀-C₄alkyl, C₁-C₆alkylsulfonyl, (C₃-C₈cycloalkyl)sulfonyl, C₁-C₆alkoxycarbonyl, C₂-C₈alkyl ether, mono- or di-(C₁-C₆alkyl)aminosulfonyl, mono- or di-(C₁-C₆alkyl)aminocarbonyl or (5- or 6-membered heterocycle)C₀-C₄alkyl; each of which is substituted with from 0 to 6 substituents independently chosen from halogen, hydroxy, oxo, cyano, amino, aminosulfonyl, aminocarbonyl, C₁-C₆alkyl, C₁-C₆alkoxy, C₁-C₆alkylsulfonyl, mono- or di-(C₁-C₆alkyl)aminosulfonyl, mono- or di-(C₁-C₆alkyl)amino and mono- or di-(C₁-C₆alkyl)aminocarbonyl; or
(iii) taken together with one or two of R₂, R₅ or R_{B} to form a 4- to 10-membered heterocycle that is substituted with from 0 to 4 substituents independently chosen from R_{D};
R₅ is:
(i) hydrogen, cyano or aminocarbonyl;
(ii) C₁-C₈alkyl, C₁-C₈alkenyl, C₁-C₈alkynyl, (C₃-C₈cycloalkyl)C₀-C₄alkyl, C₁-C₆alkylsulfonyl, (C₃-C₈cycloalkyl)sulfonyl, C₁-C₆alkoxycarbonyl, C₂-C₈alkyl ether, mono- or di-(C₁-C₆alkyl)aminosulfonyl, mono- or di-(C₁-C₆alkyl)aminocarbonyl, mono- or di-(C₁-C₆alkyl)aminoC₀-C₄alkyl, phenylC₀-C₄alkyl or (5- or 6-membered heterocycle)C₀-C₄alkyl; each of which is substituted with from 0 to 6 substituents independently chosen from phenyl and R_{E}; or
(iii) taken together with R₄ or R_{D} to form an optionally substituted 4- to 10-membered heterocycloalkyl;
R₆ is:
(i) hydrogen, hydroxy, halogen, cyano, amino, aminocarbonyl, aminosulfonyl or —COOH;
(ii) C₁-C₈alkyl, C₂-C₈alkenyl, C₂-C₈alkynyl, (C₃-C₈cycloalkyl)C₀-C₄alkyl, C₁-C₆alkoxy, C₁-C₆alkylthio, C₁-C₆alkylsulfinyl, C₁-C₆alkylsulfonyl, (C₃-C₈cycloalkyl)sulfonyl, C₁-C₆alkoxycarbonyl, mono- or di-(C₁-C₆alkyl)aminosulfonyl, mono- or di-(C₁-C₆alkyl)aminocarbonyl, mono- or di-(C₁-C₆alkyl)aminoC₀-C₄alkyl or (5- or 6-membered heterocycle)C₀-C₄alkyl; each of which is substituted with from 0 to 6 substituents independently chosen from R_{E}; or
(iii) taken together with one or two of R₂, R₇, R₈ or R_{B} to form a 4- to 10-membered carbocycle or heterocycle that is substituted with from 0 to 3 substituents independently chosen from R_{D}; and
R₇ and R₈ are independently:
(i) hydrogen, hydroxy, halogen, cyano, amino, aminocarbonyl, aminosulfonyl or —COOH;
(ii) C₁-C₈alkyl, C₁-C₈alkenyl, C₁-C₈alkynyl, (C₃-C₈cycloalkyl)C₀-C₄alkyl, C₁-C₆alkoxy, C₁-C₈alkylthio, C₁-C₈alkylsulfinyl, C₁-C₆alkylsulfonyl, (C₃-C₈cycloalkyl)sulfonyl, C₁-C₆alkoxycarbonyl, mono- or di-(C₁-C₆alkyl)amino, mono- or di-(C₁-C₆alkyl)aminosulfonyl, mono- or di-(C₁-C₆alkyl)aminocarbonyl, phenylC₀-C₄alkyl or (5- or 6-membered heterocycle)C₀-C₄alkyl; each of which is substituted with from 0 to 6 substituents independently chosen from phenyl and R_{E}; or
(iii) taken together with R₆ to form an optionally substituted carbocycle or heterocycle;
or R₇ and R₈ are taken together to form a 4- to 8-membered carbocycle or heterocycle that is substituted with from 0 to 3 substituents independently chosen from R_{D}.

84.-107. (canceled)

108. A compound of the Formula: 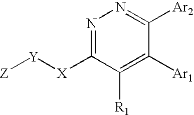
or a pharmaceutically acceptable salt thereof, wherein:
Ar₁ and Ar₂ are independently chosen from 5- to 10-membered carbocycles and heterocycles, each of which is substituted with from 0 to 6 substituents independently chosen from R_{A}; such that at least one of Ar₁ and Ar₂ is an optionally substituted 5- or 6-membered heterocycle;
X is O, N(R₂) or SOₘN(R₂);
Y is C₁-C₄alkylene that is unsubstituted or substituted with one or two substituents independently chosen from R_{B};
Z is hydrogen, O(R₃), S(O)ₘ(R₄), N(R₄)(R₅), S(O)ₘN(R₄)(R₅) or C(R₆)(R₇)(R₈); wherein m is 0, 1 or 2;
Each R_{A} is independently chosen from:
(i) halogen, hydroxy, cyano, amino, nitro, aminocarbonyl, aminosulfonyl and —COOH; and
(ii) C₁-C₆alkyl, C₂-C₆alkenyl, C₂-C₆alkynyl, (C₃-C₈cycloalkyl)C₀-C₄alkyl, C₁-C₆alkoxy, C₁-C₆alkylthio, C₁-C₆alkylsulfinyl, C₁-C₆alkoxycarbonyl, C₁-C₆alkylsulfonylC₀-C₄alkyl, mono- or di-(C₁-C₆alkyl)aminoC₀-C₄alkyl, mono- or di-(C₁-C₆alkyl)aminosulfonylC₀-C₄alkyl, mono- or di-(C₁-C₆alkyl)aminocarbonylC₀-C₄alkyl, phenylC₀-C₄alkyl, (4- to 8-membered heterocycle)C₀-C₄alkyl and (4- to 8-membered heterocycle)C₁-C₄alkoxy; each of which is substituted with from 0 to 6 substituents independently chosen from R_{E};
or two R_{A} groups attached to adjacent ring carbon atoms are taken together to form a fused 5- to 7-membered carbocycle or heterocycle that is substituted with from 0 to 6 substituents independently chosen from R_{E};
Each R_{B} is:
(i) halogen, hydroxy, —COOH, aminocarbonyl, C₁-C₄alkyl, C₁-C₄alkoxy, C₁-C₄haloalkyl, C₁-C₄aminoalkyl, mono- or di-(C₁-C₆alkyl)aminoC₀-C₄alkyl, or mono- or di-(C₁-C₆alkyl)aminocarbonyl; or
(ii) taken together with R₃, R₄ or R₆ to form a 4- to 10-membered carbocycle or heterocycle that is substituted with from 0 to 4 substituents independently chosen from R_{D};
or two R_{B} are taken together to form a C₃-C₈cycloalkyl or a 4- to 8-membered heterocycloalkyl;
Each R_{D} is independently chosen from:
(i) hydroxy, halogen, cyano, amino, oxo, nitro, —COOH, aminocarbonyl and aminosulfonyl; and
(ii) C₁-C₆alkyl, C₁-C₆alkenyl, C₁-C₆alkynyl, C₃-C₈cycloalkylC₀-C₄alkyl, C₁-C₆alkoxy, C₁-C₆alkylthio, C₁-C₆alkyl ether, mono- or di-(C₁-C₆alkyl)amino, mono- or di-(C₁-C₆alkyl)aminocarbonyl, mono- or di-(C₁-C₆alkyl)aminosulfonyl, C₁-C₆alkylsulfinyl, C₁-C₆alkylsulfonyl, (4- to 8-membered heterocycle)C₀-C₄alkyl and phenylC₀-C₄alkyl, each of which is substituted with from 0 to 4 substituents independently chosen from R_{E};
Each R_{E} is independently chosen from oxo, halogen, hydroxy, cyano, amino, nitro, aminocarbonyl, aminosulfonyl, —COOH, C₁-C₆alkyl, C₁-C₆haloalkyl, C₁-C₆alkoxy, C₁-C₆haloalkoxy, C₁-C₆alkylthio, C₁-C₆alkoxycarbonyl, C₁-C₆alkanoyloxy, C₃-C₆alkanone, mono- or di-(C₁-C₆alkyl)amino, C₁-C₆alkylsulfonyl, mono- or di-(C₁-C₆alkyl)aminosulfonyl and mono- or di-(C₁-C₆alkyl)aminocarbonyl;
R₁ is:
(i) hydrogen, halogen, hydroxy, cyano, amino, nitro, —COOH, aminocarbonyl or aminosulfonyl;
(ii) C₁-C₆alkyl, C₂-C₆alkenyl, C₂-C₆alkynyl, (C₃-C₈cycloalkyl)C₀-C₄alkyl, C₁-C₆alkoxy, C₁-C₆alkoxycarbonyl, C₁-C₆alkylsulfonylC₀-C₄alkyl, mono- or di-(C₁-C₆alkyl)aminoC₀-C₄alkyl, or mono- or di-(C₁-C₆alkyl)aminocarbonylC₀-C₄alkyl or a (4- to 8-membered heterocycle)C₀-C₄alkyl; each of which is substituted with from 0 to 6 substituents independently chosen from R_{E}; or
(iii) taken together with R₂ or R₉ to form a fused 5- to 8-membered carbocycle or heterocycle that is substituted with from 0 to 4 substituents independently chosen from R_{E};
R₂ is:
(i) hydrogen or aminocarbonyl;
(ii) C₁-C₆alkyl, C₂-C₆alkenyl, C₂-C₆alkynyl, (C₃-C₈cycloalkyl)C₀-C₄alkyl, C₁-C₆alkyl ether, C₁-C₆alkoxycarbonyl, mono- or di-(C₁-C₄alkyl)aminocarbonyl, mono- or di-(C₁-C₄alkyl)aminosulfonyl, mono- or di-(C₁-C₄alkyl)aminoC₀-C₄alkyl, C₁-C₆alkylsulfonyl or (4- to 8-membered heterocycle)C₀-C₄alkyl; each of which is substituted with from 0 to 6 substituents independently chosen from R_{E};
(iii) taken together with R₃, R₄ or R₆ to form a 4- to 10-membered heterocycle that is substituted with from 0 to 3 substituents independently chosen from R_{D}; or
(iv) taken together with R₁ to form an optionally substituted fused 5- to 8-membered heterocycle;
R₃ is:
(i) hydrogen;
(ii) C₁-C₈alkyl, C₁-C₈alkenyl, C₁-C₈alkynyl, C₁-C₆alkanone, C₂-C₈alkyl ether, mono- or di-(C₁-C₆alkyl)aminoC₁-C₄alkyl, (C₃-C₈cycloalkyl)C₀-C₄alkyl, mono- or di-(C₁-C₆alkyl) amino carbonyl, (5- to 7-membered heterocycle) —C(═O)—, phenylC₀-C₄alkyl or (4- to 8-membered heterocycle)C₀-C₄alkyl; each of which is substituted with from 0 to 6 substituents independently chosen from R_{E}; or
(iii) taken together with R₂, R₉ or R_{B} to form a 4- to 10-membered heterocycle that is substituted with from 0 to 4 substituents independently chosen from R_{D};
R₄ is:
(i) hydrogen;
(ii) C₁-C₈alkyl, C₁-C₈alkenyl, C₁-C₈alkynyl, (C₃-C₈cycloalkyl)C₀-C₄alkyl, C₁-C₆alkylsulfonyl, (C₃-C₈cycloalkyl)sulfonyl, C₁-C₆alkoxycarbonyl, C₂-C₈alkyl ether, mono- or di-(C₁-C₆alkyl)aminosulfonyl, mono- or di-(C₁-C₆alkyl)aminocarbonyl or (5- or 6-membered heterocycle)C₀-C₄alkyl; each of which is substituted with from 0 to 6 substituents independently chosen from halogen, hydroxy, oxo, cyano, amino, aminosulfonyl, aminocarbonyl, C₁-C₆alkyl, C₁-C₆alkoxy, C₁-C₆alkylsulfonyl, mono- or di-(C₁-C₆alkyl)aminosulfonyl, mono- or di-(C₁-C₆alkyl)amino and mono- or di-(C₁-C₆alkyl)aminocarbonyl; or
(iii) taken together with one or two of R₂, R₅, R₉ or R_{B} to form a 4- to 10-membered heterocycle that is substituted with from 0 to 4 substituents independently chosen from R_{D};
R₅ is:
(i) hydrogen, cyano or aminocarbonyl;
(ii) C₁-C₈alkyl, C₁-C₈alkenyl, C₁-C₈alkynyl, (C₃-C₈cycloalkyl)C₀-C₄alkyl, C₁-C₆alkylsulfonyl, (C₃-C₈cycloalkyl)sulfonyl, C₁-C₆alkoxycarbonyl, C₂-C₈alkyl ether, mono- or di-(C₁-C₆alkyl)aminosulfonyl, mono- or di-(C₁-C₆alkyl)aminocarbonyl, mono- or di-(C₁-C₆alkyl)aminoC₀-C₄alkyl, phenylC₀-C₄alkyl or (5- or 6-membered heterocycle)C₀-C₄alkyl; each of which is substituted with from 0 to 6 substituents independently chosen from phenyl and R_{E}; or
(iii) taken together with R₄ to form an optionally substituted 4- to 10-membered heterocycloalkyl;
R₆ is:
(i) hydrogen, hydroxy, halogen, cyano, amino, aminocarbonyl, aminosulfonyl or —COOH;
(ii) C₁-C₈alkyl, C₁-C₈alkenyl, C₁-C₈alkynyl, (C₃-C₈cycloalkyl)C₀-C₄alkyl, C₁-C₆alkoxy, C₁-C₆alkylthio, C₁-C₆alkylsulfinyl, C₁-C₆alkylsulfonyl, (C₃-C₈cycloalkyl)sulfonyl, C₁-C₆alkoxycarbonyl, mono- or di-(C₁-C₆alkyl)amino, mono- or di-(C₁-C₆alkyl)aminosulfonyl, mono- or di-(C₁-C₆alkyl)aminocarbonyl or (5- or 6-membered heterocycle)C₀-C₄alkyl; each of which is substituted with from 0 to 6 substituents independently chosen from R_{E}; or
(iii) taken together with one or two of R₂, R₇, R₈, R₉ or R_{B} to form a 4- to 10-membered cycloalkyl or heterocycle that is substituted with from 0 to 3 substituents independently chosen from R_{D}; and
R₇ is:
(i) hydrogen, hydroxy, halogen, cyano, amino, aminocarbonyl, aminosulfonyl or —COOH;
(ii) C₁-C₈alkyl, C₁-C₈alkenyl, C₁-C₈alkynyl, (C₃-C₈cycloalkyl)C₀-C₄alkyl, C₁-C₆alkoxy, C₁-C₈alkylthio, C₁-C₈alkylsulfinyl, C₁-C₆alkylsulfonyl, (C₃-C₈cycloalkyl)sulfonyl, mono- or di-(C₁-C₆alkyl)amino, mono- or di-(C₁-C₆alkyl)aminosulfonyl, or mono- or di-(C₁-C₆alkyl)aminocarbonyl, each of which is substituted with from 0 to 4 substituents independently chosen from R_{E}; such that R₇ is not C₁-C₆hydroxyalkyl; or
(iii) taken together with R₆ or R₈ to form an optionally substituted cycloalkyl or heterocycle;
R₈ is:
(i) halogen, cyano, amino, aminosulfonyl or —COOH;
(ii) C₁-C₈alkyl, C₁-C₈alkenyl, C₁-C₈alkynyl, (C₃-C₈cycloalkyl)C₀-C₄alkyl, C₁-C₆alkoxy, C₁-C₆alkanoyl, C₁-C₈alkylthio, C₁-C₈alkylsulfinyl, C₁-C₆alkylsulfonyl, (C₃-C₈cycloalkyl)sulfonyl, mono- or di-(C₁-C₆alkyl)amino, mono- or di-(C₁-C₆alkyl)aminosulfonyl, or mono- or di-(C₁-C₆alkyl)aminocarbonyl, each of which is substituted with from 0 to 4 substituents independently chosen from hydroxy, halogen, cyano, amino, nitro, aminocarbonyl, aminosulfonyl, —COOH, C₁-C₆alkyl, C₁-C₆haloalkyl, C₁-C₆alkoxy, C₁-C₆haloalkoxy, C₁-C₆alkylthio, C₁-C₆alkoxycarbonyl, C₁-C₆alkanoyloxy, C₃-C₆alkanone, mono- or di-(C₁-C₆alkyl)amino, C₁-C₆alkylsulfonyl, mono- or di-(C₁-C₆alkyl)aminosulfonyl, and mono- or di-(C₁-C₆alkyl)aminocarbonyl; or
(iii) taken together with R₆ or R₇ to form an optionally substituted cycloalkyl or heterocycle.

109.-131. (canceled)

132. A pharmaceutical composition, comprising at least one compound or salt according to claim 1, in combination with a physiologically acceptable carrier or excipient.

133. A pharmaceutical composition according to claim 132, wherein the composition is formulated as an indictable fluid, an aerosol, a cream, a gel, a pill, a capsule, a syrup or a transdermal patch.

134. A pharmaceutical composition, comprising:
(i) a first agent that is a compound or salt according to claim 1:
(ii) a second agent that is suitable for treating an appetite disorder, obesity, an addictive disorder, asthma, liver cirrhosis, sepsis, irritable bowel disease, Crohn's disease, depression, schizophrenia, a memory disorder, a cognitive disorder, a movement disorder, a metabolic disorder or bone loss; and
(iii) a physiologically acceptable carrier or excipient.

135. A pharmaceutical composition according to claim 134, wherein the second agent is an anti-obesity agent selected from an MCH receptor antagonist, an apo-B/MTP inhibitor, a 11β-hydroxy steroid dehydrogenase-1 inhibitor, peptide YY₃-36 or an analog thereof, a MCR-4 agonist, a CCK-A agonist, a monoamine reuptake inhibitor, a sympathomimetic agent, a 3 adrenergic receptor agonist, a dopamine agonist, a melanocyte-stimulating hormone receptor analog, a 5-HT2c receptor agonist, leptin or an analog thereof, a leptin receptor agonist, a galanin antagonist, a lipase inhibitor, a bombesin agonist, a neuropeptide-Y receptor antagonist, a thyromimetic agent, dehydroepiandrosterone or analog thereof, a glucocorticoid receptor antagonist, an orexin receptor antagonist, a glucagon-like peptide-1 receptor agonist, a ciliary neurotrophic factor, a human agouti-related protein antagonist, a ghrelin receptor antagonist, a histamine 3 receptor antagonist, or a neuromedin U receptor agonist.

136. A pharmaceutical composition according to claim 135, wherein the anti-obesity agent is phentermine, orlistat or sibutramine.

137. A pharmaceutical composition according to claim 134, wherein the second agent is a nicotine receptor partial agonist, an opioid antagonist or a dopaminergic agent.

138. A pharmaceutical composition according to claim 134, wherein the second agent is suitable for treating an addictive disorder, and wherein the agent is selected from methadone, LAAM, naltrexone, ondansetron, sertraline, fluoxetine, diazepam, chlordiazepoxide, varenicline and buproprion.

139. A packaged pharmaceutical preparation, comprising:
(a) a pharmaceutical composition according to claim 132 in a container; and
(b) instructions for using the composition to treat an appetite disorder, obesity, an addictive disorder, asthma, liver cirrhosis, sepsis, irritable bowel disease, Crohn's disease, depression, schizophrenia, a memory disorder, a cognitive disorder, a movement disorder, a metabolic disorder or bone loss.

140. A method for treating a condition responsive to CB1 modulation in a patient, in a patient, comprising administering to the patient a therapeutically effective amount of at least one compound or salt according to claim 1.

141. A method according to claim 140, wherein the condition is an appetite disorder, obesity, an addictive disorder, asthma, liver cirrhosis, sepsis, irritable bowel disease, a metabolic disorder or Crohn's disease.

142. A method according to claim 140, wherein the condition is obesity, bulimia, alcohol dependency or nicotine dependency.

143. A method according to claim 142, wherein the condition is obesity.

144. A method according to claim 140, wherein the condition is depression, schizophrenia, a memory disorder, a cognitive disorder, a movement disorder or bone loss.

145. A method for suppressing appetite in a patient, comprising administering to the patient an appetite reducing amount of at least one compound or salt according to claim 1, and thereby suppressing appetite in the patient.

146.-150. (canceled)
